(19) United States
(12) Patent Application Publication
(10) Pub. No.: US 2026/0109976 A1
(43) Pub. Date: Apr. 23, 2026

(54) SYNTHETIC ENGINEERED RNA MOLECULES AND RELATED METHODS

(71) Applicant: RNAV8 Bio, Inc., Laguna Niguel, CA (US)

(72) Inventors: Kristine Bielecki, Laguna Niguel, CA (US); Devan Shah, Laguna Niguel, CA (US)

(21) Appl. No.: 19/221,434

(22) Filed: May 28, 2025

Related U.S. Application Data
(60) Provisional application No. 63/652,501, filed on May 28, 2024.

(51) Int. Cl. C12N 15/11; A61K 9/51; A61K 31/7105; A61K 47/12; C12N 15/10

(52) U.S. Cl. CPC C12N 15/11; A61K 9/5123; A61K 31/7105; A61K 47/12; C12N 15/10

(57) ABSTRACT

Provided herein are heterologous engineered mRNA molecules; and methods of making and using said synthetic engineered mRNAs to increase expression profiles.

### CROSS REFERENCE TO RELATED APPLICATIONS

This application claims the benefit of U.S. Provisional Application No. 63/652,501, filed on May 28, 2024 and is incorporated by reference in its entirety.

### REFERENCE TO SEQUENCE LISTING

The Sequence Listing submitted with the instant application as xml file entitled 131199-0005UT01.xml, dated Aug. 28, 2025, with a file size of 561,447 bytes is hereby incorporated by reference in its entirety.

### BACKGROUND

Messenger RNA (mRNA) may be used as a gene delivery molecule, for example, in the field of therapeutics. As a source of gene products, mRNA has several benefits including that entry to a nucleus is not required and that mRNA also has an insignificant possibility of integrating into the host cell genome. For a given gene, the untranslated gene regions (UTRs), including the 5′ and 3′ UTRs, are regions involved in the regulation of expression. The 5′ UTR is a regulatory region of every mRNA situated upstream of all protein coding sequences that are translated into protein. 5′ UTRs may contain various regulatory elements, e.g., 5′ cap structure, G-quadruplex structure (G4), stem-loop structure, RNA binding protein sequence motifs, and internal ribosome entry sites (IRES), which play a major role in the control of translation initiation. The 3′ UTR, situated downstream of the protein coding sequence, has been discovered to be involved in numerous regulatory processes such as transcript cleavage, stability and polyadenylation, translation, and mRNA localization. The 3′ UTR can provide a binding site for numerous regulatory proteins and small non-coding RNAs, e.g., microRNAs. Despite significant clinical progress in cell and gene therapies, maximizing protein expression in order to enhance potency remains a major challenge.

### SUMMARY

Provided herein are synthetic engineered RNAs (e.g., mRNAs) to increase protein expression by optimizing translation through the engineering of 5′ untranslated regions (5′ UTRs) and/or 3′ untranslated regions (3′ UTRs) to provide novel 5′ UTRs, 3′ UTRs and 5′/3′ UTR pairs (UPs) that enhance protein expression. In certain embodiments, the relevant components of an mRNA molecule include at least a coding region (CDS or ORF) encoding a heterologous polypeptide, a 5′UTR, a 3′UTR, a 5′ cap and a poly-A tail. Improving upon this wild type modular structure, the present invention expands the scope of functionality of traditional mRNA molecules by providing synthetic engineered RNA constructs which maintain a modular organization, but which comprise one or more non-naturally occurring structural and/or chemical modifications or alterations which impart useful properties to the invention engineered mRNA constructs, such as increased polypeptide expression.

Accordingly, provided herein are synthetic engineered mRNA constructs, comprising an open reading frame (ORF) operably linked to a heterologous 5′ untranslated region (UTR) and a heterologous 3′ UTR,
wherein the 5′ UTR is selected from the group consisting of: SEQ ID NOs: 1-123; and/or
wherein the 3′ UTR is selected from the group consisting of: SEQ ID NOs: 124-438.
   In certain embodiments, the 5′ UTR is selected from the group consisting of: SEQ ID NOs: 22, 32-34, 35-68, 70-71, 74-75, 90, 92, 97, 103, 111, 115, and 121-122. In other embodiments, the 3′ UTR is selected from the group consisting of: SEQ ID NOs: 145, 150-185, 189-197, 199-201, 203-204, 206-209, 211-323, 335-345, 347, 349-350, 352-422, and 428-438. In a particular embodiment, the 5′ UTR and 3′ UTR are set forth as numbered UTR pairs (UP) in rows of Table 4, and are selected from the group consisting of: UP001-UP043.

More particularly, provided herein are the following aspects of the invention:
Aspect 1. A synthetic engineered mRNA, comprising an open reading frame (ORF) operably linked to a heterologous 5′ untranslated region (UTR) and a heterologous 3′ UTR, wherein the 5′ UTR and 3′ UTR are set forth as UTR pairs in rows of the following table, and are selected from the group consisting of:

| Registry ID | 5′UTR | 3′UTR |
|---|---|---|
| UP001 | 5UTR022/SEQ ID NO: 20 | 3UTR005/SEQ ID NO: 128 |
| UP002 | 5UTR022/SEQ ID NO: 20 | 3UTR011/SEQ ID NO: 134 |
| UP003 | 5UTR024/SEQ ID NO: 22 | 3UTR022/SEQ ID NO: 145 |
| UP004 | 5UTR024/SEQ ID NO: 22 | 3UTR112/SEQ ID NO: 189 |
| UP005 | 5UTR024/SEQ ID NO: 22 | 3UTR113/SEQ ID NO: 190 |
| UP006 | 5UTR024/SEQ ID NO: 22 | 3UTR122/SEQ ID NO: 199 |
| UP007 | 5UTR024/SEQ ID NO: 22 | 3UTR126/SEQ ID NO: 203 |
| UP008 | 5UTR024/SEQ ID NO: 22 | 3UTR137/SEQ ID NO: 214 |
| UP009 | 5UTR024/SEQ ID NO: 22 | 3UTR141/SEQ ID NO: 218 |
| UP010 | 5UTR024/SEQ ID NO: 22 | 3UTR143/SEQ ID NO: 220 |
| UP011 | 5UTR024/SEQ ID NO: 22 | 3UTR185/SEQ ID NO: 262 |
| UP012 | 5UTR024/SEQ ID NO: 22 | 3UTR187/SEQ ID NO: 264 |
| UP013 | 5UTR024/SEQ ID NO: 22 | 3UTR188/SEQ ID NO: 265 |
| UP014 | 5UTR024/SEQ ID NO: 22 | 3UTR190/SEQ ID NO: 267 |
| UP015 | 5UTR024/SEQ ID NO: 22 | 3UTR192/SEQ ID NO: 269 |
| UP016 | 5UTR024/SEQ ID NO: 22 | 3UTR195/SEQ ID NO: 272 |
| UP017 | 5UTR024/SEQ ID NO: 22 | 3UTR200/SEQ ID NO: 277 |
| UP018 | 5UTR024/SEQ ID NO: 22 | 3UTR201/SEQ ID NO: 278 |
| UP019 | 5UTR024/SEQ ID NO: 22 | 3UTR262/SEQ ID NO: 339 |
| UP020 | 5UTR030/SEQ ID NO: 27 | 3UTR112/SEQ ID NO: 189 |
| UP021 | 5UTR030/SEQ ID NO: 27 | 3UTR113/SEQ ID NO: 190 |
| UP022 | 5UTR073/SEQ ID NO: 69 | 3UTR076/SEQ ID NO: 186 |
| UP023 | 5UTR073/SEQ ID NO: 69 | 3UTR077/SEQ ID NO: 187 |
| UP024 | 5UTR077/SEQ ID NO: 72 | 3UTR005/SEQ ID NO: 128 |
| UP025 | 5UTR093/SEQ ID NO: 88 | 3UTR022/SEQ ID NO: 145 |
| UP026 | 5UTR103/SEQ ID NO: 98 | 3UTR113/SEQ ID NO: 190 |
| UP027 | 5UTR111/SEQ ID NO: 106 | 3UTR113/SEQ ID NO: 190 |
| UP028 | 5UTR103/SEQ ID NO: 98 | 3UTR022/SEQ ID NO: 145 |
| UP029 | 5UTR104/SEQ ID NO: 99 | 3UTR022/SEQ ID NO: 145 |
| UP030 | 5UTR111/SEQ ID NO: 106 | 3UTR022/SEQ ID NO: 145 |
| UP031 | 5UTR117/SEQ ID NO: 112 | 3UTR022/SEQ ID NO: 145 |
| UP032 | 5UTR093/SEQ ID NO: 88 | 3UTR356/SEQ ID NO: 429 |
| UP033 | 5UTR103/SEQ ID NO: 98 | 3UTR112/SEQ ID NO: 189 |
| UP034 | 5UTR111/SEQ ID NO: 106 | 3UTR112/SEQ ID NO: 189 |
| UP035 | 5UTR123/SEQ ID NO: 117 | 3UTR112/SEQ ID NO: 189 |
| UP036 | 5UTR093/SEQ ID NO: 88 | 3UTR113/SEQ ID NO: 190 |
| UP037 | 5UTR104/SEQ ID NO: 99 | 3UTR113/SEQ ID NO: 190 |
| UP038 | 5UTR105/SEQ ID NO: 100 | 3UTR113/SEQ ID NO: 190 |
| UP039 | 5UTR024/SEQ ID NO: 22 | 3UTR261/SEQ ID NO: 338 |
| UP040 | 5UTR093/SEQ ID NO: 88 | 3UTR188/SEQ ID NO: 265 |
| UP041 | 5UTR103/SEQ ID NO: 98 | 3UTR113/SEQ ID NO: 190 |
| UP042 | 5UTR093/SEQ ID NO: 88 | 3UTR357/SEQ ID NO: 430 |
| UP043 | 5UTR129/SEQ ID NO: 123 | 3UTR357/SEQ ID NO: 430. |

Aspect 2. A synthetic engineered mRNA, comprising an open reading frame (ORF) operably linked to a heterologous 5′ untranslated region (UTR) and a heterologous 3′ UTR, wherein the 5′ UTR is selected from the group consisting of: SEQ ID NOs: 22, 32-34, 35-68, 70-71, 74-75, 90, 92, 97, 103, 111, 115, and 121-122.
Aspect 3. The synthetic engineered mRNA of Aspect 2, wherein the 3′ UTR is selected from the group consisting of: SEQ ID NOs: 124-438.
Aspect 4. The synthetic engineered mRNA of Aspect 3, wherein the 3′ UTR is selected from the group consisting of: SEQ ID NOs: 145, 150-185, 189-197, 199-201, 203-204, 206-209, 211-323, 335-345, 347, 349-350, 352-422, and 428-438.
Aspect 5. A synthetic engineered mRNA, comprising an open reading frame (ORF) operably linked to a heterologous 5′ untranslated region (UTR) and a heterologous 3′ UTR, wherein the 3′ UTR is selected from the group consisting of: SEQ ID NOs: 145, 150-185, 189-197, 199-201, 203-204, 206-209, 211-323, 335-345, 347, 349-350, 352-422, and 428-438.
Aspect 6. The synthetic engineered mRNA of Aspect 5, wherein the 5′ UTR is selected from the group consisting of: SEQ ID NOs: 1-123.
Aspect 7. The synthetic engineered mRNA of Aspect 6, wherein the 5′ UTR is selected from the group consisting of: SEQ ID NOs: 22, 32-34, 35-68, 70-71, 74-75, 90, 92, 97, 103, 111, 115, and 121-122.
Aspect 8. A synthetic engineered 5′ UTR selected from the group consisting of: SEQ ID NOs: 22, 32-34, 35-68, 70-71, 74-75, 90, 92, 97, 103, 111, 115, and 121-122.
Aspect 9. A synthetic engineered 3′ UTR selected from the group consisting of: SEQ ID NOs: 145, 150-185, 189-197, 199-201, 203-204, 206-209, 211-323, 335-345, 347, 349-350, 352-422, and 428-438.
Aspect 10. A synthetic engineered mRNA, comprising an open reading frame (ORF) operably linked to a heterologous 5′ untranslated region (UTR) and a heterologous 3′ UTR, wherein the 5′ UTR is selected from the group consisting of: SEQ ID NOs: 1-123; and/or wherein the 3′ UTR is selected from the group consisting of: SEQ ID NOs: 124-438.
Aspect 11. The synthetic engineered mRNA of Aspect 10, wherein the 5′ UTR is selected from the group consisting of: SEQ ID NOs: 22, 32-34, 35-68, 70-71, 74-75, 90, 92, 97, 103, 111, 115, and 121-122.
Aspect 12. The synthetic engineered mRNA of Aspect 10, wherein the 3′ UTR is selected from the group consisting of: SEQ ID NOs: 145, 150-185, 189-197, 199-201, 203-204, 206-209, 211-323, 335-345, 347, 349-350, 352-422, and 428-438.
Aspect 13. The synthetic engineered mRNA of Aspect 10, wherein the 5′ UTR and 3′ UTR are set forth as numbered UTR pairs (UP) in rows of Table 4, and are selected from the group consisting of: UP001-UP043.
Aspect 14. A synthetic engineered mRNA, comprising an open reading frame (ORF) operably linked to a heterologous 5′ untranslated region (UTR) and a heterologous 3′ UTR, wherein the 5′ UTR is selected from the group consisting of: SEQ ID NOs: 1-123.
Aspect 15. A synthetic engineered mRNA, comprising an open reading frame (ORF) operably linked to a heterologous 5′ untranslated region (UTR) and a heterologous 3′ UTR, wherein the 3′ UTR is selected from the group consisting of: SEQ ID NOs: 124-438.
Aspect 16. The synthetic engineered mRNA of Aspects 1-15, wherein the mRNA further comprises a 5′ cap structure.
Aspect 17. The synthetic engineered mRNA of Aspect 16, wherein the 5′ cap structure is selected from Cap 1, Cap 2, or m6A Cap 1.
Aspect 18. The synthetic engineered mRNA of Aspects 1-17, wherein the mRNA further comprises a 3′ poly A tail region.
Aspect 19. The synthetic engineered mRNA of Aspects 18, wherein the 3′ poly A tail is a length selected from at least 30, 40, 50, 60, 70, 80, 90, 100, 120, 140, 160, 180, or 200 nucleosides.
Aspect 20. A composition comprising the synthetic engineered mRNA of Aspects 1-19, formulated in a lipid nanoparticle (LNP) carrier.
Aspect 21. A lipid nanoparticle (LNP) comprising a synthetic engineered mRNA, wherein the mRNA comprises
   (a) a 5′ untranslated region (5′UTR);
   (b) a CDS region encoding a heterologous polypeptide;
   (c) a 3′ untranslated region (3′UTR); and
   (d) a 3′ poly A tail region,
      wherein the 5′ UTR is selected from the group consisting of: SEQ ID NOs: 1-123, or
      wherein the 3′ UTR is selected from the group consisting of: SEQ ID NOs: 124-438.
Aspect 22. The lipid nanoparticle of Aspect 21, comprising a cationic or ionizable lipid.
Aspect 23. The lipid nanoparticle of Aspects 21-22, wherein the cationic lipid is ALC-0315, DLin-MC3-DMA, DLin-DMA, C12-200, or DLin-KC2-DMA.
Aspect 24. The lipid nanoparticle of Aspects 21-23, comprising a PEG lipid.
Aspect 25. The lipid nanoparticle of Aspects 21-24, wherein the heterologous polypeptide is selected from a vaccine, a therapeutic protein, gene-editing protein, a regulatory protein, a chimeric antigen receptor, or a reporter gene.
Aspect 26. The lipid nanoparticle of Aspects 21-25, wherein the CDS region encoding the heterologous polypeptide is codon optimized.
Aspect 27. The lipid nanoparticle of Aspects 21-26, wherein the mRNA further comprises a 5′ cap structure.
Aspect 28. The lipid nanoparticle of Aspect 27, wherein the 5′ cap structure is selected from Cap 1, Cap 2, or m6A Cap 1.
Aspect 29. The lipid nanoparticle of Aspects 21-28, wherein the 3′ poly A tail is a length selected from at least 30, 40, 50, 60, 70, 80, 90, 100, 120, 140, 160, 180, or 200 nucleosides.
Aspect 30. A method of expressing an engineered synthetic mRNA in a cell, said method comprising introducing the engineered mRNA of Aspects 1-19 or the LPN of Aspects 20-29 into said cell.
Aspect 31. A method of making a synthetic engineered mRNA, said method comprising constructing a: (a) a 5′ untranslated region (5′UTR); (b) a CDS region encoding a heterologous polypeptide; (c) a 3′ untranslated region (3′UTR); and (d) a 3′ poly A tail region,
wherein the 5′ UTR is selected from the group consisting of: SEQ ID NOs: 1-123, or
wherein the 3′ UTR is selected from the group consisting of: SEQ ID NOs: 124-438; and
wherein said constructing is by one or more of IVT, chemical synthesis, and/or host cell expression.

### BRIEF DESCRIPTION OF THE DRAWINGS

FIG. 1 shows UTR expression improvements in HEK293 cells greater than comparative literature screens and internal comparisons. Plasmid DNA was used as a template for generating mRNA through in vitro transcription (IVT). Following IVT, a 5′ Cap reaction and 3′ Tail reaction was carried out as described in Examples 3-5. Lipofectamine MessengerMax transfection was carried out and the Mean Fluorescence Intensity (MFI) Fluorescent readout (GFP) was obtained as described in Example 7. Timepoints were taken over a one week timeframe and the area under the curve was plotted (AUC) and normalized to that of P013. The results indicate that expressions levels for 5UTR022, 3UTR005 and 3UTR011 exceeded that of the control. UTR Expression improvements greater thatn comparative literature screens and internal commercial comparatives. EXP2300095.
Best % improvement improvement from switching to artificial 5′ UTR-Luc at 123% using mRNA transfection. Sultana et al, Mol. Ther. Methods. Clin. Dev 2020
Best % improvement from 5′ UTR-GFP screen ˜150% of internal standard UTR (human beta-globin) using mRNA transfection. Linareas-Fernandez et al, Nuc. Acids. 2021
Best % improvement from 5′ UTR-GFP screen ˜150% of internal standard UTR (pVAX1 human CMV) plasmid transfection. Choi et al, Nat. Comm. 2021
Best % improvement from 3′ UTR-Luc screen ˜350% of internal standard UTR mRNA transfection. Von Niessen et al, Mol. Ther. 2019 (BioNTec)

FIG. 2 shows the results of HEK293 cells 24 Hours Post Lipofectamine Messenger Max Transfection. The mRNAs were prepared using IVT, including a 5′ Cap reaction, but no 3′ Tail reaction (except for the mRNA generated from p183) as described in Examples 3-5. Plasmid p183 was enzymatically tailed and it has been found that enzymatic tailing and 80As encoded in the plasmid are equivalent (see FIG. 4 p183 vs. p270). Lipofectamine MessengerMax transfection was carried out and the Mean Fluorescence Intensity (MFI) Fluorescent readout (GFP) was obtained after 24 hours as described in Example 7. HEK293 24 Hours Post Transfection. Designed UTR variants of 3UTR022 to ablate certain RBP binding sequence motifs and include others None surpassed the internal UP003 (p183). EXP24000025 All samples on a single 24 well plate; Plates (ie 3 biological replicates); Each well read 3 times (technical replicates).

FIG. 3 shows the results of HepG2 cells 24 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 24 hours as described in Example 7. HepG2 24 Hours Post Transfection. Designed UTR variants of 3UTR022 to ablate certain RBP binding sequence motifs and include others None surpassed the internal UP003 (p183). EXP24000025 All samples on a single 24 well plate; 3 Plates (ie 3 biological replicates); Each well read 3 times (technical replicates).

FIG. 4 shows the results of HepG2 cells 24 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 24 hours as described in Example 7. The results indicate that expressions levels for UP014, UP015, UP016, UP017, UP018, UP011 and UP013 exceeded that of the control. HepG2 24 Hours Post Transfection. Two Fold Improvement Over Previous Internal Benchmark (UP003) EXP24000030.

FIG. 5 shows the results of HepG2 cells 21 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 21 hours as described in Example 7. HepG2 21 Hours Post Transfection. Designed UTR variants of 3UTR022 to ablate certain RBP binding sequence motifs and include RBP1; None surpassed the UP003 (p270) benchmark.

FIG. 6 shows the results of HepG2 cells 21 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 21 hours as described in Example 7. HepG2 21 Hours Post Transfection. Designed UTR variants of 3UTR022 to ablate certain RBP binding sequence motifs and include RBP2; None surpassed the internal UP003 (p270); EXP24000036.

FIG. 7 shows the results of HepG2 cells 21 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 21 hours as described in Example 7. HepG2 21 Hours Post Transfection. Designed UTR variants of 3UTR022 to ablate certain RBP binding sequence motifs and include RBP3; None surpassed the UP003 (p270) benchmark; EXP24000036.

FIG. 8 shows the results of HepG2 cells 21 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 21 hours as described in Example 7. The results indicate that expressions levels for UP011 (p295) and UP013 (p298) exceeded that of the control UP003 (p270). HepG2 21 Hours Post Transfection. Designed UTR variants of 3UTR022 to ablate certain RBP binding sequence motifs and include RBP4 UP011 (p295) and UP013 (p298) surpassed the UP003 (p270) benchmark EXP24000036.

FIG. 9 shows the results of HepG2 cells 21 Hours Post Lipofectamine Messenger Max Transfection. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Mean Fluorescence Intensity (MFI) Fluorescent readout (GFP) was obtained after 21 hours as described in Example 7. The results indicate that expressions levels for UP015 (p302) exceeded that of the control UP003 (p270). HepG2 21 Hours Post Transfection. Designed UTR variants of 3UTR022 to ablate certain RBP binding sequence motifs and include RBP5 UP015 (p302) surpassed the UP003 (p270) benchmark EXP24000036.

FIG. 10 shows UTR Effects on Primary T cell Expression Over 12 Days. The mRNAs were prepared using IVT, including a 5′ Cap reaction, but no 3′ Tail reaction (as plasmids encoded the poly A tail) as described in Examples 3-5. Transfection via electroporation was carried out and the Hibit readout was obtained over the course of 12 days as described in Example 7. Maintained 66% of Expression Magnitude 5 Days Post Electroporation; Therapeutically relevant CDS assayed using HiBit tag; EXP24000067.

FIG. 11 shows Therapeutically Relevant Wild-Type CDS Time course in HepG2 including Wild-Type UTR Controls as well as a Codon Optimization Control. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained over the course of 2-48 hours as described in Example 7. The results indicate that expressions levels for UP003, UP004, UP005, UP006, UP020, and UP025 exceeded that of the controls. The commercially available codon optimization did not yield expression improvements superior to the UTR engineering approaches described herein. Therapeutically Relevant CDS Timecourse in HepG2 with WT UTR Controls and Codon Optimization Control; EXP000097.

FIG. 12 shows 3.7× Improvement over existing UTRs for therapeutically relevant CDS046. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained over the course of 12-48 hours as described in Example 7. The results indicate that expressions levels for UP015, UP028, UP029, UP030, and UP031 exceeded that of the control. 3.7× improvement over esisting UTRs for therapeutically relevant CDS046; HiBit assay in THP1 cells; EXP24000107.

FIG. 13 shows Fold improvements over existing UTRs for therapeutically relevant CDS046. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained over the course of 2-48 hours as described in Example 7. The results indicate that expressions levels for UP028, UP029, UP030, and UP031 exceeded that of the control. Fold improvements over existing UTRs for therapeutically relevant CDS046; HIBIT assay in HepG2 cells; EXP24000107.

FIG. 14 shows the results of HiBit Assay of CDS054 in HepG2 cells. The mRNAs were prepared using IVT, including a 5′ Cap reaction and 3′ Tail reaction as described in Examples 3-5. Lipofectamine MessengerMax transfection was carried out and the Hibit readout was obtained over the course of 5-50 hours as described in Example 7. The results indicate that expressions levels for UP003, UP05, UP025, UP026, UP027, UP036, UP037 and UP038 exceeded that of the control. HiBit Assay of CDS053 in HepG2; All 3UTR113 paried with various 5′UTRs; Shows with a good 3′UTR the 5′UTR has an impact; EXP24000112.

FIG. 15 shows the results of a Lipofectamine Messenger Max Transfection in HepG2 cells HiBit Readout at 12 hours. The mRNAs were prepared using IVT, including a 5′ Cap reaction and 3′ Tail reaction as described in Examples 3-5. Lipofectamine MessengerMax transfection was carried out and the Hibit readout was obtained at 12 hours as described in Example 7. The results indicate that expressions levels for UP004, UP006, UP020, and UP025 exceeded that of the control. Transfection in HepG2; HiBit Readout at 12 Hrs; EXP24000128. Therapeutically relevant ORF transfected with Messenger Max (MM) at either 1.0 ug or 1.5 ug per well seeded the night before at 20,000 HepG2 Cells per well.

FIG. 16 shows the results of a Lipofectamine Messenger Max Transfection in HepG2 cells HiBit Readout at 24 hours. The mRNAs were prepared using IVT, including a 5′ Cap reaction and 3′ Tail reaction as described in Examples 3-5. Lipofectamine MessengerMax transfection was carried out and the Hibit readout was obtained at 24 hours as described in Example 7. The results indicate that expressions levels for UP004, UP006, UP020, and UP025 exceeded that of the control. Transfection in HepG2; HiBit Readout at 24 Hrs; EXP24000128. Therapeutically relevant ORF transfected with Messenger Max (MM) at either 1.0 ug or 1.5 ug per well seeded the night before at 20,000 HepG2 Cells per well.

FIG. 17 shows that In Vitro HepG2 data translates to In Vivo expression profiles. This figure corresponds to the in vitro data from previous FIGS. 14 and 15 alongside the in vivo data, which indicates that the data trend remains the same for both in vitro and in vivo. The mRNAs were prepared using IVT, including a 5′ Cap reaction and 3′ Tail reaction as described in Examples 3-5. In vivo formulation of lipid nanoparticle (LNP)-encapsulated human mRNA was conducted as described in Example 10; and the Hibit readout was obtained at 12 and 24 hours as described in Example 7. The results indicate that expressions levels for UP004, UP006, UP020, and UP025 exceeded that of the control by 82-475 fold depending on dose, timepoint, and assay readout. In vitro HepG2 data translates to in vivo expression profiles Female WT FVB, Tail vein injection, 1 mg/kg, Concentration 0.1 mg/ml.

FIG. 18 shows the results of a Lipofectamine Messenger Max Transfection in THP-1 cells HiBit Readout at 12 hours. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained at 12 hours as described in Example 7. The results indicate that expressions levels for UP039, UP040, and UP041 exceeded that of the control. Transfection in THP-1; HiBit Readout at 12 Hrs; P621 was internal Benchmark Control; UTRs with additional optimized Kozak; EXP25000036.

FIG. 19 shows the results of a Lipofectamine Messenger Max Transfection in HEK293 cells HiBit Readout at 12 hours. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained at 12 hours as described in Example 7. The results indicate that expressions levels for UP039, UP040, and UP041 exceeded that of the control. Transfection in HEK293; HiBit Readout at 12 Hrs; P621 was internal Benchmark Control; UTRs with additional optimized Kozak; EXP25000036.

FIG. 20 shows the results of a Lipofectamine Messenger Max Transfection in HepG2 cells HiBit Readout at 12 hours. The mRNAs were prepared using IVT, including a 5′ Cap reaction, but no 3′ Tail reaction (as plasmids encoded the polyA tail) as described in Examples 3-5. Lipofectamine MessengerMax transfection was carried out and the Hibit readout was obtained at 12 hours as described in Example 7. The results indicate that expressions levels for UP039, UP040, and UP041 exceeded that of the control. Transfection in HepG2; HiBit Readout at 12 Hrs; P621 was internal Benchmark Control; UTRs with additional optimized Kozak; EXP25000036.

### DETAILED DESCRIPTION

Provided herein, are synthetic engineered mRNAs, comprising an open reading frame (ORF) operably linked to a heterologous 5′ untranslated region (UTR) and a heterologous 3′ UTR, wherein the 5′ UTR is selected from the group consisting of: SEQ ID NOs: 1-123. SEQ ID NOs: 1-123 correspond to the 5′ UTR Registry ID numbers set forth hereinbelow in Table 1. In particular embodiments, when the 5′UTR corresponds to SEQ ID NOs: 1-123, the 3′UTR can be any 3′UTR known to those of skill in the art, including the 3′ UTR sequences set forth in Table 2. In particular embodiments of the engineered RNAs, the ORF (also referred to herein as a CDS) can be any coding sequence (CDS) encoding a heterologous polypeptide of interest, such as a vaccine, a therapeutic protein, gene-editing protein, a regulatory protein, a chimeric antigen receptor, a reporter gene, and the like.

Also provided herein, are synthetic engineered mRNAs, comprising an open reading frame (ORF) operably linked to a heterologous 5′ untranslated region (UTR) and a heterologous 3′ UTR, wherein the 3′ UTR is selected from the group consisting of: SEQ ID NOs: 124-438. Likewise, SEQ ID NOs: 124-438 correspond to the 3′ UTR Registry ID numbers set forth hereinbelow in Table 2. In particular embodiments, when the 3′UTR corresponds to SEQ ID NOs: 124-438, the 5′UTR can be any 5′UTR known to those of skill in the art, including the 5′ UTR sequences set forth in Table 1. In particular embodiments of the engineered RNAs, the ORF (also referred to herein as a CDS) can be any coding sequence (CDS) encoding a heterologous polypeptide of interest.

Also provided herein are synthetic engineered mRNA constructs, comprising an open reading frame (ORF) operably linked to a heterologous 5′ untranslated region (UTR) and a heterologous 3′ UTR,
wherein the 5′ UTR is selected from the group consisting of: SEQ ID NOs: 1-123; and/or
wherein the 3′ UTR is selected from the group consisting of: SEQ ID NOs: 124-438.
   In certain embodiments, the 5′ UTR is selected from the group consisting of: SEQ ID NOs: 22, 32-34, 35-68, 70-71, 74-75, 90, 92, 97, 103, 111, 115, and 121-122. These correspond to 5′ UTR Registry #s: 24, 35-37, 29-72, 74-75, 79-90, 95, 97, 102, 108, 116, 120, and 127-128; and are non-naturally occurring engineered synthetic 5′ UTRs. In particular embodiments, the 3′ UTR is selected from the group consisting of: SEQ ID NOs: 145, 150-185, 189-197, 199-201, 203-204, 206-209, 211-323, 335-345, 347, 349-350, 352-422, and 428-438. These correspond to 3′ UTR Registry #s: 22, 31-53, 58-59, 64-74, 112-120, 122-124, 126-127, 129-132, 134-245, 258-268, 270, 272-273, 275-348, and 355-365; and are non-naturally occurring engineered synthetic 3′ UTRs.

In a particular embodiment, the 5′ UTR and 3′ UTR are set forth as numbered UTR pairs (UP) from the rows of Table 4, and are selected from the group consisting of: UP001-UP043. For example, from Table 4, UP001 corresponds to the pair combination of 5′UTR022 (SEQ ID NO: 20) with 3UTR005 (SEQ ID NO:128) within the same invention synthetic engineered mRNA construct. Likewise, UP002 corresponds to the pair combination of 5′UTR022 (SEQ ID NO:20) with 3UTR011 (SEQ ID NO:134) within the same invention synthetic engineered mRNA construct; UP003 corresponds to the pair combination of 5′UTR024 (SEQ ID NO:22) with 3UTR022 (SEQ ID NO:145) within the same invention synthetic engineered mRNA construct . . . and UP043 corresponds to the pair combination of 5′UTR129 (SEQ ID NO: 123) with 3UTR357 (SEQ ID NO:430) within the same invention synthetic engineered mRNA construct.

Also provided herein are synthetic engineered mRNA constructs, comprising an open reading frame (ORF) operably linked to a heterologous 5′ untranslated region (UTR) and a heterologous 3′ UTR, wherein the 5′ UTR is selected from the group consisting of: SEQ ID NOs: 22, 32-34, 35-68, 70-71, 74-75, 90, 92, 97, 103, 111, 115, and 121-122. In particular embodiments, when the 5′UTR corresponds to SEQ ID NOs: SEQ ID NOs: 22, 32-34, 35-68, 70-71, 74-75, 90, 92, 97, 103, 111, 115, and 121-122, the 3′UTR can be any 3′UTR known to those of skill in the art, including the 3′ UTR sequences set forth in Table 2. In particular embodiments, the 3′ UTR is selected from the group consisting of: SEQ ID NOs: 124-438. In other embodiments, the 3′ UTR is selected from the group consisting of: SEQ ID NOs: 145, 150-185, 189-197, 199-201, 203-204, 206-209, 211-323, 335-345, 347, 349-350, 352-422, and 428-438.

Also provided herein are synthetic engineered mRNA constructs, comprising an open reading frame (ORF) operably linked to a heterologous 5′ untranslated region (UTR) and a heterologous 3′ UTR, wherein the 3′ UTR is selected from the group consisting of: SEQ ID NOs: 145, 150-185, 189-197, 199-201, 203-204, 206-209, 211-323, 335-345, 347, 349-350, 352-422, and 428-438. In particular embodiments, when the 3′UTR corresponds to SEQ ID NOs: 145, 150-185, 189-197, 199-201, 203-204, 206-209, 211-323, 335-345, 347, 349-350, 352-422, and 428-438, the 5′UTR can be any 5′UTR known to those of skill in the art, including the 5′ UTR sequences set forth in Table 1. In some embodiments, the 5′ UTR is selected from the group consisting of: SEQ ID NOs: 1-123. In other embodiments, the 5′ UTR is selected from the group consisting of: SEQ ID NOs: 22, 32-34, 35-68, 70-71, 74-75, 90, 92, 97, 103, 111, 115, and 121-122.

Accordingly, in certain embodiments of the invention synthetic engineered mRNA constructs, the 5′ UTR is selected from the group consisting of: SEQ ID NOs: 22, 32-34, 35-68, 70-71, 74-75, 90, 92, 97, 103, 111, 115, and 121-122, and the 3′ UTR is selected from the group consisting of: SEQ ID NOs: 145, 150-185, 189-197, 199-201, 203-204, 206-209, 211-323, 335-345, 347, 349-350, 352-422, and 428-438. In these embodiments, both the 5′ UTRs and the 3′ UTRs are non-naturally occurring synthetically engineered UTRs.

Also provided herein are synthetic engineered 5′ UTRs selected from the group consisting of: SEQ ID NOs: 22, 32-34, 35-68, 70-71, 74-75, 90, 92, 97, 103, 111, 115, and 121-122. In certain embodiments, the invention 5′ UTRs can be used by those of skill in the art in any engineered mRNA construct comprising a 5′ Cap, a 5′ UTR, an ORF or CDS, a 3′ UTR, and a poly A tail region.

Also provided herein are synthetic engineered 3′ UTR selected from the group consisting of: SEQ ID NOs: 145, 150-185, 189-197, 199-201, 203-204, 206-209, 211-323, 335-345, 347, 349-350, 352-422, and 428-438. In certain embodiments, the invention 3′ UTRs can be used by those of skill in the art in any engineered mRNA construct comprising a 5′ Cap, a 5′ UTR, an ORF or CDS, a 3′ UTR, and a poly A tail region.

Accordingly, in particular embodiments, the invention engineered mRNAs provided herein further comprises a 5′ cap structure. In particular embodiments, the Cap structure is selected from Cap 1, Cap 2, or m6A Cap 1. In a particular embodiment, the 5′ cap structure is Cap 1. In other embodiments, the invention engineered mRNA further comprises a 3′ poly A tail region. In a particular embodiment, the 3′ poly A tail is a length selected from at least 30, 40, 50, 60, 70, 80, 90, 100, 120, 140, 160, 180, or 200 nucleosides. In another embodiment, the 3′ poly A tail is at least 30 nucleosides. In another embodiment, the 3′ poly A tail is at least 40 nucleosides. In another embodiment, the 3′ poly A tail is at least 60 nucleosides. In another embodiment, the 3′ poly A tail is at least 80 nucleosides. In another embodiment, the 3′ poly A tail is at least 100 nucleosides. In another embodiment, the 3′ poly A tail is at least 150 nucleosides. In particular embodiments, the invention engineered mRNAs provided herein further comprises a 5′ cap structure and a 3′ poly A tail region.

As used herein the term “operably linked” or “flanked by” refers to the sequential and function arrangement between a 5′ UTR, open reading frame (ORF), and 3′ UTR according to the present disclosure, wherein at least the 5′ UTR modulates translation of said ORF.

As used herein, the term “heterologous” in reference to an untranslated region such as a 5′UTR or 3′UTR means a region of nucleic acid, particularly untranslated nucleic acid which is not naturally found with the coding region encoded on the same or instant polynucleotide, primary construct or mRNA. Homologous UTRs for example would represent those UTRs which are naturally found associated with the coding region of the mRNA, such as the wild type UTR.

As used herein, the term “homology” refers to the overall relatedness between polymeric molecules, e.g. between nucleic acid molecules, such as the engineered mRNA constructs provided herein. In some embodiments, polymeric molecules are considered to be “homologous” to one another if their sequences are at least at least 70%, at least 75%, at least 80%, at least 85%, at least 90%, at least 95%, at least 96%, at least 97%, at least 98%, or at least 99% identical. In some embodiments, polymeric molecules are considered to be “homologous” to one another if their sequences are at least at least 70%, at least 75%, at least 80%, at least 85%, at least 90%, at least 95%, at least 96%, at least 97%, at least 98%, or at least 99% similar. The term “homologous” necessarily refers to a comparison between at least two sequences (polynucleotide).

### Untranslated Regions (UTRs)

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure.

As used herein, the phrase “Untranslated regions” or “UTRs” refers to nucleic acid sections of a polynucleotide before a start codon (5′ UTR) and after a stop codon (3′ UTR) that are not translated. In particular embodiments, a polynucleotide (e.g., a ribonucleic acid (RNA), e.g., an engineered messenger RNA (mRNA)) of the invention comprising an open reading frame (ORF) encoding a heterologous protein, such as a vaccine, a therapeutic protein, gene-editing protein, a regulatory protein, a chimeric antigen receptor, a reporter gene, and the like; and further comprises an invention UTR (e.g., a 5′ UTR or functional fragment thereof, a 3′ UTR or functional fragment thereof, or a combination thereof). In another embodiment, the invention synthetic engineered mRNA further comprises a 5′ cap structure and a 3′ poly A tail region.

Cis-acting RNA elements can also affect translation elongation, being involved in numerous frameshifting events. Internal ribosome entry sequences (IRES) represent another type of cis-acting RNA element that are typically located in 5′ UTRs, but have also been found within the coding region of naturally-occurring mRNAs. In cellular mRNAs, IRES often coexist with the 5′-cap structure and provide mRNAs with the functional capacity to be translated under conditions in which cap-dependent translation is compromised. Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation. Additional exemplary translational regulatory activities provided by components, structures, elements, motifs, and/or specific sequences comprising polynucleotides (e.g., mRNA) include, but are not limited to, mRNA stabilization or destabilization, translational activation, and translational repression. Studies have shown that naturally occurring, cis-acting RNA elements can confer their respective functions when used to modify, by incorporation into, heterologous.

### Modified Polynucleotides Comprising Functional RNA Elements

Provided herein are synthetic engineered mRNA polynucleotides comprising a modification (e.g., an RNA element), wherein the modification provides a desired translational regulatory activity. In particular embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In particular embodiments, the desired translational regulatory activity is a cis-acting regulatory activity. In particular embodiments, the desired translational regulatory activity is an increase in the residence time of the 43S pre-initiation complex (PIC) or ribosome at, or proximal to, the initiation codon. In particular embodiments, the desired translational regulatory activity is an increase in the initiation of polypeptide synthesis at or from the initiation codon. In particular embodiments, the desired translational regulatory activity is an increase in the amount of polypeptide translated from the full open reading frame. In particular embodiments, the desired translational regulatory activity is an increase in the fidelity of initiation codon decoding by the PIC or ribosome. In particular embodiments, the desired translational regulatory activity is inhibition or reduction of leaky scanning by the PIC or ribosome. In particular embodiments, the desired translational regulatory activity is a decrease in the rate of decoding the initiation codon by the PIC or ribosome. In particular embodiments, the desired translational regulatory activity is inhibition or reduction in the initiation of polypeptide synthesis at any codon within the mRNA other than the initiation codon. In particular embodiments, the desired translational regulatory activity is inhibition or reduction of the amount of polypeptide translated from any open reading frame within the mRNA other than the full open reading frame. In particular embodiments, the desired translational regulatory activity is inhibition or reduction in the production of aberrant translation products. In particular embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity. In some aspects, the disclosure provides an mRNA that comprises an RNA element that comprises a sequence and/or an RNA secondary structure(s) that promotes the translational fidelity of the mRNA.

In particular embodiments, the RNA element comprises natural and/or modified nucleotides. In particular embodiments, the RNA element comprises a sequence of linked nucleotides, or derivatives or analogs thereof, that provides a desired translational regulatory activity as described herein. In particular embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

### 5′ UTR

In some aspects, provided herein is an mRNA having one or more structural modifications that inhibits leaky scanning and/or promotes the translational fidelity of mRNA translation, wherein at least one of the structural modifications is a GC-rich RNA element. In some aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In particular embodiments, the sequence comprises a repeating GC-motif [CCG]n, wherein n=1, 2, 3, 4 or 5. In particular embodiments, the sequence comprises a repeating GC-motif [CCG]n, wherein n=1, 2, or 3. In particular embodiments, the sequence comprises a repeating GC-motif [CCG]n, wherein n=1. In particular embodiments, the sequence comprises a repeating GC-motif [CCG]n, wherein n=2. In particular embodiments, the sequence comprises a repeating GC-motif [CCG]n, wherein n=3. In particular embodiments, the sequence comprises a repeating GC-motif [CCG]n, wherein n=4. In particular embodiments, the sequence comprises a repeating GC-motif [CCG]n, wherein n=5.

In another aspect, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs. The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these positions would be relatively common. Studies have shown that more footprints are generated at positions where the PIC and/or ribosome exhibits decreased processivity and fewer footprints where the PIC and/or ribosome exhibits increased processivity. In particular embodiments, residence time or the time of occupancy of the PIC or ribosome at a discrete position or location along a polynucleotide comprising any one or more of the RNA elements described herein is determined by ribosome profiling.

In the invention synthetic engineered mRNA provided here, the UTRs are heterologous to the coding region in a polynucleotide. In particular embodiments, the UTR is heterologous to the ORF encoding a heterologous protein, such as a vaccine, a therapeutic protein, gene-editing protein, a regulatory protein, a chimeric antigen receptor, a reporter gene, and the like. In particular embodiments, the polynucleotide comprises two or more 5′ UTRs or functional fragments thereof, each of which has the same or different nucleotide sequences. In particular embodiments, the polynucleotide comprises two or more 3′ UTRs or functional fragments thereof, each of which has the same or different nucleotide sequences. In other embodiments, at least one UTR is heterologous to the ORF encoding a heterologous protein, such as a vaccine, a therapeutic protein, gene-editing protein, a regulatory protein, a chimeric antigen receptor, a reporter gene, and the like.

In particular embodiments, the 5′ UTR or functional fragment thereof, 3′ UTR or functional fragment thereof, or any combination thereof is sequence optimized.

In particular embodiments, the 5′UTR or functional fragment thereof, 3′ UTR or functional fragment thereof, or any combination thereof comprises at least one chemically modified nucleobase, e.g., N1methyl pseudouridine (m1ΨITP), Pseudouridine (ΨTP), N6-Methyladenosine (m6ATP), N1-Methyladenosine (m1ATP), 5-methylcytidine (m5CTP), 5-Methoxycytidine (5moCTP), 5-Hydroxymethylcytidine (hm5CTP), N4Acetylcytidine (ac4CTP), N1-methylpseudouracil or 5-methoxyuracil, and the like.

UTRs can have features that provide a regulatory role, e.g., increased or decreased stability, localization and/or translation efficiency. An invention engineered synthetic mRNA comprising an invention UTR can be administered to a cell, tissue, or organism, and one or more regulatory features can be measured using routine methods as set forth in the Examples herein. In particular embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTRs also have been known to form secondary structures that are involved in elongation factor binding.

In particular embodiments, the 5′ UTR and the 3′ UTR can be heterologous. In particular embodiments, the 5′ UTR can be derived from a different species than the 3′ UTR. In particular embodiments, the 3′ UTR can be derived from a different species than the 5′ UTR.

Additionally, one or more non-naturally occurring synthetic engineered UTRs provided herein can be used in combination with one or more non-synthetic UTRs. See, e.g., Mandal and Rossi, Nat. Protoc. 2013 8(3):568-82, the contents of which are incorporated herein by reference in their entirety. ####

In other embodiments, UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In particular embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see U.S. Pat. No. 10,106,800, the contents of which are incorporated herein by reference in its entirety).

In certain embodiments, the engineered RNAs of the invention comprise a 5′ UTR and/or a 3′ UTR selected from any of the UTRs disclosed herein. In particular embodiments, the 5′ UTR comprises any one of the exemplary 5′ UTR sequences set forth as SEQ ID NOs: 1-123 in the Sequence Listing herein. In particular embodiments, the 3′ UTR comprises any one of the exemplary 3′ UTR sequences set forth as SEQ ID NOs: 124-438 in the Sequence Listing herein. In more particular embodiments, the engineered mRNAs of the invention comprise one or more of the 5′ UTR sequences set forth as SEQ ID NOs: 22, 32-34, 35-68, 70-71, 74-75, 90, 92, 97, 103, 111, 115, and 121-122, in combination with one or more the 3′ UTR sequences set forth as SEQ ID NOs: 145, 150-185, 189-197, 199-201, 203-204, 206-209, 211-323, 335-345, 347, 349-350, 352-422, and 428-438. In these embodiments, both the 5′ UTRs and the 3′ UTRs are non-naturally occurring synthetically engineered UTRs.

The polynucleotides of the invention can comprise combinations of features. For example, the ORF can be flanked by a 5′UTR that comprises a strong Kozak translational initiation signal and/or a 3′UTR comprising an oligo (dT) sequence for templated addition of a poly-A tail.

A 5′UTR can comprise a first polynucleotide fragment and a second polynucleotide fragment from the same and/or different UTRs (see, e.g., U.S. Pat. No. 8,835,621, herein incorporated by reference in its entirety).

Other non-UTR sequences can be used as regions or subregions within the engineered mRNA polynucleotides of the invention. For example, introns or portions of intron sequences can be incorporated into the polynucleotides of the invention. Incorporation of intronic sequences can increase protein production as well as polynucleotide expression levels. In particular embodiments, the polynucleotide comprises a synthetic 5′ UTR in combination with a non-synthetic 3′ UTR.

In particular embodiments, the UTR can also include at least one translation enhancer polynucleotide, translation enhancer element, or translational enhancer elements (collectively, “TEE.” which refers to nucleic acid sequences that increase the amount of polypeptide or protein produced from a polynucleotide. As a non-limiting example, the TEE can be located between the transcription promoter and the start codon. In particular embodiments, the 5′ UTR further comprises a TEE.

### 3′ UTRs

In certain embodiments, an engineered mRNA polynucleotide of the present invention (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like, further comprises a 3′ UTR.

3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the invention comprises a binding site for regulatory proteins or microRNAs.

### Regions Having a 5′ Cap

In particular embodiments, the inventions engineered mRNA, such as those described in Table 1, further comprise a 5′ Cap, such the that the final engineered mRNA comprises: (a) a 5′ untranslated region (5′UTR), wherein the 5′ UTR further comprises a 5′ Cap; (b) a CDS region encoding a heterologous polypeptide; (c) a 3′ untranslated region (3′UTR); and (d) a 3′ poly A tail region. As set forth herein, the CDS or ORF segment encodes a heterologous protein, such as a vaccine, a therapeutic protein, gene-editing protein, a regulatory protein, a chimeric antigen receptor, a reporter gene, and the like.

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly-A binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In particular embodiments, the polynucleotides of the present invention (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like), incorporate a cap moiety.

In particular embodiments, polynucleotides of the present invention (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like) comprise a nonhydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

Polynucleotides of the invention (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like) can also be capped post-manufacture (whether IVT or chemical synthesis), using enzymes, in order to generate functional 5′-cap structures. In particular embodiments, functional 5′-cap structures used herein, outperforms the corresponding endogenous, wild-type, natural or physiological feature in one or more respects. Non-limiting examples of functional 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-Omethyltransferase enzyme can create a canonical 5′-5′-triphosphate linkage between the 5′terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)ppp(5′)NlmpN2mp (cap 2).

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoroguanosine, 7-deaza-guanosine, 8-oxo-guanosine, 2-amino-guanosine, LNA-guanosine, and 2azido-guanosine. In particular embodiments, the Cap structure is selected from Cap 1, Cap 2, or m6A Cap 1. In another embodiment, the Cap structure is selected from Cap 1. Additional Cap structures for use herein are described in U.S. Pat. No. 9,597,380, which is incorporated herein by reference in its entirety for all purposes.

### Poly-A Tails

In particular embodiments, an invention engineered mRNA construct sequence encoding a heterologous protein, such as a vaccine, a therapeutic protein, gene-editing protein, a regulatory protein, a chimeric antigen receptor, a reporter gene, and the like, further comprises a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is at least 40 nucleotides in length. In another embodiment, the poly-A tail is at least 60 nucleotides in length. In another embodiment, the poly-A tail is at least 80 nucleotides in length. In another embodiment, the poly-A tail is at least 100 nucleotides in length. In another embodiment, the poly-A tail is at least 120 nucleotides in length.

Poly-A Tails can Also be Added after the Construct is Exported from the Nucleus.

According to the present invention, terminal groups on the poly-A tail can be incorporated for stabilization. Polynucleotides of the present invention can include des-3′ hydroxyl tails. They can also include structural moieties or 2′-Omethyl.

The polynucleotides of the present invention can be designed to encode transcripts with alternative poly-A tail structures including histone mRNA. Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly-A tail, the function of which is instead assumed by a stable stem-loop structure and its cognate stem-loop binding protein (SLBP); the latter carries out the same functions as those of PABP on polyadenylated mRNAs.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In particular embodiments, the poly-A tail or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In particular embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Methods of Regulating Expression from an mRNA

An aspect of the invention heterologous engineered mRNAs is related to methods of regulating expression from an mRNA, including in a tissue-specific manner (e.g., cells in vivo and in vitro, such as stem cells or lymphocytes), untranslated region (UTR) sequences for enhancing protein synthesis from mRNAs of interest, such as, for example, therapeutic mRNAs, and methods of using the same as therapeutic agents. In particular embodiments of invention heterologous engineered mRNAs, the UTRs are provided, for example, to increase translation and mRNA stability. In other embodiments, 5′- and 3′-UTRs, for example, can be used to improve translation and mRNA stability of heterologous mRNA and of transcribed mRNA for a therapy.

According to an aspect of the disclosure, provided herein are compositions and methods for increasing protein synthesis by increasing both the time that the mRNA remains in translating polysomes (message stability) and the rate at which ribosomes initiate translation on the message (message translation efficiency).

Accordingly, provided herein is a method of expressing an engineered synthetic mRNA in a cell, said method comprising introducing the invention engineered mRNA or the invention LPNs into said cell.

By increasing the upper limit of mRNA half-life, the quantity of protein delivered may be dramatically increased. For example, endogenous mRNAs show a wide range of relative stabilities. The most stable endogenous mRNAs have half-lives of from 40 to 60 hours. RNA stability may also be increased in a tissue-specific manner.

Moreover, UTR sequences can modulate mRNA stability through a variety of mechanisms, including mRNA binding proteins, miRNA, and secondary structures, which inhibit nucleolytic degradation.

An aspect of the disclosure is related to increase expression from an mRNA construct, e.g., by decreasing the rate of mRNA degradation to increase both the duration and the magnitude of protein synthesis produced from an mRNA dose. An aspect of the disclosure is related to mRNA including, for example, a heterologous or hybrid sequence, which may include an open reading frame (ORF) for a target protein of interest coupled (upstream of the target of interest) to a heterologous UTR derived from another naturally occurring or engineered gene. An aspect of the disclosure is related to mRNA that can include a poly-adenosine region (poly-A tail) downstream of the target of the ORF.

In particular embodiments of the engineered heterologous mRNA, the mRNA may include a structural or chemical modification. As used herein, the phrase “structural or chemical modification”, or grammatical variations thereof, in the context of mRNA refers to chemically modified ribonucleosides. In particular embodiments, invention engineered mRNA can contain naturally occurring ribonucleosides or chemically modified ribonucleosides, i.e., modified mRNA (modRNA). In certain embodiments, modRNA can be prepared to include one or more pseudouridine residues, such as N1methyl pseudouridine (m1ΨTP), Pseudouridine (ΨTP), N6-Methyladenosine (m6ATP), N1-Methyladenosine (m1ATP), 5-methylcytidine (m5CTP), 5-Methoxycytidine (5moCTP), 5-Hydroxymethylcytidine (hm5CTP), N4Acetylcytidine (ac4CTP), and the like. In other embodiments, Uridine and/or Cytidine can be replaced with 2-thiouridine and/or 5-methylcytidine to increase stability of the mRNA.

For example, the nucleoside modified in the mRNA can be a uridine (U), a cytidine (C), an adenine (A), or guanine (G). The modified nucleoside may include, for example, m5C (5methylcytidine), m6A (N6-methyladenosine), s2U (2-thiouridien), Ψ (pseudouridine) or Urn (2O-methyluridine). Example modifications of nucleosides in the mRNA molecule may also include pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5taurinomethyluridine, 1-taurinomethyl pseudouridine, 5-taurinomethyl-2-thio uridine, 1 taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1 methyl-1-deaza pseudouridine, dihydrouridine, dihydropseudouridine, 2-thio dihydrouridine, 2thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1 methyl pseudoisocytidine, pyrrolo-cytidine, pyrrolo-pseudoisocytidine, 2-thio cytidine, 2-thio-5methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebula ne, 5-aza zebula ne, 5methyl zebulahne, 5-aza-2-thio zebulahne, 2-thio zebulahne, 2-methoxy cytidine, 2-methoxy-5-methyl cytidine, 4-methoxy pseudoisocytidine, 4-methoxy-1-methyl pseudoisocytidine, 2aminopuhne, 2,6-diaminopuhne, 7-deaza adenine, 7-deaza-8-aza adenine, 7-deaza-2-aminopuhne, 7-deaza-8-aza-2-aminopurine, 7-deaza-2,6-diaminopuhne, 7-deaza-8-aza-2,6-diaminopuhne, 1 methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio adenine, 2methoxy adenine, inosine, 1-methyl inosine, wyosine, wybutosine, 7-deaza guanosine, 7-deaza8-aza guanosine, 6-thio guanosine, 6-thio-7-deaza guanosine, 6-thio-7-deaza-8-aza guanosine, 7methyl guanosine, 6-thio-7-methyl guanosine, 7-methylinosine, 6-methoxy guanosine, 1 methylguanosine, N2-methylguanosine, N2,N2-dimethylguanosine, 8-oxo guanosine, 7-methyl-8oxo guanosine, 1-methyl-6-thio guanosine, N2-methyl-6-thio guanosine, and N2,N2-dimethyl-6thio guanosine. In another embodiment, the modifications are independently selected from the group consisting of 5-methylcytosine, pseudouridine and 1-methylpseudouridine.

In other embodiments of the invention engineered mRNAs, the modified nucleobase in the mRNA may be a modified uracil including, for example, pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho5U), 5-aminoallyl uridine, 5-halo uridine (e.g., 5-iodom uridine or 5-bromo uridine), 3-methyl uridine (m3U), 5methoxy uridine (mo5U), uridine 5-oxyacetic acid (cmo5U), uridine 5-oxyacetic acid methyl ester (mcmo5U), 5-carboxymethyl uridine (cm5U), 1-carboxymethyl pseudouridine, 5carboxyhydroxymethyl uridine (chm5U), 5-carboxyhydroxymethyl uridine methyl ester (mchm5U), 5-methoxycarbonylmethyl uridine (mcm5U), 5-methoxycarbonylmethyl-2-thio uridine (mcm5s2U), 5-aminomethyl-2-thio uridine (nm5s2U), 5-methylaminomethyl uridine (mnm5U), 5-methylaminomethyl-2-thio uridine (mnm5s2U), 5-methylaminomethyl-2-seleno uridine (mnm5se2U), 5-carbamoylmethyl uridine (ncm5U), 5-carboxymethylaminomethyl uridine (cmnm5U), 5-carboxymethylaminomethyl-2-thio uridine (cmnm5s2U), 5-propynyl uridine, 1 propynyl pseudouridine, 5-taurinomethyl uridine (Tcm5U), 1-taurinomethyl pseudouridine, 5taurinomethyl-2-thio uridine (Tm5s2U), 1-taurinomethyl-4-thio pseudouridine, 5-methyl uridine (m5U, e.g., having the nucleobase deoxythymine), 1-methyl pseudouridine (Γη1 ψ), 5-methyl-2thio uridine (m5s2U), 1-methyl-4-thio pseudouridine (m1s4ψ), 4-thio-1-methyl pseudouridine, 3-methyl pseudouridine (Γη3ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m5D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N1-methyl pseudouridine, 3-(3-amino-3carboxypropyl) uridine (acp3U), 1-methyl-3-(3-amino-3-carboxypropyl) pseudouridine (acp3i|j), 5-(isopentenylaminomethyl) uridine (inm5U), 5-(isopentenylaminomethyl)-2-thio uridine (inm5s2U), .alpha-thio uridine, 2′-0-methyl uridine (Urn), 5,2′-0-dimethyl uridine (m5Um), 2′-0methyl pseudouridine (ψm), 2-thio-2′-0-methyl uridine (s2Um), 5-methoxycarbonylmethyl-2′-0methyl uridine (mcm5Um), 5-carbamoylmethyl-2′-0-methyl uridine (ncm5Um), 5carboxymethylaminomethyl-2′-0-methyl uridine (cmnm5Um), 3,2′-0-dimethyl uridine (m3Um), 5-(isopentenylaminomethyl)-2′-0-methyl uridine (inm5Um), 1-thio uridine, deoxythymidine, 2′F-ara uridine, 2′-F uridine, 2′-OH-ara uridine, 5-(2-carbomethoxyvinyl) uridine, and 5-[3-(1-Epropenylamino) uridine.

In other embodiments of the invention engineered mRNAs, the modified nucleobase may be a modified cytosine including, for example, 5-aza cytidine, 6-aza cytidine, pseudoisocytidine, 3-methyl cytidine (m3C), N4-acetyl cytidine (act), 5-formyl cytidine (f5C), N4-methyl cytidine (m4C), 5-methyl cytidine (m5C), 5-halo cytidine (e.g., 5-iodo cytidine), 5hydroxymethyl cytidine (hm5C), 1-methyl pseudoisocytidine, pyrrolo-cytidine, pyrrolopseudoisocytidine, 2-thio cytidine (s2C), 2-thio-5-methyl cytidine, 4-thio pseudoisocytidine, 4thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2thio zebularine, 2-methoxy cytidine, 2-methoxy-5-methyl cytidine, 4-methoxy pseudoisocytidine, 4-methoxy-1-methyl pseudoisocytidine, lysidine (k2C), alpha-thio cytidine, 2′-0methyl cytidine (Cm), 5,2′-0-dimethyl cytidine (m5Cm), N4-acetyl-2′-0-methyl cytidine (ac4Cm), N4,2′-0dimethyl cytidine (m4Cm), 5-formyl-2′-O-methyl cytidine (f5Cm), N4,N4,2′-0-trimethyl cytidine (m4 2Cm), 1-thio cytidine, 2′-F-ara cytidine, 2′-F cytidine, and 2′-0H-ara cytidine.

In yet other embodiments of the invention engineered mRNAs, the modified nucleobase is a modified adenine including, for example, 2-amino purine, 2,6-diamino purine, 2amino-6-halo purine (e.g., 2-amino-6-chloro purine), 6-halo purine (e.g., 6-chloro purine), 2amino-6-methyl purine, 8-azido adenosine, 7-deaza adenine, 7-deaza-8-aza adenine, 7-deaza-2 amino purine, 7-deaza-8-aza-2-amino purine, 7-deaza-2,6-diamino purine, 7-deaza-8-aza-2,6diamino purine, 1-methyl adenosine (m1A), 2-methyl adenine (m2A), N6-methyl adenosine (m6A), 2-methylthio-N6-methyl adenosine (ms2m6A), N6-isopentenyl adenosine (i6A), 2methylthio-N6-isopentenyl adenosine (ms2i6A), N6-(cis-hydroxyisopentenyl) adenosine (io6A), 2-methylthio-N6-(cis-hydroxyisopentenyl) adenosine e (ms2io6A), N6-glycinylcarbamoyl adenosine (g6A), N6-threonylcarbamoyl adenosine (t6A), N6-methyl-N6-threonylcarbamoyl adenosine (m6t6A), 2-methylthio-N6-threonylcarbamoyl adenosine (ms2g6A), N6,N6-dimethyl adenosine (m6 2A), N6-hydroxynorvalylcarbamoyl adenosine (hn6A), 2-methylthio-N6hydroxynorvalylcarbamoyl adenosine (ms2hn6A), N6-acetyl adenosine (ac6A), 7-methyl adenine, 2-methylthio adenine, 2-methoxy adenine, alpha-thio adenosine, 2′-0-methyl adenosine (Am), N6,2′-0-dimethyl adenosine (m6Am), N6,N6,2′-0-trimethyl adenosine (m6 2Am), 1,2′-0-dimethyl adenosine (m1Am), 2′-0-ribosyl adenosine (phosphate) (Ar(p)), 2-amino-N6-methyl purine, 1 thio adenosine, 8-azido adenosine, 2′-F-ara adenosine, 2′-F adenosine, 2′-OH-ara adenosine, and N6-(19-amino-pentaoxanonadecyl) adenosine.

In other embodiments of the invention engineered mRNAs, the modified nucleobase is a modified guanine including, for example, inosine (I), 1-methyl inosine (m1 l), wyosine (imG), methylwyosine (mimG), 4-demethyl wyosine (imG-14), isowyosine (imG2), wybutosine (yW), peroxywybutosine (o2yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ0), 7-aminomethyl-7-deaza guanosine (preQ-ι), archaeosine (G+), 7-deaza-8-aza guanosine, 6-thio guanosine, 6-thio-7-deaza guanosine, 6-thio-7-deaza-8-aza guanosine, 7-methyl guanosine (m7G), 6-thio-7-methyl guanosine, 7-methyl inosine, 6-methoxy guanosine, 1-methyl guanosine (m1G), N2-methylguanosine (m2G), N2,N2-dimethyl guanosine (m2 2G), N2,7-dimethyl guanosine (m2,7G), N2, N2,7-dimethyl guanosine (m2,2,7G), 8-oxo guanosine, 7-methyl-8-oxo guanosine, 1-methio guanosine, N2-methyl-6-thio guanosine, N2,N2-dimethyl-6-thio guanosine, alpha-thio guanosine, 2′-0-methyl guanosine (Gm), N2-methyl-2′-0-methyl guanosine (m2Gm), N2,N2-dimethyl-2′-0methyl guanosine (m2 2Gm), 1-methyl-2′-0-methyl guanosine (m1Gm), N2,7-dimethyl-2′-0methyl guanosine (m2,7Gm), 2′-0-methyl inosine (Im), 1,2′-0-dimethyl inosine (m1 lm), 2′-0ribosyl guanosine (phosphate) (Gr(p)), 1-thio guanosine, 06-methyl guanosine, 2′-F-ara guanosine, and 2′-F guanosine.

In other embodiments, the nucleobase of the nucleotide can be independently selected from a purine, a pyrimidine, a purine or pyrimidine analog. For example, the nucleobase can each be independently selected from adenine, cytosine, guanine, uracil or hypoxanthine. The nucleobase can also include, for example, naturally occurring and synthetic derivatives of a base, including, but not limited to, pyrazolo[3,4-d]pyrimidines, 5-methylcytosine (5-me-C), 5-hydroxymethyl cytosine, xanthine, hypoxanthine, 2-amino adenine, 6-methyl and other alkyl derivatives of adenine and guanine, 2-propyl and other alkyl derivatives of adenine and guanine, 2-thio uracil, 2-thio thymine and 2-thio cytosine, 5-propynyl uracil and cytosine, 6-azo uracil, cytosine and thymine, pseudouracil, 4-thio uracil, 8-halo (e.g., 8-bromo), 8-amino, 8-thiol, 8-thioalkyl, 8hydroxyl and other 8-substituted adenines and guanines, 5-halo particularly 5-bromo, Strifluoromethyl and other 5-substituted uracils and cytosines, 7-methyl guanine and 7-methyl adenine, 8-aza guanine and 8-aza adenine, deaza guanine, 7-deaza guanine, 3-deaza guanine, deaza adenine, 7-deaza adenine, 3-deaza adenine, pyrazolo[3,4-d]pyrimidine, imidazo[1,5-a]1,3,5 triazinones, 9-deaza purines, imidazo[4,5-d]pyrazines, thiazolo[4,5-d]pyrimidines, pyrazine-2ones, 1,2,4-triazine, pyridazine; and 1,3,5-triazine. When the nucleotides are depicted using the shorthand A, G, C. T or U, each letter refers to the representative base and/or derivatives thereof, e.g., A includes adenine or adenine analogs, e.g., 7-deaza adenine).

In particular embodiments, engineered mRNA constructs encoding a heterologous protein, such as a vaccine, a therapeutic protein, gene-editing protein, a regulatory protein, a chimeric antigen receptor, a reporter gene, and the like, are provided herein.

### Nucleic Acid Modifications:

In particular embodiments of the invention 5′ UTRs, 3′ UTRs and/or synthetic engineered mRNA constructs provided herein, different modified nucleotides can be used within therapeutic mRNAs to minimize the immune activation and/or optimize the translation efficiency (e.g., increase polypeptide expression) of mRNA to protein.

An aspect of the disclosure is related to a combination of nucleotide modifications to reduce the innate immune response and sequence optimization, in particular, within the open reading frame (ORF) of the invention engineered synthetic mRNAs encoding a heterologous protein, such as a vaccine, a therapeutic protein, gene-editing protein, a regulatory protein, a chimeric antigen receptor, a reporter gene, and the like, to enhance protein expression.

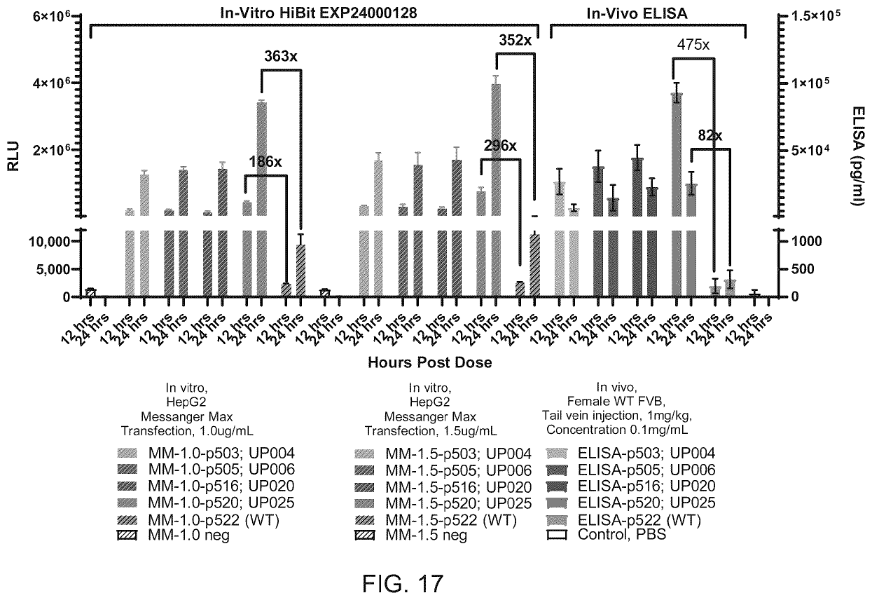

An aspect of the disclosure is related to delivery of mRNA encoding a heterologous protein, such as a vaccine, a therapeutic protein, gene-editing protein, a regulatory protein, a chimeric antigen receptor, a reporter gene, and the like, via a lipid nanoparticle (LNP) delivery system (see FIG. 17). Lipid nanoparticles (LNPs) are an ideal platform for the safe and effective delivery of mRNAs to target cells. LNPs have the unique ability to deliver nucleic acids by a mechanism involving cellular uptake, intracellular transport and endosomal release or endosomal escape.

Accordingly, provided herein is a composition comprising an invention synthetic engineered mRNA disclosed herein, formulated in a lipid nanoparticle (LNP) carrier. Also provided herein is a lipid nanoparticle (LNP) comprising a synthetic engineered mRNA, wherein the mRNA comprises
(a) a 5′ untranslated region (5′UTR);
(b) a CDS region encoding a heterologous polypeptide;
(c) a 3′ untranslated region (3′UTR); and
(d) a 3′ poly A tail region,
   wherein the 5′ UTR is selected from the group consisting of: SEQ ID NOs: 1-123, or
   wherein the 3′ UTR is selected from the group consisting of: SEQ ID NOs: 124-438.

In particular embodiments of the invention LNP, the 5′ UTR is selected from the group consisting of: SEQ ID NOs: 1-123, and the 3′ UTR is selected from the group consisting of: SEQ ID NOs: 124-438. In certain embodiments, the 5′ UTR is selected from the group consisting of: SEQ ID NOs: 22, 32-34, 35-68, 70-71, 74-75, 90, 92, 97, 103, 111, 115, and 121-122, which are non-naturally occurring engineered synthetic 5′ UTRs. In particular embodiments, the 3′ UTR is selected from the group consisting of: SEQ ID NOs: 145, 150-185, 189-197, 199-201, 203-204, 206-209, 211-323, 335-345, 347, 349-350, 352-422, and 428-438, which are non-naturally occurring engineered synthetic 3′ UTRs. Accordingly, other embodiments of the invention LNP, the 5′ UTR is selected from the group consisting of: SEQ ID NOs: 22, 32-34, 35-68, 70-71, 74-75, 90, 92, 97, 103, 111, 115, and 121-122; and the 3′ UTR is selected from the group consisting of: SEQ ID NOs: 145, 150-185, 189-197, 199-201, 203-204, 206-209, 211-323, 335-345, 347, 349-350, 352-422, and 428-438.

In certain embodiments, the LNP comprises a cationic or ionizable lipid. In particular embodiments, the cationic lipid is selected from ALC-0315, DLin-MC3-DMA, DLin-DMA, C12-200, or DLin-KC2-DMA. In another embodiments, the LNP comprises a PEG lipid. In certain embodiments, the heterologous polypeptide is selected from a vaccine, a therapeutic protein, gene-editing protein, a regulatory protein, a chimeric antigen receptor, or a reporter gene. In other embodiments, the CDS region encoding the heterologous polypeptide is codon optimized. As set forth herein, in certain embodiments, the mRNA further comprises a 5′ cap structure. In particular embodiments, the Cap structure is selected from Cap 1, Cap 2, or m6A Cap 1. In a particular embodiment, the 5′ cap structure is Cap 1. In yet other embodiments, the 3′ poly A tail is a length selected from at least 30, 40, 50, 60, 70, 80, 90, 100, 120, 140, 160, 180, or 200 nucleosides.

In particular embodiments, the instant invention utilizes ionizable amino lipid-based LNPs which have improved properties when administered in vivo. It is contemplated herein that the ionizable amino lipid-based LNPs of the invention have improved properties, for example, cellular uptake, intracellular transport and/or endosomal release or endosomal escape. LNPs administered by systemic route (e.g., intravenous (IV) administration), for example, in a first administration, can accelerate the clearance of subsequently injected LNPs, for example, in further administrations. This phenomenon is known as accelerated blood clearance (ABC) and is a challenge in a therapeutic context because repeat administration of mRNA therapeutics is in most instances essential to maintain necessary levels of protein in target tissues in subjects (e.g., subjects suffering from progressive familial intrahepatic cholestasis (PFIC)). Repeat dosing challenges can be addressed on multiple levels. mRNA engineering and/or efficient delivery by LNPs can result in increased levels and or enhanced duration of protein being expressed following a first dose of administration, which in turn, can lengthen the time between first dose and subsequent dosing. It is known that the accelerated blood clearance (ABC) phenomenon is, at least in part, transient in nature, with the immune responses underlying ABC resolving after sufficient time following systemic administration. As such, increasing the duration of protein expression and/or activity following systemic delivery of an mRNA therapeutic of the invention in one aspect, combats the ABC phenomenon. Moreover, LNPs can be engineered to avoid immune sensing and/or recognition and can thus further avoid ABC upon subsequent or repeat dosing. Exemplary aspect of the invention feature novel LNPs which have been engineered to have reduced ABC.

An aspect of the disclosure is related to methods and processes of preparing and delivering such nucleic acid to a target cell are also provided. Furthermore, kits and devices for the design, preparation, manufacture and formulation of such nucleic acids are also included in the instant disclosure.

In certain aspects, the disclosure provides a polynucleotide (e.g., a RNA, e.g., a mRNA) comprising a nucleotide sequence (e.g., an open reading frame (ORF)) encoding a heterologous protein, such as a vaccine, a therapeutic protein, gene-editing protein, a regulatory protein, a chimeric antigen receptor, a reporter gene, and the like. In particular embodiments, the heterologous protein, such as a vaccine, a therapeutic protein, gene-editing protein, a regulatory protein, a chimeric antigen receptor, a reporter gene, and the like, polypeptide of the invention ORF is a wild type full length human protein. In particular embodiments, sequence tags or amino acids, can be added to the sequences encoded by the polynucleotides of the invention (e.g., at the N-terminal or C-terminal ends), e.g., for localization. In particular embodiments, amino acid residues located at the carboxy, amino terminal, or internal regions of a polypeptide of the invention can optionally be deleted providing for fragments.

### Polynucleotides and Open Reading Frames (ORFs)

The instant invention features engineered mRNAs, e.g., heterologous engineered mRNAs, for use in treating or preventing disease. The invention engineered synthetic mRNAs provided herein for use can be administered to subjects and encode human a heterologous protein, such as a vaccine, a therapeutic protein, gene-editing protein, a regulatory protein, a chimeric antigen receptor, a reporter gene, and the like protein in vivo. Accordingly, the invention relates to polynucleotides, e.g., mRNA, comprising an open reading frame of linked nucleosides encoding a heterologous protein, such as a vaccine, a therapeutic protein, gene-editing protein, a regulatory protein, a chimeric antigen receptor, a reporter gene, and the like, isoforms thereof, functional fragments thereof, and fusion proteins comprising a heterologous protein, such as a vaccine, a therapeutic protein, gene-editing protein, a regulatory protein, a chimeric antigen receptor, a reporter gene, and the like. In particular embodiments, the open reading frame is sequence-optimized. In particular embodiments, the invention provides sequence-optimized polynucleotides comprising nucleotides encoding a heterologous protein, such as a vaccine, a therapeutic protein, gene-editing protein, a regulatory protein, a chimeric antigen receptor, a reporter gene, and the like, or sequence having high sequence identity with those sequence optimized polynucleotides.

In particular embodiments, the invention 5′ UTRs, 3′ UTRs and/or synthetic engineered mRNA constructs provided herein increases protein expression levels and/or detectable bile transport levels in cells when a heterologous protein, such as a vaccine, a therapeutic protein, gene-editing protein, a regulatory protein, a chimeric antigen receptor, a reporter gene, and the like, is introduced in those cells, e.g., by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, compared to heterologous protein, such as a vaccine, a therapeutic protein, gene-editing protein, a regulatory protein, a chimeric antigen receptor, a reporter gene, and the like, protein expression levels and/or detectable bile transport levels in the cells prior to the administration of the invention synthetic engineered mRNAs. Heterologous protein, such as a vaccine, a therapeutic protein, gene-editing protein, a regulatory protein, a chimeric antigen receptor, a reporter gene, and the like, protein expression levels and/or bile transport activity can be measured according to methods known in the art. In particular embodiments, the invention 5′ UTRs, 3′ UTRs and/or synthetic engineered mRNA constructs provided herein is introduced to the cells in vitro. In particular embodiments, the invention 5′ UTRs, 3′ UTRs and/or synthetic engineered mRNA constructs provided herein is introduced to the cells in vivo.

### Signal Sequences

In some embodiments, the invention 5′ UTRs, 3′ UTRs and/or synthetic engineered mRNA constructs provided herein can also comprise nucleotide sequences that encode additional features that facilitate trafficking of the encoded polypeptides to therapeutically relevant sites. One such feature that aids in protein trafficking is the signal sequence, or targeting sequence. The peptides encoded by these signal sequences are known by a variety of names, including targeting peptides, transit peptides, and signal peptides. In particular embodiments, the invention synthetic engineered mRNA construct comprises a nucleotide sequence (e.g., an ORF) that encodes a signal peptide operably linked to a nucleotide sequence that encodes a heterologous protein, such as a vaccine, a therapeutic protein, gene-editing protein, a regulatory protein, a chimeric antigen receptor, a reporter gene, and the like.

In particular embodiments, the “signal sequence” or “signal peptide” is a polynucleotide or polypeptide, respectively, which is from about 30-210, e.g., about 45-80 or 1560 nucleotides (e.g., about 20, 30, 40, 50, 60, or 70 amino acids) in length that, optionally, is incorporated at the 5′ (or N-terminus) of the coding region or the polypeptide, respectively. Addition of these sequences results in trafficking the encoded polypeptide to a desired site, such as the endoplasmic reticulum or the mitochondria through one or more targeting pathways. Some signal peptides are cleaved from the protein, for example by a signal peptidase after the proteins are transported to the desired site.

### Fusion Proteins

In particular embodiments, the heterologous engineered mRNA polynucleotide of the invention (e.g., a RNA, e.g., an mRNA) can comprise more than one nucleic acid sequence (e.g., an ORF) encoding a polypeptide of interest. In particular embodiments, the polynucleotide of the invention can comprise more than one ORF, for example, a first ORF encoding a heterologous protein, such as a vaccine, a therapeutic protein, gene-editing protein, a regulatory protein, a chimeric antigen receptor, a reporter gene, and the like (a first polypeptide of interest), a functional fragment, or a variant thereof; and a second ORF expressing a second polypeptide of interest. In particular embodiments, two or more polypeptides of interest can be genetically fused, i.e., two or more polypeptides can be encoded by the same ORF. In particular embodiments, the polynucleotide can comprise a nucleic acid sequence encoding a linker (e.g., a G4S peptide linker or another linker known in the art) between two or more polypeptides of interest

### Linkers and Cleavable Peptides

In certain embodiments, the invention engineered synthetic mRNAs of the provided herein encode more than one a heterologous protein, such as a vaccine, a therapeutic protein, gene-editing protein, a regulatory protein, a chimeric antigen receptor, a reporter gene, and the like, referred to herein as multimer constructs. In certain embodiments of the multimer constructs, the mRNA further encodes a linker located between each domain. The linker can be, for example, a cleavable linker or protease-sensitive linker. In certain embodiments, the linker is selected from the group consisting of F2A linker, P2A linker, T2A linker, ATP8B1A linker, and combinations thereof. In a particular embodiment, the linker is an F2A linker.

Sequence Optimization of Engineered mRNA Encoding a Therapeutic Polypeptide

In particular embodiments, the invention engineered mRNA is sequence optimized. In particular embodiments, the heterologous engineered mRNA comprises a nucleotide sequence (e.g., an ORF) encoding a heterologous protein, such as a vaccine, a therapeutic protein, gene-editing protein, a regulatory protein, a chimeric antigen receptor, a reporter gene, and the like, a 5′-UTR, a 3′-UTR, the 5′ UTR or 3′ UTR optionally comprising at least one microRNA binding site, optionally a nucleotide sequence encoding a linker, a poly-A tail, or any combination thereof, in which the ORF(s) are sequence optimized.

Those of skill in the area will appreciate that coding sequence optimization (also sometimes referred to codon optimization) methods are well-known in the art (and discussed in more detail below) and can be useful to achieve one or more desired results. These results can include, e.g., matching codon frequencies in certain tissue targets and/or host organisms to ensure proper folding; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide. Sequence optimization tools, algorithms and services are known in the art, non-limiting examples include services from GeneArt (Life Technologies), DNA2.0 (Menlo Park Calif.) and/or proprietary methods.

In particular embodiments, the engineered mRNAs of the invention comprise a nucleotide sequence (e.g., a nucleotide sequence (e.g., an ORF) encoding a heterologous protein, such as a vaccine, a therapeutic protein, gene-editing protein, a regulatory protein, a chimeric antigen receptor, a reporter gene, and the like; a 5′-UTR, a 3′-UTR, a microRNA binding site, a nucleic acid sequence encoding a linker, or any combination thereof) that is sequence-optimized according to a method comprising: substituting at least one codon in a reference nucleotide sequence (e.g., an ORF encoding a therapeutic polypeptide) with an alternative codon to increase or decrease uridine content to generate a uridine-modified sequence; substituting at least one codon in a reference nucleotide sequence with an alternative codon having a higher codon frequency in the synonymous codon set; substituting at least one codon in a reference nucleotide sequence with an alternative codon to increase G/C content; or a combination thereof.

Features, which can be considered beneficial in particular embodiments of the invention, can be encoded by or within regions of the polynucleotide and such regions can be upstream (5′) to, downstream (3′) to, or within the region that encodes a heterologous protein, such as a vaccine, a therapeutic protein, gene-editing protein, a regulatory protein, a chimeric antigen receptor, a reporter gene, and the like. These regions can be incorporated into the polynucleotide before and/or after sequence-optimization of the protein encoding region or open reading frame (ORF). Examples of such features include, but are not limited to, untranslated regions (UTRs), Kozak sequences, poly-A tail, and detectable tags and can include multiple cloning sites that can have desired recognition, such as for BspQI, LguI, SapI, EamII04, XbaI, and the like.

In particular embodiments, the polynucleotide of the invention comprises a 5′ UTR, a 3′ UTR and/or a microRNA binding site. In particular embodiments, the polynucleotide comprises two or more 5′ UTRs and/or 3′ UTRs, which can be the same or different sequences. In particular embodiments, the polynucleotide comprises two or more microRNA binding sites, which can be the same or different sequences. Any portion of the 5′ UTR, and/or 3′ UTR, including none, can be sequence-optimized and can independently contain one or more different structural or chemical modifications, before and/or after sequence optimization.

In particular embodiments, after optimization, the polynucleotide encoding an invention engineered mRNA construct can be reconstituted and transformed into a vector such as, but not limited to, plasmids, viruses, cosmids, and artificial chromosomes. For example, the optimized polynucleotide can be reconstituted and transformed into chemically competent E. coli, yeast, neurospora, maize, drosophila, etc. where high copy plasmid-like or chromosome structures occur by methods described herein.

In particular embodiments, an engineered mRNA of the present disclosure, for example a polynucleotide comprising an mRNA nucleotide sequence encoding a heterologous protein, such as a vaccine, a therapeutic protein, gene-editing protein, a regulatory protein, a chimeric antigen receptor, a reporter gene, and the like, comprises from 5′ to 3′ end: a 5′ cap provided herein, for example, Cap 1; a 5′ UTR, such as one of the 5′ UTR sequences provided herein in Table 1, for example, SEQ ID NOs: 1-123; an open reading frame encoding a heterologous protein, such as a vaccine, a therapeutic protein, gene-editing protein, a regulatory protein, a chimeric antigen receptor, a reporter gene, and the like; or a sequence optimized nucleic acid sequence encoding such; at least one stop codon (if not present at 5′ terminus of 3′UTR); a 3′ UTR, such as the sequences provided herein in Table 2, for example, SEQ ID NOs: 124-438; and a polyA tail.

In certain embodiments, all uracils in the polynucleotide are N1-methylpseudouracil. In certain embodiments, all uracils in the polynucleotide are 5-methoxyuracil.

In particular embodiments, the percentage of uracil or thymine nucleobases in a sequence-optimized nucleotide sequence is modified (e.g., reduced) with respect to the percentage of uracil or thymine nucleobases in the reference wild-type nucleotide sequence. Such a sequence is referred to herein as an uracil-modified or thymine-modified sequence. The percentage of uracil or thymine content in a nucleotide sequence can be determined by dividing the number of uracils or thymines in a sequence by the total number of nucleotides and multiplying by 100. In particular embodiments, the sequence-optimized nucleotide sequence has a lower uracil or thymine content than the uracil or thymine content in the reference wild-type sequence. In particular embodiments. the uracil or thymine content in a sequence-optimized nucleotide sequence of the invention is greater than the uracil or thymine content in the reference wild-type sequence and still maintain beneficial effects, e.g., increased expression and/or reduced Toll-Like Receptor (TLR) response when compared to the reference wild-type sequence.

### Modified Nucleotide Sequences Encoding Therapeutic Protein Polypeptides

As set forth throughout herein, in particular embodiments, the engineered mRNAs of the invention comprises a chemically modified nucleobase, such as for example, N1methyl pseudouridine (m1ΨTP), Pseudouridine (ΨTP), N6-Methyladenosine (m6ATP), N1-Methyladenosine (m1ATP), 5-methylcytidine (m5CTP), 5-Methoxycytidine (5moCTP), 5-Hydroxymethylcytidine (hm5CTP), N4Acetylcytidine (ac4CTP), and the like; or a chemically modified uracil, e.g., pseudouracil, N1-methylpseudouracil, 5-methoxyuracil, or the like. In particular embodiments, the mRNA is a uracil-modified sequence comprising an ORF encoding a heterologous protein, such as a vaccine, a therapeutic protein, gene-editing protein, a regulatory protein, a chimeric antigen receptor, a reporter gene, and the like, wherein the heterologous engineered mRNA comprises a chemically modified nucleobase, for example, a chemically modified uracil, e.g., pseudouracil, N1-methylpseudouracil, or 5-methoxyuracil, N1methyl pseudouridine (m1ΨTP), Pseudouridine (ΨTP), N6-Methyladenosine (m6ATP), N1-Methyladenosine (m1ATP), 5-methylcytidine (m5CTP), 5-Methoxycytidine (5moCTP), 5-Hydroxymethylcytidine (hm5CTP), N4Acetylcytidine (ac4CTP), and the like.

In certain aspects of the invention, when the modified uracil base is connected to a ribose sugar, as it is in polynucleotides, the resulting modified nucleoside or nucleotide is referred to as modified uridine. In particular embodiments, modified uracil in the invention engineered mRNA polynucleotide is at least about 25%, at least about 30%, at least about 40%, at least about 50%, at least about 60%, at least about 70%, at least about 80%, at least 90%, at least 95%, at least 99%, or about 100% modified uracil. In one embodiment, uracil in the polynucleotide is at least 95% modified uracil. In another embodiment, uracil in the polynucleotide is 100% modified uracil.

In particular embodiments, the uracil content in the ORF of the invention engineered mRNA encoding a heterologous protein, such as a vaccine, a therapeutic protein, gene-editing protein, a regulatory protein, a chimeric antigen receptor, a reporter gene, and the like, is less than about 30%, about 25%, about 20%, about 15%, or about 10% of the total nucleobase content in the ORF. In particular embodiments, the uracil content in the ORF is between about 10% and about 20% of the total nucleobase content in the ORF. In other embodiments, the uracil content in the ORF is between about 10% and about 25% of the total nucleobase content in the ORF. In one embodiment, the uracil content in the ORF of the mRNA encoding a heterologous protein, such as a vaccine, a therapeutic protein, gene-editing protein, a regulatory protein, a chimeric antigen receptor, a reporter gene, and the like, is less than about 20% of the total nucleobase content in the open reading frame. In this context, the term “uracil” can refer to modified uracil and/or naturally occurring uracil.

In further embodiments, the ORF of the mRNA encoding a heterologous protein, such as a vaccine, a therapeutic protein, gene-editing protein, a regulatory protein, a chimeric antigen receptor, a reporter gene, and the like, having modified uracil and adjusted uracil content has increased Cytosine (C), Guanine (G), or Guanine/Cytosine (G/C) content (absolute or relative). In particular embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wildtype ORF. In particular embodiments, the G, the C, or the G/C content in the ORF is less than about 100%, less than about 90%, less than about 85%, or less than about 80% of the theoretical maximum G, C, or G/C content of the corresponding wild type nucleotide sequence encoding a heterologous protein, such as a vaccine, a therapeutic protein, gene-editing protein, a regulatory protein, a chimeric antigen receptor, a reporter gene, and the like. In particular embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a heterologous protein, such as a vaccine, a therapeutic protein, gene-editing protein, a regulatory protein, a chimeric antigen receptor, a reporter gene, and the like, comprises modified uracil and has an adjusted uracil content containing less uracil pairs (UU) and/or uracil triplets (UUU) and/or uracil quadruplets (UUUU) than the corresponding wild-type nucleotide sequence encoding the heterologous protein, such as a vaccine, a therapeutic protein, gene-editing protein, a regulatory protein, a chimeric antigen receptor, a reporter gene, and the like. In particular embodiments, the ORF of the mRNA encoding a heterologous protein, such as a vaccine, a therapeutic protein, gene-editing protein, a regulatory protein, a chimeric antigen receptor, a reporter gene, and the like, contains no uracil pairs and/or uracil triplets and/or uracil quadruplets. In particular embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding a heterologous protein, such as a vaccine, a therapeutic protein, gene-editing protein, a regulatory protein, a chimeric antigen receptor, a reporter gene, and the like. In a particular embodiment, the ORF of the mRNA encoding a heterologous protein, such as a vaccine, a therapeutic protein, gene-editing protein, a regulatory protein, a chimeric antigen receptor, a reporter gene, and the like, contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding a heterologous protein, such as a vaccine, a therapeutic protein, gene-editing protein, a regulatory protein, a chimeric antigen receptor, a reporter gene, and the like, contains no nonphenylalanine uracil pairs and/or triplets.

In further embodiments, the ORF of the mRNA encoding a heterologous protein, such as a vaccine, a therapeutic protein, gene-editing protein, a regulatory protein, a chimeric antigen receptor, a reporter gene, and the like, comprises modified uracil and has an adjusted uracil content containing less uracil-rich clusters than the corresponding wild-type nucleotide sequence encoding the heterologous protein. In particular embodiments, the ORF of the mRNA encoding a heterologous protein, such as a vaccine, a therapeutic protein, gene-editing protein, a regulatory protein, a chimeric antigen receptor, a reporter gene, and the like, contains uracil-rich clusters that are shorter in length than corresponding uracil-rich clusters in the corresponding wild-type nucleotide sequence encoding the heterologous protein.

### Methods for Modifying Polynucleotides

Provided herein are heterologous engineered mRNA polynucleotides comprising a polynucleotide described herein. The modified polynucleotides can be chemically modified and/or structurally modified. When the polynucleotides of the present invention are chemically and/or structurally modified, the polynucleotides can be referred to as “modified polynucleotides” or when RNA, as “modified RNA” or “modRNA”.

The present disclosure provides for modified nucleosides and nucleotides of a polynucleotide (e.g., RNA polynucleotides, such as mRNA polynucleotides) encoding a heterologous protein, such as a vaccine, a therapeutic protein, gene-editing protein, a regulatory protein, a chimeric antigen receptor, a reporter gene, and the like. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In particular embodiments, the modified polynucleotides contain one, two, or more (optionally different) nucleoside or nucleotide modifications. In particular embodiments, a modified polynucleotide, introduced to a cell can exhibit one or more desirable properties, e.g., improved protein expression, reduced immunogenicity, or reduced degradation in the cell, as compared to an unmodified polynucleotide.

In particular embodiments, a polynucleotide of the present invention (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like) is structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “AUCG” can be chemically modified to “AU-5meC-G”. The same polynucleotide can be structurally modified from “AUCG” to “AUCCCG”. Here, the dinucleotide “CC” has been inserted, resulting in a structural modification to the polynucleotide.

### Encoded Polypeptides/Proteins

Invention synthetic engineered mRNA composition comprise, in particular embodiments, at least one nucleic acid (e.g., RNA) having an open reading frame encoding a heterologous protein or polypeptide, such as a vaccine, a therapeutic protein, gene-editing protein, a regulatory protein, a chimeric antigen receptor, a reporter gene, and the like, wherein the nucleic acid comprises nucleotides and/or nucleosides that can be standard (unmodified) or modified as is known in the art. In particular embodiments, nucleotides and nucleosides of the present disclosure comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In particular embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In particular embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

In particular embodiments, the invention 5′ UTRs, 3′ UTRs and/or synthetic engineered mRNA constructs provided herein are not chemically modified and comprises the standard ribonucleotides consisting of adenosine, guanosine, cytosine and uridine. In particular embodiments, nucleotides and nucleosides of the present disclosure comprise standard nucleoside residues such as those present in transcribed RNA (e.g. A. G. C, or U). In particular embodiments, nucleotides and nucleosides of the present disclosure comprise standard deoxyribonucleosides such as those present in DNA (e.g. dA, dG, dC, or dT).

Hence, the invention 5′ UTRs, 3′ UTRs and/or synthetic engineered mRNA constructs provided herein (e.g., DNA nucleic acids and RNA nucleic acids, such as mRNA nucleic acids) can comprise standard nucleotides and nucleosides, naturally occurring nucleotides and nucleosides, non-naturally-occurring nucleotides and nucleosides, or any combination thereof.

In particular embodiments, the invention 5′ UTRs, 3′ UTRs and/or synthetic engineered mRNA constructs provided herein comprise various (more than one) different types of standard and/or modified nucleotides and nucleosides. In particular embodiments, a particular region of a nucleic acid contains one, two or more (optionally different) types of standard and/or modified nucleotides and nucleosides.

In particular embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In particular embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in particular embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosinethymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In particular embodiments, modified nucleobases in nucleic acids (e.g., RNA nucleic acids, such as mRNA nucleic acids) comprise N1-methylpseudouridine (m1ΨTP), Pseudouridine (ΨTP), N6-Methyladenosine (m6ATP), N1-Methyladenosine (m1ATP), 5-methylcytidine (m5CTP), 5-Methoxycytidine (5moCTP), 5-Hydroxymethylcytidine (hm5CTP), N4Acetylcytidine (ac4CTP), N1-methyl-pseudouridine (m1ψ), 1-ethylpseudouridine (e1ψ), 5-methoxy-uridine (mo5U), 5-methyl-cytidine (m5C), and/or pseudouridine (ψ), and the like. In particular embodiments, modified nucleobases in nucleic acids (e.g., RNA nucleic acids, such as mRNA nucleic acids) comprise 5-methoxymethyl uridine, 5-methylthio uridine, 1methoxymethyl pseudouridine, 5-methyl cytidine, and/or 5-methoxy cytidine. In particular embodiments, the polyribonucleotide includes a combination of at least two (e.g., 2, 3, 4 or more) of any of the aforementioned modified nucleobases, including but not limited to chemical modifications.

In particular embodiments, an engineered RNA, e.g., mRNA, nucleic acid of the disclosure comprises N1-methyl-pseudouridine (m1ψ) substitutions at one or more or all uridine positions of the nucleic acid.

In particular embodiments, an engineered RNA, e.g., mRNA, nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In particular embodiments, an engineered RNA, e.g., mRNA, nucleic acid of the disclosure comprises pseudouridine (ψ) substitutions at one or more or all uridine positions of the nucleic acid.

In particular embodiments, an engineered RNA, e.g., mRNA, nucleic acid of the disclosure comprises pseudouridine (ψ) substitutions at one or more or all uridine positions of the nucleic acid and 5-methyl cytidine substitutions at one or more or all cytidine positions of the nucleic acid.

In particular embodiments, an engineered RNA, e.g., mRNA, nucleic acid of the disclosure comprises uridine at one or more or all uridine positions of the nucleic acid.

In particular embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A. G. U. C) may be uniformly modified in a nucleic acid of the disclosure, or in a predetermined sequence region thereof (e.g., in the mRNA including or excluding the poly-A tail). In particular embodiments, all nucleotides X in a nucleic acid of the present disclosure (or in a sequence region thereof) are modified nucleotides, wherein X may be any one of nucleotides A, G. U. C, or any one of the combinations A+G, A+U, A+C, G+U, G+C. U+C, A+G+U, A+G+C, G+U+C or A+G+C.

### Methods of Making Polynucleotides

The present disclosure also provides methods for making the invention synthetic engineered mRNA, e.g., mRNA, polynucleotide of the invention (e.g., an engineered mRNA comprising a nucleotide sequence encoding a heterologous protein, such as a vaccine, a therapeutic protein, gene-editing protein, a regulatory protein, a chimeric antigen receptor, a reporter gene, and the like, or a complement thereof.

In particular embodiments, an invention engineered heterologous polynucleotide (e.g., a RNA, e.g., an mRNA) provided herein, encoding a therapeutic polypeptide, can be constructed using in vitro transcription (IVT), as set forth herein and in Example 3. In other aspects, an invention engineered mRNA provided herein, and encoding a heterologous protein, such as a vaccine, a therapeutic protein, gene-editing protein, a regulatory protein, a chimeric antigen receptor, a reporter gene, and the like, can be constructed by chemical synthesis using an oligonucleotide synthesizer. In other embodiments, an invention engineered mRNA provided herein is made by one or more of the IVT, chemical synthesis, host cell expression, or any other methods well-known in the art.

Accordingly, provided herein is a method of making a synthetic engineered mRNA, said method comprising constructing a: (a) a 5′ untranslated region (5′UTR); (b) a CDS region encoding a heterologous polypeptide; (c) a 3′ untranslated region (3′UTR); and (d) a 3′ poly A tail region,
wherein the 5′ UTR is selected from the group consisting of: SEQ ID NOs: 1-123, or
wherein the 3′ UTR is selected from the group consisting of: SEQ ID NOs: 124-438; and wherein said constructing is by one or more of the IVT, chemical synthesis, and/or host cell expression.

In other embodiments, naturally occurring nucleosides, non-naturally occurring nucleosides, or combinations thereof, can totally or partially naturally replace occurring nucleosides present in the invention engineered mRNA sequences and can be incorporated into a sequence-optimized nucleotide sequence (e.g., a RNA, e.g., an mRNA) encoding a heterologous protein, such as a vaccine, a therapeutic protein, gene-editing protein, a regulatory protein, a chimeric antigen receptor, a reporter gene, and the like.

### In Vitro Transcription/Enzymatic Synthesis

The polynucleotides of the present invention disclosed herein (e.g., a polynucleotide comprising a nucleotide sequence encoding a heterologous protein, such as a vaccine, a therapeutic protein, gene-editing protein, a regulatory protein, a chimeric antigen receptor, a reporter gene, and the like, can be transcribed using an in vitro transcription (IVT) system. The system typically comprises a transcription buffer, nucleotide triphosphates (NTPs), an RNase inhibitor and a polymerase. The NTPs can be selected from, but are not limited to, those described herein including natural and unnatural (modified) NTPs. The polymerase can be selected from, but is not limited to, T7 RNA polymerase, T3 RNA polymerase and mutant polymerases such as, but not limited to, polymerases able to incorporate polynucleotides disclosed herein. See U.S. Pat. No. 8,999,380, which is herein incorporated by reference in its entirety.

Any number of RNA polymerases or variants can be used in the synthesis of the polynucleotides of the present invention. RNA polymerases can be modified by inserting or deleting amino acids of the RNA polymerase sequence. In a particular embodiment, as a nonlimiting example, the RNA polymerase can be modified to exhibit an increased ability to incorporate a 2′-modified nucleotide triphosphate compared to an unmodified RNA polymerase (see International Publication WO2008078180 and U.S. Pat. No. 8,101,385; herein incorporated by reference in their entireties).

In other embodiments as set forth herein, provided herein are engineered mRNA comprising site-specific chemical modifications of nucleotides, including 5methoxyuridine, 5methoxycytidine, and n1methylpseudouridine applied in regions of the mRNA strands. In another embodiment of the invention, Cap2 or chemically modified Cap2 is utilized in the invention engineered mRNA.

### EXAMPLES

### Example 1—Manufacture of Polynucleotides

Characterization of the polynucleotides of the disclosure are accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Publication WO2014/144711 and U.S. Pat. No. 10,590,161, the content of each of which is incorporated herein by reference in its entirety.

### Example 2—Chimeric Polynucleotide Synthesis

According to the present disclosure, two regions or parts of a chimeric polynucleotide are joined or ligated using triphosphate chemistry. A first region or part of 100 nucleotides or less is chemically synthesized with a 5′ monophosphate and terminal 3′ des0H or blocked OH, for example. If the region is longer than 80 nucleotides, it can be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups are selected from any of those known in the art.

The second region or part of the chimeric polynucleotide is synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part. In particular embodiments, a 5′ terminal cap is 7mG(5′)ppp(5′)NlmpNp.

For ligation methods, ligation with DNA T4 ligase, followed by treatment with DNase should readily avoid concatenation.

The entire chimeric polynucleotide need not be manufactured with a phosphate-sugar backbone. If one of the regions or parts encodes a polypeptide, then such region or part may comprise a phosphate-sugar backbone.

Ligation is then performed using any known click chemistry, orthoclick chemistry, solulink, or other bioconjugate chemistries known to those in the art.

### Chemical Synthesis:

The chimeric polynucleotide is made using a series of starting segments. Such segments include:
(a) a capped and protected 5′ UTR segment comprising a normal 3′OH (SEG. 1)
(b) a 5′ triphosphate segment (ORF or CDS), which may include the coding region of a polypeptide and a normal 3′OH (SEG. 2)
(c) a 5′ monophosphate segment for the 3′ UTR end of the chimeric polynucleotide (e.g., the tail) comprising cordycepin or no 3′OH (SEG. 3)

After synthesis (chemical or IVT), segment 3 (SEG. 3) may be treated with cordycepin and then with pyrophosphatase to create the 5′ monophosphate.

Segment 2 (SEG. 2) may then be ligated to SEG. 3 using RNA ligase. The ligated polynucleotide is then purified and treated with pyrophosphatase to cleave the diphosphate. The treated SEG.2-SEG. 3 construct may then be purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF or CDS (SEG. 2) and 3′UTR+Poly-A tail region (SEG. 3).

The yields of each step may be as much as 90-95%.

### Example 3—In Vitro Transcription (IVT)

The in vitro transcription reaction generates RNA polynucleotides. Such polynucleotides may comprise a region or part of the polynucleotides of the disclosure, including chemically modified RNA (e.g., mRNA) polynucleotides. The chemically modified RNA polynucleotides can be uniformly modified polynucleotides. The in vitro transcription reaction utilizes a custom mix of nucleotide triphosphates (NTPs). The NTPs may comprise chemically modified NTPs, or a mix of natural and chemically modified NTPs, or natural NTPs.

A typical in vitro transcription reaction includes the following: Linearized Template DNA, transcription buffer comprised of Tris-HCL or HEPES at pH 8.0, DTT, spermidine, custom NTPs, T7 RNA polymerase, Inorganic pyrophosphatase, and RNase inhibitor. The reaction is carried out at 25° C.-50° C. depending on the polymerase used and the length of the mRNA construct for a duration of 1-3 hours.

The crude IVT mix can be stored at 4° C. overnight for cleanup the next day. 1 U of RNase-free DNaseI is then be used to digest every 1 ug of original DNA template present in the reaction. After 15-30 minutes of incubation at 37° C., the mRNA may be purified using Ambion's MEGACLEAR™ Kit (Austin, Tex.) following the manufacturer's instructions. This kit can purify up to 500 μg of RNA.

Alternatively, the mRNA can be precipitated, without overnight storage, by adding 0.5 volume of 7.5 M LiCl Precipitation Solution (Ambion Catalog #AM9480) to reach 2.5M final LiCl concentration. Store for at least 30 minutes at −20° C. or overnight then centrifuge at ≥20,000×g for 30-60 minutes. Decant supernatant and wash three times with ice cold 70% EtOH. One wash consists of adding 1 mL ice cold 70% EtOH, inverting the tube, centrifugation for 5 minutes at 20,000×g and decanting the supernatant. Following the final wash, let pellet air dry for 5-15 minutes and resuspend in nuclease free water. Following the cleanup, the RNA polynucleotide is quantified using the NanoDrop and analyzed by agarose gel electrophoresis to confirm the RNA polynucleotide is the proper size and that no degradation of the RNA has occurred.

### Example 4—Enzymatic Capping

Enzymatic Cap 1 synthesis of mRNA using Vaccinia Capping System (NEB #M2080) and 2OMT (NEB #M0366) is performed according to the manufacturer's instructions. Capping of a RNA polynucleotide is performed using a mixture including: IVT RNA 300 μg and dH2O up to 420 μl. The mixture is incubated at 65° C. for 5 minutes to denature RNA, and then is transferred immediately to ice.

The next step in the protocol is the mixing of 10× Capping Buffer (0.5 M Tris-HCl (pH 8.0), 60 mM KCl, 12.5 mM MgCl2) (60.0 μl); 10 mM GTP (30.0 μl); 4 mM S-Adenosyl Methionine (0.2 μl); RNase Inhibitor (100 U) (2.5 μl); 50 U/μl 2′-O-Methyltransferase (30 μl); 10 U/μl Vaccinia capping enzyme (Guanylyl transferase) (30 μl); to reach a final volume of 600 μl); and incubation at 37° C. for 30 minutes. Alternatively, Faustovirus Capping Enzyme (FCE) can be used either with or in lieu of Vaccinia capping enzyme. FCE catalyzes the addition of N⁷-methylguanosine cap (m⁷G) to the 5′ end of triphosphorylated and diphosphorylated transcripts. The reaction is quenched via the addition of 6 μl 500 mM EDTA Stock to arrive at 5 mM EDTA in the final solution.

The RNA polynucleotide is then be purified using Ambion's MEGACLEAR™ Kit (Austin, Tex.) following the manufacturer's instructions. Alternatively, the mRNA can be precipitated by adding 0.5 volume of 7.5 M LiCl Precipitation Solution (Ambion Catalog #AM9480) to reach 2.5M final LiCl concentration. Store for at least 30 minutes at −20° C. or overnight then centrifuge at ≥20,000×g for 30-60 minutes. Decant supernatant and wash three times with ice cold 70% EtOH. One wash consists of adding 1 mL ice cold 70% EtOH, inverting the tube, centrifugation for 5 minutes at 20,000×g and decanting the supernatant. Following the final wash, let pellet air dry for 5-15 minutes and resuspend in nuclease free water. Following the cleanup, the RNA may be quantified using the NANODROP™ (ThermoFisher, Waltham, Mass.) and analyzed by agarose gel electrophoresis to confirm the RNA polynucleotide is the proper size and that no degradation of the RNA has occurred. The RNA polynucleotide product can also be sequenced by running a reverse-transcription-PCR to generate the cDNA for sequencing.

### Example 5—Poly-A Tailing Reaction

A poly-A tail can be included in the engineered mRNA by including a poly-T sequence in the cDNA template. Alternatively, without a poly-T in the cDNA template, a 3′ poly-A tailing reaction is performed before cleaning the final product. This is done by mixing capped IVT RNA (200 μg in 300 μl volume); RNase Inhibitor (100 U); 10× Tailing Buffer (0.5 M Tris-HCl (pH 8.0), 2.5 M NaCl, 100 mM MgCl2) (60.0 μl); 100 mM ATP (6.0 μl); 5 U/μL E. coli Poly(A) Polymerase (30 μl); dH2O up to 600 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Alternatively, the mRNA can be precipitated by adding 0.5 volume of 7.5 M LiCl Precipitation Solution (Ambion Catalog #AM9480) to reach 2.5M final LiCl concentration. Store for at least 30 minutes at −20° C. or overnight then centrifuge at ≥20,000×g for 30-60 minutes. Decant supernatant and wash three times with ice cold 70% EtOH. One wash consists of adding 1 mL ice cold 70% EtOH, inverting the tube, centrifugation for 5 minutes at 20,000×g and decanting the supernatant. Following the final wash, let pellet air dry for 5-15 minutes and resuspend in nuclease free water. Poly-A Polymerase may be a recombinant enzyme expressed in yeast.

It should be understood that the processivity or integrity of the poly-A tailing reaction may not always result in an exact size poly-A tail. Hence, poly-A tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the present disclosure.

### Example 6—Natural 5′ Caps and 5′ Cap Analogues

5′-capping of polynucleotides can be completed concomitantly during the in vitro transcription reaction using the following chemical RNA cap analogs to generate the 5′-guanosine cap structure according to manufacturer protocols: 3″-O-Me-m7G(5)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

In particular embodiments for use herein, a 5′ terminal cap is 7mG(5′)ppp(5′)NlmpNp.

When transfected into mammalian cells, the modified mRNAs have a stability of between 12-18 hours or more than 18 hours, e.g., 24, 36, 48, 60, 72 or greater than 72 hours.

### Example 7—Transient Transfection Via Lipofectamine MessengerMax

24 hours prior to the intended transfection, seed a 96 well TC treated plate with 40,000 viable cells/well in a 100 uL volume. If reading out with HiBit, use Falcon 96 well White opaque tissue culture plates (Ref #353296). If reading out fluorescent tagged proteins use 96 Well Black/Clear Bottom Plate, TC Surface (Thermo Ref #165305). If the downstream assay is an ELISA any TC treated 96 well plate can be used. HepG2 cells and Hek293 cells are passaged and diluted in DMEM (Thermo Ref #11960044) containing 10% FBS (Thermo Ref #16140071) and 1× Glutamax (Thermo Ref #35050061). THP1 cells and Jurkat cells are passaged and diluted with RPMI (Thermo Ref #11875093) containing 10% FBS (Thermo Ref #16140071) and 1× Glutamax (Thermo Ref #35050061). Cells are targeted to have greater than 90% viability at the time of seeding and not to exceed 20 passages from the initial freezer vial thaw. Incubate at 37 C and 5% CO2 for 24 hours. Dilute each mRNA sample to 1 mg/mL in nuclease free water. Make a LipoF/Optimem Master Mix for the appropriate number of samples and replicates being transfected. For every one sample in a 96 well plate, combine 49.2 uL Optimem (Thermo Ref #11058021) and 0.8 uL Lipofectamine Messenger Max (Thermo Ref #LMRNA015). Allow LipoF to incubate in Optimem for 10 min before proceeding to next step. Dilute each mRNA sample to 0.006 ug/uL with Optimem. The final volume accounts for the number of replicate wells and timepoints. If all samples had been normalized to 1 ug/uL and the desired final volume is 3.3 mL for instance this means 3280.2 uL Optimem+19.8 uL 1 ug/uL mRNA would be combined per sample. To each of these samples, add an equal volume of the LipoF/Optimem Mastermix; in the example provided this would mean 3.3 mL. The final 6.6 mL would then be mixed and allowed to incubate for 10 min at 37 C. The solution can then be pipetted onto the cells (100 uL per well if using a 96 well plate). The plates can then be placed back in the 37 C, 5% CO2 incubator until ready for the appropriate assay readout.

### Transient Transfection Via Electroporation

To efficiently transduce primary human (T-cells) from Stemcell Technologies (Cat #70024), with our caped mRNA constructs in order to test expression of HiBit tagged proteins electroporation is required.

Required Medium: ImmunoCult™-XF (StemCell Technologies Cat #100-0956) is a serum-free and xeno-free medium optimized for the in vitro culture and expansion of human T cells isolated from peripheral blood. Recombinant cytokines, required for the optimal growth and expansion of T cells, have not been added to ImmunoCult™-XF. This allows users the flexibility to prepare a medium that meets their requirements. There is no need to supplement the medium with serum. This medium supports robust T cell expansion with high viability after 10-12 days of culture. Complete ImmunoCult™-XF must be prepared fresh on each day of use.

Preparation of fresh complete ImmunoCult™-XF: Add cytokines Human Recombinant IL-2 (StemCell Technologies Catalog #78036/78145) to ImmunoCult™-XF. Mix thoroughly. Add 10 ug/ml, thus add 10 ul of IL-2 cytokine in 10 mL of media.

Cell Thawing Procedure: Warm medium in a 37° C. water bath. To thaw the primary T cells, first wipe the outside of the vial of cells with 70% ethanol or isopropanol. In a biosafety hood, twist the cap a quarter-turn to relieve internal pressure and then retighten. Quickly thaw cells in a 37° C. water bath while gently shaking the vial. Remove the vial when a small frozen cell pellet remains. Do not vortex cells. It is important to work quickly in the following steps to ensure high cell viability and recovery. Wipe the outside of the vial with 70% ethanol or isopropanol. Measure the total volume of the cell suspension using a 2 mL serological pipette. This value is used to calculate the number of cells provided. Transfer the remaining cell suspension to a 50 mL conical tube. Rinse the vial with 1 mL of medium and add it dropwise to the cells, while gently swirling the 50 mL tube. Wash by adding 15-20 mL of pre-warmed medium dropwise, while gently swirling the tube. Centrifuge the cell suspension at 300×g for 10 minutes at room temperature (15-25° C.). After centrifugation is complete, carefully remove and discard the supernatant with a pipette, leaving a small amount of medium to ensure the cell pellet is not disturbed. Resuspend the cell pellet by gently flicking the tube. Gently add 15-20 mL of pre-warmed medium to the tube. Centrifuge the cell suspension at 300×g for 10 minutes at room temperature (15-25° C.). Carefully remove the supernatant with a pipette, leaving a small amount of medium to ensure cell pellet is not disturbed. Resuspend the cell pellet by gently flicking the tube. Cell loss of up to 30% can be expected during the wash steps. Resuspend in fresh pre-warmed media targeting 3×10\hat{ }6 cells/mL (use the initial cell density and volume of the cells to estimate the resuspension volume. Measure the cell density and viability using Trypan Blue and a Countess3 instrument or similar. Follow manufacturer's instructions. Dilute viable human T cells in fresh complete ImmunoCult™-XF to 1×10\hat{ }6 cells/mL. To activate the T cells, add 25 μL/mL cells of ImmunoCult™ Human CD3/CD28/CD2 T Cell Activator (Catalog #10970). Place 10 mL volume per T75 flask. Incubate cells at 37° C. and 5% CO₂ for 3 days.

T Cell Expansion and Maintenance: On Day 3, mix the cell suspension thoroughly and perform a viable cell count. Adjust the viable cell density to ˜1-2.5×10\hat{ }5 cells/mL by adding fresh complete ImmunoCult™-XF. Incubate at 37° C. and 5% CO₂ for 2 days.

On Day 5, mix the cell suspension thoroughly and perform a viable cell count. Adjust the viable cell density to ˜1-3×10\hat{ }5 cells/mL by adding fresh complete ImmunoCult™-XF. Incubate at 37° C. and 5% CO₂ for 2 days.

On Day 7, mix the cell suspension thoroughly and perform a viable cell count. Adjust the viable cell density to ˜3-6×10\hat{ }5 cells/mL) by adding fresh complete ImmunoCult™-XF. Incubate at 37° C. and 5% CO₂ for 3 days.

Day 10: Harvest cells if the desired cell number is achieved. Do not passage further. Electroporation can be carried out on these cells at any time from Days 3-10 if the desired cell densities are reached for the transfection experiment.

Electroporation of T-cells: Obtain the necessary amount of T cells needed for the experiment. To test 20 mRNA constructs, 2M of T-cells total, with 100K per construct.

Label 1.5 mL eppendorf tubes with each mRNA construct number and add 100K of cells in each tube. Wash cells three times with OPTI-MEM and re-suspended in BTX Express EP buffer, 200 uL. This is done via centrifugation at 300×g for 10 minutes at room temperature (15-25° C.). Add 1.5 μg/ml of mRNA constructs to each tube containing cells resuspended in EP buffer at 200 uL. Mix with pipettor. Transfer the mixture containing cells and mRNA to 1 mm gap cuvette, (BTX Item #45-0125) and perform electroporation based on Program #1040 on the BTX Gemini machine.

Square Wave Electroporation Settings: Set Voltage: Set Pulse Length: Set Number of Pulses: Desired Field Strength: 200 V 1 ms 1 1800 V/cm

Post Electroporation: Transfer the cells immediately in 2 ml of pre-warmed culture media, in 24 well cell culture plates and culture in the presence of IL-2 (100 IU/ml) at 37° C. and 5% CO2 for 1-2 days. Proceed to assay readout.

### HiBit Relative Luminescence Assay

The HiBit reagents come in a Promega kit (Catalog #N3040) Ensure all reagents reach room temperature prior to use. Make a HiBit Master Mix solution such that there is sufficient final Master Mix volume to aliquot 100 μL per well in a 96 well plate. The HiBit Protein should be diluted 1:100 and the HiBit substrate diluted 1:50 using the 1×HiBit diluent provided. Make HiBit Master Mix immediately prior to intended use. Keep covered in foil at all times.

For adherent cell lines, decant the supernatant and pipette 100 uL HiBit Master Mix per well in the 96 well plate. Wrap tin foil around each plate and shake for 10 min at 600 rpm. Allow plates to sit for an additional 10 minutes. Readout on luminescence, endpoint 2 second integration, auto gain. In this case used Synergy Neo2 spectrophotometer.

For suspension cell lines that were transfected via lipofectamine, mix cell suspension and transfer 100 uL per well of a Falcon 96 well White opaque tissue culture plates (Ref #353296). Add 100 uL HiBit Master Mix per well. Wrap tin foil around each plate and shake for 10 min at 600 rpm. Allow plates to sit for an additional 10 minutes. Readout on luminescence, endpoint 2 second integration, auto gain. In this case used Synergy Neo2 spectrophotometer.

For suspension cells that were transfected via electroporation, transfer 100 uL of cells into three replicate wells of a Falcon 96 well White opaque tissue culture plates (Ref #353296) to serve as technical replicates. Add 100 uL HiBit Master Mix per well. Wrap tin foil around each plate and shake for 10 min at 600 rpm. Allow plates to sit for an additional 10 minutes. Readout on luminescence, endpoint 2 second integration, auto gain. In this case used Synergy Neo2 spectrophotometer.

### Fluorescent Tagged Relative Fluorescence Assay

Seed cells 24 hours prior in transfection using 96 Well Black/Clear Bottom Plate, TC Surface assay plates (ThermoFisherScientific Cat #165305). Transfect cells as described in Example 7. Before the desired timepoint, place 1×PBS pH 7.4 at 37° C. for 30 minutes. For adherent cell lines, aspirate/decant spent media. Add an equal volume of prewarmed 1×PBS pH 7.4 to the wells. To obtain the Mean Fluorescence Intensity (MFI), read the plate in a BioTek Synergy Neo2 multi-mode plate reader (or similar spectrophotometer). Use Fluorescence Endpoint, Excitation: 489/5, Emission: 511/10, Optics: Bottom, Gain: 100. If additional timepoints are desired, aspirate/decant the PBS and replace with prewarmed complete growth medium (DMEM+10% FBS+1× Glutamax. Place at 37 C and 5% CO2 until the next timepoint. If the cells are suspension, read without PBS exchange. The data is able to discern high from low protein expression.

### Protein Expression ELISA Assay

Polynucleotides (e.g., mRNA) encoding a polypeptide, containing any of the caps taught herein, can be transfected into cells at equal concentrations. The amount of protein secreted into the culture medium can be assayed by ELISA at 4, 6, 12, 24. 36, 48, 72, and/or 96 hours post-transfection. Synthetic polynucleotides that secrete higher levels of protein into the medium correspond to a synthetic polynucleotide with a higher translationally-competent cap structure. An example of an ELISA protocol used for one such CDS was the FastScan™ ELISA (Enzyme-Linked Immunosorbent Assay) Kits (Cell Signaling Technology Cat #29666C). ELISAs used are based on the traditional solid-phase, sandwich-based ELISA method. The sample “target” is incubated with a capture antibody conjugated with a proprietary tag and a second detection antibody linked to horse radish peroxidase (HRP). The entire complex is immobilized to a microwell via an anti-tag antibody. Wells are washed, followed by enzymatic reaction with a TMB substrate and readout of target analyte quantity by colorimetric detection. Readout absorbance at 450 nm within 30 minutes of adding the stop solution.

### Purity Analysis Synthesis

RNA (e.g., mRNA) polynucleotides encoding a polypeptide, containing any of the caps taught herein can be compared for purity using denaturing Agarose-Urea gel electrophoresis or HPLC analysis. RNA polynucleotides with a single, consolidated band by electrophoresis correspond to the higher purity product compared to polynucleotides with multiple bands or streaking bands. Chemically modified RNA polynucleotides with a single HPLC peak also correspond to a higher purity product. The capping reaction with a higher efficiency provides for a more pure polynucleotide population.

### Cytokine Analysis

RNA (e.g., mRNA) polynucleotides encoding a polypeptide, containing any of the caps taught herein can be transfected into cells at multiple concentrations. The amount of proinflammatory cytokines, such as TNF-alpha and IFN-beta, secreted into the culture medium can be assayed by ELISA at 6, 12, 24 and/or 36 hours post-transfection. RNA polynucleotides resulting in the secretion of higher levels of pro-inflammatory cytokines into the medium correspond to polynucleotides containing an immune-activating cap structure.

### Capping Reaction Efficiency

RNA (e.g., mRNA) polynucleotides encoding a polypeptide, containing any of the caps taught herein can be analyzed for capping reaction efficiency by LC-MS after nuclease treatment. Nuclease treatment of capped polynucleotides yield a mixture of free nucleotides and the capped 5′-5-triphosphate cap structure detectable by LC-MS. The amount of capped product on the LCMS spectra can be expressed as a percent of total polynucleotide from the reaction and correspond to capping reaction efficiency. The cap structure with a higher capping reaction efficiency has a higher amount of capped product by LC-MS.

### Protein Expression Assay Results

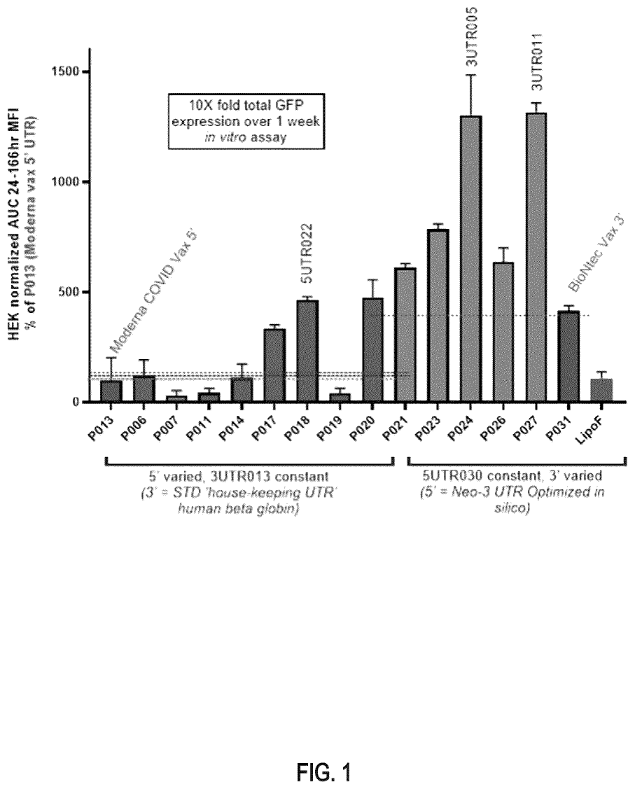

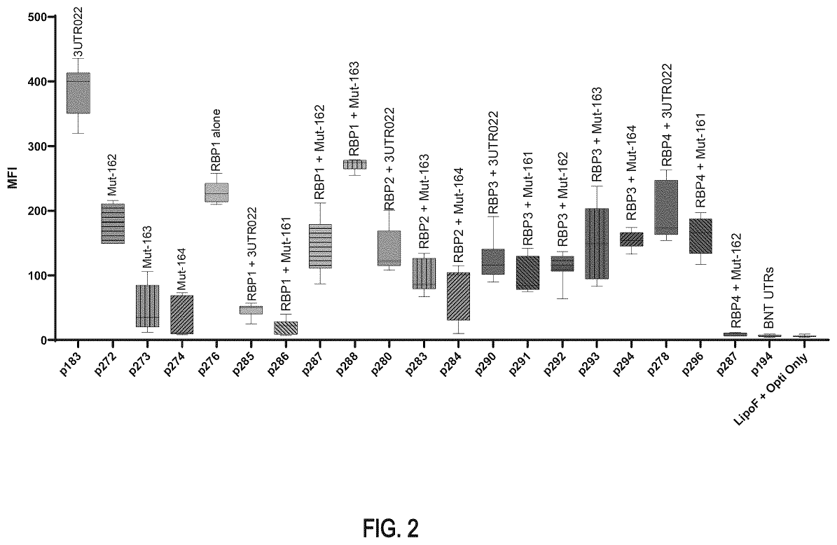

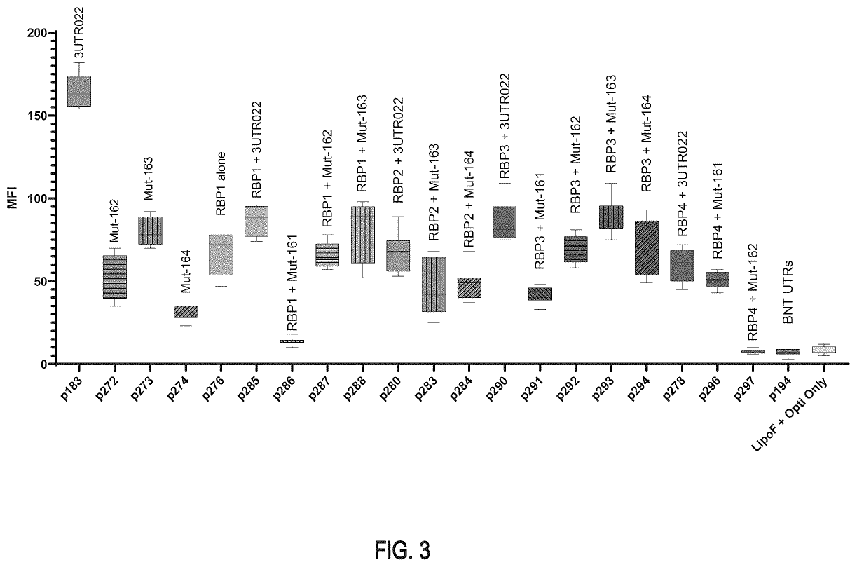

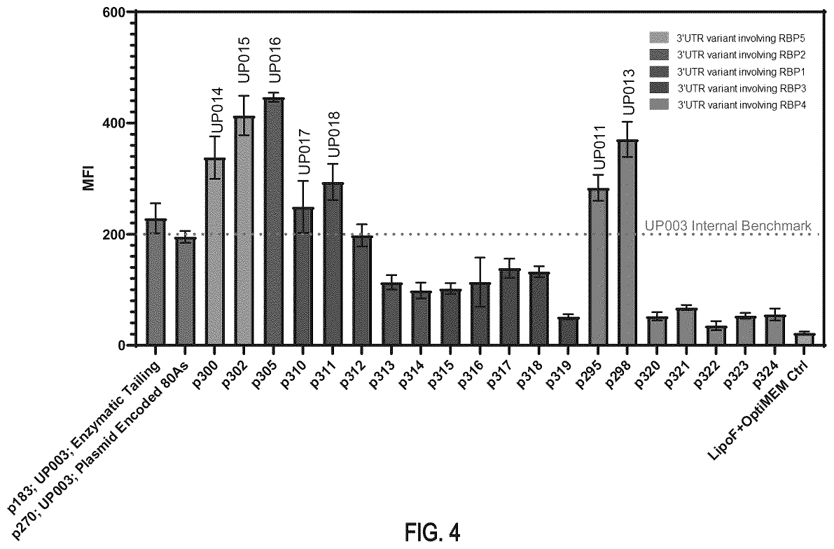

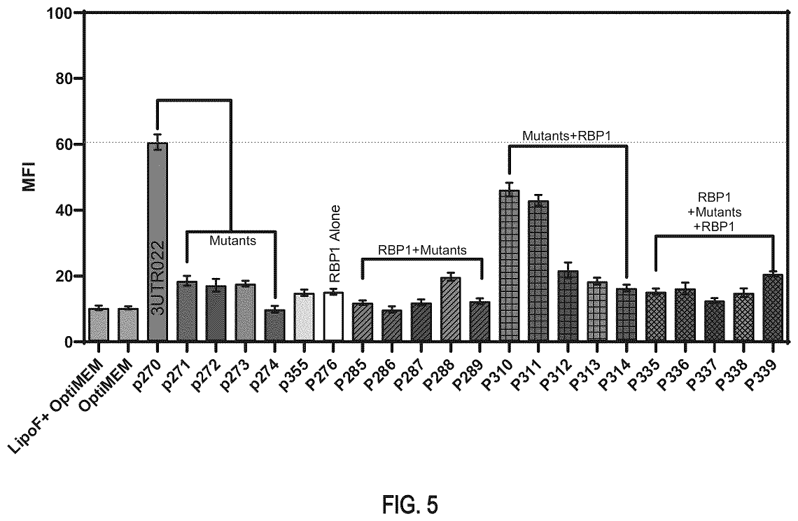

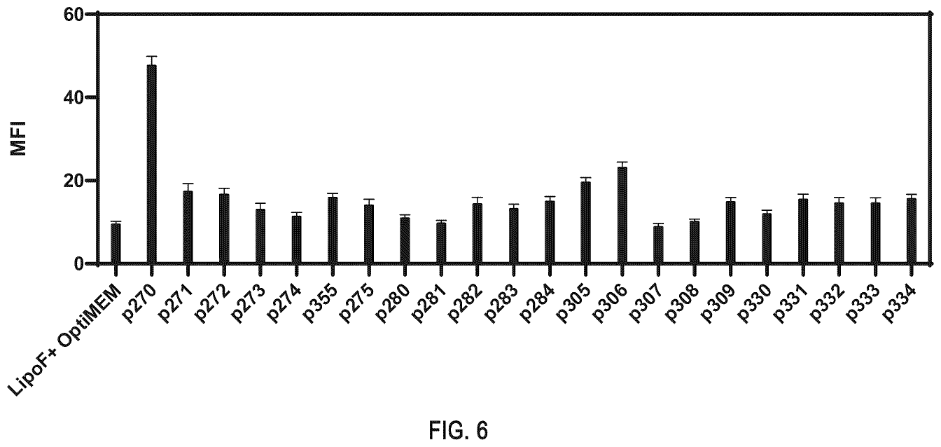

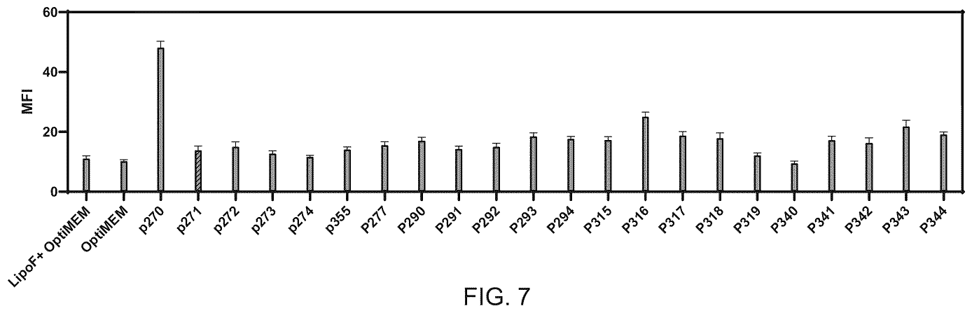

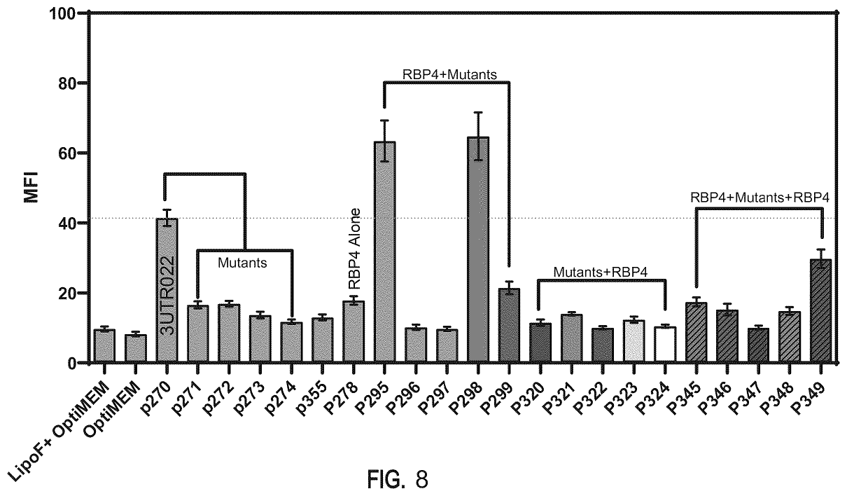

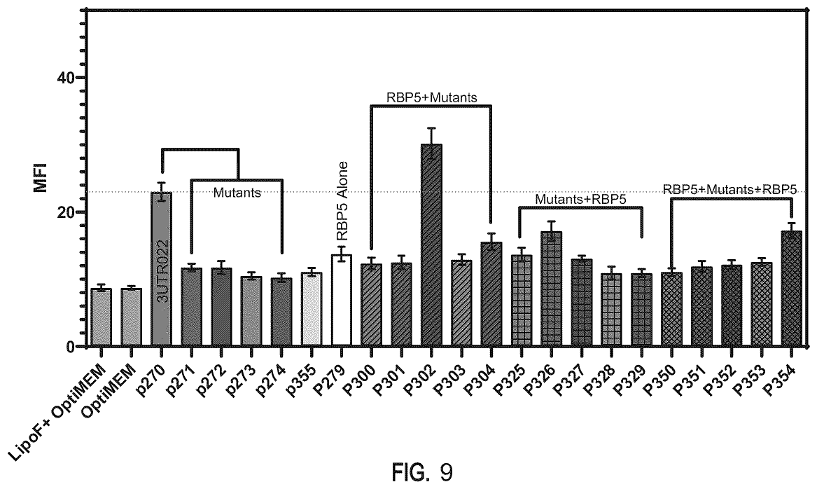

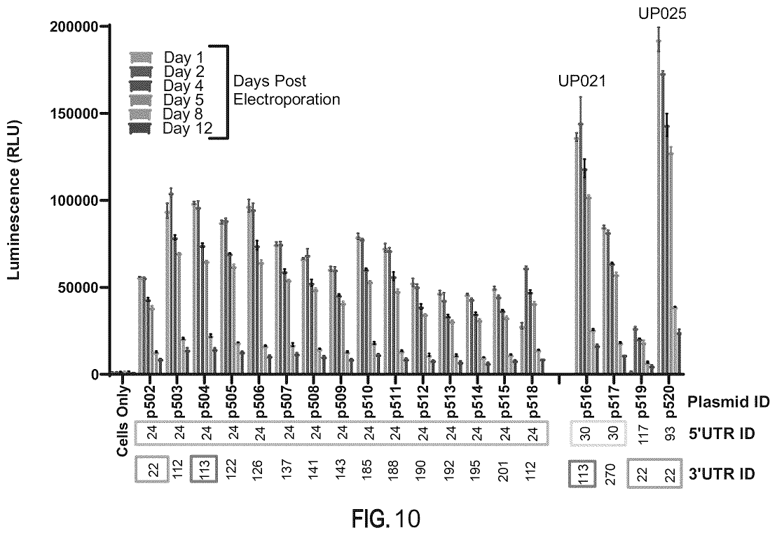

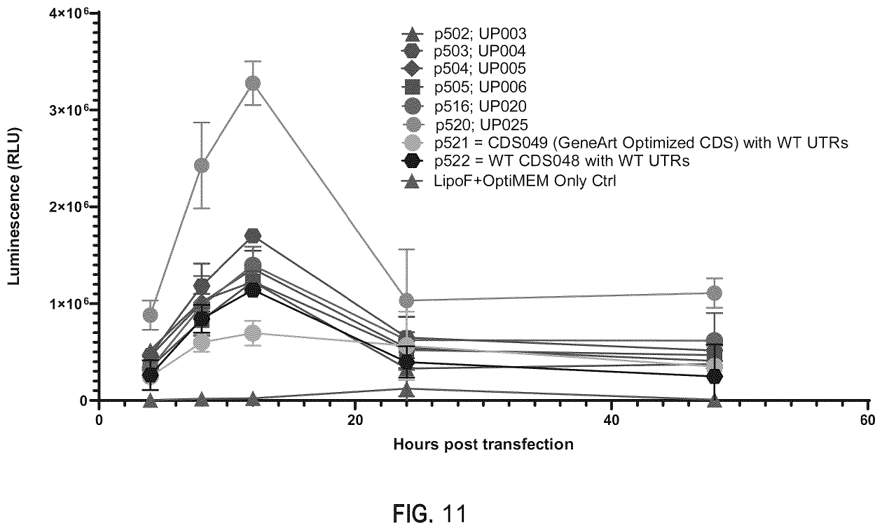

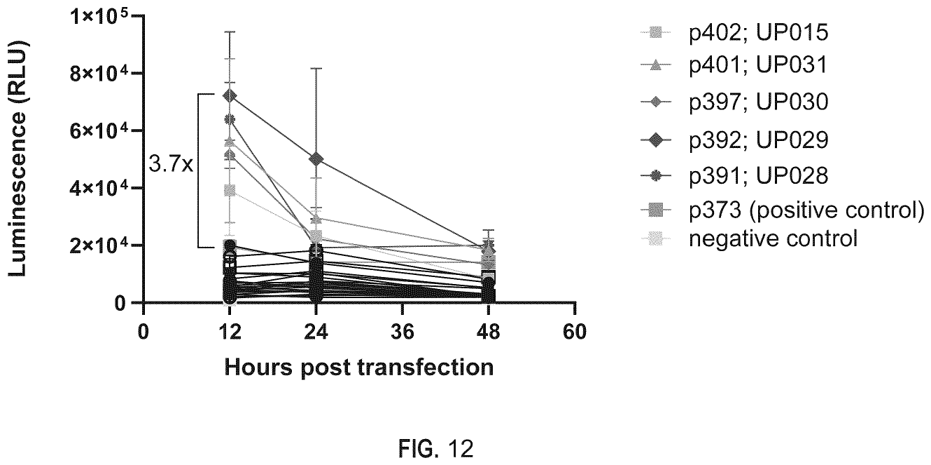

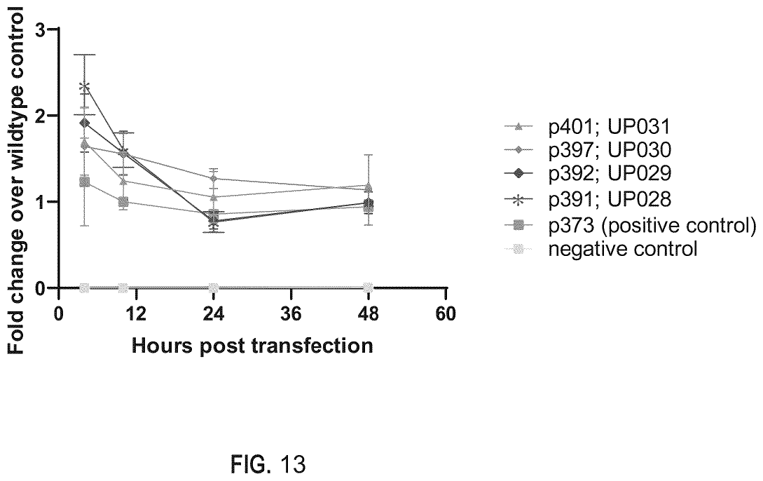

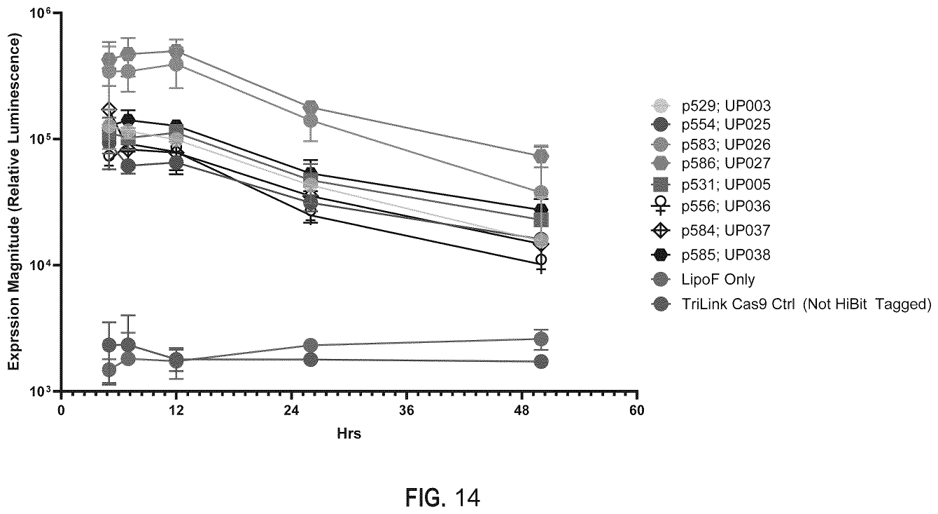

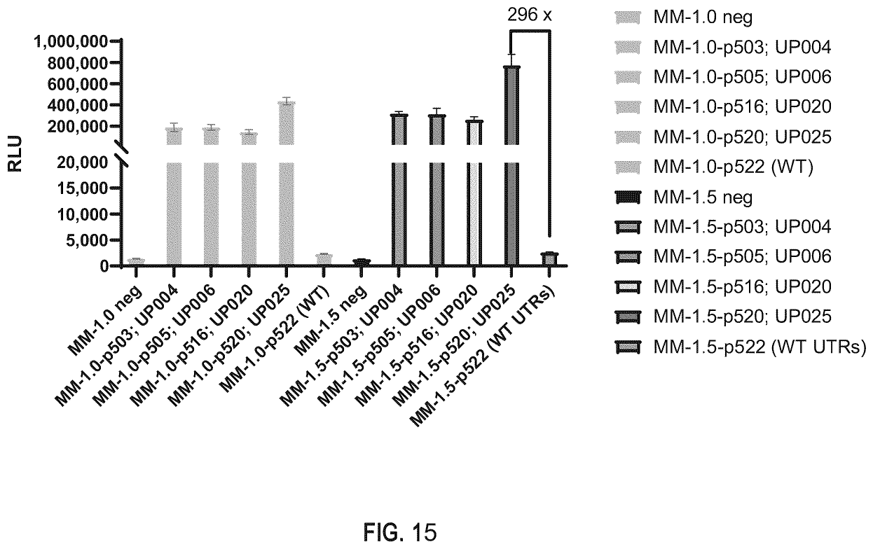

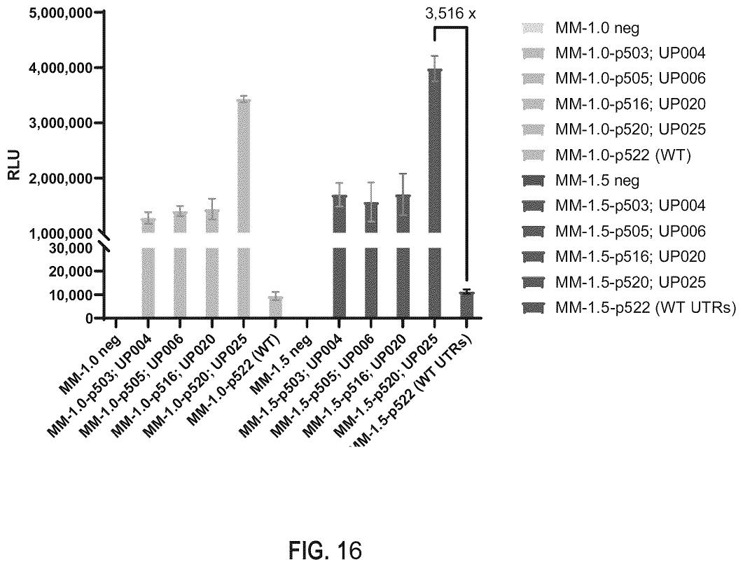

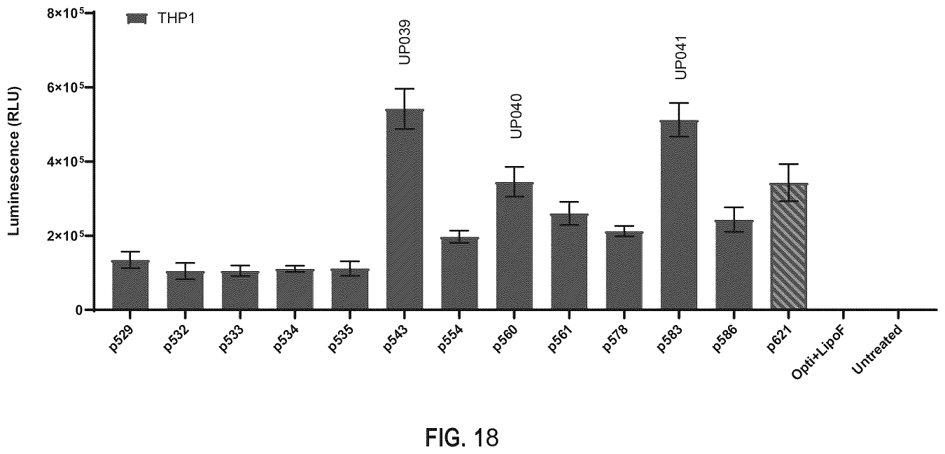

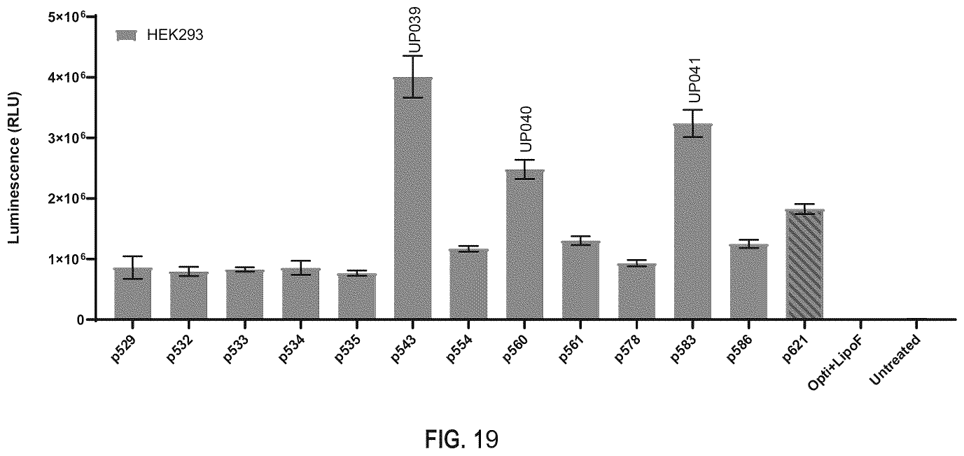

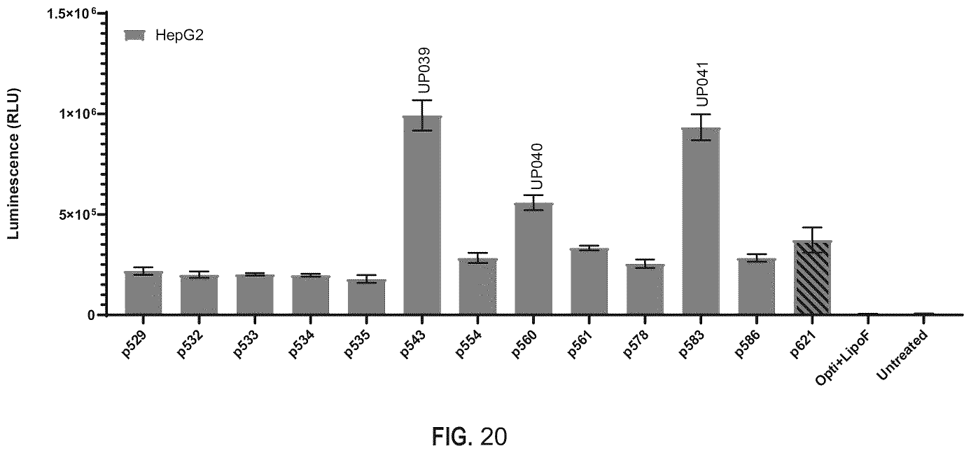

The results of various protein expression assays using various invention 5′ & 3′ UTRs and UTR pairs are shown in FIGS. 1-20. In addition, in mRNA CAR expression comparison experiments in T cells, it has been found that UTR Pairs corresponding to UP025, UP032, UP033, and UP035 showed an approximately 8-fold increase in MFI compared to industry standard benchmark beta globin UTRs; e.g., an 8-fold increase in protein expression. Likewise, in similar mRNA expression comparison experiments in Jurkat cells, it has been found that UP003, UP007. UP011, UP013, UP042 and UP043 showed an increase in protein expression of approximately 2-2.5 fold compared to an industry standard HBB control. In addition, in primary hematopoietic stem cells (HSCs). UTR Pairs corresponding to UP004, UP005, UP006, UP008, UP009, and UP025 were tested with a gene editor CDS and resulted in an increased editing efficiency in the range of 8-17% improvement compared to a benchmark control UTR pair.

### Example 8—Agarose Gel Electrophoresis of Modified RNA or RT PCR Products

Individual RNA polynucleotides (200-400 ng in a 20 μl volume) or reverse transcribed PCR products (200-400 ng) may be loaded into a well on a non-denaturing 1.2% Agarose E-Gel (Invitrogen, Carlsbad, Calif.) and run for 12-15 minutes, according to the manufacturer protocol. Alternatively, the individual RNA polynucleotides (200-400 ng in a 20 μl volume) or reverse transcribed PCR products (200-400 ng) may be assayed using a Bioanalyzer and/or Fragment analyzer.

### Example 9—Nanodrop Modified RNA Quantification and UV Spectral Data

Chemically modified RNA polynucleotides in TE buffer (1 μl) are used for Nanodrop UV absorbance readings to quantitate the yield of each polynucleotide from a chemical synthesis or in vitro transcription reaction.

### Example 10—Formulation of Modified mRNA Using Lipid Nanoparticles

RNA (e.g., mRNA) polynucleotides may be formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for lipid nanoparticle formulations may be used as a starting point. After formation of the particle, polynucleotide is added and allowed to integrate with the complex. The encapsulation efficiency is determined using a standard dye exclusion assay.

For the in vivo experiment set forth in FIG. 17. ALC-0315 lipid nanoparticles (CAS CAS #2036272-55-4, 60-90 nm size, PDI<0.2, were used prepare Lipid nanoparticle (LNP)-encapsulated modified human synthetic mRNAs with plasmids p503, p505, p516, p520 and p522 from the plasmid table set forth herein, and frozen in 10% sucrose 0.5×PBS as 5×100 uL aliquots at 1 mg/mL concentration. The mRNAs were stored at 4° C. and were utilized within 2 weeks post-formulation.

The weights for all of the female WT FVB mice were recorded before tail vein injection. Next, the respective mRNA constructs were dosed once at 1 mg/Kg. Each group consisted of 5 female FVB mice. The female WT FVB mice were sacrificed at 2 time points (12 and 24 hours after tail vein injection), and liver tissues and serum were collected at 12 and 24 hours post-injection, and the fresh liver tissues were snap-frozen in liquid nitrogen and store in freezer of −80° C., prior to readout using Hibit.

### Example 11—Methods for Segmented mRNA Modifications

For assessing the impacts of site-specific modifications, the following procedure is employed.

Various UTR sequences, such as those provided hereinabove, can be synthesized and encoded on a pDNA vector. Through in vitro transcription reactions these UTR fragments may be generated using any variety of modified NTPs. Similarly, the coding sequence, devoid of UTRs may be generated from a pDNA template with or without modified NTPs. The fragments can be sequentially assembled through the use of RNA 5′ Pyrophosphohydrolase (RppH) and T4 RNA Ligase. RppH removes pyrophosphate from the 5′-end of triphosphorylated RNA to leave a 5′ monophosphate RNA. T4 RNA Ligase 1 catalyzes the ligation of a 5′ monophosphorylterminated nucleic acid donor to a 3′ hydroxyl-terminated nucleic acid acceptor through the formation of a 3′ →5′ phosphodiester bond with hydrolysis of ATP to AMP and PPi.

A) First, the 3′UTR fragment is prepared for ligation using RppH. Next, the product is added in excess to a subsequent T4 ligation reaction containing the untreated and therefore triphosphorylated CDS IVT product. At the end of this reaction all CDS fragments should be ligated to a 3′ UTR fragment. The excess monophosphorylated 3′UTR fragments can be digested away using XRN-1, a highly processive 5′→3′ exoribonuclease requiring 5′ monophosphate. This exoribonuclease will not act on triphosphorylated species, leaving the CDS+3′UTR fragment intact. The CDS+3′UTR fragment is then treated with RppH to become monophosphorylated on the 5′end and ready to be ligated to the 3′hydroxyl end of the 5′UTR fragment.

B) Preparation of the 5′UTR fragment involves an enzymatic cap reaction using FCE and 2-OMT to arrive at a Cap-1 structure. This reaction will yield a majority of capped species and potentially some amount of uncapped species. The product is treated with RppH which converts any uncapped material from a triphosphorylated 5′ end to a monophosphorylated 5′end. RppH will have no impact on the Capped molecules. Subsequently, the mRNA will be treated with XRN-1 to remove the monophosphorylated (i.e., uncapped mRNA) species. Removal of uncapped species will decrease immune recognition of the final drug substance.

C) The purified, capped 5′UTR will then be combined with an excess of the monophosphorylated CDS+3′UTR fragment in a T4 RNA ligase reaction. Again, unused monophosphorylated CDS+3′UTR fragments will be degraded with XRN-1. The final product consists of a single strand with a unique modification of the 5′UTR, the CDS, and the 3′ UTR.

D) This product can either be polyadenylated using a polyA polymerase or entered into a subsequent T4 ligation reaction in which a synthetically made modified polyA tail is used as the 5′ monophosphoryl-terminated nucleic acid donor. PolyA polymerase reactions may utilize modified ATPs (as in Phosphodiester modifications in mRNA poly(A) tail prevent deadenylation without compromising protein expression—PubMed (nih.gov))

RP HPLC may be used to purify RNA if it is apparent via CE size that multiple UTRs were ligated.

In addition to the methods and protocols provided herein, the manufacture of polynucleotides and/or parts or regions thereof can be accomplished utilizing the methods taught in U.S. Pat. No. 10,138,507, entitled “Manufacturing Methods for Production of RNA Transcripts,” the contents of which is incorporated herein by reference in its entirety. In addition to the methods and protocols provided herein, purification methods can include those taught in U.S. Pat. Nos. 10,077,439 and 11,377,470, each of which is incorporated herein by reference in its entirety. In addition to the methods and protocols provided herein, detection and characterization methods of the polynucleotides are performed as taught in International Publication WO2014/144039, which is incorporated herein by reference in its entirety.

Of note, the example embodiments of the disclosure described above do not limit the scope of the invention since these embodiments are merely examples of the embodiments of the invention. Any equivalent embodiments are intended to be within the scope of this invention. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

TABLE 1
SEQUENCE LISTING
5′ UTRs

| SEQ ID NO | Registry ID | Sequence |
|---|---|---|
| SEQ ID NO: 1 | 5UTR002 | CTTCCTTTTGTGACTGGCGGTGAACGAGTGCGCAGTGCC |
| SEQ ID NO: 2 | 5UTR003 | aCATTtgCTTCtgacacaactGTGTTcacTAGCAacctCAAACAg |
| | | aCACC |
| SEQ ID NO: 3 | 5UTR004 | ACCGCCGAGACCGCGTCCGCCCCGCGAGCACAGAGCCTCGCCTTT |
| | | GCCGATCCGCCGCCCGTCCACACCCGCCGCCAGCTCACC |
| SEQ ID NO: 4 | 5UTR005 | cgcgTTATTgTTCtgccgGGCGGacacgtgacgcGAAGCTTACCG |
| | | CCGAGACCGCGTCCGCCCCGCGAGCACAGAGCCTCGCCTTTGCCG |
| | | ATCCGCCGCCCGTCCACACCCGCCGCCAGCTCACC |
| SEQ ID NO: 5 | 5UTR006 | tATAAAAcccGGCGGcgcaACGCGCAgccactgtcgagtcgcgtc |
| | | CACCCGCGAGcacagctTCTTTgcagctcCTTCgttgccgGTCCa |
| | | cacCCGCCaccagttCGCCCC |
| SEQ ID NO: 6 | 5UTR007 | cgcgTTATTgTTCtgccgGGCGGacacgtgacgcGAAGCTTtATA |
| | | AAAcccGGCGGcgcaACGCGCAgccactgtcgagtcgcgtcCACC |
| | | CGCGAGcacagctTCTTTgcagctcCTTCgttgccgGTCCacacC |
| | | CGCCaccagttCGCCCC |
| SEQ ID NO: 7 | 5UTR008 | ggcGAActGGTGGcGGGTGtGGACcggcaacGAAGgagctgcAAA |
| | | GAagctgtgCTCGCGGGTGgacgcgactcgacagtggcTGCGCGT |
| | | tgcgCCGCCgggTTTTATaCC |
| SEQ ID NO: 8 | 5UTR009 | cgcgTTATTgTTCtgccgGGCGGacacgtgacgcGAAGCTTggcG |
| | | AActGGTGGcGGGTGtGGACcggcaacGAAGgagctgcAAAGAag |
| | | ctgtgCTCGCGGGTGgacgcgactcgacagtggcTGCGCGTtgcg |
| | | CCGCCgggTTTTATaCC |
| SEQ ID NO: 9 | 5UTR010 | agCACCacggcagcaGGAGGtTTCggCTAAGttGGAGGtactggc |
| | | cacgactGCATGCccgcgcCCGCCaGGTGatacctCCGCCGGTGA |
| | | CCCAGGGGctctgcgacacaaggagtcTGCATGtCTAAGTGCTAg |
| | | ac |
| SEQ ID NO: 10 | 5UTR011 | attaaaggTTTATaccTTCCCaggtaACAAAccaaccaactTTCg |
| | | aTCTCTtgTAGATctgtTCTCTaaacGAActttaaAATCTgtgtg |
| | | gctgtcactcggctGCATGCTTAGTgcactcacgcagtaTAATTA |
| | | ATAActAATTActgtcgttgacaGGACacgagtaactcgtctaTC |
| | | TTCtgcaggctgcttacggtTTCGTCCGTGTTgcagccgatCATc |
| | | agcaCATcTAGGTTtcGTCCGGGTGtgaccGAAaggtaag |
| SEQ ID NO: 11 | 5UTR012 | attaaaggTTTATaccTTCCCaggtaACAAAccaaccaactTTCg |
| | | aTCTCTtgTAGATctgtTCTCTaaacGAActttaaAATCTgtgtg |
| | | gctgtcactcggctgccgcgTTATTgTTCtgccgGGCGGacacgt |
| | | gacgcgtaactAATTActgtcgttgacaGGACacgagtaactcgt |
| | | ctaTCTTCtgcaggctgcttacggtTTCGTCCGTGTTgcagccga |
| | | tCATcagcaCATcTAGGTTtcGTCCGGGTGtgaccGAAaggtaag |
| SEQ ID NO: 12 | 5UTR013 | attaaaggTTTATaccTTCCCaggtaACAAAccaaccaactTTCg |
| | | aTCTCTtgTAGATctgtTCTCTaaacGAActttaaAATCTgtgtg |
| | | gctgtcactcggctgcTTGCTTAGtgcactcacgcagtaTAATTA |
| | | ATAActAATTActgtcgttgacaGGACacgagtaactcgtctaTC |
| | | TTCtgcaggctgcttacggtTTCGTCCGTGTTgcagccgatCATc |
| | | agcaCATcTAGGTTtcGTCCGGGTGtgaccGAAaggtaag |
| SEQ ID NO: 13 | 5UTR015 | GGGAAATAAGAGAGAAAAGAAGAGTAAGAAGAAATATAAGAGCCA |
| | | CCCgcgTTATTgTTCtgccgGGCGGacacgtgacgcGAAGCTTtA |
| | | TAAAAcccGGCGGcgcaACGCGCAgccactgtcgagtcgcgtcCA |
| | | CCCGCGAGcacagctTCTTTgcagctcCTTCgttgccgGTCCaca |
| | | cCCGCCaccagttCGCCCC |
| SEQ ID NO: 14 | 5UTR016 | cgcgTTATTgTTCtgccgGGCGGacacgtgacgcGAAGCTTGGGA |
| | | AATAAGAGAGAAAAGAAGAGTAAGAAGAAATATAAGAGCCACCtA |
| | | TAAAAcccGGCGGcgcaACGCGCAgccactgtcgagtcgcgtcCA |
| | | CCCGCGAGcacagctTCTTTgcagctcCTTCgttgccgGTCCaca |
| | | CCCGCCaccagttCGCCCC |
| SEQ ID NO: 15 | 5UTR017 | CTTCCTTTTGTGACTGGCGGTGAACGAGTGCGCAGTGCCaCATTt |
| | | gCTTCtgacacaactGTGTTcacTAGCAacctCAAACAgaCACC |
| SEQ ID NO: 16 | 5UTR018 | CTTCCTTTTGTGACTGGCGGTGAACGAGTGCGCAGTGCCCgcgTT |
| | | ATTgTTCtgccgGGCGGacacgtgacgcGAAGCTTtATAAAAccc |
| | | GGCGGcgcaACGCGCAgccactgtcgagtcgcgtcCACCCGCGAG |
| | | cacagctTCTTTgcagctcCTTCgttgccgGTCCacacCCGCCac |
| | | cagttCGCCCC |
| SEQ ID NO: 17 | 5UTR019 | CTTCCTTTTGTaCATTtgCTTCtgacacaactGTGTTcacTAGCA |
| | | acctCAAACAgaCACC |
| SEQ ID NO: 18 | 5UTR020 | GGGAAATAAGAGAGAAAAGAAGAGTAAGAAGAAATATAAGAGCCA |
| | | CCCGCCTCGCCGCCTCCaCATTtgCTTCtgacacaactGTGTTca |
| | | cTAGCAacctCAAACAgaCACC |
| SEQ ID NO: 19 | 5UTR021 | ttgatCTTTTAATCTtcgttggccacAATTAaaACAAAccagatc |
| | | gtggagctgcgcgATCCCtttgcATAAAAACATAtggcTTTTGct |
| | | ATAAAAATTATgactgCAAAACACCgggcCATTAATAGcgtgcgg |
| | | agtgATTTAcgcgTTATTgTTCtgccgGGCGGacacgtgacgcgc |
| | | gtggccaaTGGGGgcgcgggCGCCGgcaacTTATTaGGTGacTGT |
| | | ACTTCACCCCCCCCTGGTGCCACCaagtTGTTACATgaAATCTgc |
| | | agtTTCaTAATTtCGGCGGGTCGggcTGGGCcggccaggcgcGGG |
| | | CTactgca |
| SEQ ID NO: 20 | 5UTR022 | gtgaAGATTgacCATctcACAAAagcTGTTAcgtgcttgtAACAC |
| | | actacgCGCCCgTTTTGtaTTCgggaAGTAGttgcgAAAACgGTC |
| | | CCcTTATTgcctgacaagCTAAGggcCACCCtTCTTTCCCCACCG |
| | | CCatc |
| SEQ ID NO: 21 | 5UTR023 | ggtAATCTgcaaATCCCtggcacCCGCCtaAAATTgCCCTCatca |
| | | acCTTCTCTCTaTTCacg |
| SEQ ID NO: 22 | 5UTR024 | GGCTCTGAAAAAAAAAAAAAGACCCAAGCTAGCTAGCGTTTAAAC |
| | | TTAAGCTAGGTACCGAGACC |
| SEQ ID NO: 23 | 5UTR025 | GGCTCTGAAAAAAAAAAAACCGaCATTtgCTTCtgacacaactGT |
| | | GTTcacTAGCAacctCAAACAgaCACC |
| SEQ ID NO: 24 | 5UTR026 | ACACGCTGGAATTCTAGTATACTAAACC |
| SEQ ID NO: 25 | 5UTR027 | aCATTtgCTTCtgacacaactGTGTTcacTAGCAacctCAAACAg |
| | | aCACCAAAAAAAAAAAA |
| SEQ ID NO: 26 | 5UTR029 | AGCCCtccaGGACaggctgCATcaGAAGAggcCATcaagcaggtc |
| | | tgTTCcaAGGGcctttgcgtcaGGTGGgctcaggaTTCcaGGGTG |
| | | gctggACCCCaggCCCCAgctctgcagcAGGGAGGACgtggctGG |
| | | GCTcgtGAAGCATGTGGGGgtgAGCCCAGGGGCCCCAaggcaGGG |
| | | CAcctggcCTTCagcctgcctcAGCCCtgcctgtCTCCCagatca |
| | | ctgtcCTTCtgcc |
| SEQ ID NO: 27 | 5UTR030 | GCTTGTCTCGCTCCGGGGAACGCTCGGAAACTCCCGGCCGCCGCC |
| | | ACCCGCGTCTGTTCTGTTACACAAGGGAAGAAAAGCCGCTGCCGC |
| | | ACTCCGAGTGT |
| SEQ ID NO: 28 | 5UTR031 | atattggagcagcAAGAggctGGGAAgcCATcacttaccttgcac |
| | | tgagAAAGAAGACAAaggcaagttgAAAAGcggagaAATAGtgGC |
| | | CCAgtggttgAAAAAttGAAGcaa |
| SEQ ID NO: 29 | 5UTR032 | atattggagcagcAAGAggctGGGAAgcCATcacttaccttgcac |
| | | tgagAAAGAAGACAAaggccagt |
| SEQ ID NO: 30 | 5UTR033 | GGGAGACTGCCACC |
| SEQ ID NO: 31 | 5UTR034 | GGGAGACTGCCAAG |
| SEQ ID NO: 32 | 5UTR035 | agCACCacggcagcaGGAGGtTTCggCTAAGttGGAGGtactggc |
| | | cacgactgcTTGCCCgcgcCCGCCaGGTGatacctCCGCCGGTGA |
| | | CCCAGGGGctctgcgacacaaggAGTCTgcTTGTCTaagTGCTAg |
| | | ac |
| SEQ ID NO: 33 | 5UTR036 | ttgatCTTTTAATCTtcgttggccacAATTAaaACAAAccagatc |
| | | gtggagctgcgcgATCCCtttgcATAAAAaCATTtggcTTTTGct |
| | | ATAAAAATTTTgactgCAAAACACCgggcCATTAATAGcgtgcgg |
| | | agtgATTTAcgcgTTATTgTTCtgccgGGCGGacacgtgacgcgc |
| | | gtggcCATTGGGGgcgcgggCGCCGgcaacTTATTaGGTGacTGT |
| | | ACTTCACCCCCCCCTGGTGCCACCaagtTGTTAcTtgaAATCTgc |
| | | agtTTCaTAATTtCGGCGGGTCGggcTGGGCcggccaggcgcGGG |
| | | CTactgca |
| SEQ ID NO: 34 | 5UTR037 | AGCCCtccaGGACaggctgCATcaGAAGAggcCATcaagcaggtc |
| | | tgTTCcaAGGGcctttgcgtcaGGTGGgctcaggaTTCcaGGGTG |
| | | gctggACCCCaggCCCCAgctctgcagcAGGGAGGACgtggctGG |
| | | GCTcgtGAAGcTtgTGGGGgtgAGCCCAGGGGCCCCAaggcaGGG |
| | | CAcctggcCTTCagcctgcctcAGCCCtgcctgtCTCCCagatca |
| | | ctgtcCTTCtgcC |
| SEQ ID NO: 35 | 5UTR039 | TGAGAAAGTGTTTAGTAGCAATGATGATTCCTGTGAGTATTAGTA |
| | | TGGTT |
| SEQ ID NO: 36 | 5UTR040 | TGAGAAAGTGTTTAGTAGCAATGATGATCTCTTTAAGTTTTAAAA |
| | | TGGTT |
| SEQ ID NO: 37 | 5UTR041 | TGAGAAAGTGTTTAGTAGCAATGATGAGCCCTTTAAATTTTAAAA |
| | | TGGTG |
| SEQ ID NO: 38 | 5UTR042 | TGAGAAAGTGTTTAGTAGCAATGATGATCTCTGTCAGTTTTAAAA |
| | | TGGTG |
| SEQ ID NO: 39 | 5UTR043 | TGAGAAAGTGTTTAGTAGCAATGATGATCGCTTTTGGTTTTAAAA |
| | | TGGTG |
| SEQ ID NO: 40 | 5UTR044 | TGAGAAAGTGTTTAGTAGCAATGATGATTCCTGTGAGTTTTAAAA |
| | | TGGTG |
| SEQ ID NO: 41 | 5UTR045 | TGAGAAAGTGTTTAGTAGCAATGATGATCTCTTGCAGTTTTAAAA |
| | | TGGTG |
| SEQ ID NO: 42 | 5UTR046 | TGAGAAAGTGTTTAGTAGCAATGATGATTCCTGTGAGTTTTAATA |
| | | TGGTT |
| SEQ ID NO: 43 | 5UTR047 | TGAGAAAGTGTTTAGTAGCAATGATGATCTCTGTCAGTTTAGTTA |
| | | TGGTT |
| SEQ ID NO: 44 | 5UTR048 | TGAGAAAGTGTTTAGTAGCAATGATGAGGATACTGATTTTTAAAA |
| | | TGGTG |
| SEQ ID NO: 45 | 5UTR049 | TGAGAAAGTGTTTAGTAGCAATGATGATTCCTGTGAGATTTAAAA |
| | | TGGTG |
| SEQ ID NO: 46 | 5UTR050 | TGAGAAAGTGTTTAGTAGCAATGATGATCGCTTATTGTTTAATTA |
| | | TGGTT |
| SEQ ID NO: 47 | 5UTR051 | TGAGAAAGTGTTTAGTAGCAATGATGAGGACATTTGTTTTTAAAA |
| | | TGGTG |
| SEQ ID NO: 48 | 5UTR052 | TGAGAAAGTGTTTAGTAGCAATGATGATCTCTGTCAGTTTAATTA |
| | | TGGTT |
| SEQ ID NO: 49 | 5UTR053 | TGAGAAAGTGTTTAGTAGCAATGATGATCTCTTTAAGTTTTAGTA |
| | | TGGTT |
| SEQ ID NO: 50 | 5UTR054 | TGAGAAAGTGTTTAGTAGCAATGATGATCGCTTACAGTTTTAAAA |
| | | TGGTG |
| SEQ ID NO: 51 | 5UTR055 | TGAGAAAGTGTTTAGTAGCAATGATGATTCCTGTGAGATTTAGTA |
| | | TGGTT |
| SEQ ID NO: 52 | 5UTR056 | TGAGAAAGTGTTTAGTAGCAATGATGATTCCTGTGAGTATTAAAA |
| | | TGGTG |
| SEQ ID NO: 53 | 5UTR057 | TGAGAAAGTGTTTAGTAGCAATGATGAACTCTGTGAGATTTAAAA |
| | | TGGTG |
| SEQ ID NO: 54 | 5UTR058 | TGAGAAAGTGTTTAGTAGCAATGATGATTCCTGTGAGATTTAGTA |
| | | TGGTG |
| SEQ ID NO: 55 | 5UTR059 | TGAGAAAGTGTTTAGTAGCAATGATGATTCCTGTGAGTTTTAGTA |
| | | TGGTT |
| SEQ ID NO: 56 | 5UTR060 | TGAGAAAGTGTTTAGTAGCAATGATGATCGCTTTTGGTTTAATTA |
| | | TGGTT |
| SEQ ID NO: 57 | 5UTR061 | TGAGAGAGTGTTTAGTAGCAATGATGATCTCTTTAAGTTTTAAAA |
| | | TGGTG |
| SEQ ID NO: 58 | 5UTR062 | TGAGAAAGTGTTTAGTAGCAATGATGAACTCTGTGAGATTTAGTA |
| | | TGGTT |
| SEQ ID NO: 59 | 5UTR063 | TGAGAAAGTGTTTAGTAGCAATGATGATTCCTGTGAGTTATAAAA |
| | | TGGTG |
| SEQ ID NO: 60 | 5UTR064 | TGAGAAAGTGTTTAGTAGCAATGATGATCTCTTTAAGTTTTAAAA |
| | | TGGTG |
| SEQ ID NO: 61 | 5UTR065 | TGAGAAAGTGTTTAGTAGCAATGATGAGGACATTTGTTTTAATTA |
| | | TGGTT |
| SEQ ID NO: 62 | 5UTR066 | TGAGAAAGTGTTTAGTAGCAATGATGATCGCTTATTGTTTAGTTA |
| | | TGGTT |
| SEQ ID NO: 63 | 5UTR067 | TGAGAAAGTGTTTAGTAGCAATGATGATTCCTGTGAATTTTAAAA |
| | | TGGTG |
| SEQ ID NO: 64 | 5UTR068 | TGAGAAAGTGTTTAGTAGCAATGATGATCTCTTTAAGTTTTAGAA |
| | | TGGTG |
| SEQ ID NO: 65 | 5UTR069 | TGAGAGAGTGTTTAGTAGCAATGATGATTCCTGTGAGTTTTAAAA |
| | | TGGTG |
| SEQ ID NO: 66 | 5UTR070 | TGAGAAAGTGTTTAGTAGCAATGATGATCCCTGTGAGATTTAGTA |
| | | TGGTT |
| SEQ ID NO: 67 | 5UTR071 | TGAGAAAGTGTTTAGTAGCAATGATGATCTCTTGCAGTTTAATTA |
| | | TGGTT |
| SEQ ID NO: 68 | 5UTR072 | TGAGAAAGTGTTTAGTAGCAATGATGATCTCTTACAGTTTTAAAA |
| | | TGGTG |
| SEQ ID NO: 69 | 5UTR073 | ACAAActTTCAGAGAcagcagagcacacaagcttCTAGGacAAGA |
| | | gccagGAAGAAaCCACCgGAAGGAAcCATctcactgtgTGTAaac |
| SEQ ID NO: 70 | 5UTR074 | CGGAGTAATGAAGATGGTACTTACGCATCGCGGCAGGATAGGAAG |
| | | GTCGG |
| SEQ ID NO: 71 | 5UTR075 | GAGTTCCATCTGAGGCCGTTTTGTCAGATGATATCACTGCCTCGC |
| | | ACCAG |
| SEQ ID NO: 72 | 5UTR077 | gtAAAATaccaacTAATTctcgTTCgaTTCCGGCGaaCATTctAT |
| | | TTTacCAACAtcggTTTTTTcAGTAGtaatactgtTTTTGTTCCC |
| | | g |
| SEQ ID NO: 73 | 5UTR078 | agaCACCtctgCCCTCacc |
| SEQ ID NO: 74 | 5UTR079 | TGAGAACATATTTAGTAGCAATGATGATATCGTATTTAGTAGTTA |
| | | TGGTT |
| SEQ ID NO: 75 | 5UTR080 | TGAGTAGAGTTGTAGTAGCAATGATGATCGCTTATTTAGTAATTA |
| | | TGGTT |
| SEQ ID NO: 76 | 5UTR081 | TGAGTAATTATTTAGTAGCAATGATGATATCGTATTTTGATATTA |
| | | TGGTT |
| SEQ ID NO: 77 | 5UTR082 | TGAGAACATATTTAGTTGCAATGATGATCGCGTATCATAATAATA |
| | | TGATT |
| SEQ ID NO: 78 | 5UTR083 | TGAGAACATATTTAGTAGCAATGATGAGGAGATATCTAATAATTA |
| | | TGGTT |
| SEQ ID NO: 79 | 5UTR084 | TGAGGAGAAATGTAGAAGATATGATGAAGCCTATTATTGATAAAA |
| | | GCATC |
| SEQ ID NO: 80 | 5UTR085 | CTCGCATATGCCCGTCTGGCTAGCTATTAAATCTACTACGCGAAC |
| | | ATAAT |
| SEQ ID NO: 81 | 5UTR086 | CCGCTTTTGGGGGACGTCCATTGGATGTTCAAGCCAGTCAGACAA |
| | | CAAAT |
| SEQ ID NO: 82 | 5UTR087 | CCCACCATGTCGCCGATTGTATGTAATGGATCGAAGTCAATGTGA |
| | | AATCA |
| SEQ ID NO: 83 | 5UTR088 | ACCACCATGGCGATGAACGCTTACATAACATGTGGCCATGGGTCT |
| | | TGCAC |
| SEQ ID NO: 84 | 5UTR089 | GGCTAAAAAAAAAAAAAGACCCAAGCTAGCTAGCGTTTAAACTTA |
| | | AGCTAGGTACCGAGACC |
| SEQ ID NO: 85 | 5UTR090 | gtgaAGATTgacCATctcACAAAagttacgtgcttgtAACACact |
| | | acgCGCCCgTTTTGtaTTCgggaAGTAGttgcgAAAACggtCCCC |
| | | TtATTGCACaagCTAAGggcCACCCtTCTTTCCCCACCGCCatc |
| SEQ ID NO: 86 | 5UTR091 | ACATAtccactcctgctctCCCTCctgcaGGTGACCCCagcc |
| SEQ ID NO: 87 | 5UTR092 | acaCATctgctcctgCTCTCTctCCTCCagcgacCCTAGcc |
| SEQ ID NO: 88 | 5UTR093 | aggatcctctgcAGGGGagctccgagtGTCCacaggaAGGGAACT |
| | | ATcagctcctggCATcTGTAagg |
| SEQ ID NO: 89 | 5UTR094 | gCCACCaagtTGTTACATgaAATCTgcagtTTCaTAATTtccgtg |
| | | GGTCGggccgGGCGGgccaggcgcTGGGCacGGTG |
| SEQ ID NO: 90 | 5UTR095 | gCCACCaagtTGTTAcGtgaAATCTgcagtTTCaTAATTtccgtg |
| | | GGTCGggccgGGCGGgccaggcgcTGGGCacGGTG |
| SEQ ID NO: 91 | 5UTR096 | AATCATCTTCttTACCCtggagCTGCTGCTGCTGctgctgcTTTT |
| | | GcTTTTGGGGCTgagtttAATAAGCGAGcgaGCGAGcaaGCGAGc |
| | | gcGGGGGgAAAAAGgcAGAGAATGtCCGCCATCTACCCTCcgctc |
| | | ctGGGCGcgCTCTCaTTCaTAGCAgccTCTTCATGAATTAcagct |
| | | gAGGGGgggcGGAGGAGGGGGGGGTAccacACAACACCCCAgcaa |
| | | aCCTCCgggcCCCCAggc |
| SEQ ID NO: 92 | 5UTR097 | AATCATCTTCttTACCCtggagCTGCTGCTGCTGctgctgcTTTT |
| | | GcTTTTGGGGCTgagtttAATAAGCGAGcgaGCGAGcaaGCGAGc |
| | | gcGGGGGgAAAAAGgcAGAGAATGtCCGCCATCTACCCTCcgctc |
| | | ctGGGCGcgCTCTCaTTCaTAGCAgccTCTTCTTGAATTAcagct |
| | | gAGGGGgggcGGAGGAGGGGgGGGTAccacACAACACCCCAgcaa |
| | | aCCTCCgggcCCCCAggc |
| SEQ ID NO: 93 | 5UTR098 | gCTCACACGcgcgcactCACACACACACACACAcacGGTGGaaGG |
| | | AGGcgaATAATAactcagccaTATTTcagCCGCCgCCGCCGGGAG |
| | | ctgcGGGCAcaGTCCGGGGAcgcgGCGAGcagcctcGGCGGccgc |
| | | aCCTCCgcaaagCGCCGcggccgctacg |
| SEQ ID NO: 94 | 5UTR099 | acTCTTgtcAGGGccgcggcaCATGGGCGGccggATGcgctgAGC |
| | | CCggcgctgcGGGGCcgcggagcgcTGGGGAgcagcggCCGCCgg |
| | | cgcggGGAGGgGGGTGGGGTGGGaCGGCGcaCCGCCtccGGTGct |
| | | ggcactAGGGGcTGGGGtCGGCGcGGTGTCTTCTGCCCTTCtgca |
| | | gccgtcgacaTTTTTTttTCTTTTTTTTTtcaATTTTGAAcATTT |
| | | TgCAAAAcgAGGGGTTCgaggcaggtGAGAGCATcctgcacgtCG |
| | | CCGgggAGCCCgcGGGCActtggcgcgCTCTCctGGGACcgtctg |
| | | cactggaAACCCGAAaGTTTTTTTTTAATatatatTTTTATGcag |
| | | ATGTATttatAAAGAtataagtaaTTTTTTTCTTCcCTTTTctcc |
| | | aCCGCCttgagaGCGAGtacTTTTGgcaaaGGACGGAGGAAAAGc |
| | | tcagcaacATTTTAGGGGgcggTTGTTTCTTTcTTATTtcTTTTT |
| | | TtaAGGGGAAAAAAtttgagtgCATcgcg |
| SEQ ID NO: 95 | 5UTR100 | actTTCCCGGTGcaCTTTTTctGGTGGgAGGGGagagcggagcag |
| | | gctcacgTGTAaccgcgcaggagCCTCCtctggcttgAGCCCttT |
| | | CTTgcATTTAgtAAAGAtaaAGACAAggAAAAGaagctggATGAT |
| | | GAGAGtaacAGCCCgacggtcCCCCAgtcggCATTccTGGGGcct |
| | | accttATGgGACAAAACCCTTCCCtATGacggagatactTTCcag |
| | | ttgGAAtac |
| SEQ ID NO: 96 | 5UTR101 | actTCTTTgggcctCATAAACAaccacaGAAccacaagttGGGTA |
| | | gcctggcagtgtcagaAGTCTgAACCCagcATAGTggtcagcagg |
| | | caGGACgAATCAcactgAATGcaaaccacaGGGTTtcgcagcgtg |
| | | gtAAAAGaAATCAttgagtCCCCCgcCTTCaGAAGAGGGTGcaTT |
| | | TTCAGGagGAAGcg |
| SEQ ID NO: 97 | 5UTR102 | actTCTTTgggcctCATAAACAaccacaGAAccacaagttGGGTA |
| | | gcctggcagtgtcagaAGTCTgAACCCagcATAGTggtcagcagg |
| | | caGGACgAATCAcacTGATTGcaaaccacaGGGTTtcgcagcgtg |
| | | gtAAAAGaAATCAttgagtCCCCCgcCTTCaGAAGAGGGTGcaTT |
| | | TTCAGGagGAAGcg |
| SEQ ID NO: 98 | 5UTR103 | ctagCTTTTCTCTTCtgtcAACCCcacacgcctttggcaca |
| SEQ ID NO: 99 | 5UTR104 | AGGGAcccgcagctcAGCTAcagcacagatcagCACC |
| SEQ ID NO: 100 | 5UTR105 | agcactgcctggctccacgtgCCTCCtggtctcagt |
| SEQ ID NO: 101 | 5UTR106 | actggAAAAGATAGTgaccttaccAGGGccaaagTTTGTagacac |
| | | aggAATTAcgaAATGgagaAGGGGgaGAAGtgAGCTAgtggcagC |
| | | ATAAAAAGaccagcagATGcCCCACagcactgcTCTTCcagaggc |
| | | AAGAccaaccaag |
| SEQ ID NO: 102 | 5UTR107 | aggcacagaCACCaaGGACAGAGAcgctggCTAGGCCGCCcTCCC |
| | | CACTGTTAccaac |
| SEQ ID NO: 103 | 5UTR108 | CTGCTGCAGGT |
| SEQ ID NO: 104 | 5UTR109 | ctcctcagCTTCaggcaCCACCactgacctGGGACagtGAAtcga |
| | | ca |
| SEQ ID NO: 105 | 5UTR110 | aGGCGGtcAGGGGaaggctcaGGAGGAGGGAgatCAACAtcaacc |
| | | tgCCCCGCCCCCTCCCCAgcctgATAAAgGTCCtgcGGGCAGGAC |
| | | aggaCCTCCcaaccaAGCCCTCCAGCAAggaTTCagagtgCCCCT |
| | | ccggcCTCGCc |
| SEQ ID NO: 106 | 5UTR111 | ctgctcagTTCATCCCtagaggcagctgctccagGAAcagaGGTG |
| | | cC |
| SEQ ID NO: 107 | 5UTR112 | gcagttCGGCGGTCCCGCGGGTCTGTCTCTTGCTTCAACAGTGTT |
| | | TGGACGGAACAgatccGGGGACTCTCTtccagCCTCCgaccgCCC |
| | | TCcgatTTCCTCTCcgcttgcaaCCTCCGGGACcaTCTTCtcggc |
| | | cATCTCCTgCTTCtGGGACctgccagCACCgtTTTTGtgGTTAGc |
| | | tcCTTCttgccaaccaacc |
| SEQ ID NO: 108 | 5UTR113 | ACTATaAATAGcagccacCTCTCcctggcagacAGGGAcccgcag |
| | | ctcAGCTAcagcacagatcagCACC |
| SEQ ID NO: 109 | 5UTR114 | AGGGAgGAAagtgaggaTTCCCtgccAAAATgcctGAGGGcTTCC |
| | | CtgcctaccacAGCCCtctGTGTTctTAAATCCTCCtgtctGAAc |
| | | agaggccAGACTctggtTTCcCCCACagcctgtctgtgtctGTCC |
| | | tctgcaaagcc |
| SEQ ID NO: 110 | 5UTR115 | aacGAActcCATctGGGATagcAATAAcctgtGAAa |
| SEQ ID NO: 111 | 5UTR116 | actggAAAAGATAGTgaccttaccAGGGccaaagTTTGTagacac |
| | | aggAATTAcGAATTGgagaAGGGGgaGAAGtgAGCTAgtggcagC |
| | | ATAAAAAGaccagcagAAGCCCcacagcacTCCTCTTCcagaggc |
| | | AAGAccaaccaag |
| SEQ ID NO: 112 | 5UTR117 | aatccttTCTTTcagctggagtgctcctcaggagccagcCCCACC |
| | | CtTAGAAaag |
| SEQ ID NO: 113 | 5UTR118 | gacagtgctgacacTACAaggctcggagctccGGGCActcagaCA |
| | | Tc |
| SEQ ID NO: 114 | 5UTR119 | GTCCtgtggcctctgcagctcagc |
| SEQ ID NO: 115 | 5UTR120 | AGGGAgGAAagtgaggaTTCCCtgccAAATTgcctGAGGGcTTCC |
| | | CtgcctaccacAGCCCtctGTGTTctTAAATCCTCCtgtctGAAc |
| | | agaggccAGACTctggtTTCcCCCACagcctgtctgtgtctGTCC |
| | | tctgcaaagcc |
| SEQ ID NO: 116 | 5UTR122 | ctTTCcggtacctgtgagtcagctAGGGGaGGGCAgCTCTCACCC |
| | | AggctgATAGTtcGGTGacctggcTTTATCTACTggATGagTTCc |
| | | gctGGGAG |
| SEQ ID NO: 117 | 5UTR123 | aactTTCcCCCCTCGGCGcCCCACcggCTCCCgcgcgcctCCCCT |
| | | cgCGCCCgagCTTCgagccaagcagcGTCCTGGGGAgcgcgtc |
| SEQ ID NO: 118 | 5UTR124 | aggtGTCCCGGGCGcgccacg |
| SEQ ID NO: 119 | 5UTR125 | CTTCctCCCTCATGccTCCCTtccTCTTaCTCTCattCATTtcAT |
| | | ACAcactggctcacacATCTACTCTCTCTCTCTatCTCTCTcaga |
| SEQ ID NO: 120 | 5UTR126 | gtTTCCCaagcaAGAGAGgttgTTGGGGAggcttgAGTCTgaCCT |
| | | CCtGTCCCttgcagCTTCtgtgCATatCCCCTtaCAAACAgATTA |
| | | GtCCCAGTCCatcacgagcagctggtttCTAAGATGcTATTTccc |
| | | gtATAAAGCATGagaccgtgacttgccAGCCCcacagAGCCCCGC |
| | | CCttGTCCatcactggCATctGGACtccagccTGGGTTGGGGcaa |
| | | agAGGGAAATGagatCATggcctAACCCtgatccTCTTgtCCCAC |
| | | a |
| SEQ ID NO: 121 | 5UTR127 | GGCTCTGAAAAAAAAAAAAAGACCCAAGCTAGCTAGCGTTTAAAC |
| | | TTAAGCTAGGTACCGAGACCaggtGTCCCGGGCGcgccacg |
| SEQ ID NO: 122 | 5UTR128 | aggtGTCCCGGGCGcgccacGGGCTCTGAAAAAAAAAAAAAGACC |
| | | CAAGCTAGCTAGCGTTTAAACTTAAGCTAGGTACCGAGACC |
| SEQ ID NO: 123 | 5UTR129 | ctTTCcggtacctgtgagtcagctAGGGGaGGGCAgCTCTCACCC |
| | | AggctgATAGTtcGGTGacctggcTTTATCTACTggATCagTTCc |
| | | gctGGGAG |

TABLE 2
3′ UTRs

| SEQ ID NO: | Registry ID | Sequence |
|---|---|---|
| SEQ ID NO: 124 | 3UTR001 | GCTCGCTTTCTTGCTGTCCAATTTCTATTAAAGGTTCCTTTGTTCCCTAA |
| | | GTCCAACTACTAAACTGGGGGATATTATGAAGGGCCTTGAGCATCTGGAT |
| | | TCTGCCTAATAAAAAACATTTATTTTCATTGCAA |
| SEQ ID NO: 125 | 3UTR002 | GCGGACTGTTACTGAGCTGCGTTTTACACCCTTTCTTTGACAAAACCTAA |
| | | CTTGCGCAGAAAAAAAAAAAATAAGAGACAACATTGGCATGGCTTTGTTT |
| | | TTTTAAATTTTTTTTAAAGTTTTTTTTTTTTTTTTTTTTTTTTTTTTTAA |
| | | GTTTTTTTGTTTTGTTTTGGCGCTTTTGACTCAGGATTTAAAAACTGGAA |
| | | CGGTGAAGGCGACAGCAGTTGGTTGGAGCAAACATCCCCCAAAGTTCTAC |
| | | AAATGTGGCTGAGGACTTTGTACATTGTTTTGTTTTTTTTTTTTTTTGGT |
| | | TTTGTCTTTTTTTAATAGTCATTCCAAGTATCCATGAAATAAGTGGTTAC |
| | | AGGAAGTCCCTCACCCTCCCAAAAGCCACCCCCACTCCTAAGAGGAGGAT |
| | | GGTCGCGTCCATGCCCTGAGTCCACCCCGGGGAAGGTGACAGCATTGCTT |
| | | CTGTGTAAATTATGTACTGCAAAAATTTTTTTAAATCTTCCGCCTTAATA |
| | | CTTCATTTTTGTTTTTAATTTCTGAATGGCCCAGGTCTGAGGCCTCCCTT |
| | | TTTTTTGTCCCCCCAACTTGATGTATGAAGGCTTTGGTCTCCCTGGGAGG |
| | | GGGTTGAGGTGTTGAGGCAGCCAGGGCTGGCCTGTACACTGACTTGAGAC |
| | | CAATAAAAGTGCACACCTTACCTTACACAAAC |
| SEQ ID NO: 126 | 3UTR003 | gtcttagcaagctctgagccaggagatggacataaaccatagcaatccaa |
| | | cgtgtaaccgcaatggggcaaacaacaggtgaaccgtgtccacgggcctg |
| | | gttaccgaaaggaaagccagtatccaacacagcaatgtgttgggggtcac |
| | | accttcggggtactcttaacgctgacactcgaaagagcagttcggcaacc |
| | | c |
| SEQ ID NO: 127 | 3UTR004 | atgaactcaatctaaattaaaaaagaaagaaatttgaaaaaactttctct |
| | | ttgccatttcttcttcttcttttttaactgaaagctgaatccttccattt |
| | | cttctgcacatctacttgcttaaattgtgggcaaaagagaaaaagaagga |
| | | ttgatcagagcattgtgcaatacagtttcattaactccttcccccgctcc |
| | | cccaaaaatttgaatttttttttcaacactcttacacctgttatggaaaa |
| | | tgtcaacctttgtaagaaaaccaaaataaaaattgaaaaataaaaaccat |
| | | aaacatttgcaccacttgtggcttttgaatatcttccacagagggaagtt |
| | | taaaacccaaacttccaaaggtttaaactacctcaaaacactttcccatg |
| | | agtgtgatccacattgttaggtgctgacctagacagagatgaactgaggt |
| | | ccttgttttgttttgttcataatacaaaggtgctaattaatagtatttca |
| | | gatacttgaagaatgttgatggtgctagaagaatttgagaagaaatactc |
| | | ctgtattgagttgtatcgtgtggtgtattttttaaaaaatttgatttagc |
| | | attcatattttccatcttattcccaattaaaagtatgcagattatttgcc |
| | | caaatcttcttcagattcagcatttgttctttgccagtctcattttcatc |
| | | ttcttccatggttccacagaagctttgtttcttgggcaagcagaaaaatt |
| | | aaattgtacctattttgtatatgtgagatgtttaaataaattgtgaaaaa |
| | | aatgaaataaagcatgtttggttttccaaaagaacatat |
| SEQ ID NO: 128 | 3UTR005 | GGTGCCTTTGAGAGTCTACTTTTGCTCTCTTCGGAAGAACCCTTAGGGGT |
| | | TCGTGCATGGGCTTGCATAGCAAGTCTAGATGCGGGTACCGTACAGTGTT |
| | | GAAAAACACTGTAAATCTCTAAAAGAGACCA |
| SEQ ID NO: 129 | 3UTR006 | gaccacacaaggcagatgggctatataaacgttttcgcttttccgtttac |
| | | gatatatagtctactcttgtgcagaatgaattctcgtaactacatagcac |
| | | aagtagatgtagttaactttaatctcacatagcaatctttaatcagtgtg |
| | | taacattagggaggacttgaaagagccaccacattttcaccgaggccacg |
| | | cggagtacgatcgagtgtacagtgaacaatgctagggagagctgcctata |
| | | tggaagagccctaatgtgtaaaattaattttagtagtgctatccccatgt |
| | | gattttaatagcttcttaggagaatgac |
| SEQ ID NO: 130 | 3UTR007 | CTAACTATTTGCTTTGTATTTTAAGATTTTGTAAATAGAAAAATATATAA |
| | | CCCCACTCGTAGGTAAGGATTTATTGTATATTTTATTTAGTTAGTTATTC |
| | | AGTACTTACGGCCCTATTACCAACGGGTATTAATCACAAACACTTTATCC |
| | | CCATAGGATTCTTTTAAATTTAAAATTTTAAATAATTAACGTCAGAGTCC |
| | | CATCGGGGCTAACAGGTTTTTCGCACTTTTCCTGCTAACTGACAGAAGTG |
| | | CAATTTGGTTTTTGATTAATAGTTGTTTTCT |
| SEQ ID NO: 131 | 3UTR008 | cctcgccccggacctgccctcccgccaggtgcacccacctgcaataaatg |
| | | cagcgaagccggga |
| SEQ ID NO: 132 | 3UTR009 | cctcgccccggacctgccctcccgccaggtgcacccacctgcaataaatg |
| | | cagcgaagccgggacctcgccccggacctgccctcccgccaggtgcaccc |
| | | acctgcaataaatgc |
| SEQ ID NO: 133 | 3UTR010 | aaagcaaaactaacatgaaacaaggctagaagtcaggtcggattaagcca |
| | | tagtacggaaaaaactatgctacctgtgagccccgtccaaggacgttaaa |
| | | agaagtcaggccatcataaatgccatagcttgagtaaactatgcagcctg |
| | | tagctccacctgagaaggtgtaaaaaatccgggaggccacaaaccatgga |
| | | agctgtacgcatggcgtagtggactagcggttagaggagacccctccctt |
| | | acaaatcgcagcaacaatgggggcccaaggcgagatgaagctgtagtctc |
| | | gctggaaggactagaggttagaggagacccccccgaaacaaaaaacagca |
| | | tattgacgctgggaaagaccagagatcctgctgtctcctcagcatcattc |
| | | caggcacagaacgccagaaaatggaatggtgctgttgaatcaacaggttc |
| | | t |
| SEQ ID NO: 134 | 3UTR011 | GGCAGCAGCGGAGGTCATGAAGGTTTTTCTTTTCCTGAGAAAACAACACG |
| | | TATTGTTTTCTCAGGTTTTGCTTTTTGGCCTTTTTCTAGCTTAAAAAAAA |
| | | AAAAAGCAAAAGATGCTGGTGGTTGGCACTCCTGGTTTCCAGGACGGGGT |
| | | TCAAATCCCTGCGGCGTCTTTGCTT |
| SEQ ID NO: 135 | 3UTR012 | gattcgtcagtagggttgtaaaggtttttcttttcctgagaaaacaacct |
| | | tttgttttctcaggttttgctttttggcctttccctagctttaaaaaaaa |
| | | aaaagcaaaagacgctggtggctggcactcctggtttccaggacggggtt |
| | | caagtccctgcggtgtctttgctt |
| SEQ ID NO: 136 | 3UTR013 | GCGGACTATGACTTAGTTGCGTTACACCCTTTCTTGACAAAACCTAACTT |
| | | GCGCAGAAAACAAGATGAGATTGGCATGGCTTTATTTGTTTTTTTTGTTT |
| | | TGTTTTGGTTTTTTTTTTTTTTTTGGCTTGACTCAGGATTTAAAAACTGG |
| | | AACGGTGAAGGTGACAGCAGTCGGTTGGAGCGAGCATCCCCCAAAGTTCA |
| | | CAATGTGGCCGAGGACTTTGATTGCACATTGTTGTTTTTTTAATAGTCAT |
| | | TCCAAATATGAGATGCGTTGTTACAGGAAGTCCCTTGCCATCCTAAAAGC |
| | | CACCCCACTTCTCTCTAAGGAGAATGGCCCAGTCCTCTCCCAAGTCCACA |
| | | CAGGGGAGGTGATAGCATTGCTTTCGTGTAAATTATGTAATGCAAAATTT |
| | | TTTTAATCTTCGCCTTAATACTTTTTTATTTTGTTTTATTTTGAATGATG |
| | | AGCCTTCGTGCCCCCCCTTCCCCCTTTTTTGTCCCCCAACTTGAGATGTA |
| | | TGAAGGCTTTTGGTCTCCCTGGGAGTGGGTGGAGGCAGCCAGGGCTTACC |
| | | TGTACACTGACTTGAGACCAGTTGAATAAAAGTGCACACCTTAAAAATGA |
| SEQ ID NO: 137 | 3UTR014 | GCTGGAGCCTCGGTGGCCATGCTTCTTGCCCCTTGGGCCTCCCCCCAGCC |
| | | CCTCCTCCCCTTCCTGCACCCGTACCCCCGTGGTCTTTGAATAAAGTCTG |
| | | AGTGGGCGGCA |
| SEQ ID NO: 138 | 3UTR015 | GCTCGCTTTCTTGCTGTCCAATTTCTATTAAAGGTTCCTTTGTTCCCTAA |
| | | GTCCAACTACTAAACTGGGGGATATTATGAAGGGCCTTGAGCATCTGGAT |
| | | TCTGCCTAATAAAAAACATTTATTTTCATTGCAATTGCCATGTGTATGTG |
| | | GGTTCGCCCACATACTCTGATGATCCCCAATCGTGGCGTGTCGGCCTGCT |
| | | TCGGCAGGCACTGGCGCCGGGATCATTCATGGCAA |
| SEQ ID NO: 139 | 3UTR016 | GCTCGCTTTCTTGCTGTCCAATTTCTATTAAAGGTTCCTTTGTTCCCTAA |
| | | GTCCAACTACTAAACTGGGGGATATTATGAAGGGCCTTGAGCATCTGGAT |
| | | TCTGCCTAATAAAAAACATTTATTTTCATTGCAAGCTCGCTTTCTTGCTG |
| | | TCCAATTTCTATTAAAGGTTCCTTTGTTCCCTAAGTCCAACTACTAAACT |
| | | GGGGGATATTATGAAGGGCCTTGAGCATCTGGATTCTGCCTAATAAAAAA |
| | | CATTTATTTTCATTGCAA |
| SEQ ID NO: 140 | 3UTR017 | TCGACAATCAACCTCTGGATTACAAAATTTGTGAAAGATTGACTGGTATT |
| | | CTTAACTATGTTGCTCCTTTTACGCTATGTGGATACGCTGCTTTAATGCC |
| | | TTTGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGTCAGG |
| | | CAACGTGGCGTGGTGTGCACTGTGTTTGCTGACGCAACCCCCACTGGTTG |
| | | GGGCATTGCCACCACCTGTCAGCTCCTTTCCGGGACTTTCGCTTTCCCCC |
| | | TCCCTATTGCCACGGCGGAACTCATCGCCGCCTGCCTTGCCCGCTGCTGG |
| | | ACAGGGGCTCGGCTGTTGGGCACTGACAATTCCGTGGTGTTGTCGGGGAA |
| | | GCTGACGTCCTTTCCATGGCTGCTCGCCTGTGTTGCCACCTGGATTCTGC |
| | | GCGGGACGTCCTTCTGCTACGTCCCTTCGGCCCTCAATCCAGCGGACCTT |
| | | CCTTCCCGCGGCCTGCTGCCGGCTCTGCGGCCTCTTCCGCGTCTTCGCCT |
| | | TCGCCCTCAGACGAGTCGGATCTCCCTTTGGGCCGCCTCCCCGCCTG |
| SEQ ID NO: 141 | 3UTR018 | GGCCAAAGGCGTCGAGTAGACGCCAACAACGGAATTGCGGGAAAGGGGTC |
| | | AACAGCCGTTCAGTACCAAGTCTCAGGGGAAACTTTGAGATGGCCTTGCA |
| | | AAGGGTATGGTAATAAGCTGACGGACATGGTCCTAACCACGCAGCCAAGT |
| | | CCTAAGTCAACAGATCTTCTGTTGATATGGATGCAGTTCAAAACCAAACC |
| | | GTCAGCGAGTAGCTGACAAAAAGAAACAACAACAACAAC |
| SEQ ID NO: 142 | 3UTR019 | tacggtaatagtgtagtcttctcatcttagtagttagctctctcttatat |
| | | taagaaaagaaaacaaaaacccccaggtcgctttattttgacctgtgtta |
| | | gggaccaaaaacggtggcagcactgtctagctgcgggcattagactggaa |
| | | aactagtgctctttgggtaaccactaaaatcccgaaagggtgggctgtgg |
| | | tgaccttccgaactaaaagatagcctccctcctcgcgcgggggggggggg |
| | | gggcctgccc |
| SEQ ID NO: 143 | 3UTR020 | TTTAACACCCTTCAGGTGTAGACCCGTCATTGTGACGCGTGGGTTGAGGT |
| | | GCCATGAATTTGTCATTCATGGTGCATTTATCTCAACAGTTTTCCCTAAC |
| | | CGCGCGTTGCGCGGCAGGGTTTTTACTCTGAGAGATAAATGCCTGCTCAC |
| | | TAAGGTCTATTAGAGACATTAGTACGATCCGGCTAATAGTCGCTTTGGAT |
| | | GACCTCCAAAGCGGCGGATTCCT |
| SEQ ID NO: 144 | 3UTR021 | CCGCTACGCCCCAATGATCCGACCAGCAAAACTCGATGTACTTCCGAGGA |
| | | ACTGATGTGCATAATGCATCAGGCTGGTACATTAGATCCCCGCTTACCGC |
| | | GGGCAATATAGCAACACTAAAAACTCGATGTACTTCCGAGGAAGCGCAGT |
| | | GCATAATGCTGCGCAGTGTTGCCACATAACCACTATATTAACCATTTATC |
| | | TAGCGGACGCCAAAAACTCAATGTATTTCTGAGGAAGCGTGGTGCATAAT |
| | | GCCACGCAGCGTCTGCATAACTTTTATTATTTCTTTTATTAATCAACAAA |
| | | ATTTTGTTTTTAACATTTC |
| SEQ ID NO: 145 | 3UTR022 | CGTCTGCATAACTTTTATTATTTCTTTTATTAATCAACAAAATTTTGTTT |
| | | TTAACATTTC |
| SEQ ID NO: 146 | 3UTR023 | taaccctacctcagtcgaattggattgggtcatactgttgtaggggtaaa |
| | | tttttctttaattcggag |
| SEQ ID NO: 147 | 3UTR024 | aggccggtcatccttttgacacttcaagtcccgaggataacctcctctcg |
| | | gggttggggggaatcttgggatccagtagtcctccttgaactccatccaa |
| | | cagggtagatttaagagtcatgagactttcattaatcatctcagttgatc |
| | | agacatggtcgtgtagattctcataacacgggagatcttctagcagtttc |
| | | agtgaccaacggtgctttccttctccaggaactgataccgaagttgttgg |
| | | acaagccaaggggtgcttcggattactctgtgcttgggcacagaaagagg |
| | | tcgtagtttgccccttgatagcagattcaacatgaattaactaagaaagg |
| | | cgatctgcctcccatgaaggacataagcaatagttcacaatcatcttgca |
| | | tctcagtgaagtgtacataactataaagggctgggtcatctaagcatttc |
| | | agtcgag |
| SEQ ID NO: 148 | 3UTR027 | acattactaatttgaatggaaaacacatggtgtgagtccaaagaaggtgt |
| | | tttcctgaagaactgtctattttctcagtcatttttaacctctagagtca |
| | | ctgatacacagaatataatcttatttatacctcagtttgcatattttttt |
| | | actatttagaatgtagccctttttgtactgatataatttagttccacaaa |
| | | tggtgggtacaaaaagtcaagtttgtggcttatggattcatataggccag |
| | | agttgcaaagatcttttccagagtatgcaactctgacgttgatcccagag |
| | | agcagcttcagtgacaaacatatcctttcaagacagaaagagacaggaga |
| | | catgagtctttgccggaggaaaagcagctcaagaacacatgtgcagtcac |
| | | tggtgtcaccctggataggcaagggataactcttctaacacaaaataagt |
| | | gttttatgtttggaataaagtcaaccttgtttctactgtttta |
| SEQ ID NO: 149 | 3UTR028 | GCTCGCTTTCTTGCTGTCCAATTTCTATTAAAGGTTCCTTTGTTCCCTAA |
| | | GTCCAACTACTAAACTGGGGGATATTATGAAGGGCCTTGAGCATCTGGAT |
| | | TCTGCCTAATAAAAAACATTTATTTTCATTGC |
| SEQ ID NO: 150 | 3UTR031 | GCTCGCTTTCTTGCTGTCCAATTTCTGGTTCCTTTGTTCCCTAAGTCCAA |
| | | CTACTAAACTGGGGGATATTATGAAGGGCCTTGAGCATCTGGATTCTGCC |
| | | TAATAAAAAACATTTATTTTCATTGCAA |
| SEQ ID NO: 151 | 3UTR032 | GCGGACTGTTACTGAGCTGCGTTTTACACCCTTTCTTTGACAAAACCTAA |
| | | CTTGCGCAGAAAAAAAAAAAATAAGAGACAACATTGGCATGGCTTTGTTT |
| | | TTTTAAATTTTTTTTAAAGTTTTTTTTTTTTTTTTTTTTTTTTTTTTTAA |
| | | GTTTTTTTGTTTTGTTTTGGCGCTTTTGACTCAGGATTTAAAAACTGGAA |
| | | CGGTGAAGGCGACAGCAGTTGGTTGGAGCAAACATCCCCCAAAGTTCTAC |
| | | AAATGTGGCTGAGGACTTTGTACATTGTTTTGTTTTTTTTTTTTTTTGGT |
| | | TTTGTCTTTTAGTCATTCCAAGTATCCATGAAATAAGTGGTTACAGGAAG |
| | | TCCCTCACCCTCCCAAAAGCCACCCCCACTCCTAAGAGGAGGATGGTCGC |
| | | GTCCATGCCCTGAGTCCACCCCGGGGAAGGTGACAGCATTGCTTCTGTGT |
| | | AAATTATGTACTGCAAAAATTTTTTTAAATCTTCCGCCTTAATACTTCAT |
| | | TTTTGTTTTCTGAATGGCCCAGGTCTGAGGCCTCCCTTTTTTTTGTCCCC |
| | | CCAACTTGATGTATGAAGGCTTTGGTCTCCCTGGGAGGGGGTTGAGGTGT |
| | | TGAGGCAGCCAGGGCTGGCCTGTACACTGACTTGAGACCAGTGCACACCT |
| | | TACCTTACACAAAC |
| SEQ ID NO: 152 | 3UTR033 | atgaactcaatctaaaaagaaagaaatttgaaaaaactttctctttgcca |
| | | tttcttcttcttcttttttaactgaaagctgaatccttccatttcttctg |
| | | cacatctacttgcttaaattgtgggcaaaagagaaaaagaaggattgatc |
| | | agagcattgtgcaatacagtttcattaactccttcccccgctcccccaaa |
| | | aatttgaatttttttttcaacactcttacacctgttatggaaaatgtcaa |
| | | cctttgtaagaaaaccaaaattgaaaaaccataaacatttgcaccacttg |
| | | tggcttttgaatatcttccacagagggaagtttaaaacccaaacttccaa |
| | | aggtttaaactacctcaaaacactttcccatgagtgtgatccacattgtt |
| | | aggtgctgacctagacagagatgaactgaggtccttgttttgttttgttc |
| | | ataatacaaaggtgctaattaatagtatttcagatacttgaagaatgttg |
| | | atggtgctagaagaatttgagaagaaatactcctgtattgagttgtatcg |
| | | tgtggtgtattttttaaaaaatttgatttagcattcatattttccatctt |
| | | attcccaagtatgcagattatttgcccaaatcttcttcagattcagcatt |
| | | tgttctttgccagtctcattttcatcttcttccatggttccacagaagct |
| | | ttgtttcttgggcaagcagaaaattgtacctattttgtatatgtgagatg |
| | | gtgaaaaaaatgagcatgtttggttttccaaaagaacatat |
| SEQ ID NO: 153 | 3UTR034 | gaccacacaaggcagatgggctatataaacgttttcgcttttccgtttac |
| | | gatatatagtctactcttgtgcagaatgaattctcgtaactacatagcac |
| | | aagtagatgtagttaacctcacatagcaatccagtgtgtaacattaggga |
| | | ggacttgaaagagccaccacattttcaccgaggccacgcggagtacgatc |
| | | gagtgtacagtgaacaatgctagggagagctgcctatatggaagagccct |
| | | aatgtggtgctatccccatgtgatagcttcttaggagaatgac |
| SEQ ID NO: 154 | 3UTR035 | CTAACTATTTGCTTTGTATTTTAAGATTTTGTAAATAGAAAAATATATAA |
| | | CCCCACTCGTAGGTAAGGAGTATATTAGTTAGTTATTCAGTACTTACGGC |
| | | CCTATTACCAACGGGTATTAATCACAAACACTTTATCCCCATAGGATTCT |
| | | TTTAAATTTAAAATTTTAAATAATTAACGTCAGAGTCCCATCGGGGCTAA |
| | | CAGGTTTTTCGCACTTTTCCTGCTAACTGACAGAAGTGCAATTTGGTTTT |
| | | TGATTAATAGTTGTTTTCT |
| SEQ ID NO: 155 | 3UTR036 | cctcgccccggacctgccctcccgccaggtgcacccacctgcgcagcgaa |
| | | gccggga |
| SEQ ID NO: 156 | 3UTR037 | cctcgccccggacctgccctcccgccaggtgcacccacctgctgcagcga |
| | | agccgggacctcgccccggacctgccctcccgccaggtgcacccacctgc |
| | | tgc |
| SEQ ID NO: 157 | 3UTR038 | GCGGACTATGACTTAGTTGCGTTACACCCTTTCTTGACAAAACCTAACTT |
| | | GCGCAGAAAACAAGATGAGATTGGCATGGCTGTTTTTTTTGTTTTGTTTT |
| | | GGTTTTTTTTTTTTTTTTGGCTTGACTCAGGATTTAAAAACTGGAACGGT |
| | | GAAGGTGACAGCAGTCGGTTGGAGCGAGCATCCCCCAAAGTTCACAATGT |
| | | GGCCGAGGACTTTGATTGCACATTGTTGTTTTAGTCATTCCAAATATGAG |
| | | ATGCGTTGTTACAGGAAGTCCCTTGCCATCCTAAAAGCCACCCCACTTCT |
| | | CTCTAAGGAGAATGGCCCAGTCCTCTCCCAAGTCCACACAGGGGAGGTGA |
| | | TAGCATTGCTTTCGTGTAAATTATGTAATGCAAAATTTTCTTCGCCTTAA |
| | | TACTTTTTGTTTGAATGATGAGCCTTCGTGCCCCCCCTTCCCCCTTTTTT |
| | | GTCCCCCAACTTGAGATGTATGAAGGCTTTTGGTCTCCCTGGGAGTGGGT |
| | | GGAGGCAGCCAGGGCTTACCTGTACACTGACTTGAGACCAGTTGAGTGCA |
| | | CACCTTAAAAATGA |
| SEQ ID NO: 158 | 3UTR039 | GCTGGAGCCTCGGTGGCCATGCTTCTTGCCCCTTGGGCCTCCCCCCAGCC |
| | | CCTCCTCCCCTTCCTGCACCCGTACCCCCGTGGTCTTTGGTCTGAGTGGG |
| | | CGGCA |
| SEQ ID NO: 159 | 3UTR040 | GCTCGCTTTCTTGCTGTCCAATTTCTGGTTCCTTTGTTCCCTAAGTCCAA |
| | | CTACTAAACTGGGGGATATTATGAAGGGCCTTGAGCATCTGGATTCTGCC |
| | | TAAACATTCATTGCAATTGCCATGTGTATGTGGGTTCGCCCACATACTCT |
| | | GATGATCCCCAATCGTGGCGTGTCGGCCTGCTTCGGCAGGCACTGGCGCC |
| | | GGGATCATTCATGGCAA |
| SEQ ID NO: 160 | 3UTR041 | GCTCGCTTTCTTGCTGTCCAATTTCTGGTTCCTTTGTTCCCTAAGTCCAA |
| | | CTACTAAACTGGGGGATATTATGAAGGGCCTTGAGCATCTGGATTCTGCC |
| | | TAAACATTCATTGCAAGCTCGCTTTCTTGCTGTCCAATTTCTGGTTCCTT |
| | | TGTTCCCTAAGTCCAACTACTAAACTGGGGGATATTATGAAGGGCCTTGA |
| | | GCATCTGGATTCTGCCTAAACATTCATTGCAA |
| SEQ ID NO: 161 | 3UTR042 | TCGACAATCAACCTCTGGATTACAAAATTTGTGAAAGATTGACTGGTATT |
| | | CTTAACTATGTTGCTCCTTTTACGCTATGTGGATACGCTGCGCCTTTGTA |
| | | TCATGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCAACGT |
| | | GGCGTGGTGTGCACTGTGTTTGCTGACGCAACCCCCACTGGTTGGGGCAT |
| | | TGCCACCACCTGTCAGCTCCTTTCCGGGACTTTCGCTTTCCCCCTCCCTA |
| | | TTGCCACGGCGGAACTCATCGCCGCCTGCCTTGCCCGCTGCTGGACAGGG |
| | | GCTCGGCTGTTGGGCACTGACAATTCCGTGGTGTTGTCGGGGAAGCTGAC |
| | | GTCCTTTCCATGGCTGCTCGCCTGTGTTGCCACCTGGATTCTGCGCGGGA |
| | | CGTCCTTCTGCTACGTCCCTTCGGCCCTCAATCCAGCGGACCTTCCTTCC |
| | | CGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCCTG |
| SEQ ID NO: 162 | 3UTR043 | GGCCAAAGGCGTCGAGTAGACGCCAACAACGGAATTGCGGGAAAGGGGTC |
| | | AACAGCCGTTCAGTACCAAGTCTCAGGGGAAACTTTGAGATGGCCTTGCA |
| | | AAGGGTATGGGCTGACGGACATGGTCCTAACCACGCAGCCAAGTCCTAAG |
| | | TCAACAGATCTTCTGTTGATATGGATGCAGTTCAAAACCAAACCGTCAGC |
| | | GAGTAGCTGACAAAAAGAAACAACAACAACAAC |
| SEQ ID NO: 163 | 3UTR044 | GGCCAAAGGCGTCGAGTAGACGCCAACAACGGAATTGCGGGAAAGGGGTC |
| | | AACAGCCGTTCAGTACCAAGTCTCAGGGGAAACTTTGAGATGGCCTTGCA |
| | | AAGGGTATGGGCTGACGGACATGGTCCTAACCACGCAGCCAAGTCCTAAG |
| | | TCAACAGATCTTCTGTTGATATGGATGCAGCCGTCAGCGAGTAGCTGAC |
| SEQ ID NO: 164 | 3UTR045 | tacggtaatagtgtagtcttctcatcttagtagttagctctctcttatat |
| | | taagaaaagaaaacaaaaacccccaggtcgcttgacctgtgttagggacc |
| | | aaaaacggtggcagcactgtctagctgcgggcattagactggaaaactag |
| | | tgctctttgggtaaccactaaaatcccgaaaggggggctgtggtgacctt |
| | | ccgaactaaaagatagcctccctcctcgcgcggggggggggcctgccc |
| SEQ ID NO: 165 | 3UTR046 | tacggtaatagtgtagtcttctcatcttagtagttagctctctcccccca |
| | | ggtcgcttgacctgtgttagggacccggtggcagcactgtctagctgcgg |
| | | gcattagactggctagtgctctttgggtaaccacttcccgaaaggggggc |
| | | tgtggtgaccttccgaactgatagcctccctcctcgcgcggggggcctgc |
| | | cc |
| SEQ ID NO: 166 | 3UTR047 | tacggtaatagtgtagtcttctcatcttagtagttagctctctcccccca |
| | | ggtcgcttgacctgtgttagggacccggtggcagcactgtctagctgcgg |
| | | gcattagactggctagtgctctttgggtaaccacttcccgaaaggggggc |
| | | tgtggtgaccttccgaactgata |
| SEQ ID NO: 167 | 3UTR048 | CCGCTACGCCCCAATGATCCGACCAGCAAAACTCGATGTACTTCCGAGGA |
| | | ACTGATGTGCATAATGCATCAGGCTGGTACATTAGATCCCCGCTTACCGC |
| | | GGGCAATATAGCAACACTAAAAACTCGATGTACTTCCGAGGAAGCGCAGT |
| | | GCATAATGCTGCGCAGTGTTGCCACATAACCACTATATTAACCATTTATC |
| | | TAGCGGACGCCAAAAACTCAATGTATTTCTGAGGAAGCGTGGTGCATAAT |
| | | GCCACGCAGCGTCTGCATAACCAACAAAATTTTGTTTTTAACATTTC |
| SEQ ID NO: 168 | 3UTR049 | CCGCTACGCCCCAATGATCCGACCAGCAAAACTCGATGTACTTCCGAGGA |
| | | ACTGATGTGCATAATGCATCAGGCTGGTACATTAGATCCCCGCTTACCGC |
| | | GGGCAATATAGCAACACTAAAAACTCGATGTACTTCCGAGGAAGCGCAGT |
| | | GCATAATGCTGCGCAGTGTTGCCACATAACCACTATATTAACCATTTATC |
| | | TAGCGGACGCCAAAAACTCAATGTATTTCTGAGGAAGCGTGGTGCATAAT |
| | | GCCACGCAGCGTCTGC |
| SEQ ID NO: 169 | 3UTR050 | CCGCTACGCCCCAATGATCCGACCAGCCTCGATGTACTTCCGAGGAACTG |
| | | ATGTGCATAATGCATCAGGCTGGTACATTAGATCCCCGCTTACCGCGGGC |
| | | AATATAGCAACACCTCGATGTACTTCCGAGGAAGCGCAGTGCATAATGCT |
| | | GCGCAGTGTTGCCACATAACCACTATATTAACCCTAGCGGACGCCCTCAA |
| | | TGCTGAGGAAGCGTGGTGCATAATGCCACGCAGCGTCTGC |
| SEQ ID NO: 170 | 3UTR051 | CGTCTGCATAACATTTCTAATCAACAAAATTTTGTTTTTAACATTTC |
| SEQ ID NO: 171 | 3UTR052 | taaccctacctcagtcgaattggattgggtcatactgttgtaggggtaaa |
| | | tttttctcggag |
| SEQ ID NO: 172 | 3UTR053 | taaccctacctcagtcgaattggattgggtcatactgttgtaggggctcg |
| | | gag |
| SEQ ID NO: 173 | 3UTR058 | acattacggaaaacacatggtgtgagtccaaagaaggtgttttcctgaag |
| | | aactgtctattttctcagtcattctctagagtcactgatacacagcctca |
| | | gtttgcgaatgtagccctttttgtactgccacaaatggtgggtacaaaaa |
| | | gtcaagtttgtggcttatggattcatataggccagagttgcaaagatctt |
| | | ttccagagtatgcaactctgacgttgatcccagagagcagcttcagtgac |
| | | aaacatatcctttcaagacagaaagagacaggagacatgagtctttgccg |
| | | gaggaaaagcagctcaagaacacatgtgcagtcactggtgtcaccctgga |
| | | taggcaagggataactcttctaacacaaaataagtggtcaaccttgtttc |
| | | tactgtttta |
| SEQ ID NO: 174 | 3UTR059 | GCTCGCTTTCTTGCTGTCCCGGTTCCTTTGTTCCCTAAGTCCAACCTAAG |
| | | GGCCTTGAGCATCTGGATTCTGCC |
| SEQ ID NO: 175 | 3UTR064 | GCTCGCTTTCTTGCTGTCCATCTGGTTCCTTTGTTCCCTAAGTCCAACCT |
| | | GGGGGATGAAGGGCCTTGAGCATCTGGATTCTGCCTGCAA |
| SEQ ID NO: 176 | 3UTR065 | GCGGACTGTTACTGAGCTGCGCACCCTTTCTTTGACAACCTAACTTGCGC |
| | | AGTAAGAGACAACATTGGCATGGCTTTGGGCGCTTGACTCAGGAACTGGA |
| | | ACGGTGAAGGCGACAGCAGTTGGTTGGAGCAAACATCCCCCAAAGTTCTA |
| | | CAAATGTGGCTGAGGACTTTGTACATTGTAGTCATTCCAAGTATCCATGA |
| | | AATAAGTGGTTACAGGAAGTCCCTCACCCTCCCAAAGCCACCCCCACTCC |
| | | TAAGAGGAGGATGGTCGCGTCCATGCCCTGAGTCCACCCCGGGGAAGGTG |
| | | ACAGCATTGCTTCTGTGTAAATTATGTACTGCATCTTCCGCCTCTGAATG |
| | | GCCCAGGTCTGAGGCCTCCCTTGTCCCCCCAACTTGATGTATGAAGGCTT |
| | | TGGTCTCCCTGGGAGGGGGTTGAGGTGTTGAGGCAGCCAGGGCTGGCCTG |
| | | TACACTGACTTGAGACCAGTGCACACCTTACCTTACACAAAC |
| SEQ ID NO: 177 | 3UTR066 | GCGGACTGTTACTGAGCTGCGCACCCTTTCTTTGACAACCTAACTTGCGC |
| | | AGTAAGAGACAACATTGGCATGGCTTTGGGCGCTTGACTCAGGAACTGGA |
| | | ACGGTGAAGGCGACAGCAGTTGGTTGGAGCAAACATCCCCCAAAGTTCTA |
| | | CAAATGTGGCTGAGGACTTTGTACATTGTAGTCATTCCAAGTATCCATGA |
| | | AATAAGTGGTTACAGGAAGTCCCTCACCCTCCCAAAGCCACCCCCACTCC |
| | | TAAGAGGAGGATGGTCGCGTCCATGCCCTGAGTCCACCCCGGGGAAGGTG |
| | | ACAGCATTGCTTCTGTGTAAATTATGTACTGCATCTTCCGCCTCTGAATG |
| | | GCCCAGGTCTGAGGCCTCCCTTGTCCCCCCAACTTGATGTATGAAGGCTT |
| | | TGGTCTCCCTGGGAGGGGGTTGAGGTGTTGAGGTGCACACCTTACCTTAC |
| | | ACAAAC |
| SEQ ID NO: 178 | 3UTR067 | GCGGACGCACCCTTTCTTTGACAACCTAACTTGCGCAGTAAGAGACAACA |
| | | TTGGCATGGCTTTGGGCGCTTGACTCAGGAACTGGAACGGTGAAGGCGAC |
| | | AGCAGTTGGTTGGAGCAAACATCCCCCAAAGTTCTACAAATGTGGCTGAG |
| | | GACTTTGTACATTGTAGTCATTCCAAGTATCCATGAAATAAGTGGTTACA |
| | | GGAAGTCCCTCACCCTCCCAAAGCCACCCCCACTCCTAAGAGGAGGATGG |
| | | TCGCGTCCATGCCCTGAGTCCACCCCGGGGAAGGTGACAGCATTGCTTCT |
| | | GTGTAAATTATGTACTGCATCTTCCGCCTCTGAATGGCCCAGGTCTGAGG |
| | | CCTCCCTTGTCCCCCCAACTTGATGTATGAAGGCTTTGGTCTCCCTGGGA |
| | | GGGGGTTGAGGTGTTGAGGTGCACACCTTACCTTACACAAAC |
| SEQ ID NO: 179 | 3UTR068 | GCGGACGCACCCTTTCTTTGACAACCTAACTTGCGCAGTAAGAGACAACA |
| | | TTGGCATGGCTTTGGGCGCTTGACTGAACGGTGAAGGCGATTGGTTGGAG |
| | | CAAACATCCCCCAAAGTTCTACAAATGTGGCTGAGGACTTTGTACATTGT |
| | | AGTCATTCCAAGTATCCATGAAATAAGTGGTTACAGGAAGTCCCTCACCC |
| | | TCCCAAAGCCACCCCCACTCCTAAGAGGAGGATGGTCGCGTCCATGCCCT |
| | | GAGTCCACCCCGGGGAAGGTGACAGCATTGCTTCTGTGTAAATTATGTAC |
| | | TGCATCTTCCGCCTCTGAATGGCCCAGGTCTGAGGCCTCCCTTGTCCCCC |
| | | CAACTTGATGTATGAAGGCTTTGGTCTCCCTGGGAGGGGGTTGAGGTGTT |
| | | GAGGTGCACACCTTACCTTACACAAAC |
| SEQ ID NO: 180 | 3UTR069 | GCGGACGCACCCTTTCTTTGACAACCTAACTTGCGCAGTAAGAGACAACA |
| | | TTGGCATGGCTTTGGGCGCTTGACTGAACGGTGAAGGCGATTGGTTGGAG |
| | | CAAACATCCCCCAAAGTTCTACAAATGTGGCTGAGGACTTTGTACATTGT |
| | | AGTCATTCCAAGTATCCATGAAATAAGTGGTTACAGGAAGTCCCTCACCC |
| | | TCCCAAAGCCACCCCCACTCCTAAGAGGAGGATGGTCGCGTCCATGCCCT |
| | | GAGTCCACCCCGGGGAAGGTGAAGGCCTCCCTTGTCCCCCCAACTTGATG |
| | | TATGAAGGCTTTGGTCTCCCTGGGAGGGGGTTGAGGTGTTGAGGTGCACA |
| | | CCTTACCTTACACAAAC |
| SEQ ID NO: 181 | 3UTR070 | CTAACTATTTGCTTTGTATTTTAAGATTTTGTAAATAGAAAAATATATAA |
| | | CCCCACTCGTAGGTAAGGAGTATATTAGTTAGTTATTCAGTACTTACGGC |
| | | CCTATTACCAACGGGTATTAATCACAAACACTTTATCCCCATAGGATTCT |
| | | CGTCAGAGTCCCATCGGGGCTAACAGGTTTTTCGCACTTTTCCTGCTAAC |
| | | TGACAGAAGTGCAATTTGGTTTTTGATTAATAGTTGTTTTCT |
| SEQ ID NO: 182 | 3UTR071 | CTAACTATTTGCTTTGTATTTTAAGATTTTGTAAATAGAAAAATATATAA |
| | | CCCCACTCGTAGGTAAGGAGTATATTAGTTAGTTATTCAGTACTTACGGC |
| | | CCTATTACCAACGGGTATTAATCACAAACACTTTATCCCCATAGGATTCT |
| | | CGTCAGAGTCCCATCGGGGCTAACAGGTTTTTCGCACTTTTCCTGCTAAC |
| | | TGACAGAAGTGCAATTTGGTAGTTGTTTTCT |
| SEQ ID NO: 183 | 3UTR072 | CTAACTATTTGCTTTGTGTAAATAGAAAAATATATAACCCCACTCGTAGG |
| | | TAAGGAGTATATTAGTTAGTTATTCAGTACTTACGGCCCTATTACCAACG |
| | | GGTATTAATCACAAACACTTTATCCCCATAGGATTCTCGTCAGAGTCCCA |
| | | TCGGGGCTAACAGGTTTTTCGCACTTTTCCTGCTAACTGACAGAAGTGCA |
| | | ATTTGGTAGTTGTTTTCT |
| SEQ ID NO: 184 | 3UTR073 | CTAACTATTTGCTTTGTGTAACCCCACTCGTAGGTAAGGAGAGTTCAGTA |
| | | CTTACGGCCCTATTACCAACGGGTATTAATCACAAACACCCATAGGATTC |
| | | TCGTCAGAGTCCCATCGGGGCTAACAGGTTTTTCGCACTTTTCCTGCTAA |
| | | CTGACAGAAGTGCAATTTGGTAGTTGTTTTCT |
| SEQ ID NO: 185 | 3UTR074 | GCGGACTATGACTTAGTTGCGTTACACCCTTTCTTGACAAAACCTAACTT |
| | | GCGCAGAAAACAAGATGAGATTGGCATGGCTGTTTTTTTTGTTTTGTTTT |
| | | GGTTTTTTTTTTTTTTTTGGCTTGACTCAGGAACTGGAACGGTGAAGGTG |
| | | ACAGCAGTCGGTTGGAGCGAGCATCCCCCAAAGTTCACAATGTGGCCGAG |
| | | GACGCACATTGTTGTCATTCCAAATATGAGATGCGTTGTTACAGGAAGTC |
| | | CCTTGCCATCCTAAAAGCCACCCCACTTCTCTCTAAGGAGAATGGCCCAG |
| | | TCCTCTCCCAAGTCCACACAGGGGAGGTGATAGCATTGCTTTCGTGTAAA |
| | | TTAGCAAAATTTTCTTCGCCCTTTTTGGATGAGCCTTCGTGCCCCCCCTT |
| | | CCCCCTTTTTTGTCCCCCAACTTGAGATGTATGAAGGCTTTTGGTCTCCC |
| | | TGGGAGTGGGTGGAGGCAGCCAGGGCTTACCTGTACACTGACTTGAGACC |
| | | AGTTGAGTGCACACGA |
| SEQ ID NO: 186 | 3UTR076 | aaaaattcattctctgtggtatccaagaatcagtgaagatgccagtgaaa |
| | | cttcaagcaaatctacttcaacacttcatgtattgtgtgggtctgttgta |
| | | gggttgccagatgcaatacaagattcctggttaaatttgaatttcagtaa |
| | | acaatgaatagtttttcattgtaccatgaaatatccagaacatacttata |
| | | tgtaaagtattatttatttgaatctacaaaaaacaacaaataatttttaa |
| | | atataaggattttcctagatattgcacgggagaatatacaaatagcaaaa |
| | | ttgaggccaagggccaagagaatatccgaactttaatttcaggaattgaa |
| | | tgggtttgctagaatgtgatatttgaagcatcacataaaaatgatgggac |
| | | aataaattttgccataaagtcaaatttagctggaaatcctggattttttt |
| | | ctgttaaatctggcaaccctagtctgctagccaggatccacaagtccttg |
| | | ttccactgtgccttggtttctcctttatttctaagtggaaaaagtattag |
| | | ccaccatcttacctcacagtgatgttgtgaggacatgtggaagcacttta |
| | | agttttttcatcataacataaattattttcaagtgtaacttattaaccta |
| | | tttattatttatgtatttatttaagcatcaaatatttgtgcaagaatttg |
| | | gaaaaatagaagatgaatcattgattgaatagttataaagatgttatagt |
| | | aaatttattttattttagatattaaatgatgttttattagataaatttca |
| | | atcagggtttttagattaaacaaacaaacaattgggtacccagttaaatt |
| | | ttcatttcagataaacaacaaataattttttagtataagtacattattgt |
| | | ttatctgaaattttaattgaactaacaatcctagtttgatactcccagtc |
| | | ttgtcattgccagctgtgttggtagtgctgtgttgaattacggaataatg |
| | | agttagaactattaaaacagccaaaactccacagtcaatattagtaattt |
| | | cttgctggttgaaacttgtttattatgtacaaatagattcttataatatt |
| | | atttaaatgactgcatttttaaatacaaggctttatatttttaactttaa |
| | | gatgtttttatgtgctctccaaattttttttactgtttctgattgtatgg |
| | | aaatataaaagtaaatatgaaacatttaaaatataatttgttgtcaaagt |
| | | aa |
| SEQ ID NO: 187 | 3UTR077 | gttatatattttttaatttaaatttttcatttatcctgagacatataatc |
| | | caaagtcagcctataaatttctttctgttgctaaaaatcgtcattaggta |
| | | tctgcctttttggttaaaaaaaaaaggaatagcatcaatagtgagtttgt |
| | | tgtactcatgaccagaaagaccatacatagtttgcccaggaaattctggg |
| | | tttaagcttgtgtcctatactcttagtaaagttctttgtcactcccagta |
| | | gtgtcctattttagatgataatttctttgatctccctatttatagttgag |
| | | aatatagagcatttctaacacatgaatgtcaaagactatattgacttttc |
| | | aagaaccctactttccttcttattaaacatagctcatctttatattttta |
| | | attttattttagggctgagaattcataaaaaaattcattctctgtggtat |
| | | ccaagaatcagtgaagatgccagtgaaacttcaagcaaatctacttcaac |
| | | acttcatgtattgtgtgggtctgttgtagggttgccagatgcaatacaag |
| | | attcctggttaaatttgaatttcagtaaacaatgaatagtttttcattgt |
| | | accatgaaatatccagaacatacttatatgtaaagtattatttatttgaa |
| | | tctacaaaaaacaacaaataatttttaaatataaggattttcctagatat |
| | | tgcacgggagaatatacaaatagcaaaattgaggccaagggccaagagaa |
| | | tatccgaactttaatttcaggaattgaatgggtttgctagaatgtgatat |
| | | ttgaagcatcacataaaaatgatgggacaataaattttgccataaagtca |
| | | aatttagctggaaatcctggatttttttctgttaaatctggcaaccctag |
| | | tctgctagccaggatccacaagtccttgttccactgtgccttggtttctc |
| | | ctttatttctaagtggaaaaagtattagccaccatcttacctcacagtga |
| | | tgttgtgaggacatgtggaagcactttaagttttttcatcataacataaa |
| | | ttattttcaagtgtaacttattaacctatttattatttatgtatttattt |
| | | aagcatcaaatatttgtgcaagaatttggaaaaatagaagatgaatcatt |
| | | gattgaatagttataaagatgttatagtaaatttattttattttagatat |
| | | taaatgatgttttattagataaatttcaatcagggtttttagattaaaca |
| | | aacaaacaattgggtacccagttaaattttcatttcagataaacaacaaa |
| | | taattttttagtataagtacattattgtttatctgaaattttaattgaac |
| | | taacaatcctagtttgatactcccagtcttgtcattgccagctgtgttgg |
| | | tagtgctgtgttgaattacggaataatgagttagaactattaaaacagcc |
| | | aaaactccacagtcaatattagtaatttcttgctggttgaaacttgttta |
| | | ttatgtacaaatagattcttataatattatttaaatgactgcatttttaa |
| | | atacaaggctttatatttttaactttaagatgtttttatgtgctctccaa |
| | | attttttttactgtttctgattgtatggaaatataaaagtaaatatgaaa |
| | | catttaaaatataatttgttgtcaaagtaa |
| SEQ ID NO: 188 | 3UTR078 | ggctcccgtcctgctttggcagtgccatgtaaatccccactgggaccaac |
| | | cctggggaaggagccagtttgccggatacaaactggtattctgttctgga |
| | | ggaaagggaggagtggaggtgggctgggccctctcttctcacctttgttt |
| | | tttgttggagtgtttctaataaacttggattctctaaccttta |
| SEQ ID NO: 189 | 3UTR112 | ATGTCTCCAGTTACAACTCCGCAGTGGATGTGAAGAAGCGGTGCCTTTGA |
| | | GAGTCTACTTTTGCTCTCTTCGGAAGAACCCTTAGGGGTTCGTGCATGGG |
| | | CTTGCATAGCAAGTCTAGATGCGGGTACCGTACAGTGTTGAAAAACACTG |
| | | TAAATCTCTAAAAGAGACCA |
| SEQ ID NO: 190 | 3UTR113 | ATGTCTCCAGTTACAACTCCGCAGTGGATGTGAAGAAGCGGCAGCAGCGG |
| | | AGGTCATGAAGGTTTTTCTTTTCCTGAGAAAACAACACGTATTGTTTTCT |
| | | CAGGTTTTGCTTTTTGGCCTTTTTCTAGCTTAAAAAAAAAAAAAGCAAAA |
| | | GATGCTGGTGGTTGGCACTCCTGGTTTCCAGGACGGGGTTCAAATCCCTG |
| | | CGGCGTCTTTGCTT |
| SEQ ID NO: 191 | 3UTR114 | ATGTCTCCAGTTACAACTCCGCAGTGGATGTGAAGAAGCCGTCTGCATAA |
| | | CTTTTATTATTTCTTTTATTAATCAACAAAATTTTGTTTTTAACATTTC |
| SEQ ID NO: 192 | 3UTR115 | ATGTCTCCAGTTACAACTCCGCAGTGGATGTGAAGAAGCCCGCTACGCCC |
| | | CAATGATCCGACCAGCAAAACTCGATGTACTTCCGAGGAACTGATGTGCA |
| | | TAATGCATCAGGCTGGTACATTAGATCCCCGCTTACCGCGGGCAATATAG |
| | | CAACACTAAAAACTCGATGTACTTCCGAGGAAGCGCAGTGCATAATGCTG |
| | | CGCAGTGTTGCCACATAACCACTATATTAACCATTTATCTAGCGGACGCC |
| | | AAAAACTCAATGTATTTCTGAGGAAGCGTGGTGCATAATGCCACGCAGCG |
| | | TCTGC |
| SEQ ID NO: 193 | 3UTR116 | ATGTCTCCAGTTACAACTCCGCAGTGGATGTGAAGAAGCCCGCTACGCCC |
| | | CAATGATCCGACCAGCCTCGATGTACTTCCGAGGAACTGATGTGCATAAT |
| | | GCATCAGGCTGGTACATTAGATCCCCGCTTACCGCGGGCAATATAGCAAC |
| | | ACCTCGATGTACTTCCGAGGAAGCGCAGTGCATAATGCTGCGCAGTGTTG |
| | | CCACATAACCACTATATTAACCCTAGCGGACGCCCTCAATGCTGAGGAAG |
| | | CGTGGTGCATAATGCCACGCAGCGTCTGC |
| SEQ ID NO: 194 | 3UTR117 | gcttcctagatagaaaccaaagcagtgcaagattcagttcaaggtcctga |
| | | aaaaagaaaaacattttactctgtgtaccttgtgtctttctaaatttctc |
| | | tctccaaaataaagttcaagcattaaa |
| SEQ ID NO: 195 | 3UTR118 | gcttcctagatagaaaccaaagcagtgcaagattcagttcaaggtcctga |
| | | aaaaagaaaaacattttactctgtgtaccttgtgtctttctaaatttctc |
| | | tctccaa |
| SEQ ID NO: 196 | 3UTR119 | ATGTCTCCAGTTACAACTCCGCAGTGGATGTGAAGAAGCgcttcctagat |
| | | agaaaccaaagcagtgcaagattcagttcaaggtcctgaaaaaagaaaaa |
| | | cattttactctgtgtaccttgtgtctttctaaatttctctctccaaaata |
| | | aagttcaagcattaaa |
| SEQ ID NO: 197 | 3UTR120 | ATGTCTCCAGTTACAACTCCGCAGTGGATGTGAAGAAGCgcttcctagat |
| | | agaaaccaaagcagtgcaagattcagttcaaggtcctgaaaaaagaaaaa |
| | | cattttactctgtgtaccttgtgtctttctaaatttctctctccaa |
| SEQ ID NO: 198 | 3UTR121 | gggatgagaacagagagaaatatattcataatttactttatgacctagaa |
| | | ggaaactgtcgtgtgtcctatacattgccatcaactttgtttcctcatct |
| | | caaataaagtcctttcagcaa |
| SEQ ID NO: 199 | 3UTR122 | GGGATgaGAAcAGAGAGAAATAtaTTCaTAATTtacTTTATgaccTAGAA |
| | | gGAAactgTCGTGTGtcctATACAttgcCATcaacttTGTTTcctCATct |
| | | ca |
| SEQ ID NO: 200 | 3UTR123 | ATGTCTCCAGTTACAACTCCGCAGTGGATGTGAAGAAGCgggatgagaac |
| | | agagagaaatatattcataatttactttatgacctagaaggaaactgtcg |
| | | tgtgtcctatacattgccatcaactttgtttcctcatctcaaataaagtc |
| | | ctttcagcaa |
| SEQ ID NO: 201 | 3UTR124 | ATGTCTCCAGTTACAACTCCGCAGTGGATGTGAAGAAGCgggatgagaac |
| | | agagagaaatatattcataatttactttatgacctagaaggaaactgtcg |
| | | tgtgtcctatacattgccatcaactttgtttcctcatctca |
| SEQ ID NO: 202 | 3UTR125 | gacagagctctgcggtgtcagggcgagaacccatcttccaaccccggcta |
| | | tttggagacggaaaaactggaattctaacaaggaggagaggagactaaat |
| | | cacatcaatttgcaa |
| SEQ ID NO: 203 | 3UTR126 | gacagagctctgcGGTGtcaggGCGAGAACCCaTCTTCcAACCCcggcTA |
| | | TTTggagacggAAAAActgGAAttCTAACaaGGAGGagaggag |
| SEQ ID NO: 204 | 3UTR127 | ATGTCTCCAGTTACAACTCCGCAGTGGATGTGAAGAAGCgacagagctct |
| | | gcggtgtcagggcgagaacccatcttccaaccccggctatttggagacgg |
| | | aaaaactggaattctaacaaggaggagaggag |
| SEQ ID NO: 205 | 3UTR128 | tgccatttgggcttatttagaaaaaagggtaagctagagagaaaaagaaa |
| | | gaactgtccgtcccccttccgccttctcccttctctcacccccaccctag |
| | | cctccaccatccccgcacaaagcggctctaaacctcaggccacatctttt |
| | | ccaaggcaaaccctgttcaggctggctcgtaggcctgccgctttgatgga |
| | | ggaggtattgtaagctttccattttctataagaaaaaggaaaagttgagg |
| | | ggggggcattagtgctgatagctgtgtgtgttagcttgtatatatatttt |
| | | taaaaatctacctgttcctgacttaaaacaaaaggaaagaaactaccttt |
| | | ttataatgcacaactgttgatggtaggctgtatagtttttagtctgtgta |
| | | gttaatttaatttgcagtttgtgcggcagattgctctgccaagatacttg |
| | | aacactgtgttttattgtggtaattatgttttgtgattcaaacttctgtg |
| | | tactgggtgatgcacccattgtgattgtggaagatagaattcaatttgaa |
| | | ctcaggttgtttatgaggggaaaaaaacagttgcatagagtatagctctg |
| | | tagtggaatatgtcttctgtataactaggctgttaacctatgattgtaaa |
| | | gtagctgtaagaatttcccagtgaaataaaaaaaaattttaagtgttctc |
| | | ggggatgcatagattcatcattttctccaccttaaaaatgcgggcattta |
| | | agtctgtccattatctatatagtcctgtcttgtctattgtatatataatc |
| | | tatatgattaaagaaaatatgcataatcagacaagcttgaatattgtttt |
| | | tgcaccagacgaacagtgaggaaattcggagctatacatatgtgcagaag |
| | | gttactacctagggtttatgcttaattttaattggaggaaatgaatgctg |
| | | attgtaacggagttaattttattgataataaattatacactatgaaaccg |
| | | ccattgggctactgtagatttgtatccttgatgaatctggggtttccatc |
| | | agactgaacttacactgtatattttgcaatagttacctcaaggcctactg |
| | | accaaattgttgtgttgagatgatatttaactttttgccaaataaaatat |
| | | attgattcttttcta |
| SEQ ID NO: 206 | 3UTR129 | tgccatttgggcttatttagaaaaaagggtaagctagagagaaaaagaaa |
| | | gaactgtccgtcccccttccgccttctcccttctctcacccccaccctag |
| | | cctccaccatccccgcacaaagcggctctaaacctcaggccacatctttt |
| | | ccaaggcaaaccctgttcaggctggctcgtaggcctgccgctttgatgga |
| | | ggaggtattgtaagctttccattttctataagaaaaaggaaaagttgagg |
| | | ggggggcattagtgctgatagctgtgtgtgttagcttgtatatatatttt |
| | | taaaaatctacctgttcctgacttaaaacaaaaggaaagaaactaccttt |
| | | ttataatgcacaactgttgatggtaggctgtatagtttttagtctgtgta |
| | | gttaatttaatttgcagtttgtgcggcagattgctctgccaagatacttg |
| | | aacactgtgttttattgtggtaattatgttttgtgattcaaacttctgtg |
| | | tactgggtgatgcacccattgtgattgtggaagatagaattcaatttgaa |
| | | ctcaggttgtttatgaggggaaaaaaacagttgcatagagtatagctctg |
| | | tagtggaatatgtcttctgtataactaggctgttaacctatgattgtaaa |
| | | gtagctgtaagaatttcccagtgaaataaaaaaaaattttaagtgttctc |
| | | ggggatgcatagattcatcattttctccaccttaaaaatgcgggcattta |
| | | agtctgtccattatctatatagtcctgtcttgtctattgtatatataatc |
| | | tatatgattaaagaaaatatgcataatcagacaagcttgaatattgtttt |
| | | tgcaccagacgaacagtgaggaaattcggagctatacatatgtgcagaag |
| | | gttactacctagggtttatgcttaattttaattggaggaaatgaatgctg |
| | | attgtaacggagttaattttattgataataaattatacactatgaaaccg |
| | | ccattgggctactgtagatttgtatccttgatgaatctggggtttccatc |
| | | agactgaacttacactgtatattttgcaatagttacctcaaggcctactg |
| | | accaaattgttgtgttgagatgatatttaactttttgcca |
| SEQ ID NO: 207 | 3UTR130 | tgccatttgggcttatttagaaaaaagggtaagctagagagaaaaagaaa |
| | | gaactgtccgtcccccttccgccttctcccttctctcacccccaccctag |
| | | cctccaccatccccgcacaaagcggctctaaacctcaggccacatctttt |
| | | ccaaggcaaaccctgttcaggctggctcgtaggcctgccgctttgatgga |
| | | ggaggtattgtaagctttccattttctataagaaaaaggaaaagttgagg |
| | | ggggggcattagtgctgatagctgtgtgtgttagcttgtatatatatttt |
| | | taaaaatctacctgttcctgacttaaaacaaaaggaaagaaactaccttt |
| | | ttataatgcacaactgttgatggtaggctgtatagtttttagtctgtgta |
| | | gttaatttaatttgcagtttgtgcggcagattgctctgccaagatacttg |
| | | aacactgtgttttattgtggtaattatgttttgtgattcaaacttctgtg |
| | | tactgggtgatgcacccattgtgattgtggaagatagaattcaatttgaa |
| | | ctcaggttgtttatgaggggaaaaaaacagttgcatagagtatagctctg |
| | | tagtggaatatgtcttctgtataactaggctgttaacctatgattgtaaa |
| | | gtagctgtaagaatttcccagtgaaataaaaaaaaattttaagtgttctc |
| | | ggggatgcatagattcatcattttctccaccttaaaaatgcgggcattta |
| | | agtctgtccattatctatatagtcctgtcttgtctattgtatatataatc |
| | | tatatgattaaagaaaatatgcataatcagacaagcttgaatattgtttt |
| | | tgcaccagacgaacagtgaggaaattcggagctatacatatgtgcagaag |
| | | gttactacctagggtttatgcttaattttaattggaggaaatgaatgctg |
| | | attgtaacggagttaattttattgat |
| SEQ ID NO: 208 | 3UTR131 | tgccatttgggcttatttagaaaaaagggtaagctagagagaaaaagaaa |
| | | gaactgtccgtcccccttccgccttctcccttctctcacccccaccctag |
| | | cctccaccatccccgcacaaagcggctctaaacctcaggccacatctttt |
| | | ccaaggcaaaccctgttcaggctggctcgtaggcctgccgctttgatgga |
| | | ggaggtattgtaagctttccattttctataagaaaaaggaaaagttgagg |
| | | ggggggcattagtgctgatagctgtgtgtgttagcttgtatatatatttt |
| | | taaaaatctacctgttcctgacttaaaacaaaaggaaagaaactaccttt |
| | | ttataatgcacaactgttgatggtaggctgtatagtttttagtctgtgta |
| | | gttaatttaatttgcagtttgtgcggcagattgctctgccaagatacttg |
| | | aacactgtgttttattgtggtaattatgttttgtgattcaaacttctgtg |
| | | tactgggtgatgcacccattgtgattgtggaagatagaattcaatttgaa |
| | | ctcaggttgtttatgaggggaaaaaaacagttgcatagagtatagctctg |
| | | tagtggaatatgtcttctgtataactaggctgttaacctatgattgtaaa |
| | | gtagctgtaagaatttcccagtga |
| SEQ ID NO: 209 | 3UTR132 | ATGTCTCCAGTTACAACTCCGCAGTGGATGTGAAGAAGCtgccatttggg |
| | | cttatttagaaaaaagggtaagctagagagaaaaagaaagaactgtccgt |
| | | cccccttccgccttctcccttctctcacccccaccctagcctccaccatc |
| | | cccgcacaaagcggctctaaacctcaggccacatcttttccaaggcaaac |
| | | cctgttcaggctggctcgtaggcctgccgctttgatggaggaggtattgt |
| | | aagctttccattttctataagaaaaaggaaaagttgaggggggggcatta |
| | | gtgctgatagctgtgtgtgttagcttgtatatatatttttaaaaatctac |
| | | ctgttcctgacttaaaacaaaaggaaagaaactacctttttataatgcac |
| | | aactgttgatggtaggctgtatagtttttagtctgtgtagttaatttaat |
| | | ttgcagtttgtgcggcagattgctctgccaagatacttgaacactgtgtt |
| | | ttattgtggtaattatgttttgtgattcaaacttctgtgtactgggtgat |
| | | gcacccattgtgattgtggaagatagaattcaatttgaactcaggttgtt |
| | | tatgaggggaaaaaaacagttgcatagagtatagctctgtagtggaatat |
| | | gtcttctgtataactaggctgttaacctatgattgtaaagtagctgtaag |
| | | aatttcccagtga |
| SEQ ID NO: 210 | 3UTR133 | gatggcatttttgcaggctggctttggaatagatggacagtttgtttcct |
| | | gtctgatagcaccacacgcaaaccaacctttctgacatcagcactttacc |
| | | agaggcataaacacaactgactcccattttggtgtgcatctgtgtgtgtg |
| | | tgcgtgtatatgtgcttgtgctcatgtgtgtggtcagcggtatgtgcgtg |
| | | tgcgtgttcctttgctcttgccattttaaggtagccctctcatcgtcttt |
| | | tagttccaacaaagaaaggtgccatgtctttactagactgaggagccctc |
| | | tcgegggtctcccatcccctccctccttcactcctgcctcctcagctttg |
| | | cttcatgttcgagcttacctactcttccaggactctctgcttggattcac |
| | | taaaaagggccctggtaaaatagtggatctcagtttttaagagtacaagc |
| | | tcttgtttctgtttagtccgtaagttaccatgctaatgaggtgcacacaa |
| | | taacttagcactactccgcagctctagtcctttataagttgctttcctct |
| | | tactttcagttttggtgataatcgtcttcaaattaaagtgctgtttagat |
| | | ttattagatcccatatttacttactgctatctactaagtttccttttaat |
| | | tctaccaaccccagataagtaagagtactattaatagaacacagagtgtg |
| | | tttttgcactgtctgtacctaaagcaataatcctattgtacgctagagca |
| | | tgctgcctgagtattactagtggacgtaggatattttccctacctaagaa |
| | | tttcactgtcttttaaaaaacaaaaagtaaagtaatgcatttgagcatgg |
| | | ccagactattccctaggacaaggaagcagagggaaatgggaggtctaagg |
| | | atgaggggttaatttatcagtacatgagccaaaaactgcgtcttggatta |
| | | gcctttgacattgatgtgttcggttttgttgttccccttccctcacaccc |
| | | tgcctcgcccccacttttctagttaactttttccatatccctcttgacat |
| | | tcaaaacagttacttaagattcagttttcccactttttggtaatatatat |
| | | atttttgtgaattatactttgttgtttttaaaaagaaaatcagttgatta |
| | | agttaataagttgatgttttctaaggccctttttcctagtggtgtcattt |
| | | ttgaatgcctcataaattaatgattctgaagcttatgtttcttattctct |
| | | gtttgcttttgaacgtatgtgctcttataaagtggacttctgaaaaatga |
| | | atgtaaaagacactggtgtatctcagaaggggatggtgttgtcacaaact |
| | | gtggttaatccaatcaatttaaatgtttactatagaccaaaaggagagat |
| | | tattaaatcgtttaatgtttatacagagtaattataggaagttctttttt |
| | | gtacagtatttttcagatataaatactgacaatgtattttggaagacata |
| | | tattatatatagaaaagaggagaggaaaactattccatgttttaaaatta |
| | | tatagcaaagatatatattcaccaatgttgtacagagaagaagtgcttgg |
| | | gggtttttgaagtctttaatattttaagccctatcactgacacatcagca |
| | | tgttttctgctttaaattaaaattttatgacagtatcgaggcttgtgatg |
| | | acgaatcctgctctaaaatacacaaggagctttcttgtttcttattaggc |
| | | ctcagaaagaagtcagttaacgtcacccaaaagcacaaaatggattttag |
| | | tcaaatatttattggatgatacagtgttttttaggaaaagcatctgccac |
| | | aaaaatgttcacttcgaaattctgagttcctggaatggcacgttgctgcc |
| | | agtgccccagacagttcttttctaccctgcgggcccgcacgttttatgag |
| | | gttgatatcggtgctatgtgtttggtttataatttgatagatgtttgact |
| | | ttaaagatgattgttcttttgtttcattaagttgtaaaatgtcaagaaat |
| | | tctgctgttacgacaaagaaacattttacgctagattaaaatatcctttc |
| | | atcaatgggattttctagtttcctgccttcagagtatctaatcctttaat |
| | | gatctggtggtctcctcgtcaatccatcagcaatgcttctctcatagtgt |
| | | catagacttgggaaacccaaccagtaggatatttctacaaggtgttcatt |
| | | ttgtcacaagctgtagataacagcaagagatgggggtgtattggaattgc |
| | | aatacattgttcaggtgaataataaaatcaaaaacttttgcaatcttaag |
| | | cagagataaataaaagatagcaatatgagacacaggtggacgtagagttg |
| | | gcctttttacaggcaaagaggcgaattgtagaattgttagatggcaatag |
| | | tcattaaaaacatagaaaaatgatgtctttaagtggagaattgtggaagg |
| | | attgtaacatggaccatccaaatttatggccgtatcaaatggtagctgaa |
| | | aaaactatatttgagcactggtctctcttggaattagatgtttatatcaa |
| | | atgagcatctcaaatgttttctgcagaaaaaaataaaaagattctaataa |
| | | aatgtattctcttgtgtgccaggagaggtttcagaaacctacctcgtctt |
| | | acaaatttaaacactttggagtctgtacaggtgccttatatgtaggtcat |
| | | tgtcacgatacacacacacgaacactccctctggactggctgcctctcca |
| | | tccagggcagttaactagcaaacaaggcagatctgcttcatggagcggga |
| | | ggccatggcttgactctgagtgatttgggtcaaccggagtcagacgcatg |
| | | tctgcacgctgcagctattatgagagtccctttgtcatttttcacctttt |
| | | catcctaagcatctttcagagattaattatttggccattaacaatgaatc |
| | | caaatcatatcatactgacatcatctagacatgatttggaaggaacagct |
| | | taggacctcctgatgaggtcacattgttgtttcttttaactagacttggc |
| | | aaagaaaggcaaaaattgaccagcctatctttctgctggtgctgccttaa |
| | | ggaggtagtttgttgaggggagggctgtagatcattacttctttctcttc |
| | | aggaagtggccactttgaaccattcaaataccacattaggcaagactgtg |
| | | ataggccttttgtcttcaaatacaacaggcctccactgacccatccctca |
| | | aagcagaaggaccctttgaggagagtacagatgggattccacagtggggt |
| | | gggtggaatggaaacctgtactagaccacccagaggttccttctaaccca |
| | | ctggtttggtggggaactcacagtaattccaaatgtacaatcagatgtct |
| | | agggtctgttttcggaagaagcaagaattatcagtggcaccctccccact |
| | | gcccccagtgtaaaacaatagacattctgtgaaatgcaaagctattcttt |
| | | ggtttttctagtagtttatctcattttaccctattcttcctttaaggaaa |
| | | actcaatctttatcacagtcaattagagcgatcccaaggcatgggaccag |
| | | gcctgcttgcctatgtgtgatggcaattggagatctggatttagcactgg |
| | | ggtctcagcaccctgcaggtgtctgagactaagtgatctgccctccaggt |
| | | ggcgatcaccttctgctcctaggtacccccactggcaaggccaaggtctc |
| | | ctccacgttttttctgcaattaataatgtcatttaaaaaatgagcaaagc |
| | | cttatccgaatcggatatagcaactaaagtcaatacattttgcaggaggc |
| | | taagtgtaagagtgtgtgtgtgtgtgtgtgcgtgcatgtgtgtgtgtgtg |
| | | tatgtgtgtgaataagtcgacataaagtctttaattttgagcaccttacc |
| | | aaacataacaataatccattatccttttggcaacaccacaaagatcgcat |
| | | ctgttaaacaggtacaagttgacatgaggttagtttaattgtacaccatg |
| | | atattggtggtatttatgctgttaagtccaaacctttatctgtctgttat |
| | | tcttaatgttgaataaactttgaattttttcctttctttcatgtattttt |
| | | attaacagttggctagcaatggtattctgttcccacctcggtagcaaaga |
| | | gaccatttgtagagattattacctagataataaaatgataatactatata |
| | | attagtaa |
| SEQ ID NO: 211 | 3UTR 134 | gatggcatttttgcaggctggctttggaatagatggacagtttgtttcct |
| | | gtctgatagcaccacacgcaaaccaacctttctgacatcagcactttacc |
| | | agaggcataaacacaactgactcccattttggtgtgcatctgtgtgtgtg |
| | | tgcgtgtatatgtgcttgtgctcatgtgtgtggtcagcggtatgtgcgtg |
| | | tgcgtgttcctttgctcttgccattttaaggtagccctctcatcgtcttt |
| | | tagttccaacaaagaaaggtgccatgtctttactagactgaggagccctc |
| | | tegcgggtctcccatcccctccctccttcactcctgcctcctcagctttg |
| | | cttcatgttcgagcttacctactcttccaggactctctgcttggattcac |
| | | taaaaagggccctggtaaaatagtggatctcagtttttaagagtacaagc |
| | | tcttgtttctgtttagtccgtaagttaccatgctaatgaggtgcacacaa |
| | | taacttagcactactccgcagctctagtcctttataagttgctttcctct |
| | | tactttcagttttggtgataatcgtcttcaa |
| SEQ ID NO: 212 | 3UTR135 | ATGTCTCCAGTTACAACTCCGCAGTGGATGTGAAGAAGCgatggcatttt |
| | | tgcaggctggctttggaatagatggacagtttgtttcctgtctgatagca |
| | | ccacacgcaaaccaacctttctgacatcagcactttaccagaggcataaa |
| | | cacaactgactcccattttggtgtgcatctgtgtgtgtgtgcgtgtatat |
| | | gtgcttgtgctcatgtgtgtggtcagcggtatgtgcgtgtgcgtgttcct |
| | | ttgctcttgccattttaaggtagccctctcatcgtcttttagttccaaca |
| | | aagaaaggtgccatgtctttactagactgaggagccctctcgcgggtctc |
| | | ccatcccctccctccttcactcctgcctcctcagctttgcttcatgttcg |
| | | agcttacctactcttccaggactctctgcttggattcactaaaaagggcc |
| | | ctggtaaaatagtggatctcagtttttaagagtacaagctcttgtttctg |
| | | tttagtccgtaagttaccatgctaatgaggtgcacacaataacttagcac |
| | | tactccgcagctctagtcctttataagttgctttcctcttactttcagtt |
| | | ttggtgataatcgtcttcaa |
| SEQ ID NO: 213 | 3UTR136 | ggcagaagtcagttcttctgtccatccctctccccagccaggatagagct |
| | | atcttttccatctcatcctcagaagagactcagaagaaagatgacagccc |
| | | tcagaatgcacgttatgaggaaggcagaatgtgggtctgtaattcctccg |
| | | tgtcccttctccccctctgcaaaccgtcgtaacaataatagttcctaaca |
| | | catgggacaattgtgagg |
| SEQ ID NO: 214 | 3UTR137 | ATGTCTCCAGTTACAACTCCGCAGTGGATGTGAAGAAGCggcaGAAGtca |
| | | gTTCTTCtGTCCATCCCtcTCCCCAgccaggATAGAgctatCTTTTcCAT |
| | | ctCATcctcaGAAGAGACTcaGAAGAAagATGacAGCCCtcagAATGcac |
| | | gttATGagGAAGgcagAATGTGGGTctgTAATTCCTCCgtGTCCCTTCtc |
| | | CCCCTctgcaaaccgtegTAACAATAATAgTTCctAACACatGGGACaat |
| | | tgtgagg |
| SEQ ID NO: 215 | 3UTR138 | ggcgccaggcctggcccggctgggccccgcgggccgccgccttcgcctcc |
| | | gggcgcgcgggcctcctgttcgcgacaagcccgccgggatcccgggccct |
| | | gggcccggccaccgtcctggggccgagggcgcccgacggccaggatctcg |
| | | ctgtaggtcaggcccgcgcagcctcctgcgcccagaagcccacgccgccg |
| | | ccgtctgctgggccccggccctcgcggaggtgtccgaggcgacgcacctc |
| | | gagggtgtccgccggccccagcacccaggggacgcgctggaaagcaaaca |
| | | ggaagattcccggagggaaactgtgaatgcttctg |
| SEQ ID NO: 216 | 3UTR139 | gcttcctagatagaaaccaaagcagtgcaagattcagttcaaggtcctga |
| | | aaaaagaaaaacattttactctgtgtaccttgtgtctttctaaatttctc |
| | | tctccaaCGTCTGCATAACTTTTATTATTTCTTTTATTAATCAACAAAAT |
| | | TTTGTTTTTAACATTTC |
| SEQ ID NO: 217 | 3UTR140 | ATGTCTCCAGTTACAACTCCGCAGTGGATGTGAAGAAGCgcttcctagat |
| | | agaaaccaaagcagtgcaagattcagttcaaggtcctgaaaaaagaaaaa |
| | | cattttactctgtgtaccttgtgtctttctaaatttctctctccaaCGTC |
| | | TGCATAACTTTTATTATTTCTTTTATTAATCAACAAAATTTTGTTTTTAA |
| | | CATTTC |
| SEQ ID NO: 218 | 3UTR141 | gggatgagaacagagagaaatatattcataatttactttatgacctagaa |
| | | ggaaactgtcgtgtgtcctatacattgccatcaactttgtttcctcatct |
| | | caCGTCTGCATAACTTTTATTATTTCTTTTATTAATCAACAAAATTTTGT |
| | | TTTTAACATTTC |
| SEQ ID NO: 219 | 3UTR142 | ATGTCTCCAGTTACAACTCCGCAGTGGATGTGAAGAAGCgggatgagaac |
| | | agagagaaatatattcataatttactttatgacctagaaggaaactgtcg |
| | | tgtgtcctatacattgccatcaactttgtttcctcatctcaCGTCTGCAT |
| | | AACTTTTATTATTTCTTTTATTAATCAACAAAATTTTGTTTTTAACATTT |
| | | C |
| SEQ ID NO: 220 | 3UTR143 | gacagagctctgcggtgtcagggcgagaacccatcttccaaccccggcta |
| | | tttggagacggaaaaactggaattctaacaaggaggagaggagCGTCTGC |
| | | ATAACTTTTATTATTTCTTTTATTAATCAACAAAATTTTGTTTTTAACAT |
| | | TTC |
| SEQ ID NO: 221 | 3UTR 144 | ATGTCTCCAGTTACAACTCCGCAGTGGATGTGAAGAAGCgacagagctct |
| | | gcggtgtcagggcgagaacccatcttccaaccccggctatttggagacgg |
| | | aaaaactggaattctaacaaggaggagaggagCGTCTGCATAACTTTTAT |
| | | TATTTCTTTTATTAATCAACAAAATTTTGTTTTTAACATTTC |
| SEQ ID NO: 222 | 3UTR145 | tgccatttgggcttatttagaaaaaagggtaagctagagagaaaaagaaa |
| | | gaactgtccgtcccccttccgccttctcccttctctcacccccaccctag |
| | | cctccaccatccccgcacaaagcggctctaaacctcaggccacatctttt |
| | | ccaaggcaaaccctgttcaggctggctcgtaggcctgccgctttgatgga |
| | | ggaggtattgtaagctttccattttctataagaaaaaggaaaagttgagg |
| | | ggggggcattagtgctgatagctgtgtgtgttagcttgtatatatatttt |
| | | taaaaatctacctgttcctgacttaaaacaaaaggaaagaaactaccttt |
| | | ttataatgcacaactgttgatggtaggctgtatagtttttagtctgtgta |
| | | gttaatttaatttgcagtttgtgcggcagattgctctgccaagatacttg |
| | | aacactgtgttttattgtggtaattatgttttgtgattcaaacttctgtg |
| | | tactgggtgatgcacccattgtgattgtggaagatagaattcaatttgaa |
| | | ctcaggttgtttatgaggggaaaaaaacagttgcatagagtatagctctg |
| | | tagtggaatatgtcttctgtataactaggctgttaacctatgattgtaaa |
| | | gtagctgtaagaatttcccagtgaCGTCTGCATAACTTTTATTATTTCTT |
| | | TTATTAATCAACAAAATTTTGTTTTTAACATTTC |
| SEQ ID NO: 223 | 3UTR146 | gatggcatttttgcaggctggctttggaatagatggacagtttgtttcct |
| | | gtctgatagcaccacacgcaaaccaacctttctgacatcagcactttacc |
| | | agaggcataaacacaactgactcccattttggtgtgcatctgtgtgtgtg |
| | | tgcgtgtatatgtgcttgtgctcatgtgtgtggtcagcggtatgtgcgtg |
| | | tgcgtgttcctttgctcttgccattttaaggtagccctctcatcgtcttt |
| | | tagttccaacaaagaaaggtgccatgtctttactagactgaggagccctc |
| | | tcgcgggtctcccatcccctccctccttcactcctgcctcctcagctttg |
| | | cttcatgttcgagcttacctactcttccaggactctctgcttggattcac |
| | | taaaaagggccctggtaaaatagtggatctcagtttttaagagtacaagc |
| | | tcttgtttctgtttagtccgtaagttaccatgctaatgaggtgcacacaa |
| | | taacttagcactactccgcagctctagtcctttataagttgctttcctct |
| | | tactttcagttttggtgataatcgtcttcaaCGTCTGCATAACTTTTATT |
| | | ATTTCTTTTATTAATCAACAAAATTTTGTTTTTAACATTTC |
| SEQ ID NO: 224 | 3UTR147 | ATGTCTCCAGTTACAACTCCGCAGTGGATGTGAAGAAGCgatggcatttt |
| | | tgcaggctggctttggaatagatggacagtttgtttcctgtctgatagca |
| | | ccacacgcaaaccaacctttctgacatcagcactttaccagaggcataaa |
| | | cacaactgactcccattttggtgtgcatctgtgtgtgtgtgcgtgtatat |
| | | gtgcttgtgctcatgtgtgtggtcagcggtatgtgcgtgtgcgtgttcct |
| | | ttgctcttgccattttaaggtagccctctcatcgtcttttagttccaaca |
| | | aagaaaggtgccatgtctttactagactgaggagccctctcgcgggtctc |
| | | ccatcccctccctccttcactcctgcctcctcagctttgcttcatgttcg |
| | | agcttacctactcttccaggactctctgcttggattcactaaaaagggcc |
| | | ctggtaaaatagtggatctcagtttttaagagtacaagctcttgtttctg |
| | | tttagtccgtaagttaccatgctaatgaggtgcacacaataacttagcac |
| | | tactccgcagctctagtcctttataagttgctttcctcttactttcagtt |
| | | ttggtgataatcgtcttcaaCGTCTGCATAACTTTTATTATTTCTTTTAT |
| | | TAATCAACAAAATTTTGTTTTTAACATTTC |
| SEQ ID NO: 225 | 3UTR148 | ggcagaagtcagttcttctgtccatccctctccccagccaggatagagct |
| | | atcttttccatctcatcctcagaagagactcagaagaaagatgacagccc |
| | | tcagaatgcacgttatgaggaaggcagaatgtgggtctgtaattcctccg |
| | | tgtcccttctccccctctgcaaaccgtcgtaacaataatagttcctaaca |
| | | catgggacaattgtgaggCGTCTGCATAACTTTTATTATTTCTTTTATTA |
| | | ATCAACAAAATTTTGTTTTTAACATTTC |
| SEQ ID NO: 226 | 3UTR 149 | ATGTCTCCAGTTACAACTCCGCAGTGGATGTGAAGAAGCggcagaagtca |
| | | gttcttctgtccatccctctccccagccaggatagagctatcttttccat |
| | | ctcatcctcagaagagactcagaagaaagatgacagccctcagaatgcac |
| | | gttatgaggaaggcagaatgtgggtctgtaattcctccgtgtcccttctc |
| | | cccctctgcaaaccgtcgtaacaataatagttcctaacacatgggacaat |
| | | tgtgaggCGTCTGCATAACTTTTATTATTTCTTTTATTAATCAACAAAAT |
| | | TTTGTTTTTAACATTTC |
| SEQ ID NO: 227 | 3UTR150 | CGTCTGCATAACTTTTATTATTTCTTTTATTAATCAACAAAATTTTGTTT |
| | | TTAACATTTCATGTCTCCAGTTACAACTCCGCAGTGGATGTGAAGAAGC |
| SEQ ID NO: 228 | 3UTR151 | ATGTCTCCAGTTACAACTCCGCAGTGGATGTGAAGAAGCaaaaaaCGTCT |
| | | GCATAACTTTTATTATTTCTTTTATTAATCAACAAAATTTTGTTTTTAAC |
| | | ATTTC |
| SEQ ID NO: 229 | 3UTR152 | CGTCTGCATAACTTTTATTATTTCTTTTATTAATCAACAAAATTTTGTTT |
| | | TTAACATTTCaaaaaaATGTCTCCAGTTACAACTCCGCAGTGGATGTGAA |
| | | GAAGC |
| SEQ ID NO: 230 | 3UTR153 | ATGTCTCCAGTTACAACTCCGCAGTGGATGTGAAGAAGCtgccatttggg |
| | | cttatttagaaaaaagggtaagctagagagaaaaagaaagaactgtccgt |
| | | cccccttccgccttctcccttctctcacccccaccctagcctccaccatc |
| | | cccgcacaaagcggctctaaacctcaggccacatcttttccaaggcaaac |
| | | cctgttcaggctggctcgtaggcctgccgctttgatggaggaggtattgt |
| | | aagctttccattttctataagaaaaaggaaaagttgaggggggggcatta |
| | | gtgctgatagctgtgtgtgttagcttgtatatatatttttaaaaatctac |
| | | ctgttcctgacttaaaacaaaaggaaagaaactacctttttataatgcac |
| | | aactgttgatggtaggctgtatagtttttagtctgtgtagttaatttaat |
| | | ttgcagtttgtgcggcagattgctctgccaagatacttgaacactgtgtt |
| | | ttattgtggtaattatgttttgtgattcaaacttctgtgtactgggtgat |
| | | gcacccattgtgattgtggaagatagaattcaatttgaactcaggttgtt |
| | | tatgaggggaaaaaaacagttgcatagagtatagctctgtagtggaatat |
| | | gtcttctgtataactaggctgttaacctatgattgtaaagtagctgtaag |
| | | aatttcccagtgaCGTCTGCATAACTTTTATTATTTCTTTTATTAATCAA |
| | | CAAAATTTTGTTTTTAACATTTC |
| SEQ ID NO: 231 | 3UTR154 | ATGTCTCCAGTTACAACTCCGCAGTGGATGTGAAGAAGCggcgccaggcc |
| | | tggcccggctgggccccgcgggccgccgccttcgcctccgggcgcgcggg |
| | | cctcctgttcgcgacaagcccgccgggatcccgggccctgggcccggcca |
| | | ccgtcctggggccgagggcgcccgacggccaggatctcgctgtaggtcag |
| | | gccegcgcagcctcctgcgcccagaagcccacgccgccgccgtctgctgg |
| | | gccccggccctcgcggaggtgtccgaggcgacgcacctcgagggtgtccg |
| | | ccggccccagcacccaggggacgcgctggaaagcaaacaggaagattccc |
| | | ggagggaaactgtgaatgcttctg |
| SEQ ID NO: 232 | 3UTR155 | ggcgccaggcctggcccggctgggccccgcgggccgccgccttcgcctcc |
| | | gggcgcgcgggcctcctgttcgcgacaagcccgccgggatcccgggccct |
| | | gggcccggccaccgtcctggggccgagggcgcccgacggccaggatctcg |
| | | ctgtaggtcaggcccgcgcagcctcctgcgcccagaagcccacgccgccg |
| | | ccgtctgctggegccccggccctcgcggaggtgtccgaggcgacgcacct |
| | | cgagggtgtccgccggccccagcacccaggggacgcgctggaaagcaaac |
| | | aggaagattcccggagggaaactgtgaatgcttctgCGTCTGCATAACTT |
| | | TTATTATTTCTTTTATTAATCAACAAAATTTTGTTTTTAACATTTC |
| SEQ ID NO: 233 | 3UTR156 | ATGTCTCCAGTTACAACTCCGCAGTGGATGTGAAGAAGCggcgccaggcc |
| | | tggcccggctgggccccgcgggccgccgccttcgcctccgggcgcgcggg |
| | | cctcctgttcgcgacaagcccgccgggatcccgggccctgggcccggcca |
| | | ccgtcctggggccgagggcgcccgacggccaggatctcgctgtaggtcag |
| | | gccegcgcagcctcctgcgcccagaagcccacgccgccgccgtctgctgg |
| | | cgccccggccctcgcggaggtgtccgaggcgacgcacctcgagggtgtcc |
| | | gccggccccagcacccaggggacgcgctggaaagcaaacaggaagattcc |
| | | cggagggaaactgtgaatgcttctgCGTCTGCATAACTTTTATTATTTCT |
| | | TTTATTAATCAACAAAATTTTGTTTTTAACATTTC |
| SEQ ID NO: 234 | 3UTR157 | tgccatttgggcgaaaaaagggtaagctagagagaaaaagaaagaactgt |
| | | ccgtcccccttccgccttctcccttctctcacccccaccctagcctccac |
| | | catccccgcacaaagcggctctaaacctcaggccacatcttttccaaggc |
| | | aaaccctgttcaggctggctcgtaggcctgccgctttgatggaggaggta |
| | | ttgtaagctttccattttctataagaaaaaggaaaagttgaggggggggc |
| | | attagtgctgatagctgtgtgtgttagcttgtatatatatttttaaaaat |
| | | ctacctgttcctgacttaaaacaaaaggaaagaaactacctttttataat |
| | | gcacaactgttgatggtaggctgtatagtttttagtctgtgtagttgcag |
| | | tttgtgcggcagattgctctgccaagatacttgaacactgtgttttattg |
| | | tggtaattatgttttgtgattcaaacttctgtgtactgggtgatgcaccc |
| | | attgtgattgtggaagatagaattcaatttgaactcaggttgtttatgag |
| | | gggaaaaaaacagttgcatagagtatagctctgtagtggaatatgtcttc |
| | | tgtataactaggctgttaacctatgattgtaaagtagctgtaagaatttc |
| | | ccagtga |
| SEQ ID NO: 235 | 3UTR158 | ATGTCTCCAGTTACAACTCCGCAGTGGATGTGAAGAAGCtgccatttggg |
| | | cgaaaaaagggtaagctagagagaaaaagaaagaactgtccgtccccctt |
| | | ccgccttctcccttctctcacccccaccctagcctccaccatccccgcac |
| | | aaagcggctctaaacctcaggccacatcttttccaaggcaaaccctgttc |
| | | aggctggctcgtaggcctgccgctttgatggaggaggtattgtaagcttt |
| | | ccattttctataagaaaaaggaaaagttgaggggggggcattagtgctga |
| | | tagctgtgtgtgttagcttgtatatatatttttaaaaatctacctgttcc |
| | | tgacttaaaacaaaaggaaagaaactacctttttataatgcacaactgtt |
| | | gatggtaggctgtatagtttttagtctgtgtagttgcagtttgtgcggca |
| | | gattgctctgccaagatacttgaacactgtgttttattgtggtaattatg |
| | | ttttgtgattcaaacttctgtgtactgggtgatgcacccattgtgattgt |
| | | ggaagatagaattcaatttgaactcaggttgtttatgaggggaaaaaaac |
| | | agttgcatagagtatagctctgtagtggaatatgtcttctgtataactag |
| | | gctgttaacctatgattgtaaagtagctgtaagaatttcccagtga |
| SEQ ID NO: 236 | 3UTR159 | tgccatttgggcgaaaaaagggtaagctagagagaaaaagaaagaactgt |
| | | ccgtcccccttccgccttctcccttctctcacccccaccctagcctccac |
| | | catccccgcacaaagcggctctaaacctcaggccacatcttttccaaggc |
| | | aaaccctgttcaggctggctcgtaggcctgccgctttgatggaggaggta |
| | | ttgtaagctttccattttctataagaaaaaggaaaagttgaggggggggc |
| | | attagtgctgatagctgtgtgtgttagcttgtatatatatttttaaaaat |
| | | ctacctgttcctgacttaaaacaaaaggaaagaaactacctttttataat |
| | | gcacaactgttgatggtaggctgtatagtttttagtctgtgtagttgcag |
| | | tttgtgcggcagattgctctgccaagatacttgaacactgtgttttattg |
| | | tggtaattatgttttgtgattcaaacttctgtgtactgggtgatgcaccc |
| | | attgtgattgtggaagatagaattcaatttgaactcaggttgtttatgag |
| | | gggaaaaaaacagttgcatagagtatagctctgtagtggaatatgtcttc |
| | | tgtataactaggctgttaacctatgattgtaaagtagctgtaagaatttc |
| | | ccagtgaCGTCTGCATAACTTTTATTATTTCTTTTATTAATCAACAAAAT |
| | | TTTGTTTTTAACATTTC |
| SEQ ID NO: 237 | 3UTR160 | ATGTCTCCAGTTACAACTCCGCAGTGGATGTGAAGAAGCtgccatttggg |
| | | cgaaaaaagggtaagctagagagaaaaagaaagaactgtccgtccccctt |
| | | ccgccttctcccttctctcacccccaccctagcctccaccatccccgcac |
| | | aaagcggctctaaacctcaggccacatcttttccaaggcaaaccctgttc |
| | | aggctggctcgtaggcctgccgctttgatggaggaggtattgtaagcttt |
| | | ccattttctataagaaaaaggaaaagttgaggggggggcattagtgctga |
| | | tagctgtgtgtgttagcttgtatatatatttttaaaaatctacctgttcc |
| | | tgacttaaaacaaaaggaaagaaactacctttttataatgcacaactgtt |
| | | gatggtaggctgtatagtttttagtctgtgtagttgcagtttgtgcggca |
| | | gattgctctgccaagatacttgaacactgtgttttattgtggtaattatg |
| | | ttttgtgattcaaacttctgtgtactgggtgatgcacccattgtgattgt |
| | | ggaagatagaattcaatttgaactcaggttgtttatgaggggaaaaaaac |
| | | agttgcatagagtatagctctgtagtggaatatgtcttctgtataactag |
| | | gctgttaacctatgattgtaaagtagctgtaagaatttcccagtgaCGTC |
| | | TGCATAACTTTTATTATTTCTTTTATTAATCAACAAAATTTTGTTTTTAA |
| | | CATTTC |
| SEQ ID NO: 238 | 3UTR161 | CGTCTGgATAACTTTTATTATTTCTTTTATTAATCAACAAAATTTTGTTT |
| | | TTAACATTTC |
| SEQ ID NO: 239 | 3UTR162 | CGTCTGgATAACTTTTATTATTTCTTTTtTTAATCAACAAAATTTTGTTT |
| | | TTAACATTTC |
| SEQ ID NO: 240 | 3UTR163 | CGTCTGgATAACTTTTATTATTTCTTTTtTTtATCAACAAAATTTTGTTT |
| | | TaAACATTTC |
| SEQ ID NO: 241 | 3UTR164 | CGTCTGgATAACTTTTATTATTTCTTTTATTAtTCAACAAATTTTTGTTT |
| | | TaAACATTTC |
| SEQ ID NO: 242 | 3UTR165 | TATAG |
| SEQ ID NO: 243 | 3UTR166 | ACCAGATCCCGGAGTTGGAAAACAATGAAAAGGCCCCCAAGGTAGTT |
| SEQ ID NO: 244 | 3UTR167 | ATTCCAAATGTGAATATATagTTT |
| SEQ ID NO: 245 | 3UTR168 | TGTGTTCAGAAAACTAGGCAGGAAAGTAGGAAAAGATCTGTTAATCG |
| SEQ ID NO: 246 | 3UTR169 | AATCA |
| SEQ ID NO: 247 | 3UTR170 | TATAGCGTCTGCATAACTTTTATTATTTCTTTTATTAATCAACAAAATTT |
| | | TGTTTTTAACATTTC |
| SEQ ID NO: 248 | 3UTR171 | TATAGCGTCTGgATAACTTTTATTATTTCTTTTATTAATCAACAAAATTT |
| | | TGTTTTTAACATTTC |
| SEQ ID NO: 249 | 3UTR172 | TATAGCGTCTGgATAACTTTTATTATTTCTTTTTTTAATCAACAAAATTT |
| | | TGTTTTTAACATTTC |
| SEQ ID NO: 250 | 3UTR173 | TATAGCGTCTGgATAACTTTTATTATTTCTTTTTTTTATCAACAAAATTT |
| | | TGTTTTaAACATTTC |
| SEQ ID NO: 251 | 3UTR174 | TATAGCGTCTGgATAACTTTTATTATTTCTTTTATTATTCAACAAATTTT |
| | | TGTTTTaAACATTTC |
| SEQ ID NO: 252 | 3UTR175 | ACCAGATCCCGGAGTTGGAAAACAATGAAAAGGCCCCCAAGGTAGTTCGT |
| | | CTGCATAACTTTTATTATTTCTTTTATTAATCAACAAAATTTTGTTTTTA |
| | | ACATTTC |
| SEQ ID NO: 253 | 3UTR176 | ACCAGATCCCGGAGTTGGAAAACAATGAAAAGGCCCCCAAGGTAGTTCGT |
| | | CTGgATAACTTTTATTATTTCTTTTATTAATCAACAAAATTTTGTTTTTA |
| | | ACATTTC |
| SEQ ID NO: 254 | 3UTR177 | ACCAGATCCCGGAGTTGGAAAACAATGAAAAGGCCCCCAAGGTAGTTCGT |
| | | CTGgATAACTTTTATTATTTCTTTTTTTAATCAACAAAATTTTGTTTTTA |
| | | ACATTTC |
| SEQ ID NO: 255 | 3UTR178 | ACCAGATCCCGGAGTTGGAAAACAATGAAAAGGCCCCCAAGGTAGTTCGT |
| | | CTGgATAACTTTTATTATTTCTTTTTTTTATCAACAAAATTTTGTTTTaA |
| | | ACATTTC |
| SEQ ID NO: 256 | 3UTR179 | ACCAGATCCCGGAGTTGGAAAACAATGAAAAGGCCCCCAAGGTAGTTCGT |
| | | CTGgATAACTTTTATTATTTCTTTTATTATTCAACAAATTTTTGTTTTaA |
| | | ACATTTC |
| SEQ ID NO: 257 | 3UTR180 | ATTCCAAATGTGAATATATagTTTCGTCTGCATAACTTTTATTATTTCTT |
| | | TTATTAATCAACAAAATTTTGTTTTTAACATTTC |
| SEQ ID NO: 258 | 3UTR181 | ATTCCAAATGTGAATATATagTTTCGTCTGgATAACTTTTATTATTTCTT |
| | | TTATTAATCAACAAAATTTTGTTTTTAACATTTC |
| SEQ ID NO: 259 | 3UTR182 | ATTCCAAATGTGAATATATagTTTCGTCTGgATAACTTTTATTATTTCTT |
| | | TTTTTAATCAACAAAATTTTGTTTTTAACATTTC |
| SEQ ID NO: 260 | 3UTR183 | ATTCCAAATGTGAATATATagTTTCGTCTGgATAACTTTTATTATTTCTT |
| | | TTTTTTATCAACAAAATTTTGTTTTaAACATTTC |
| SEQ ID NO: 261 | 3UTR184 | ATTCCAAATGTGAATATATagTTTCGTCTGgATAACTTTTATTATTTCTT |
| | | TTATTATTCAACAAATTTTTGTTTTaAACATTTC |
| SEQ ID NO: 262 | 3UTR185 | TGTGTTCAGAAAACTAGGCAGGAAAGTAGGAAAAGATCTGTTAATCGCGT |
| | | CTGCATAACTTTTATTATTTCTTTTATTAATCAACAAAATTTTGTTTTTA |
| | | ACATTTC |
| SEQ ID NO: 263 | 3UTR186 | TGTGTTCAGAAAACTAGGCAGGAAAGTAGGAAAAGATCTGTTAATCGCGT |
| | | CTGgATAACTTTTATTATTTCTTTTATTAATCAACAAAATTTTGTTTTTA |
| | | ACATTTC |
| SEQ ID NO: 264 | 3UTR187 | TGTGTTCAGAAAACTAGGCAGGAAAGTAGGAAAAGATCTGTTAATCGCGT |
| | | CTGgATAACTTTTATTATTTCTTTTTTTAATCAACAAAATTTTGTTTTTA |
| | | ACATTTC |
| SEQ ID NO: 265 | 3UTR188 | TGTGTTCAGAAAACTAGGCAGGAAAGTAGGAAAAGATCTGTTAATCGCGT |
| | | CTGgATAACTTTTATTATTTCTTTTTTTTATCAACAAAATTTTGTTTTaA |
| | | ACATTTC |
| SEQ ID NO: 266 | 3UTR189 | TGTGTTCAGAAAACTAGGCAGGAAAGTAGGAAAAGATCTGTTAATCGCGT |
| | | CTGgATAACTTTTATTATTTCTTTTATTATTCAACAAATTTTTGTTTTaA |
| | | ACATTTC |
| SEQ ID NO: 267 | 3UTR190 | AATCACGTCTGCATAACTTTTATTATTTCTTTTATTAATCAACAAAATTT |
| | | TGTTTTTAACATTTC |
| SEQ ID NO: 268 | 3UTR191 | AATCACGTCTGgATAACTTTTATTATTTCTTTTATTAATCAACAAAATTT |
| | | TGTTTTTAACATTTC |
| SEQ ID NO: 269 | 3UTR192 | AATCACGTCTGgATAACTTTTATTATTTCTTTTTTTAATCAACAAAATTT |
| | | TGTTTTTAACATTTC |
| SEQ ID NO: 270 | 3UTR193 | AATCACGTCTGgATAACTTTTATTATTTCTTTTTTTTATCAACAAAATTT |
| | | TGTTTTaAACATTTC |
| SEQ ID NO: 271 | 3UTR194 | AATCACGTCTGgATAACTTTTATTATTTCTTTTATTATTCAACAAATTTT |
| | | TGTTTTaAACATTTC |
| SEQ ID NO: 272 | 3UTR195 | CGTCTGCATAACTTTTATTATTTCTTTTATTAATCAACAAAATTTTGTTT |
| | | TTAACATTTCTATAG |
| SEQ ID NO: 273 | 3UTR 196 | CGTCTGgATAACTTTTATTATTTCTTTTATTAATCAACAAAATTTTGTTT |
| | | TTAACATTTCTATAG |
| SEQ ID NO: 274 | 3UTR197 | CGTCTGgATAACTTTTATTATTTCTTTTTTTAATCAACAAAATTTTGTTT |
| | | TTAACATTTCTATAG |
| SEQ ID NO: 275 | 3UTR198 | CGTCTGgATAACTTTTATTATTTCTTTTTTTTATCAACAAAATTTTGTTT |
| | | TaAACATTTCTATAG |
| SEQ ID NO: 276 | 3UTR 199 | CGTCTGgATAACTTTTATTATTTCTTTTATTATTCAACAAATTTTTGTTT |
| | | TaAACATTTCTATAG |
| SEQ ID NO: 277 | 3UTR200 | CGTCTGCATAACTTTTATTATTTCTTTTATTAATCAACAAAATTTTGTTT |
| | | TTAACATTTCACCAGATCCCGGAGTTGGAAAACAATGAAAAGGCCCCCAA |
| | | GGTAGTT |
| SEQ ID NO: 278 | 3UTR201 | CGTCTGgATAACTTTTATTATTTCTTTTATTAATCAACAAAATTTTGTTT |
| | | TTAACATTTCACCAGATCCCGGAGTTGGAAAACAATGAAAAGGCCCCCAA |
| | | GGTAGTT |
| SEQ ID NO: 279 | 3UTR202 | CGTCTGgATAACTTTTATTATTTCTTTTTTTAATCAACAAAATTTTGTTT |
| | | TTAACATTTCACCAGATCCCGGAGTTGGAAAACAATGAAAAGGCCCCCAA |
| | | GGTAGTT |
| SEQ ID NO: 280 | 3UTR203 | CGTCTGgATAACTTTTATTATTTCTTTTTTTTATCAACAAAATTTTGTTT |
| | | TaAACATTTCACCAGATCCCGGAGTTGGAAAACAATGAAAAGGCCCCCAA |
| | | GGTAGTT |
| SEQ ID NO: 281 | 3UTR204 | CGTCTGgATAACTTTTATTATTTCTTTTATTATTCAACAAATTTTTGTTT |
| | | TaAACATTTCACCAGATCCCGGAGTTGGAAAACAATGAAAAGGCCCCCAA |
| | | GGTAGTT |
| SEQ ID NO: 282 | 3UTR205 | CGTCTGCATAACTTTTATTATTTCTTTTATTAATCAACAAAATTTTGTTT |
| | | TTAACATTTCATTCCAAATGTGAATATATagTTT |
| SEQ ID NO: 283 | 3UTR206 | CGTCTGgATAACTTTTATTATTTCTTTTATTAATCAACAAAATTTTGTTT |
| | | TTAACATTTCATTCCAAATGTGAATATATagTTT |
| SEQ ID NO: 284 | 3UTR207 | CGTCTGgATAACTTTTATTATTTCTTTTTTTAATCAACAAAATTTTGTTT |
| | | TTAACATTTCATTCCAAATGTGAATATATagTTT |
| SEQ ID NO: 285 | 3UTR208 | CGTCTGgATAACTTTTATTATTTCTTTTTTTTATCAACAAAATTTTGTTT |
| | | TaAACATTTCATTCCAAATGTGAATATATagTTT |
| SEQ ID NO: 286 | 3UTR209 | CGTCTGgATAACTTTTATTATTTCTTTTATTATTCAACAAATTTTTGTTT |
| | | TaAACATTTCATTCCAAATGTGAATATATagTTT |
| SEQ ID NO: 287 | 3UTR210 | CGTCTGCATAACTTTTATTATTTCTTTTATTAATCAACAAAATTTTGTTT |
| | | TTAACATTTCTGTGTTCAGAAAACTAGGCAGGAAAGTAGGAAAAGATCTG |
| | | TTAATCG |
| SEQ ID NO: 288 | 3UTR211 | CGTCTGgATAACTTTTATTATTTCTTTTATTAATCAACAAAATTTTGTTT |
| | | TTAACATTTCTGTGTTCAGAAAACTAGGCAGGAAAGTAGGAAAAGATCTG |
| | | TTAATCG |
| SEQ ID NO: 289 | 3UTR212 | CGTCTGgATAACTTTTATTATTTCTTTTTTTAATCAACAAAATTTTGTTT |
| | | TTAACATTTCTGTGTTCAGAAAACTAGGCAGGAAAGTAGGAAAAGATCTG |
| | | TTAATCG |
| SEQ ID NO: 290 | 3UTR213 | CGTCTGgATAACTTTTATTATTTCTTTTTTTTATCAACAAAATTTTGTTT |
| | | TaAACATTTCTGTGTTCAGAAAACTAGGCAGGAAAGTAGGAAAAGATCTG |
| | | TTAATCG |
| SEQ ID NO: 291 | 3UTR214 | CGTCTGgATAACTTTTATTATTTCTTTTATTATTCAACAAATTTTTGTTT |
| | | TaAACATTTCTGTGTTCAGAAAACTAGGCAGGAAAGTAGGAAAAGATCTG |
| | | TTAATCG |
| SEQ ID NO: 292 | 3UTR215 | CGTCTGCATAACTTTTATTATTTCTTTTATTAATCAACAAAATTTTGTTT |
| | | TTAACATTTCAATCA |
| SEQ ID NO: 293 | 3UTR216 | CGTCTGgATAACTTTTATTATTTCTTTTATTAATCAACAAAATTTTGTTT |
| | | TTAACATTTCAATCA |
| SEQ ID NO: 294 | 3UTR217 | CGTCTGgATAACTTTTATTATTTCTTTTTTTAATCAACAAAATTTTGTTT |
| | | TTAACATTTCAATCA |
| SEQ ID NO: 295 | 3UTR218 | CGTCTGgATAACTTTTATTATTTCTTTTTTTTATCAACAAAATTTTGTTT |
| | | TaAACATTTCAATCA |
| SEQ ID NO: 296 | 3UTR219 | CGTCTGgATAACTTTTATTATTTCTTTTATTATTCAACAAATTTTTGTTT |
| | | TaAACATTTCAATCA |
| SEQ ID NO: 297 | 3UTR220 | TATAGCGTCTGCATAACTTTTATTATTTCTTTTATTAATCAACAAAATTT |
| | | TGTTTTTAACATTTCTATAG |
| SEQ ID NO: 298 | 3UTR221 | TATAGCGTCTGgATAACTTTTATTATTTCTTTTATTAATCAACAAAATTT |
| | | TGTTTTTAACATTTCTATAG |
| SEQ ID NO: 299 | 3UTR222 | TATAGCGTCTGgATAACTTTTATTATTTCTTTTTTTAATCAACAAAATTT |
| | | TGTTTTTAACATTTCTATAG |
| SEQ ID NO: 300 | 3UTR223 | TATAGCGTCTGgATAACTTTTATTATTTCTTTTTTTTATCAACAAAATTT |
| | | TGTTTTaAACATTTCTATAG |
| SEQ ID NO: 301 | 3UTR224 | TATAGCGTCTGgATAACTTTTATTATTTCTTTTATTATTCAACAAATTTT |
| | | TGTTTTaAACATTTCTATAG |
| SEQ ID NO: 302 | 3UTR225 | ACCAGATCCCGGAGTTGGAAAACAATGAAAAGGCCCCCAAGGTAGTTCGT |
| | | CTGCATAACTTTTATTATTTCTTTTATTAATCAACAAAATTTTGTTTTTA |
| | | ACATTTCACCAGATCCCGGAGTTGGAAAACAATGAAAAGGCCCCCAAGGT |
| | | AGTT |
| SEQ ID NO: 303 | 3UTR226 | ACCAGATCCCGGAGTTGGAAAACAATGAAAAGGCCCCCAAGGTAGTTCGT |
| | | CTGgATAACTTTTATTATTTCTTTTATTAATCAACAAAATTTTGTTTTTA |
| | | ACATTTCACCAGATCCCGGAGTTGGAAAACAATGAAAAGGCCCCCAAGGT |
| | | AGTT |
| SEQ ID NO: 304 | 3UTR227 | ACCAGATCCCGGAGTTGGAAAACAATGAAAAGGCCCCCAAGGTAGTTCGT |
| | | CTGgATAACTTTTATTATTTCTTTTTTTAATCAACAAAATTTTGTTTTTA |
| | | ACATTTCACCAGATCCCGGAGTTGGAAAACAATGAAAAGGCCCCCAAGGT |
| | | AGTT |
| SEQ ID NO: 305 | 3UTR228 | ACCAGATCCCGGAGTTGGAAAACAATGAAAAGGCCCCCAAGGTAGTTCGT |
| | | CTGgATAACTTTTATTATTTCTTTTTTTTATCAACAAAATTTTGTTTTaA |
| | | ACATTTCACCAGATCCCGGAGTTGGAAAACAATGAAAAGGCCCCCAAGGT |
| | | AGTT |
| SEQ ID NO: 306 | 3UTR229 | ACCAGATCCCGGAGTTGGAAAACAATGAAAAGGCCCCCAAGGTAGTTCGT |
| | | CTGgATAACTTTTATTATTTCTTTTATTATTCAACAAATTTTTGTTTTaA |
| | | ACATTTCACCAGATCCCGGAGTTGGAAAACAATGAAAAGGCCCCCAAGGT |
| | | AGTT |
| SEQ ID NO: 307 | 3UTR230 | ATTCCAAATGTGAATATATagTTTCGTCTGCATAACTTTTATTATTTCTT |
| | | TTATTAATCAACAAAATTTTGTTTTTAACATTTCATTCCAAATGTGAATA |
| | | TATagTTT |
| SEQ ID NO: 308 | 3UTR231 | ATTCCAAATGTGAATATATagTTTCGTCTGgATAACTTTTATTATTTCTT |
| | | TTATTAATCAACAAAATTTTGTTTTTAACATTTCATTCCAAATGTGAATA |
| | | TATagTTT |
| SEQ ID NO: 309 | 3UTR232 | ATTCCAAATGTGAATATATagTTTCGTCTGgATAACTTTTATTATTTCTT |
| | | TTTTTAATCAACAAAATTTTGTTTTTAACATTTCATTCCAAATGTGAATA |
| | | TATagTTT |
| SEQ ID NO: 310 | 3UTR233 | ATTCCAAATGTGAATATATagTTTCGTCTGgATAACTTTTATTATTTCTT |
| | | TTTTTTATCAACAAAATTTTGTTTTaAACATTTCATTCCAAATGTGAATA |
| | | TATagTTT |
| SEQ ID NO: 311 | 3UTR234 | ATTCCAAATGTGAATATATagTTTCGTCTGgATAACTTTTATTATTTCTT |
| | | TTATTATTCAACAAATTTTTGTTTTaAACATTTCATTCCAAATGTGAATA |
| | | TATagTTT |
| SEQ ID NO: 312 | 3UTR235 | TGTGTTCAGAAAACTAGGCAGGAAAGTAGGAAAAGATCTGTTAATCGCGT |
| | | CTGCATAACTTTTATTATTTCTTTTATTAATCAACAAAATTTTGTTTTTA |
| | | ACATTTCTGTGTTCAGAAAACTAGGCAGGAAAGTAGGAAAAGATCTGTTA |
| | | ATCG |
| SEQ ID NO: 313 | 3UTR236 | TGTGTTCAGAAAACTAGGCAGGAAAGTAGGAAAAGATCTGTTAATCGCGT |
| | | CTGgATAACTTTTATTATTTCTTTTATTAATCAACAAAATTTTGTTTTTA |
| | | ACATTTCTGTGTTCAGAAAACTAGGCAGGAAAGTAGGAAAAGATCTGTTA |
| | | ATCG |
| SEQ ID NO: 314 | 3UTR237 | TGTGTTCAGAAAACTAGGCAGGAAAGTAGGAAAAGATCTGTTAATCGCGT |
| | | CTGgATAACTTTTATTATTTCTTTTTTTAATCAACAAAATTTTGTTTTTA |
| | | ACATTTCTGTGTTCAGAAAACTAGGCAGGAAAGTAGGAAAAGATCTGTTA |
| | | ATCG |
| SEQ ID NO: 315 | 3UTR238 | TGTGTTCAGAAAACTAGGCAGGAAAGTAGGAAAAGATCTGTTAATCGCGT |
| | | CTGgATAACTTTTATTATTTCTTTTTTTTATCAACAAAATTTTGTTTTaA |
| | | ACATTTCTGTGTTCAGAAAACTAGGCAGGAAAGTAGGAAAAGATCTGTTA |
| | | ATCG |
| SEQ ID NO: 316 | 3UTR239 | TGTGTTCAGAAAACTAGGCAGGAAAGTAGGAAAAGATCTGTTAATCGCGT |
| | | CTGgATAACTTTTATTATTTCTTTTATTATTCAACAAATTTTTGTTTTaA |
| | | ACATTTCTGTGTTCAGAAAACTAGGCAGGAAAGTAGGAAAAGATCTGTTA |
| | | ATCG |
| SEQ ID NO: 317 | 3UTR240 | AATCACGTCTGCATAACTTTTATTATTTCTTTTATTAATCAACAAAATTT |
| | | TGTTTTTAACATTTCAATCA |
| SEQ ID NO: 318 | 3UTR241 | AATCACGTCTGgATAACTTTTATTATTTCTTTTATTAATCAACAAAATTT |
| | | TGTTTTTAACATTTCAATCA |
| SEQ ID NO: 319 | 3UTR242 | AATCACGTCTGgATAACTTTTATTATTTCTTTTTTTAATCAACAAAATTT |
| | | TGTTTTTAACATTTCAATCA |
| SEQ ID NO: 320 | 3UTR243 | AATCACGTCTGgATAACTTTTATTATTTCTTTTTTTTATCAACAAAATTT |
| | | TGTTTTaAACATTTCAATCA |
| SEQ ID NO: 321 | 3UTR244 | AATCACGTCTGgATAACTTTTATTATTTCTTTTATTATTCAACAAATTTT |
| | | TGTTTTaAACATTTCAATCA |
| SEQ ID NO: 322 | 3UTR245 | TATAGACCAGATCCCGGAGTTGGAAAACAATGAAAAGGCCCCCAAGGTAG |
| | | TTATTCCAAATGTGAATATATagTTTTGTGTTCAGAAAACTAGGCAGGAA |
| | | AGTAGGAAAAGATCTGTTAATCGAATCACGTCTGCATAACTTTTATTATT |
| | | TCTTTTATTAATCAACAAAATTTTGTTTTTAACATTTC |
| SEQ ID NO: 323 | 3UTR246 | catcacatttaaaagcatctcagcctaccatgagaataagagaaagaaaa |
| | | tgaagatcaaaagcttattcatctgtttttctttttcgttggtgtaaagc |
| | | caacaccctgtctaaaaaacataaatttctttaatcattttgcctctttt |
| | | ctctgtgcttcaattaataaaaaatggaaagaatctaatagagtggtaca |
| | | gcactgttatttttcaaagatgtgttgctatcctgaaaattctgtaggtt |
| | | ctgtggaagttccagtgttctctcttattccacttcggtagaggatttct |
| | | agtttcttgtgggctaattaaataaatcattaatactcttctaagttatg |
| | | gattataaacattcaaaataatattttgacattatgataattctgaataa |
| | | aagaacaaaaacca |
| SEQ ID NO: 324 | 3UTR247 | gcttcctcttcactctgctctcaggagatctggctgtgaggccctcaggg |
| | | cagggatacaaagcggggagagggtacacaatgggtatctaataaatact |
| | | taagaggtggaa |
| SEQ ID NO: 325 | 3UTR248 | caggacacagccttggatcaggacagagacttgggggccatcctgcccct |
| | | ccaacccgacatgtgtacctcagctttttccctcacttgcatcaataaag |
| | | cttctgtgtttggaacagctaa |
| SEQ ID NO: 326 | 3UTR249 | tgcaaggctggccggaagcccttgcctgaaagcaagatttcagcctggaa |
| | | gagggcaaagtggacgggagtggacaggagtggatgcgataagatgtggt |
| | | ttgaagctgatgggtgccagccctgcattgctgagtcaatcaataaagag |
| | | ctttcttttgaccca |
| SEQ ID NO: 327 | 3UTR250 | agtgtccagaccattgtcttccaaccccagctggcctctagaacacccac |
| | | tggccagtcctagagctcctgtccctacccactctttgctacaataaatg |
| | | ctgaatgaatcca |
| SEQ ID NO: 328 | 3UTR251 | ctgcctctcgctcctcaacccctcccctccatccctggccccctccctgg |
| | | atgacattaaagaagggttgagctggtccctgcctgcatgtgactgtaaa |
| | | tccctcccatgttttctctgagtctccctttgcctgctgaggctgtatgt |
| | | gggctccaggtaacagtgctgtcttcgggccccctgaactgtgttcatgg |
| | | agcatctggctgggtaggcacatgctgggcttgaatccaggggggactga |
| | | atcctcagcttacggacctgggcccatctgtttctggagggctccagtct |
| | | tccttgtcctgtcttggagtccccaagaaggaatcacaggggaggaacca |
| | | gataccagccatgaccccaggctccaccaagcatcttcatgtccccctgc |
| | | tcatcccccactcccccccacccagagttgctcatcctgccagggctggc |
| | | tgtgcccaccccaaggctgccctcctgggggccccagaactgcctgatcg |
| | | tgccgtggcccagttttgtggcatctgcagcaacacaagagagaggacaa |
| | | tgtcctcctcttgacccgctgtcacctaaccagactcgggccctgcacct |
| | | ctcaggcacttctggaaaatgactgaggcagattcttcctgaagcccatt |
| | | ctccatggggcaacaaggacacctattctgtccttgtccttccatcgctg |
| | | ccccagaaagcctcacatatctccgtttagaatcaggtcccttctcccca |
| | | gatgaagaggagggtctctgctttgttttctctatctcctcctcagactt |
| | | gaccaggcccagcaggccccagaagaccattaccctatatcccttctcct |
| | | ccctagtcacatggccataggcctgctgatggctcaggaaggccattgca |
| | | aggactcctcagctatgggagaggaagcacatcacccattgacccccgca |
| | | acccctccctttcctcctctgagtcccgactggggccacatgcagcctga |
| | | cttctttgtgcctgttgctgtccctgcagtcttcagagggccaccgcagc |
| | | tccagtgccacggcaggaggctgttcctgaatagcccctgtggtaagggc |
| | | caggagagtccttccatcctccaaggccctgctaaaggacacagcagcca |
| | | ggaagtcccctgggcccctagctgaaggacagcctgctccctccgtctct |
| | | accaggaatggccttgtcctatggaaggcactgccccatcccaaactaat |
| | | ctaggaatcactgtctaaccactcactgtcatgaatgtgtacttaaagga |
| | | tgaggttgagtcataccaaatagtgatttcgatagttcaaaatggtgaaa |
| | | ttagcaattctacatgattcagtctaatcaatggataccgactgtttccc |
| | | acacaagtctcctgttctcttaagcttactcactgacagcctttcactct |
| | | ccacaaatacattaaagatatggccatcaccaagccccctaggatgacac |
| | | cagacctgagagtctgaagacctggatccaagttctgacttttccccctg |
| | | acagctgtgtgaccttcgtgaagtcgccaaacctctctgagccccagtca |
| | | ttgctagtaagacctgcctttgagttggtatgatgttcaagttagataac |
| | | aaaatgtttatacccattagaacagagaataaatagaactacatttcttg |
| | | ca |
| SEQ ID NO: 329 | 3UTR252 | ggacctgaagggtgacatcccaggaggggcctctgaaatttcccacaccc |
| | | cagcgcctgtgctgaggactccctccatgtggccccaggtgccaccaata |
| | | aaaatcctacagaaaa |
| SEQ ID NO: 330 | 3UTR253 | gacctcaatACCCCaagtCCACCtgCCTATcCATcctGCGAGctcctTGG |
| | | GTcctgcAATCTccaGGGCTgCCCCTgTAGGTTgcttaaaAGGGAcagta |
| | | TTCtcagtgCTCTCctACCCCacctCATGCctggccCCCCTccagGCATG |
| | | CtggCCTCCcAATAAAgctGGACaaGAAGcTGCTAtga |
| SEQ ID NO: 331 | 3UTR254 | gagcCTTCtgAGCCCagcgaCTTCtgaAGGGCCCCTtgcaaagtAATAGg |
| | | gCTTCtgcCTAAGcctctCCCTCcagccAATAGgcagcttTCTTaACTAT |
| | | cCTAACaagccttGGACcaAATGgaAATAAAgCTTTTTgATGca |
| SEQ ID NO: 332 | 3UTR255 | gcttcctcttcactctgctctcaggagacctggctatgaggccctcgggg |
| | | cagggatacaaagttagtgaggtctatgtccagagaagctgagatatggc |
| | | atataataggcatctaataaatgcttaagaggtggaa |
| SEQ ID NO: 333 | 3UTR256 | agctaaggttggatgcatggttgcatggatttggggtgtgctatgagggg |
| | | tggtgtatccttgggagagatataaagtggagggagggagccgtccggtc |
| | | agtagggcaccaatcccacctccttcattacctcctggccatgattctcc |
| | | tgggagataattctgctctctggagatgttggtaggaaagtttcaagtta |
| | | cgcagctgagaaacagggaccaaatagtgctcctgggtgcattgtcaccg |
| | | tgggtggccactcaagggtccaagcctctagggccatccttgggctaaca |
| | | actggggtgggtgtgagcaggtggaaggagcctcagcccatgccattacc |
| | | tcctgcttccttatcaggctgtgtgttaattctgggccagtctacaccct |
| | | cccacggggtggaaatggcctggaggatgtgagggcacccctcctctgaa |
| | | gatccctgtacacgtggtgttgggactggaaccattatgcggccccatag |
| | | gcctcaggagtcatcccagaagcagtggctgggaggtggtgtcctaagta |
| | | aggatctgtgcagaggacaaataaatcagtttttgatttgtcttgaaa |
| SEQ ID NO: 334 | 3UTR257 | cagtgcacaatATTTTCCCAtcTGTATtaTTTTTTTTCagcaTGTATtac |
| | | ttGACAAAgagacactgtgcagaGGGTGaccacAGTCTgtaatTCCCCAC |
| | | TTCaatACAAAGGGTGtcgttCTTTTcCAACAaAATAGcaATCCCTTTTA |
| | | TttCATTgCTTTTgaCTTTTcaatGGGTGtcCTAGGaacCTTTTagAAAG |
| | | AAATGgactttCATcctggAAATAtattaactgttAAAAAGAAAACattg |
| | | AAAATGTGTTtAGACAAcgtCATCCCCTggcagGCTAAagtgcTGTATcc |
| | | ttTAGTAAAATTGGAGGtagCAAACACTAAGgtgAAAAGATAATgatctC |
| | | ATTgTTTATTaaccTGTATtcTGTTTaCATgTCTTTAAAACagtggtTCT |
| | | TAAATTgtaagctcaggTTCaaaGTGTTggtAATGccTGATTcacaactt |
| | | tgaGAAGgTAGCActgGAGAGaattgGAAtGGGTGgcggTAATTGGTGat |
| | | actTCTTTgAATGTAGATTTCcAATCAcaTCTTTagtgtctGAAtatatc |
| | | caaatGTTTTaggaTGTATGTtaCTTCttAGAGAGaAATAAAgcATTTTt |
| | | ggGAAGAA |
| SEQ ID NO: 335 | 3UTR258 | agctaaggttggatgcatggttgcatggatttggggtgtgctatgagggg |
| | | tggtgtatccttgggagagatataaagtggagggagggagccgtccggtc |
| | | agtagggcaccaatcccacctccttcattacctcctggccatgattctcc |
| | | tgggagataattctgctctctggagatgttggtaggaaagtttcaagtta |
| | | cgcagctgagaaacagggaccaaatagtgctcctgggtgcattgtcaccg |
| | | tgggtggccactcaagggtccaagcctctagggccatccttgggctaaca |
| | | actggggtgggtgtgagcaggtggaaggagcctcagcccatgccattacc |
| | | tcctgcttccttatcaggctgtgtgttaattctgggccagtctacaccct |
| | | cccacggggtggaaatggcctggaggatgtgagggcacccctcctctgaa |
| | | gatccctgtacacgtggtgttgggactggaaccattatgcggccccatag |
| | | gcctcaggagtcatcccagaagcagtggctgggaggtggtgtcctaagta |
| | | aggatctgtgcagaggaca |
| SEQ ID NO: 336 | 3UTR259 | cagtgcacaatATTTTCCCAtcTGTATtaTTTTTTTTCagcaTGTATtac |
| | | ttGACAAAgagacactgtgcagaGGGTGaccacAGTCTgtaatTCCCCAC |
| | | TTCaatACAAAGGGTGtcgttCTTTTcCAACAaAATAGcaATCCCTTTTA |
| | | TttCATTgCTTTTgaCTTTTcaatGGGTGtcCTAGGaacCTTTTagAAAG |
| | | AAATGgactttCATcctggAAATAtattaactgttAAAAAGAAAACattg |
| | | AAAATGTGTTtAGACAAcgtCATCCCCTggcagGCTAAagtgcTGTATcc |
| | | ttTAGTAAAATTGGAGGtagCAAACACTAAGgtgAAAAGATAATgatctC |
| | | ATTgTTTATTaaccTGTATtcTGTTTaCATgTCTTTAAAACagtggtTCT |
| | | TAAATTgtaagctcaggTTCaaaGTGTTggtAATGccTGATTcacaactt |
| | | tgaGAAGgTAGCActgGAGAGaattgGAAtGGGTGgcggTAATTGGTGat |
| | | actTCTTTgAATGTAGATTTCcAATCAcaTCTTTagtgtctGAAtatatc |
| | | caaatGTTTTaggaTGTATGTtaCTTCttAGAGAGa |
| SEQ ID NO: 337 | 3UTR260 | gcttcctcttcactctgctctcaggagatctggctgtgaggccctcaggg |
| | | cagggatacaaagcggggagagggtacacaatgggtatct |
| SEQ ID NO: 338 | 3UTR261 | gacctcaatACCCCaagtCCACCtgCCTATcCATcctGCGAGctcctTGG |
| | | GTcctgcAATCTccaGGGCTgCCCCTgTAGGTTgcttaaaAGGGAcagta |
| | | TTCtcagtgCTCTCctACCCCacctCATGCctggccCCCCTccagGCATG |
| | | CtggCCTCCc |
| SEQ ID NO: 339 | 3UTR262 | aaagcatctcagcctaccatgagaataagagaaagaaaatgaagatcaaa |
| | | agcttattcatctgtttttctttttcgttggtgtaaagccaacaccctgt |
| | | ctaaaaaacataaatttctttaatcattttgcctcttttctctgtgcttc |
| | | aatt |
| SEQ ID NO: 340 | 3UTR263 | caggacacagccttggatcaggacagagacttgggggccatcctgcccct |
| | | ccaacccgacatgtgtacctcagctttttccctcacttgcatc |
| SEQ ID NO: 341 | 3UTR264 | tgcaaggctggccggaagcccttgcctgaaagcaagatttcagcctggaa |
| | | gagggcaaagtggacgggagtggacaggagtggatgcgataagatgtggt |
| | | ttgaagctgatgggtgccagccctgcattgctgagtcaatca |
| SEQ ID NO: 342 | 3UTR265 | agtgtccagaccattgtcttccaaccccagctggcctctagaacacccac |
| | | tggccagtcctagagctcctgtccctacccactctttgctac |
| SEQ ID NO: 343 | 3UTR266 | gcttcctcttcactctgctctcaggagacctggctatgaggccctcgggg |
| | | cagggatacaaagttagtgaggtctatgtccagagaagctgagatatggc |
| | | atataataggcatct |
| SEQ ID NO: 344 | 3UTR267 | ggacctgaagggtgacatcccaggaggggcctctgaaatttcccacaccc |
| | | cagcgcctgtgctgaggactccctccatgtggccccaggtgccacc |
| SEQ ID NO: 345 | 3UTR268 | gagcCTTCtgAGCCCagcgaCTTCtgaAGGGCCCCTtgcaaagtAATAGg |
| | | gCTTCtgcCTAAGcctctCCCTCcagccAATAGgcagcttTCTTaACTAT |
| | | cCTAACaagccttGGACcaAATGga |
| SEQ ID NO: 346 | 3UTR269 | actaagttaaatatttctgcacagtgttcccatggccccttgcatttcct |
| | | tcttaactctctgttacacgtcattgaaactacacttttttggtctgttt |
| | | ttgtgctagactgtaagttccttgggggcagggcctttgtctgtctcatc |
| | | tctgtattcccaaatgcctaacagtacagagccatgactcaataaataca |
| | | tgttaaatggatgaatgaa |
| SEQ ID NO: 347 | 3UTR270 | actaagttaaatatttctgcacagtgttcccatggccccttgcatttcct |
| | | tcttaactctctgttacacgtcattgaaactacacttttttggtctgttt |
| | | ttgtgctagactgtaagttccttgggggcagggcctttgtctgtctcatc |
| | | tctgtattcccaaatgcctaacagtacagagccatgactc |
| SEQ ID NO: 348 | 3UTR271 | aaaattaactgctaacttctattgacccacaaagtttcagaaattctctg |
| | | aaagtttcttccttttttctcttactatatttattgatttcaagtcttct |
| | | attaaggacatttagccttcaatggaaattaaaactcatttaggactgta |
| | | tttccaaattactgatatcagagttatttaaaaattgtttatttgaggag |
| | | ataacatttcaactttgttcctaaatatataataataaaatgattgactt |
| | | tatttgcatttttatgaccacttgtcatttattttgtcttcgtaaattat |
| | | tttcattatatcaaatattttagtatgtacttaataaaataggagaacat |
| | | tttagagtttcaaattcccaggtattttccttgtttattacccctaaatc |
| | | attcctatttaattcttctttttaaatggagaaaattatgtctttttaat |
| | | atggtttttgttttgttatatattcacaggctggagacgtttaaaagacc |
| | | gtttcaaaagagatttacttttttaaaggactttatctgaacagagagat |
| | | ataatatttttcctattggacaatggacttgcaaagcttcacttcatttt |
| | | aagagcaaaagaccccatgttgaaaactccataacagttttatgctgatg |
| | | ataatttatctac |
| SEQ ID NO: 349 | 3UTR272 | aaaattaactgctaacttctattgacccacaaagtttcagaaattctctg |
| | | aaagtttcttccttttttctcttactatATTTAttgatttcaagtcttct |
| | | attaaggacATTTAgccttcaatggaa |
| SEQ ID NO: 350 | 3UTR273 | aaaattaactgctaacttctattgacccacaaagtttcagaaattctctg |
| | | aaagtttcttccttttttctcttactatTtttAttgatttcaagtcttct |
| | | attaaggacaAttagccttcaatggaa |
| SEQ ID NO: 351 | 3UTR274 | ggggccttctgacatgagtctggcctggccccacctcctagttcctcata |
| | | ataaagacagattgcttcttcgcttctcactgaggggccttctgacatga |
| | | gtctggcctggccccacctccccagtttctcataataaagacagattgct |
| | | tcttcacttgaa |
| SEQ ID NO: 352 | 3UTR275 | GGGGCCTTCtgaCATgAGTCTggcctggcCCCACctCCTAGTTCctCAT |
| SEQ ID NO: 353 | 3UTR276 | TGTGTTCAGAAAACTAGGCAGGAAAGTAGGAAAAGATCTGTTAATCGAAT |
| | | CACGTCTGCATAACTTTTATTATTTCTTTTATTAATCAACAAAATTTTGT |
| | | TTTTAACATTTCTATAGACCAGATCCCGGAGTTGGAAAACAATGAAAAGG |
| | | CCCCCAAGGTAGTT |
| SEQ ID NO: 354 | 3UTR277 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTTTATAG |
| SEQ ID NO: 355 | 3UTR278 | CGTCTGCATAACTTTTATTATTTCTTTTATTAATCAACAAAATTTTGTTT |
| | | TTAACATTTCTGTGTTCAGAAAACTAGGCAGGAAAGTAGGAAAAGATCTG |
| | | TTAATCGAATCACGTCTGCATAACTTTTATTATTTCTTTTATTAATCAAC |
| | | AAAATTTTGTTTTTAACATTTCTATAGACCAGATCCCGGAGTTGGAAAAC |
| | | AATGAAAAGGCCCCCAAGGTAGTTCGTCTGCATAACTTTTATTATTTCTT |
| | | TTATTAATCAACAAAATTTTGTTTTTAACATTTC |
| SEQ ID NO: 356 | 3UTR279 | CGTCTGCATAACTTTTATTATTTCTTTTATTAATCAACAAAATTTTGTTT |
| | | TTAACATTTCAATCATGTGTTCAGAAAACTAGGCAGGAAAGTAGGAAAAG |
| | | ATCTGTTAATCGCGTCTGCATAACTTTTATTATTTCTTTTATTAATCAAC |
| | | AAAATTTTGTTTTTAACATTTCACCAGATCCCGGAGTTGGAAAACAATGA |
| | | AAAGGCCCCCAAGGTAGTTTATAGCGTCTGCATAACTTTTATTATTTCTT |
| | | TTATTAATCAACAAAATTTTGTTTTTAACATTTC |
| SEQ ID NO: 357 | 3UTR280 | AATCATGTGTTCAGAAAACTAGGCAGGAAAGTAGGAAAAGATCTGTTAAT |
| | | CGAATCACGTCTGCATAACTTTTATTATTTCTTTTATTAATCAACAAAAT |
| | | TTTGTTTTTAACATTTCTATAGACCAGATCCCGGAGTTGGAAAACAATGA |
| | | AAAGGCCCCCAAGGTAGTTAATCA |
| SEQ ID NO: 358 | 3UTR281 | TGTGTTCAGAAAACTAGGCAGGAAAGTAGGAAAAGATCTGTTAATCGAAT |
| | | CATGTGTTCAGAAAACTAGGCAGGAAAGTAGGAAAAGATCTGTTAATCGC |
| | | GTCTGCATAACTTTTATTATTTCTTTTATTAATCAACAAAATTTTGTTTT |
| | | TAACATTTCACCAGATCCCGGAGTTGGAAAACAATGAAAAGGCCCCCAAG |
| | | GTAGTTTATAGTGTGTTCAGAAAACTAGGCAGGAAAGTAGGAAAAGATCT |
| | | GTTAATCG |
| SEQ ID NO: 359 | 3UTR282 | ACCAGATCCCGGAGTTGGAAAACAATGAAAAGGCCCCCAAGGTAGTTAAT |
| | | CATGTGTTCAGAAAACTAGGCAGGAAAGTAGGAAAAGATCTGTTAATCGC |
| | | GTCTGCATAACTTTTATTATTTCTTTTATTAATCAACAAAATTTTGTTTT |
| | | TAACATTTCACCAGATCCCGGAGTTGGAAAACAATGAAAAGGCCCCCAAG |
| | | GTAGTTTATAGACCAGATCCCGGAGTTGGAAAACAATGAAAAGGCCCCCA |
| | | AGGTAGTT |
| SEQ ID NO: 360 | 3UTR283 | TATAGTGTGTTCAGAAAACTAGGCAGGAAAGTAGGAAAAGATCTGTTAAT |
| | | CGAATCACGTCTGCATAACTTTTATTATTTCTTTTATTAATCAACAAAAT |
| | | TTTGTTTTTAACATTTCTATAGACCAGATCCCGGAGTTGGAAAACAATGA |
| | | AAAGGCCCCCAAGGTAGTTTATAG |
| SEQ ID NO: 361 | 3UTR284 | TGTGTTCAGAAAACTAGGCAGGAAAGTAGGAAAAGATCTGTTAATCGCGT |
| | | CTGCATAACTTTTATTATTTCTTTTATTAATCAACAAAATTTTGTTTTTA |
| | | ACATTTCACCAGATCCCGGAGTTGGAAAACAATGAAAAGGCCCCCAAGGT |
| | | AGTT |
| SEQ ID NO: 362 | 3UTR285 | TGTGTTCAGAAAACTAGGCAGGAAAGTAGGAAAAGATCTGTTAATCGAAT |
| | | CACGTCTGCATAACTTTTATTATTTCTTTTATTAATCAACAAAATTTTGT |
| | | TTTTAACATTTC |
| SEQ ID NO: 363 | 3UTR286 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTC |
| SEQ ID NO: 364 | 3UTR287 | TGTGTTCAGAAAACTAGGCAGGAAAGTAGGAAAAGATCTGTTAATCGAAT |
| | | CACGTCTGCATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 365 | 3UTR288 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 366 | 3UTR289 | TGTGTTCAGAAAACTAGGCAGGAAAGTAGGAAAAGATCTGTTAATCGCGT |
| | | CTGgATAACTTTTATTATTTCTTTTTTTtATCAACAAAATTTTGTTTTAA |
| | | ACATTTCACCAGATCCCGGAGTTGGAAAACAATGAAAAGGCCCCCAAGGT |
| | | AGTT |
| SEQ ID NO: 367 | 3UTR290 | TGTGTTCAGAAAACTAGGCAGGAAAGTAGGAAAAGATCTGTTAATCGAAT |
| | | CACGTCTGgATAACTTTTATTATTTCTTTTTTTtATCAACAAAATTTTGT |
| | | TTTAAACATTTC |
| SEQ ID NO: 368 | 3UTR291 | AATCATGTGTTCAGAAAACTAGGCAGGAAAGTAGGAAAAGATCTGTTAAT |
| | | CGCGTCTGgATAACTTTTATTATTTCTTTTTTTtATCAACAAAATTTTGT |
| | | TTTAAACATTTC |
| SEQ ID NO: 369 | 3UTR292 | TGTGTTCAGAAAACTAGGCAGGAAAGTAGGAAAAGATCTGTTAATCGAAT |
| | | CACGTCTGgATAACTTTTATTATTTCTTTTTTTtATCAACAAAATTTTGT |
| | | TTTAAACATTTCACCAGATCCCGGAGTTGGAAAACAATGAAAAGGCCCCC |
| | | AAGGTAGTT |
| SEQ ID NO: 370 | 3UTR293 | AATCATGTGTTCAGAAAACTAGGCAGGAAAGTAGGAAAAGATCTGTTAAT |
| | | CGCGTCTGgATAACTTTTATTATTTCTTTTTTTtATCAACAAAATTTTGT |
| | | TTTAAACATTTCACCAGATCCCGGAGTTGGAAAACAATGAAAAGGCCCCC |
| | | AAGGTAGTT |
| SEQ ID NO: 371 | 3UTR294 | TGTGTTCAGAAAACTAGGCAGGAAAGTAGGAAAAGATCTGTTAATCGCGT |
| | | CTGgATAACTTTTATTATTTCTTTTTTTAATCAACAAAATTTTGTTTTTA |
| | | ACATTTCACCAGATCCCGGAGTTGGAAAACAATGAAAAGGCCCCCAAGGT |
| | | AGTT |
| SEQ ID NO: 372 | 3UTR295 | TGTGTTCAGAAAACTAGGCAGGAAAGTAGGAAAAGATCTGTTAATCGAAT |
| | | CACGTCTGgATAACTTTTATTATTTCTTTTTTTAATCAACAAAATTTTGT |
| | | TTTTAACATTTC |
| SEQ ID NO: 373 | 3UTR296 | AATCATGTGTTCAGAAAACTAGGCAGGAAAGTAGGAAAAGATCTGTTAAT |
| | | CGCGTCTGgATAACTTTTATTATTTCTTTTTTTAATCAACAAAATTTTGT |
| | | TTTTAACATTTC |
| SEQ ID NO: 374 | 3UTR297 | TGTGTTCAGAAAACTAGGCAGGAAAGTAGGAAAAGATCTGTTAATCGAAT |
| | | CACGTCTGgATAACTTTTATTATTTCTTTTTTTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 375 | 3UTR298 | AATCATGTGTTCAGAAAACTAGGCAGGAAAGTAGGAAAAGATCTGTTAAT |
| | | CGCGTCTGgATAACTTTTATTATTTCTTTTTTTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 376 | 3UTR299 | TGTGTTCAGAAAACTAGGCAGGAAAGTAGGAAAAGATCTGTTAATCGCGT |
| | | CTGCATAACTTTTATTATTTCTTTTATTAATCAACAAAATTTTGTTTTTA |
| | | ACATTTCTATAGACCAGATCCCGGAGTTGGAAAACAATGAAAAGGCCCCC |
| | | AAGGTAGTT |
| SEQ ID NO: 377 | 3UTR300 | TGTGTTCAGAAAACTAGGCAGGAAAGTAGGAAAAGATCTGTTAATCGAAT |
| | | CACGTCTGCATAACTTTTATTATTTCTTTTATTAATCAACAAAATTTTGT |
| | | TTTTAACATTTCTATAG |
| SEQ ID NO: 378 | 3UTR301 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTCTATAG |
| SEQ ID NO: 379 | 3UTR303 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTCTATAGACCAGATCCCGGAGTTGGAAAACAATGAAAAGG |
| | | CCCCCAAGGTAGTT |
| SEQ ID NO: 380 | 3UTR304 | TGTGTTCAGAAAACTAGGCAGGAAAGTAGGAAAAGATCTGTTAATCGCGT |
| | | CTGCATAACTTTTATTATTTCTTTTATTAATCAACAAAATTTTGTTTTTA |
| | | ACATTTCACCAGATCCCGGAGTTGGAAAACAATGAAAAGGCCCCCAAGGT |
| | | AGTTACCAGATCCCGGAGTTGGAAAACAATGAAAAGGCCCCCAAGGTAGT |
| | | T |
| SEQ ID NO: 381 | 3UTR307 | TGTGTTCAGAAAACTAGGCAGGAAAGTAGGAAAAGATCTGTTAATCGAAT |
| | | CACGTCTGCATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTTACCAGATCCCGGAGTTGGAAAACAATGAAAAGGCCCCCAAG |
| | | GTAGTT |
| SEQ ID NO: 382 | 3UTR308 | AATCATGTGTTCAGAAAACTAGGCAGGAAAGTAGGAAAAGATCTGTTAAT |
| | | CGCGTCTGCATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTTACCAGATCCCGGAGTTGGAAAACAATGAAAAGGCCCCCAAG |
| | | GTAGTT |
| SEQ ID NO: 383 | 3UTR309 | TGTGTTCAGAAAACTAGGCAGGAAAGTAGGAAAAGATCTGTTAATCGCGT |
| | | CTGgATAACTTTTATTATTTCTTTTATTAATCAACAAAATTTTGTTTTTA |
| | | ACATTTCACCAGATCCCGGAGTTGGAAAACAATGAAAAGGCCCCCAAGGT |
| | | AGTTACCAGATCCCGGAGTTGGAAAACAATGAAAAGGCCCCCAAGGTAGT |
| | | T |
| SEQ ID NO: 384 | 3UTR310 | TGTGTTCAGAAAACTAGGCAGGAAAGTAGGAAAAGATCTGTTAATCGAAT |
| | | CACGTCTGgATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 385 | 3UTR311 | AATCATGTGTTCAGAAAACTAGGCAGGAAAGTAGGAAAAGATCTGTTAAT |
| | | CGCGTCTGgATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTT |
| SEQ ID NO: 386 | 3UTR312 | TGTGTTCAGAAAACTAGGCAGGAAAGTAGGAAAAGATCTGTTAATCGAAT |
| | | CACGTCTGgATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTTACCAGATCCCGGAGTTGGAAAACAATGAAAAGGCCCCCAAG |
| | | GTAGTT |
| SEQ ID NO: 387 | 3UTR313 | AATCATGTGTTCAGAAAACTAGGCAGGAAAGTAGGAAAAGATCTGTTAAT |
| | | CGCGTCTGgATAACTTTTATTATTTCTTTTATTAATCAACAAAATTTTGT |
| | | TTTTAACATTTCACCAGATCCCGGAGTTGGAAAACAATGAAAAGGCCCCC |
| | | AAGGTAGTTACCAGATCCCGGAGTTGGAAAACAATGAAAAGGCCCCCAAG |
| | | GTAGTT |
| SEQ ID NO: 388 | 3UTR314 | TGTGTTCAGAAAACTAGGCAGGAAAGTAGGAAAAGATCTGTTAATCGATG |
| | | TCTCCAGTTACAACTCCGCAGTGGATGTGAAGAAGCGGTGCCTTTGAGAG |
| | | TCTACTTTTGCTCTCTTCGGAAGAACCCTTAGGGGTTCGTGCATGGGCTT |
| | | GCATAGCAAGTCTAGATGCGGGTACCGTACAGTGTTGAAAAACACTGTAA |
| | | ATCTCTAAAAGAGACCAACCAGATCCCGGAGTTGGAAAACAATGAAAAGG |
| | | CCCCCAAGGTAGTT |
| SEQ ID NO: 389 | 3UTR315 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCGGTGCCTTT |
| | | GAGAGTCTACTTTTGCTCTCTTCGGAAGAACCCTTAGGGGTTCGTGCATG |
| | | GGCTTGCATAGCAAGTCTAGATGCGGGTACCGTACAGTGTTGAAAAACAC |
| | | TGTAAATCTCTAAAAGAGACCA |
| SEQ ID NO: 390 | 3UTR316 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCGGTGCCTTT |
| | | GAGAGTCTACTTTTGCTCTCTTCGGAAGAACCCTTAGGGGTTCGTGCATG |
| | | GGCTTGCATAGCAAGTCTAGATGCGGGTACCGTACAGTGTTGAAAAACAC |
| | | TGTAAATCTCTAAAAGAGACCA |
| SEQ ID NO: 391 | 3UTR317 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCGGTGCCTTT |
| | | GAGAGTCTACTTTTGCTCTCTTCGGAAGAACCCTTAGGGGTTCGTGCATG |
| | | GGCTTGCATAGCAAGTCTAGATGCGGGTACCGTACAGTGTTGAAAAACAC |
| | | TGTAAATCTCTAAAAGAGACCAACCAGATCCCGGAGTTGGAAAACAATGA |
| | | AAAGGCCCCCAAGGTAGTT |
| SEQ ID NO: 392 | 3UTR318 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCGGTGCCTTT |
| | | GAGAGTCTACTTTTGCTCTCTTCGGAAGAACCCTTAGGGGTTCGTGCATG |
| | | GGCTTGCATAGCAAGTCTAGATGCGGGTACCGTACAGTGTTGAAAAACAC |
| | | TGTAAATCTCTAAAAGAGACCAACCAGATCCCGGAGTTGGAAAACAATGA |
| | | AAAGGCCCCCAAGGTAGTT |
| SEQ ID NO: 393 | 3UTR319 | TGTGTTCAGAAAACTAGGCAGGAAAGTAGGAAAAGATCTGTTAATCGATG |
| | | TCTCCAGTTACAACTCCGCAGTGGATGTGAAGAAGCGGCAGCAGCGGAGG |
| | | TCATGAAGGTTTTTCTTTTCCTGAGAAAACAACACGTATTGTTTTCTCAG |
| | | GTTTTGCTTTTTGGCCTTTTTCTAGCTTAAAAAAAAAAAAAGCAAAAGAT |
| | | GCTGGTGGTTGGCACTCCTGGTTTCCAGGACGGGGTTCAAATCCCTGCGG |
| | | CGTCTTTGCTTACCAGATCCCGGAGTTGGAAAACAATGAAAAGGCCCCCA |
| | | AGGTAGTT |
| SEQ ID NO: 394 | 3UTR320 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCGGCAGCAGC |
| | | GGAGGTCATGAAGGTTTTTCTTTTCCTGAGAAAACAACACGTATTGTTTT |
| | | CTCAGGTTTTGCTTTTTGGCCTTTTTCTAGCTTAAAAAAAAAAAAAGCAA |
| | | AAGATGCTGGTGGTTGGCACTCCTGGTTTCCAGGACGGGGTTCAAATCCC |
| | | TGCGGCGTCTTTGCTT |
| SEQ ID NO: 395 | 3UTR321 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCGGCAGCAGC |
| | | GGAGGTCATGAAGGTTTTTCTTTTCCTGAGAAAACAACACGTATTGTTTT |
| | | CTCAGGTTTTGCTTTTTGGCCTTTTTCTAGCTTAAAAAAAAAAAAAGCAA |
| | | AAGATGCTGGTGGTTGGCACTCCTGGTTTCCAGGACGGGGTTCAAATCCC |
| | | TGCGGCGTCTTTGCTT |
| SEQ ID NO: 396 | 3UTR322 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCGGCAGCAGC |
| | | GGAGGTCATGAAGGTTTTTCTTTTCCTGAGAAAACAACACGTATTGTTTT |
| | | CTCAGGTTTTGCTTTTTGGCCTTTTTCTAGCTTAAAAAAAAAAAAAGCAA |
| | | AAGATGCTGGTGGTTGGCACTCCTGGTTTCCAGGACGGGGTTCAAATCCC |
| | | TGCGGCGTCTTTGCTTACCAGATCCCGGAGTTGGAAAACAATGAAAAGGC |
| | | CCCCAAGGTAGTT |
| SEQ ID NO: 397 | 3UTR323 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCGGCAGCAGC |
| | | GGAGGTCATGAAGGTTTTTCTTTTCCTGAGAAAACAACACGTATTGTTTT |
| | | CTCAGGTTTTGCTTTTTGGCCTTTTTCTAGCTTAAAAAAAAAAAAAGCAA |
| | | AAGATGCTGGTGGTTGGCACTCCTGGTTTCCAGGACGGGGTTCAAATCCC |
| | | TGCGGCGTCTTTGCTTACCAGATCCCGGAGTTGGAAAACAATGAAAAGGC |
| | | CCCCAAGGTAGTT |
| SEQ ID NO: 398 | 3UTR324 | TGTGTTCAGAAAACTAGGCAGGAAAGTAGGAAAAGATCTGTTAATCGGGG |
| | | ATgaGAAcAGAGAGAAATAtaTTCaTAATTtacTTTATgaccTAGAAgGA |
| | | AactgTCGTGTGtcctATACAttgcCATcaacttTGTTTcctCATctcaA |
| | | CCAGATCCCGGAGTTGGAAAACAATGAAAAGGCCCCCAAGGTAGTT |
| SEQ ID NO: 399 | 3UTR325 | TGTGTTCAGAAAACTAGGCAGGAAAGTAGGAAAAGATCTGTTAATCGAAT |
| | | CAGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctca |
| SEQ ID NO: 400 | 3UTR326 | AATCATGTGTTCAGAAAACTAGGCAGGAAAGTAGGAAAAGATCTGTTAAT |
| | | CGGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctca |
| SEQ ID NO: 401 | 3UTR327 | TGTGTTCAGAAAACTAGGCAGGAAAGTAGGAAAAGATCTGTTAATCGAAT |
| | | CAGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaACCAGATCCCGGAGTTGGAAAACAATGAAAAGGCCCCCAAGGTAGT |
| | | T |
| SEQ ID NO: 402 | 3UTR328 | AATCATGTGTTCAGAAAACTAGGCAGGAAAGTAGGAAAAGATCTGTTAAT |
| | | CGGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaACCAGATCCCGGAGTTGGAAAACAATGAAAAGGCCCCCAAGGTAGT |
| | | T |
| SEQ ID NO: 403 | 3UTR329 | TGTGTTCAGAAAACTAGGCAGGAAAGTAGGAAAAGATCTGTTAATCGGGG |
| | | ATgaGAAcAGAGAGAAATAtaTTCaTAATTtacTTTATgaccTAGAAgGA |
| | | AactgTCGTGTGtcctATACAttgcCATcaacttTGTTTcctCATctcaC |
| | | GTCTGCATAACTTTTATTATTTCTTTTATTAATCAACAAAATTTTGTTTT |
| | | TAACATTTCACCAGATCCCGGAGTTGGAAAACAATGAAAAGGCCCCCAAG |
| | | GTAGTT |
| SEQ ID NO: 404 | 3UTR330 | TGTGTTCAGAAAACTAGGCAGGAAAGTAGGAAAAGATCTGTTAATCGAAT |
| | | CAGGGAtgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaCGTCTGCATAACTTTTATTATTTCTTTTATTAATCAACAAAATTTT |
| | | GTTTTTAACATTTC |
| SEQ ID NO: 405 | 3UTR331 | AATCATGTGTTCAGAAAACTAGGCAGGAAAGTAGGAAAAGATCTGTTAAT |
| | | CGGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaCGTCTGCATAACTTTTATTATTTCTTTTATTAATCAACAAAATTTT |
| | | GTTTTTAACATTTC |
| SEQ ID NO: 406 | 3UTR332 | TGTGTTCAGAAAACTAGGCAGGAAAGTAGGAAAAGATCTGTTAATCGAAT |
| | | CAGGGAtgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaCGTCTGCATAACTTTTATTATTTCTTTTATTAATCAACAAAATTTT |
| | | GTTTTTAACATTTCACCAGATCCCGGAGTTGGAAAACAATGAAAAGGCCC |
| | | CCAAGGTAGTT |
| SEQ ID NO: 407 | 3UTR333 | AATCATGTGTTCAGAAAACTAGGCAGGAAAGTAGGAAAAGATCTGTTAAT |
| | | CGGGGATgaGAAcAGAGAGAAATAtaTTCaTAATTtacTTTATgaccTAG |
| | | AAgGAAactgTCGTGTGtcctATACAttgcCATcaacttTGTTTcctCAT |
| | | ctcaCGTCTGCATAACTTTTATTATTTCTTTTATTAATCAACAAAATTTT |
| | | GTTTTTAACATTTCACCAGATCCCGGAGTTGGAAAACAATGAAAAGGCCC |
| | | CCAAGGTAGTT |
| SEQ ID NO: 408 | 3UTR334 | TGTGTTCAGAAAACTAGGCAGGAAAGTAGGAAAAGATCTGTTAATCGgac |
| | | agagctctgcGGTGtcaggGCGAGAACCCaTCTTCcAACCCcggcTATTT |
| | | ggagacggAAAAActgGAAttCTAACaaGGAGGagaggagACCAGATCCC |
| | | GGAGTTGGAAAACAATGAAAAGGCCCCCAAGGTAGTT |
| SEQ ID NO: 409 | 3UTR335 | TGTGTTCAGAAAACTAGGCAGGAAAGTAGGAAAAGATCTGTTAATCGAAT |
| | | CAgacagagctctgcGGTGtcaggGCGAGAACCCaTCTTCcAACCCcggc |
| | | TATTTggagacggAAAAActgGAAttCTAACaaGGAGGagaggag |
| SEQ ID NO: 410 | 3UTR336 | AATCATGTGTTCAGAAAACTAGGCAGGAAAGTAGGAAAAGATCTGTTAAT |
| | | CGgacagagctctgcGGTGtcaggGCGAGAACCCaTCTTCcAACCCcggc |
| | | TATTTggagacggAAAAActgGAAttCTAACaaGGAGGagaggag |
| SEQ ID NO: 411 | 3UTR337 | TGTGTTCAGAAAACTAGGCAGGAAAGTAGGAAAAGATCTGTTAATCGAAT |
| | | CAgacagagctctgcGGTGtcaggGCGAGAACCCaTCTTCcAACCCcggc |
| | | TATTTggagacggAAAAActgGAAttCTAACaaGGAGGagaggagACCAG |
| | | ATCCCGGAGTTGGAAAACAATGAAAAGGCCCCCAAGGTAGTT |
| SEQ ID NO: 412 | 3UTR338 | AATCATGTGTTCAGAAAACTAGGCAGGAAAGTAGGAAAAGATCTGTTAAT |
| | | CGgacagagctctgcGGTGtcaggGCGAGAACCCaTCTTCcAACCCcggc |
| | | TATTTggagacggAAAAActgGAAttCTAACaaGGAGGagaggagACCAG |
| | | ATCCCGGAGTTGGAAAACAATGAAAAGGCCCCCAAGGTAGTT |
| SEQ ID NO: 413 | 3UTR339 | TGTGTTCAGAAAACTAGGCAGGAAAGTAGGAAAAGATCTGTTAATCGgac |
| | | agagctctgcGGTGtcaggGCGAGAACCCaTCTTCcAACCCcggcTATTT |
| | | ggagacggAAAAActgGAAttCTAACaaGGAGGagaggagCGTCTGCATA |
| | | ACTTTTATTATTTCTTTTATTAATCAACAAAATTTTGTTTTTAACATTTC |
| | | ACCAGATCCCGGAGTTGGAAAACAATGAAAAGGCCCCCAAGGTAGTT |
| SEQ ID NO: 414 | 3UTR340 | TGTGTTCAGAAAACTAGGCAGGAAAGTAGGAAAAGATCTGTTAATCGAAT |
| | | CAgacagagctctgcGGTGtcaggGCGAGAACCCaTCTTCcAACCCcggc |
| | | TATTTggagacggAAAAActgGAAttCTAACaaGGAGGagaggagCGTCT |
| | | GCATAACTTTTATTATTTCTTTTATTAATCAACAAAATTTTGTTTTTAAC |
| | | ATTTC |
| SEQ ID NO: 415 | 3UTR341 | AATCATGTGTTCAGAAAACTAGGCAGGAAAGTAGGAAAAGATCTGTTAAT |
| | | CGgacagagctctgcGGTGtcaggGCGAGAACCCaTCTTCcAACCCcggc |
| | | TATTTggagacggAAAAActgGAAttCTAACaaGGAGGagaggagCGTCT |
| | | GCATAACTTTTATTATTTCTTTTATTAATCAACAAAATTTTGTTTTTAAC |
| | | ATTTC |
| SEQ ID NO: 416 | 3UTR342 | TGTGTTCAGAAAACTAGGCAGGAAAGTAGGAAAAGATCTGTTAATCGAAT |
| | | CAgacagagctctgcGGTGtcaggGCGAGAACCCaTCTTCcAACCCcggc |
| | | TATTTggagacggAAAAActgGAAttCTAACaaGGAGGagaggagCGTCT |
| | | GCATAACTTTTATTATTTCTTTTATTAATCAACAAAATTTTGTTTTTAAC |
| | | ATTTCACCAGATCCCGGAGTTGGAAAACAATGAAAAGGCCCCCAAGGTAG |
| | | TT |
| SEQ ID NO: 417 | 3UTR343 | AATCATGTGTTCAGAAAACTAGGCAGGAAAGTAGGAAAAGATCTGTTAAT |
| | | CGgacagagctctgcGGTGtcaggGCGAGAACCCaTCTTCcAACCCcggc |
| | | TATTTggagacggAAAAActgGAAttCTAACaaGGAGGagaggagCGTCT |
| | | GCATAACTTTTATTATTTCTTTTATTAATCAACAAAATTTTGTTTTTAAC |
| | | ATTTCACCAGATCCCGGAGTTGGAAAACAATGAAAAGGCCCCCAAGGTAG |
| | | TT |
| SEQ ID NO: 418 | 3UTR344 | TGTGTTCAGAAAACTAGGCAGGAAAGTAGGAAAAGATCTGTTAATCGATG |
| | | TCTCCAGTTACAACTCCGCAGTGGATGTGAAGAAGCggcaGAAGtcagTT |
| | | CTTCtGTCCATCCCtcTCCCCAgccaggATAGAgctatCTTTTcCATctC |
| | | ATcctcaGAAGAGACTcaGAAGAAagATGacAGCCCtcagAATGcacgtt |
| | | ATGagGAAGgcagAATGTGGGTctgTAATTCCTCCgtGTCCCTTCtcCCC |
| | | CTctgcaaaccgtcgTAACAATAATAgTTCctAACACatGGGACaattgt |
| | | gaGGACCAGATCCCGGAGTTGGAAAACAATGAAAAGGCCCCCAAGGTAGT |
| | | T |
| SEQ ID NO: 419 | 3UTR345 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCggcaGAAGt |
| | | cagTTCTTCtGTCCATCCCtcTCCCCAgccaggATAGAgctatCTTTTcC |
| | | ATctCATcctcaGAAGAGACTcaGAAGAAagATGacAGCCCtcagAATGc |
| | | acgttATGagGAAGgcagAATGTGGGTctgTAATTCCTCCgtGTCCCTTC |
| | | tcCCCCTctgcaaaccgtcgTAACAATAATAgTTCctAACACatGGGACa |
| | | attgtgagg |
| SEQ ID NO: 420 | 3UTR346 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCggcaGAAGt |
| | | cagTTCTTCtGTCCATCCCtcTCCCCAgccaggATAGAgctatCTTTTcC |
| | | ATctCATcctcaGAAGAGACTcaGAAGAAagATGacAGCCCtcagAATGc |
| | | acgttATGagGAAGgcagAATGTGGGTctgTAATTCCTCCgtGTCCCTTC |
| | | tcCCCCTctgcaaaccgtcgTAACAATAATAgTTCctAACACatGGGACa |
| | | attgtgagg |
| SEQ ID NO: 421 | 3UTR347 | TGTGTTCAGAAAACTAGGCAGGAAAGTAGGAAAAGATCTGTTAATCGAAT |
| | | CAATGTCTCCAGTTACAACTCCGCAGTGGATGTGAAGAAGCggcaGAAGt |
| | | cagTTCTTCtGTCCATCCCtcTCCCCAgccaggATAGAgctatCTTTTcC |
| | | ATctCATcctcaGAAGAGACTcaGAAGAAagATGacAGCCCtcagAATGc |
| | | acgttATGagGAAGgcagAATGTGGGTctgTAATTCCTCCgtGTCCCTTC |
| | | tcCCCCTctgcaaaccgtcgTAACAATAATAgTTCctAACACatGGGACa |
| | | attgtgaGGACCAGATCCCGGAGTTGGAAAACAATGAAAAGGCCCCCAAG |
| | | GTAGTT |
| SEQ ID NO: 422 | 3UTR348 | AATCATGTGTTCAGAAAACTAGGCAGGAAAGTAGGAAAAGATCTGTTAAT |
| | | CGATGTCTCCAGTTACAACTCCGCAGTGGATGTGAAGAAGCggcaGAAGt |
| | | cagTTCTTCtGTCCATCCCtcTCCCCAgccaggATAGAgctatCTTTTcC |
| | | ATctCATcctcaGAAGAGACTcaGAAGAAagATGacAGCCCtcagAATGc |
| | | acgttATGagGAAGgcagAATGTGGGTctgTAATTCCTCCgtGTCCCTTC |
| | | tcCCCCTctgcaaaccgtcgTAACAATAATAgTTCctAACACatGGGACa |
| | | attgtgaGGACCAGATCCCGGAGTTGGAAAACAATGAAAAGGCCCCCAAG |
| | | GTAGTT |
| SEQ ID NO: 423 | 3UTR350 | acctgagactggtggcttctagaagcagccattaccaactgtaccttccc |
| | | ttcttgctcagccaataaatatatcctctttcactca |
| SEQ ID NO: 424 | 3UTR351 | ccctgtgcaacagccactacattacttcaaactgagatccttccttttga |
| | | gggagcaagtccttccctttcattttttccagtcttcctccctgtgtatt |
| | | cattctcatgattattattttagtgggggggggtgggaaagattactttt |
| | | tctttatgtgtttgacgggaaaaaaactaggtaaaatctacagtacacca |
| | | caagggtcacaatactgttgtgcgcacatcgcggtagggcgtggaaaggg |
| | | gcaggccagagctacccgcagagttctcagaatcatgctgagagagctgg |
| | | aggcacccatgccatctcaacctcttccccgcccgttttacaaaggggga |
| | | ggctaaagcccagagacagcttgatcaaaggcacacagcaagtcagggtt |
| | | ggagcagtagctggagggaccttgtctcccagctcagggctctttcctcc |
| | | acaccattcaggtctttctttccgaggcccctgtctcagggtgaggtgct |
| | | tgagtctccaacggcaagggaacaagtacttcttgatacctgggatactg |
| | | tgcccagagcctcgaggaggtaatgaattaaagaagagaactgcctttgg |
| | | cagagttctataatgtaaacaatatcagactttttttttttataatcaag |
| | | cctaaaattgtatagacctaaaataaaatgaagtggtgagcttaaccctg |
| | | gaaaatgaatccctctatctctaaagaaaatctctgtgaaacccctatgt |
| | | ggaggcggaattgctctcccagcccttgcattgcagaggggcccatgaaa |
| | | gaggacaggctacccctttacaaatagaatttgagcatcagtgaggttaa |
| | | actaaggccctcttgaatctctgaatttgagatacaaacatgttcctggg |
| | | atcactgatgactttttatactttgtaaagacaattgttggagagcccct |
| | | cacacagccctggcctctgctcaactagcagatacagggatgaggcagac |
| | | ctgactctcttaaggaggctgagagcccaaactgctgtcccaaacatgca |
| | | cttccttgcttaaggtatggtacaagcaatgcctgcccattggagagaaa |
| | | aaacttaagtagataaggaaataagaaccactcataattcttcaccttag |
| | | gaataatctcctgttaatatggtgtacattcttcctgattattttctaca |
| | | catacatgtaaaatatgtctttcttttttaaatagggttgtactatgctg |
| | | ttatgagtggctttaatgaataaacatttgtagcatcctctttaatgggt |
| | | aaacagca |
| SEQ ID NO: 425 | 3UTR352 | gcagagaatacggttttggtgtcctgctacaaaaagacatcggtcagtaa |
| | | cgagcacgatgtggaaaaatgagagaagggacacattcaaccctggagag |
| | | ttcaatggctgctgaagctgcctgcttttcactgctgcaaggcctttctg |
| | | tgtgtgatgtgcatgggagcaacttgttcgtgggtcatcgggaatactag |
| | | ggagaaggtttcattgcccccagggcacttcacagagtgtgctggaggac |
| | | tgagtaagaaatgctgcccatgccaccgcttccggctcctgtgctttccc |
| | | tgaactgggacctttagtggtggccatttagccaccatctttgcaggttg |
| | | ctttgccctggtagggcagtaacattgggtcctgggtctttcatggggtg |
| | | atgctgggctggctccctcttggtcttcccaggctggggctgaccttcct |
| | | cgcagagaggccaggtgcaggttgggaatgaggcttgctgagaggggctg |
| | | tccagttcccagaaggcatatcagtctctgagggcttcctttggggccgg |
| | | gaacttgcgggtttgaggataggagttcacttcatcttctcagctcccat |
| | | ttctactcttaagtttctcagctcccatttctactctcccatggcttaat |
| | | gcttctttcattttctgtttgttttatacaaatgtcttagttgtacaaat |
| | | aaagtcccaggttaaagataacaaacggctcctgtgacataaacgtgcga |
| | | aagcccatctacagcaaagacatcagtgctccgtggaacagaaaccagaa |
| | | ggagaaaatttgtacatcctccttttgcacctgagatcctcatttgcccg |
| | | acgttgtaggtgtggagagtcctagagaggctaggaagcgcccagggcac |
| | | ccagagccaggctgcaggtgcctcaggcccctctcccagcccactcccaa |
| | | gctgaactagcacgtgttcatttctaccgcgggttgaaccgcagggatcc |
| | | ctggcttcaagtcaggcaccaaacagaaggtaggaggcatgaggggttct |
| | | gtcacatgtctcttcattttcttattgattcttagccttgacagttggag |
| | | aaggaaaaagcaaggagaaagtgctgagcaggagtggagagtgaagagca |
| | | ggacctcgtgcctgggtttgaaaaagtgcctgagacccatcttagccgcc |
| | | cctacctgagctttagcagcctaggagctattgcaccataaaacactgca |
| | | acctgaggctgcattaacagaagcaccacgtccaggtccagggagaatcc |
| | | agcctcacctgcttttcctggccagctcaaagaaggacattgacaaatca |
| | | ggttatgttcagttacctgtgacaaccacagaatgtaagaaatgttacaa |
| | | aaaaaacaggaaaggaaagacgactctggggaaggggcagtgtgaccttc |
| | | tcctggcatctggggtgcggccgcagcagaatgaggtgtggtgggtggag |
| | | ttagaaaggcatagatgcagtgttaccggaaggagggccttgagtgtaag |
| | | ttgtccaggtccttggcattttgaacaaagaactgaacaaaacatacaaa |
| | | gtaataaaggaataaaagcagcaaaaggaagaatttattgaagtgagaaa |
| | | gcactccacatggtaggagtggaccctaagcagatagcccatgggcccaa |
| | | ttgcaaagttttctgggttttaagtaccccttttgaggtacctgttggct |
| | | accccttatctgggtgaaggatttggtccatggctaatgaaaggctgagg |
| | | tgaattgacgccctatgcggatgaagggatggcccgtgcttggtctgtgg |
| | | tcaatccagggccgtctccctttccatctgagatgcagtggaagggggag |
| | | ggttgtagggagagtagcctttgatccttggttactcagtgtggggagat |
| | | ggggtttttccttttggtgtatgttaattggccttagatgccctgccccc |
| | | agacccaggtgttttccgtttgctccagctttgagaagtcagcacaaatt |
| | | ggcctcagattccctgcccccagacgtaggtgtttctccttcattcagca |
| | | cgaattggccttagatgccctgcccccagacccaggtgttttccatttga |
| | | tccagctttgagaagtcagcacaaattggcctcagattccctgcccccgg |
| | | acatagatgtttctccttgattcagcacgaattggccttagattccctgc |
| | | ccccagaccctagactcctgcctcagcaagaggtgttcaaaagaaacaaa |
| | | agctctgcccatcagctctgaggacaagggacaggctgccttccaaggct |
| | | gaggggaggggtgaaagaattggggtcagctggaccagactcctctaggg |
| | | ttcgtgtttcaggtgggcccctcacccggcccatcacaaacaaataagag |
| | | attaaggcctgcagtgcacatggtctgccctccttgaaggctcacaccca |
| | | gtgcacagcggaagtaagaacaggccacaaggcctcttgtcccactgcag |
| | | gctttgaaatttgaccgtctccctgctgctgtctttgcaattgcatgtac |
| | | ttgttcacaagttctcatggggttgggctgggggcaggggtggtttgcgc |
| | | tcctctgtcagttttctgtgcagcaggcacctgccgaggcgggctcagct |
| | | ccggctgcccagggagctggagcaggctgggctgccaatggtgggggctg |
| | | cggtgagaaggtctgcacccagcacctgacctttgtttgaagaaggagct |
| | | gagctgctactacactctatagggccattatgatgaaatatgccccccaa |
| | | actccatccagctatcttttgactagcaattccacttgttggcatttatc |
| | | tgaaagaaatacatcaaaaagtggcatgcacaaagaaatacgcatcagat |
| | | tgttccttctagtgttgcttactagctaaaaatgggaaataatccaaatg |
| | | tccatcaaaagggactggtcagataaattcagacacagtctaataagggg |
| | | gtttaggcggctgctaaggaatgagctagatccatatatgctgatatcaa |
| | | atgatggttatgacaaatgcatatatttttaaaacatctgtatagattat |
| | | tcttaatacaatatatggcacaatatgtagtcactgttaaaattcaagca |
| | | caaaagaaaacaagcaaaacaaatatatgttgttagaagttaggatacag |
| | | ttacccttggtccagtggtggtcattaggagggagcatgaggtttgaagg |
| | | tgagcatttctgcttctcaatctgggaatcacttatatgagtgtgtttca |
| | | ttagtaaaagtcatcaagctgtacactcagatatgttgactttagagcta |
| | | tggatatagatgtaaattatacttcaagaaagagactttttaaaatccaa |
| | | gtacagaacattgtagaaatgtgtaaaattggtatttggaagtttcttca |
| | | gcaagcacatgggattacagctcacgcctgtaatcccagtactttgggag |
| | | gcccacacagaatgatcacttgagctcaggagtttgagagcagcctaggc |
| | | aacatagcaagacctcgactctacaaaaaatttttaaatttttttctggg |
| | | catggtggtgcttgcctgtagtcccagctattagggaggatgaggtggga |
| | | ggatctcctgaccccaggagtttcagactgcagtgagctgtgatcacgcc |
| | | actgcactccagcctgggcaacacagacagaccttgtctcaaaaataaat |
| | | tttttctaaaaagtttcttgtttttttttcttcccatattttttaaaaca |
| | | taaaattgaaatcttgagaatataatacataaaatagagaatatagaaaa |
| | | ttacatcttctaatattgttctgcagatttgatcttttttctctaataat |
| | | aaatcaaggcctccgggtatatttgtttgtatatgtacaggaaaaggtct |
| | | gtaaggatttactttcagctgttaattgcagttactctgcggaatttcca |
| | | cttctgctttgtacagttccttgtgtgaatttttacatcatgtattactt |
| | | ttgtagttggggaaaaagcaattaaaagtttggaaaaaaa |
| SEQ ID NO: 426 | 3UTR353 | agtgtaagaaacccagactgaacttaccgtgagcgacaaagatgatttaa |
| | | aagggaagtctagagttcctagtctccctcacagcacagagaagacaaaa |
| | | ttagcaaaaccccactacacagtctgcaagattctgaaacattgctttga |
| | | ccactcttcctgagttcagtggcactcaacatgagtcaagagcatcctgc |
| | | ttctaccatgtggatttggtcacaaggtttaaggtgacccaatgattcag |
| | | ctatttaaaaaaaaaagaggaaagaatgaaagagtaaaggaaatgattga |
| | | ggagtgaggaaggcaggaagagagcatgagaggaaagaaagaaaggaaaa |
| | | aaaaaatgatagttgccattattaggatttaatatatatccagtgctttg |
| | | caagtgctctgcgcaccttgtctcactccatcctgacaataatcctggga |
| | | ggtgtgtgcaattactacgactactctcttttttatagatcattaaattc |
| | | agaactaaggagttaagtaacttgtccaagttgttcacacagtgaaggga |
| | | ggggccaagatatgatggctgggagtctaattgcagttccctgagccatg |
| | | tgcctttctcttcactgaggactgccccattcttgagtgccaaacgtcac |
| | | tagtaacagggtgtgcctagataatttatgatccaaactgagtcagtttg |
| | | gaaagtgaaagggaaacttacatataatccctccgggacaatgagcaaaa |
| | | actaggactgtccccagacaaatgtgaacatacatatcatcacttaaatt |
| | | aaaatggctatgagaaagaaagagggggagaaacagtcttgcgggtgtga |
| | | agtcccatgaccagccatgtcaaaagaaggtaaagaagtcaagaaaaagc |
| | | catgaagcccatttggtttcatttttctgaaaataggctcaagagggaat |
| | | aaattagaaactcacaatttctcttgtttgttaccaagacagtgattctc |
| | | ttgctgctaccacccaactgcatccgtccatgatctcagaggaaactgtc |
| | | gctgaccctggacatgggtacgtttgacgagtgagaggaggcatgacccc |
| | | tcccatgtgtatagacactaccccaacctaaattcatccctaaattgtcc |
| | | caagttctccagcaatagaggctgccacaaacttcagggagaaagagtta |
| | | caagtacatgcaatgagtgaactgactgtggctacaatcttgaagatata |
| | | cggaagagacgtattattaatgcttgacatatatcatcttgcctttcttg |
| | | gtctagactgacttctaatgactaactcaaagtcaaggcaactgagtaat |
| | | gtcagctcagcaaagtgcagcaaacccatctcccacaggcctccaaaccc |
| | | tggctgttcacagaaccacaaagggcagatgctgcacagaaaactagaga |
| | | aggggtcataggttcatggttttgtttgagatttgttgctactgtttttc |
| | | tgttttgaattttcttctttgttctgtttttactttatttagggggacta |
| | | ggtgtttctgatattttagttttcttgtttgttttgttttgtgttgtctg |
| | | tgaatggggttttaactgtggatgaatggaccttatctgttggcttaaag |
| | | gactggtaagatcagaccatcttattcttcaggtgaatgttttactttcc |
| | | aaagtgctctcctctgcaccagcagtaataaatacaatgccataatccct |
| | | taggtttgcctagtgcttttgcaattttcaaagcacttccataagcattc |
| | | cttccacctccttgataggcatttatggaaagcctgctacatgtcaatca |
| | | tactgttaggcacaggggacctaaagacacataaaaggatggcattctgc |
| | | ctcataaattgcaaaacctaatgaaagtgactgcttggtaaacaaattat |
| | | tattatattataaaatgctataaaagagccatattgaaagtgccctgttg |
| | | gagacagggcaaatgccacaaaaatgatgtaaatttacatggaggaaaag |
| | | tagaatctgcctggtttgtaggcagcagaagacatttttcatcagtgggc |
| | | aggtgttctttaccttttgtagaaatgggagtcaagtctcaaataggagg |
| | | ctccacaaaatctcatgccaggtctctgataccttattcacagaagttct |
| | | ttgaagtatttattgttattttctttgacttatgggaaaactgggacaca |
| | | ggaagacaggtaaattacccaacctcacacgttaagtcagaactgggagc |
| | | cataattttgtatccctggtataaatagacaatctcttgaagaaatgaag |
| | | agatgaccatagaaaaacatcgagatatctccagctctaaaatcctttgt |
| | | ttcaatgttgtttggcatatgttatctttggaatttagtgtctgagcctc |
| | | tgtctgttactgtagtatttaaaatgcatgtattataatcatataatcat |
| | | aactgctgttaattcttgattatatacctagggacaatgtgtaatgtaag |
| | | attactaattggttctgcccaatctcctttcagattttattaggaaaaaa |
| | | aaataaacctcctgatcggagacaatgtattaatcagaagtgtaaactgc |
| | | cagttctatatagcatgaaatgaaaagacagctaatttggtccaacaaac |
| | | atgactgggtctagggcacccaggctgattcagctgatttcctaccagcc |
| | | tttgcctcttccttcaatgtggtttccatgggaatttgcttcagaaaagc |
| | | caagtatgggctgttcagaggtgcacacctgcattttcttagctcttcta |
| | | gaggggctaagagacttggtacgggccaggaagaatatgtggcagagctc |
| | | ctggaaatgatgcagattaggtggcatttttgtcagctctgtggtttatt |
| | | gttgggactattctttaaaatatccattgttcactacagtgaagatctct |
| | | gatttaaccgtgtactatccacatgcattacaaacatttcgcagagctgc |
| | | ttagtatataagcgtacaatgtatgtaataaccatctcatatttaattaa |
| | | atggtatagaagaaca |
| SEQ ID NO: 427 | 3UTR354 | ggtgaggggccttgaagctgggagtggggtttagggacgcgggtctctgc |
| | | gtgcatcctaagctctgagagcaaacctccctgcagggtcttgcttttaa |
| | | gtccaaagcctgagcccaccaaactctcctacttcttcctgttacaaatt |
| | | cctcttgtgcaataataatggcctgaaacgctgtaaaatatcctcatttc |
| | | agccgcctcagttgaacttctcccctatgaggtaggaagaacagttgttt |
| | | agaaacgaagaaactgaggccacacagctaatgagtgaggaagagagaca |
| | | cttgtgtacaccacatgccttgtgttgtacttctctcaccgtgtaacctc |
| | | ctcatgtcctctctccccagtacggctctcttagctcagtagaaagaaga |
| | | cattacactcatattacaccccaatcctggctagagtctccgcaccctcc |
| | | tcccccagggtccccagtcggtcttgctgacaactgcatcctgttccatc |
| | | accatcaaaaaaaaactccaggctgggtgcgggggctcacacctgtaatc |
| | | ccagcactttgggaggcagaggcaggaggagcacaggagctggagaccag |
| | | cctgggcaacacaggagaccccgcctctacaaaaagtgaaaaaattagcc |
| | | agtgtgtgctgcacacctgtagtcccagctacttaagaggctgagatggg |
| | | aggatcgcttgagccctggaatgttgaggctacaatgagctgtgattgtg |
| | | tcactgcactccagcctggaagacaaagcaagatcctgtctcaaataata |
| | | aaaaaaataagaactccagggtacatttgctcctagaactctaccacata |
| | | gccccaaacagagccatcaccatcacatccctaacagtcctgggtcttcc |
| | | tcagtgtccagcctgacttctgttcttcctcattccagatctgcaagatt |
| | | gtaagacagcctgtgctccctcgctccttcctctgcattgcccctcttct |
| | | ccctctccaaacagagggaactctcccacccccaaggaggtgaaagctgc |
| | | taccacctctgtgcccccccgcaatgccaccaactggatgggatcctacc |
| | | cgaatttatgattaagattgctgaagagctgccaaacactgctgccaccc |
| | | cctctgttcccttattgctgcttgtcactgcctgacattcacggcagagg |
| | | caaggctgctgcagcctcccctggctgtgcacattccctcctgctcccca |
| | | gagactgcctccgccatcccacagatgatggatcttcagtgggttctctt |
| | | gggctctaggtcctggagaatgttgtgagggtttatttttttttaatagt |
| | | gttcataaagaaatacatagtattcttcttctcaagacgtggggggaaat |
| | | tatctcattatcgaggccctgctatgctgtgtgtctgggcgtgttgtatg |
| | | tcctgctgccgatgccttcattaaaatgatttggaagagcagagactgtg |
| | | cctctgtttgactgggtttggtaggagtcattttctgcttgctggtgatc |
| | | actagctgggcagagaaaaaccaaggcatttgtctatgatgctgtccagg |
| | | aagcctcattcaacaagctgcctaagtcaacctcttcttggaataacctc |
| | | taaaagcttccgcttagcaggctatgctgagggccaggaaaacccaccta |
| | | ccagcttggacccctcctctcccactctcatgccacgccacgggacc |
| SEQ ID NO: 428 | 3UTR355 | ATGTCTCCAGTTACAACTCCGCAGTGGATGTGAAGAAGCgacagagctct |
| | | gcGGTGtcaggGCGAGAACCCaTCTTCcAACCCcggcTATTTggagacgg |
| | | AAAAActgGAAttCTAACaaGGAGGagaggagATGTCTCCAGTTACAACT |
| | | CCGCAGTGGATGTGAAGAAGC |
| SEQ ID NO: 429 | 3UTR356 | ATGTCTCCAGTTACAACTCCGCAGTGGATGTGAAGAAGCGGTGCCTTTGA |
| | | GAGTCTACTTTTGCTCTCTTCGGAAGAACCCTTAGGGGTTCGTGCATGGG |
| | | CTTGCATAGCAAGTCTAGATGCGGGTACCGTACAGTGTTGAAAAACACTG |
| | | TAAATCTCTAAAAGAGACCAATGTCTCCAGTTACAACTCCGCAGTGGATG |
| | | TGAAGAAGC |
| SEQ ID NO: 430 | 3UTR357 | acctgagactggtggcttctagaagcagccattaccaactgtaccttccc |
| | | ttcttgctcagcc |
| SEQ ID NO: 431 | 3UTR358 | GGTGCCAGATTAATTGCTGTTTATTAATGGAAATTAATGTTTCGCGGTTT |
| | | TGCTAGGTTTTTTTAACATTCCAGCCAGTGTAAGGACGGAATTGTGATAG |
| | | GGTAAAAATGTAGTCCTGCTAAAGAGAGTCCTGCTGTCCGAATGCTGTAG |
| | | CTGGTGAAAGAGACCTGCG |
| SEQ ID NO: 432 | 3UTR359 | GGTGCCAGCATAACAAAAAGTTTTGCTATTAATAAAAATGCTTTTTGTTT |
| | | TTCGTGCGTCCTGCTTTTTAACATTCCAGAAAGATCCCAGTTTTAACATT |
| | | AAAAATGCTTGTAGTCCTGCTAAAGAGAGTCCAAAAAAAAAAGTGTAGCT |
| | | GGTGAAAGAGACCTGCG |
| SEQ ID NO: 433 | 3UTR360 | GGTGGTTTTCTGCGTCTACTTTTGCTGCTAGAAAGATCATTAATCCAGTT |
| | | TTGTTTTGTCCAAAAAATTAATCCAGATTAATGTCCTGCTTTTTGTTTTT |
| | | GCTAAAAATGCTTGTAGTCCTGCTAAAGAGAGTCCTGCTGTCCGAATGCT |
| | | GTAGCTGGTGAAAGAGACCTGCG |
| SEQ ID NO: 434 | 3UTR361 | GGTGTGCTAGAAAAACGCGAGGAATTGCACAGGAAAAAATGCTTTTTGTT |
| | | TTTGCTTTTTAACATTATTAATCCAGGGAAGAACAGGAATTGCACTTTTA |
| | | ACATTAAAAAATGTCTCCAGTTACAACTCCGCAGTGGATGTGAAGAAGCG |
| | | CTGGTGCCAGAGGACCTGCGTGTAAGGA |
| SEQ ID NO: 435 | 3UTR362 | GGTGATTAATTGCTTCTACTTTTGCTGGATGCTGGAAGAACTGCTCGCGT |
| | | TTAACTGCTTGTACCAGATTAATGGAAGAACTGCTGTTTTGCTTTTTAAC |
| | | ATTAAAAAGGAAGTCCTGCTAAAGAGAGTCCTGCTGTCCGAATGCTGTAG |
| | | CTGGTGAAAGAGACCTGCG |
| SEQ ID NO: 436 | 3UTR363 | GGTGCCAGGTCCTCTACTTTTGCAAAGATCAAGATCAAAAATGCTCGCGT |
| | | GCTTCTACTTTTGCGTCCAATCATGCTTGTAGGAAGTTTTTTTAACATTA |
| | | AAAATTTTGTTTTTGCTAAAGAGAGTCCTGCTGTCCGAATGCTGTAGCTG |
| | | GTGAAAGAGACCTGCG |
| SEQ ID NO: 437 | 3UTR364 | ATGTCTCCAGTTACAACTCCGCAGTGGATGTGAAGAAGCAAGATCTTTTA |
| | | ACATTGTCCAGTCCTGCTGGAAGAACTGCTTGTAGGAATTTTGTTTTGGA |
| | | AAAAAATGCTGGAAGAACTTTTGTTTTTGCTAAAGAGAGTCCTGCTGTCC |
| | | GAATGCTGTAGCTGGTGAAAGAGACCTGCG |
| SEQ ID NO: 438 | 3UTR365 | ATGTCTCCAGTTACAACTCCGCAGAAAGAGAATTAATAAAGAGATTTTAA |
| | | CATTTCTACTTTTGCGTCCCCAGGGAAGAACTGTAGTCCAAAGATCTGCT |
| | | AGCATAACTCGTGCGGAAAAAAATTTTGTTTTTGCTAAAAAGTCCTGCTG |
| | | TCCGAATGCTGTAGCTGGTGAAAGAGACCTGCG |

TABLE 3
Plasmids used to assess UTR Pair (UP) Expression Levels:

| Registry ID | 5′UTR | Coding Sequences | 3′ UTR |
|---|---|---|---|
| P001 | 5UTR033 | CDS012 | 3UTR028 |
| P002 | 5UTR034 | CDS012 | 3UTR028 |
| P003 | 5UTR030 | CDS001 | 3UTR013 |
| P006 | 5UTR003 | CDS012 | 3UTR013 |
| P007 | 5UTR004 | CDS012 | 3UTR013 |
| P008 | 5UTR005 | CDS012 | 3UTR013 |
| P009 | 5UTR010 | CDS012 | 3UTR013 |
| P010 | 5UTR011 | CDS012 | 3UTR013 |
| P011 | 5UTR012 | CDS012 | 3UTR013 |
| P012 | 5UTR013 | CDS012 | 3UTR013 |
| P013 | 5UTR014 | CDS012 | 3UTR013 |
| P014 | 5UTR015 | CDS012 | 3UTR013 |
| P015 | 5UTR017 | CDS012 | 3UTR013 |
| P016 | 5UTR018 | CDS012 | 3UTR013 |
| P017 | 5UTR020 | CDS012 | 3UTR013 |
| P018 | 5UTR022 | CDS012 | 3UTR013 |
| P019 | 5UTR023 | CDS012 | 3UTR013 |
| P020 | 5UTR024 | CDS012 | 3UTR013 |
| P021 | 5UTR025 | CDS012 | 3UTR013 |
| P022 | 5UTR029 | CDS012 | 3UTR013 |
| P023 | 5UTR030 | CDS012 | 3UTR004 |
| P024 | 5UTR030 | CDS012 | 3UTR005 |
| P025 | 5UTR030 | CDS012 | 3UTR006 |
| P026 | 5UTR030 | CDS012 | 3UTR007 |
| P027 | 5UTR030 | CDS012 | 3UTR011 |
| P028 | 5UTR030 | CDS012 | 3UTR014 |
| P029 | 5UTR030 | CDS012 | 3UTR015 |
| P030 | 5UTR030 | CDS012 | 3UTR017 |
| P031 | 5UTR030 | CDS012 | 3UTR025 |
| P032 | 5UTR030 | CDS012 | 3UTR026 |
| P045 | 5UTR030 | CDS014 | 3UTR002 |
| P052 | 5UTR030 | CDS014 | 3UTR010 |
| P053 | 5UTR030 | CDS014 | 3UTR013 |
| P069 | 5UTR030 | CDS014 | 3UTR038 |
| P093 | 5UTR030 | CDS014 | 3UTR074 |
| P096 | 5UTR030 | CDS015 | 3UTR013 |
| P098 | 5UTR030 | CDS017 | 3UTR013 |
| P099 | 5UTR030 | CDS018 | 3UTR013 |
| P100 | 5UTR030 | CDS019 | 3UTR013 |
| P102 | 5UTR030 | CDS021 | 3UTR013 |
| P103 | 5UTR030 | CDS022 | 3UTR013 |
| P104 | 5UTR030 | CDS023 | 3UTR013 |
| P134 | 5UTR080 | CDS011 | 3UTR030 |
| P135 | 5UTR030 | CDS012 | 3UTR101 |
| P136 | 5UTR086 | CDS014 | 3UTR030 |
| P137 | 5UTR079 | CDS014 | 3UTR030 |
| P138 | 5UTR087 | CDS012 | 3UTR075 |
| P139 | 5UTR084 | CDS014 | 3UTR030 |
| P140 | 5UTR030 | CDS012 | 3UTR096 |
| P141 | 5UTR080 | CDS012 | 3UTR030 |
| P142 | 5UTR079 | CDS012 | 3UTR030 |
| P143 | 5UTR030 | CDS012 | 3UTR092 |
| P144 | 5UTR030 | CDS012 | 3UTR082 |
| P145 | 5UTR088 | CDS012 | 3UTR030 |
| P146 | 5UTR087 | CDS011 | 3UTR030 |
| P147 | 5UTR088 | CDS011 | 3UTR030 |
| P148 | 5UTR087 | CDS012 | 3UTR030 |
| P149 | 5UTR030 | CDS012 | 3UTR110 |
| P150 | 5UTR086 | CDS011 | 3UTR030 |
| P151 | 5UTR030 | CDS012 | 3UTR098 |
| P152 | 5UTR082 | CDS012 | 3UTR030 |
| P153 | 5UTR083 | CDS014 | 3UTR030 |
| P154 | 5UTR083 | CDS012 | 3UTR030 |
| P156 | 5UTR086 | CDS012 | 3UTR030 |
| P157 | 5UTR030 | CDS012 | 3UTR097 |
| P158 | 5UTR079 | CDS011 | 3UTR030 |
| P159 | 5UTR088 | CDS012 | 3UTR030 |
| P160 | 5UTR030 | CDS012 | 3UTR087 |
| P162 | 5UTR082 | CDS011 | 3UTR030 |
| P163 | 5UTR087 | CDS014 | 3UTR030 |
| P164 | 5UTR081 | CDS012 | 3UTR030 |
| P165 | 5UTR085 | CDS011 | 3UTR030 |
| P166 | 5UTR085 | CDS014 | 3UTR030 |
| P167 | 5UTR083 | CDS011 | 3UTR030 |
| P168 | 5UTR084 | CDS012 | 3UTR030 |
| P169 | 5UTR030 | CDS012 | 3UTR102 |
| P170 | 5UTR081 | CDS011 | 3UTR030 |
| P172 | 5UTR030 | CDS012 | 3UTR090 |
| P173 | 5UTR085 | CDS012 | 3UTR030 |
| P174 | 5UTR022 | CDS012 | 3UTR021 |
| P175 | 5UTR022 | CDS012 | 3UTR022 |
| P176 | 5UTR022 | CDS012 | 3UTR029 |
| P177 | 5UTR022 | CDS012 | 3UTR048 |
| P178 | 5UTR022 | CDS012 | 3UTR049 |
| P179 | 5UTR022 | CDS012 | 3UTR050 |
| P180 | 5UTR022 | CDS012 | 3UTR051 |
| P181 | 5UTR022 | CDS012 | 3UTR075 |
| P182 | 5UTR024 | CDS012 | 3UTR021 |
| P183 | 5UTR024 | CDS012 | 3UTR022 |
| P184 | 5UTR024 | CDS012 | 3UTR049 |
| P185 | 5UTR024 | CDS012 | 3UTR050 |
| P186 | 5UTR024 | CDS012 | 3UTR051 |
| P187 | 5UTR024 | CDS012 | 3UTR029 |
| P188 | 5UTR024 | CDS012 | 3UTR075 |
| P189 | 5UTR024 | CDS012 | 3UTR048 |
| P190 | 5UTR030 | CDS012 | 3UTR113 |
| P191 | 5UTR030 | CDS012 | 3UTR112 |
| P192 | 5UTR030 | CDS012 | 3UTR005 |
| P193 | 5UTR030 | CDS012 | 3UTR011 |
| P194 | 5UTR038 | CDS012 | 3UTR075 |
| P195 | 5UTR038 | CDS012 | 3UTR075 |
| P200 | 5UTR024 | CDS030 | 3UTR005 |
| P201 | 5UTR024 | CDS030 | 3UTR011 |
| P202 | 5UTR024 | CDS030 | 3UTR022 |
| P203 | 5UTR024 | CDS030 | 3UTR049 |
| P204 | 5UTR024 | CDS030 | 3UTR050 |
| P205 | 5UTR024 | CDS030 | 3UTR112 |
| P206 | 5UTR024 | CDS030 | 3UTR113 |
| P207 | 5UTR024 | CDS030 | 3UTR114 |
| P208 | 5UTR024 | CDS030 | 3UTR115 |
| P209 | 5UTR024 | CDS030 | 3UTR116 |
| P211 | 5UTR024 | CDS031 | 3UTR112 |
| P212 | 5UTR024 | CDS031 | 3UTR113 |
| P213 | 5UTR024 | CDS031 | 3UTR118 |
| P214 | 5UTR024 | CDS031 | 3UTR120 |
| P215 | 5UTR024 | CDS031 | 3UTR139 |
| P216 | 5UTR024 | CDS031 | 3UTR140 |
| P217 | 5UTR024 | CDS031 | 3UTR122 |
| P218 | 5UTR024 | CDS031 | 3UTR124 |
| P219 | 5UTR024 | CDS031 | 3UTR141 |
| P220 | 5UTR024 | CDS031 | 3UTR142 |
| P221 | 5UTR024 | CDS031 | 3UTR126 |
| P222 | 5UTR024 | CDS031 | 3UTR127 |
| P223 | 5UTR024 | CDS031 | 3UTR143 |
| P224 | 5UTR024 | CDS031 | 3UTR144 |
| P225 | 5UTR024 | CDS031 | 3UTR131 |
| P226 | 5UTR024 | CDS031 | 3UTR132 |
| P227 | 5UTR024 | CDS031 | 3UTR145 |
| P228 | 5UTR024 | CDS031 | 3UTR153 |
| P229 | 5UTR024 | CDS031 | 3UTR134 |
| P230 | 5UTR024 | CDS031 | 3UTR135 |
| P231 | 5UTR024 | CDS031 | 3UTR146 |
| P232 | 5UTR024 | CDS031 | 3UTR147 |
| P233 | 5UTR024 | CDS031 | 3UTR136 |
| P234 | 5UTR024 | CDS031 | 3UTR137 |
| P235 | 5UTR024 | CDS031 | 3UTR148 |
| P236 | 5UTR024 | CDS031 | 3UTR149 |
| P237 | 5UTR024 | CDS031 | 3UTR138 |
| P238 | 5UTR024 | CDS031 | 3UTR154 |
| P239 | 5UTR024 | CDS031 | 3UTR155 |
| P240 | 5UTR024 | CDS031 | 3UTR156 |
| P241 | 5UTR073 | CDS031 | 3UTR022 |
| P242 | 5UTR091 | CDS031 | 3UTR022 |
| P243 | 5UTR092 | CDS031 | 3UTR022 |
| P244 | 5UTR093 | CDS031 | 3UTR022 |
| P245 | 5UTR095 | CDS031 | 3UTR022 |
| P246 | 5UTR097 | CDS031 | 3UTR022 |
| P247 | 5UTR098 | CDS031 | 3UTR022 |
| P248 | 5UTR102 | CDS031 | 3UTR022 |
| P249 | 5UTR024 | CDS031 | 3UTR157 |
| P250 | 5UTR024 | CDS031 | 3UTR158 |
| P251 | 5UTR024 | CDS031 | 3UTR159 |
| P252 | 5UTR024 | CDS031 | 3UTR160 |
| P254 | 5UTR024 | CDS033 & CDS012 | 3UTR005 |
| P255 | 5UTR024 | CDS033 & CDS012 | 3UTR011 |
| P256 | 5UTR024 | CDS033 & CDS012 | 3UTR022 |
| P257 | 5UTR024 | CDS033 & CDS012 | 3UTR049 |
| P258 | 5UTR024 | CDS033 & CDS012 | 3UTR050 |
| P259 | 5UTR024 | CDS033 & CDS012 | 3UTR112 |
| P260 | 5UTR024 | CDS033 & CDS012 | 3UTR113 |
| P261 | 5UTR024 | CDS033 & CDS012 | 3UTR114 |
| P262 | 5UTR024 | CDS033 & CDS012 | 3UTR115 |
| P263 | 5UTR024 | CDS033 & CDS012 | 3UTR116 |
| P269 | 5UTR024 | CDS033 & CDS012 | 3UTR092 |
| P270 | 5UTR024 | CDS012 | 3UTR022 |
| P271 | 5UTR024 | CDS012 | 3UTR161 |
| P272 | 5UTR024 | CDS012 | 3UTR162 |
| P273 | 5UTR024 | CDS012 | 3UTR163 |
| P274 | 5UTR024 | CDS012 | 3UTR164 |
| P275 | 5UTR024 | CDS012 | 3UTR165 |
| P276 | 5UTR024 | CDS012 | 3UTR166 |
| P277 | 5UTR024 | CDS012 | 3UTR167 |
| P278 | 5UTR024 | CDS012 | 3UTR168 |
| P279 | 5UTR024 | CDS012 | 3UTR169 |
| P280 | 5UTR024 | CDS012 | 3UTR170 |
| P281 | 5UTR024 | CDS012 | 3UTR171 |
| P282 | 5UTR024 | CDS012 | 3UTR172 |
| P283 | 5UTR024 | CDS012 | 3UTR173 |
| P284 | 5UTR024 | CDS012 | 3UTR174 |
| P285 | 5UTR024 | CDS012 | 3UTR175 |
| P286 | 5UTR024 | CDS012 | 3UTR176 |
| P287 | 5UTR024 | CDS012 | 3UTR177 |
| P288 | 5UTR024 | CDS012 | 3UTR178 |
| P289 | 5UTR024 | CDS012 | 3UTR179 |
| P290 | 5UTR024 | CDS012 | 3UTR180 |
| P291 | 5UTR024 | CDS012 | 3UTR181 |
| P292 | 5UTR024 | CDS012 | 3UTR182 |
| P293 | 5UTR024 | CDS012 | 3UTR183 |
| P294 | 5UTR024 | CDS012 | 3UTR184 |
| P295 | 5UTR024 | CDS012 | 3UTR185 |
| P296 | 5UTR024 | CDS012 | 3UTR186 |
| P297 | 5UTR024 | CDS012 | 3UTR187 |
| P298 | 5UTR024 | CDS012 | 3UTR188 |
| P299 | 5UTR024 | CDS012 | 3UTR189 |
| P300 | 5UTR024 | CDS012 | 3UTR190 |
| P301 | 5UTR024 | CDS012 | 3UTR191 |
| P302 | 5UTR024 | CDS012 | 3UTR192 |
| P303 | 5UTR024 | CDS012 | 3UTR193 |
| P304 | 5UTR024 | CDS012 | 3UTR194 |
| P305 | 5UTR024 | CDS012 | 3UTR195 |
| P306 | 5UTR024 | CDS012 | 3UTR196 |
| P307 | 5UTR024 | CDS012 | 3UTR197 |
| P308 | 5UTR024 | CDS012 | 3UTR198 |
| P309 | 5UTR024 | CDS012 | 3UTR199 |
| P310 | 5UTR024 | CDS012 | 3UTR200 |
| P311 | 5UTR024 | CDS012 | 3UTR201 |
| P312 | 5UTR024 | CDS012 | 3UTR202 |
| P313 | 5UTR024 | CDS012 | 3UTR203 |
| P314 | 5UTR024 | CDS012 | 3UTR204 |
| P315 | 5UTR024 | CDS012 | 3UTR205 |
| P316 | 5UTR024 | CDS012 | 3UTR206 |
| P317 | 5UTR024 | CDS012 | 3UTR207 |
| P318 | 5UTR024 | CDS012 | 3UTR208 |
| P319 | 5UTR024 | CDS012 | 3UTR209 |
| P320 | 5UTR024 | CDS012 | 3UTR210 |
| P321 | 5UTR024 | CDS012 | 3UTR211 |
| P322 | 5UTR024 | CDS012 | 3UTR212 |
| P323 | 5UTR024 | CDS012 | 3UTR213 |
| P324 | 5UTR024 | CDS012 | 3UTR214 |
| P325 | 5UTR024 | CDS012 | 3UTR215 |
| P326 | 5UTR024 | CDS012 | 3UTR216 |
| P327 | 5UTR024 | CDS012 | 3UTR217 |
| P328 | 5UTR024 | CDS012 | 3UTR218 |
| P329 | 5UTR024 | CDS012 | 3UTR219 |
| P330 | 5UTR024 | CDS012 | 3UTR220 |
| P331 | 5UTR024 | CDS012 | 3UTR221 |
| P332 | 5UTR024 | CDS012 | 3UTR222 |
| P333 | 5UTR024 | CDS012 | 3UTR223 |
| P334 | 5UTR024 | CDS012 | 3UTR224 |
| P335 | 5UTR024 | CDS012 | 3UTR225 |
| P336 | 5UTR024 | CDS012 | 3UTR226 |
| P337 | 5UTR024 | CDS012 | 3UTR227 |
| P338 | 5UTR024 | CDS012 | 3UTR228 |
| P339 | 5UTR024 | CDS012 | 3UTR229 |
| P340 | 5UTR024 | CDS012 | 3UTR230 |
| P341 | 5UTR024 | CDS012 | 3UTR231 |
| P342 | 5UTR024 | CDS012 | 3UTR232 |
| P343 | 5UTR024 | CDS012 | 3UTR233 |
| P344 | 5UTR024 | CDS012 | 3UTR234 |
| P345 | 5UTR024 | CDS012 | 3UTR235 |
| P346 | 5UTR024 | CDS012 | 3UTR236 |
| P347 | 5UTR024 | CDS012 | 3UTR237 |
| P348 | 5UTR024 | CDS012 | 3UTR238 |
| P349 | 5UTR024 | CDS012 | 3UTR239 |
| P350 | 5UTR024 | CDS012 | 3UTR240 |
| P351 | 5UTR024 | CDS012 | 3UTR241 |
| P352 | 5UTR024 | CDS012 | 3UTR242 |
| P353 | 5UTR024 | CDS012 | 3UTR243 |
| P354 | 5UTR024 | CDS012 | 3UTR244 |
| P355 | 5UTR024 | CDS012 | 3UTR245 |
| P356 | 5UTR038 | CDS028 | 3UTR030 |
| P357 | 5UTR024 | CDS028 & CDS012 | 3UTR022 |
| P358 | 5UTR024 | CDS038 | 3UTR022 |
| P359 | 5UTR024 | CDS037 | 3UTR022 |
| P360 | 5UTR024 | CDS037 | 3UTR022 |
| P361 | 5UTR024 | CDS037 & CDS012 | 3UTR022 |
| P362 | 5UTR024 | CDS037 & CDS012 | 3UTR022 |
| P363 | 5UTR024 | CDS037 & CDS012 | 3UTR022 |
| P364 | 5UTR024 | CDS039 & CDS012 | 3UTR022 |
| P365 | 5UTR024 | CDS012 | 3UTR022 |
| P367 | 5UTR024 | CDS041 | 3UTR022 |
| P368 | 5UTR024 | CDS041 | 3UTR022 |
| P369 | 5UTR024 | CDS041 | 3UTR022 |
| P370 | 5UTR024 | CDS041 & CDS012 | 3UTR022 |
| P371 | 5UTR024 | CDS041 & CDS012 | 3UTR022 |
| P372 | 5UTR024 | CDS041 & CDS012 | 3UTR022 |
| P373 | 5UTR024 | CDS046 | 3UTR022 |
| P374 | 5UTR024 | CDS046 | 3UTR112 |
| P375 | 5UTR024 | CDS046 | 3UTR113 |
| P376 | 5UTR024 | CDS046 | 3UTR122 |
| P377 | 5UTR024 | CDS046 | 3UTR126 |
| P378 | 5UTR024 | CDS046 | 3UTR137 |
| P379 | 5UTR024 | CDS046 | 3UTR141 |
| P380 | 5UTR024 | CDS046 | 3UTR143 |
| P381 | 5UTR024 | CDS046 | 3UTR260 |
| P382 | 5UTR024 | CDS046 | 3UTR261 |
| P383 | 5UTR024 | CDS046 | 3UTR263 |
| P384 | 5UTR024 | CDS046 | 3UTR264 |
| P385 | 5UTR024 | CDS046 | 3UTR265 |
| P386 | 5UTR024 | CDS046 | 3UTR266 |
| P387 | 5UTR024 | CDS046 | 3UTR267 |
| P388 | 5UTR024 | CDS046 | 3UTR268 |
| P389 | 5UTR024 | CDS046 | 3UTR270 |
| P390 | 5UTR024 | CDS046 | 3UTR275 |
| P391 | 5UTR103 | CDS046 | 3UTR022 |
| P392 | 5UTR104 | CDS046 | 3UTR022 |
| P393 | 5UTR105 | CDS046 | 3UTR022 |
| P394 | 5UTR107 | CDS046 | 3UTR022 |
| P395 | 5UTR109 | CDS046 | 3UTR022 |
| P396 | 5UTR110 | CDS046 | 3UTR022 |
| P397 | 5UTR111 | CDS046 | 3UTR022 |
| P398 | 5UTR112 | CDS046 | 3UTR022 |
| P399 | 5UTR113 | CDS046 | 3UTR022 |
| P400 | 5UTR116 | CDS046 | 3UTR022 |
| P401 | 5UTR117 | CDS046 | 3UTR022 |
| P402 | 5UTR024 | CDS046 | 3UTR192 |
| P403 | 5UTR024 | CDS046 | 3UTR195 |
| P404 | 5UTR024 | CDS041 & CDS012 | 3UTR185 |
| P405 | 5UTR024 | CDS041 & CDS012 | 3UTR185 |
| P406 | 5UTR024 | CDS041 & CDS012 | 3UTR188 |
| P407 | 5UTR024 | CDS041 & CDS012 | 3UTR188 |
| P408 | 5UTR024 | CDS041 & CDS012 | 3UTR190 |
| P409 | 5UTR024 | CDS041 & CDS012 | 3UTR190 |
| P410 | 5UTR024 | CDS041 & CDS012 | 3UTR192 |
| P411 | 5UTR024 | CDS041 & CDS012 | 3UTR192 |
| P412 | 5UTR024 | CDS041 & CDS012 | 3UTR195 |
| P413 | 5UTR024 | CDS041 & CDS012 | 3UTR195 |
| P414 | 5UTR024 | CDS041 & CDS012 | 3UTR200 |
| P415 | 5UTR024 | CDS041 & CDS012 | 3UTR200 |
| P416 | 5UTR024 | CDS041 & CDS012 | 3UTR201 |
| P417 | 5UTR024 | CDS041 & CDS012 | 3UTR201 |
| P418 | 5UTR024 | CDS041 & CDS012 | 3UTR276 |
| P419 | 5UTR024 | CDS041 & CDS012 | 3UTR277 |
| P420 | 5UTR024 | CDS041 & CDS012 | 3UTR278 |
| P421 | 5UTR024 | CDS041 & CDS012 | 3UTR279 |
| P422 | 5UTR024 | CDS041 & CDS012 | 3UTR280 |
| P423 | 5UTR024 | CDS041 & CDS012 | 3UTR281 |
| P424 | 5UTR024 | CDS041 & CDS012 | 3UTR282 |
| P425 | 5UTR024 | CDS041 & CDS012 | 3UTR283 |
| P426 | 5UTR024 | CDS047 | 3UTR022 |
| P427 | 5UTR024 | CDS047 | 3UTR022 |
| P428 | 5UTR024 | CDS047 | 3UTR022 |
| P429 | 5UTR024 | CDS047 & CDS012 | 3UTR022 |
| P430 | 5UTR024 | CDS047 & CDS012 | 3UTR022 |
| P431 | 5UTR024 | CDS047 & CDS012 | 3UTR022 |
| P432 | 5UTR024 | CDS012 | 3UTR348 |
| P433 | 5UTR024 | CDS012 | 3UTR347 |
| P434 | 5UTR024 | CDS012 | 3UTR346 |
| P435 | 5UTR024 | CDS012 | 3UTR345 |
| P436 | 5UTR024 | CDS012 | 3UTR344 |
| P437 | 5UTR024 | CDS012 | 3UTR343 |
| P438 | 5UTR024 | CDS012 | 3UTR342 |
| P439 | 5UTR024 | CDS012 | 3UTR341 |
| P440 | 5UTR024 | CDS012 | 3UTR340 |
| P441 | 5UTR024 | CDS012 | 3UTR339 |
| P442 | 5UTR024 | CDS012 | 3UTR338 |
| P443 | 5UTR024 | CDS012 | 3UTR337 |
| P444 | 5UTR024 | CDS012 | 3UTR336 |
| P445 | 5UTR024 | CDS012 | 3UTR335 |
| P446 | 5UTR024 | CDS012 | 3UTR334 |
| P447 | 5UTR024 | CDS012 | 3UTR333 |
| P448 | 5UTR024 | CDS012 | 3UTR332 |
| P449 | 5UTR024 | CDS012 | 3UTR331 |
| P450 | 5UTR024 | CDS012 | 3UTR330 |
| P451 | 5UTR024 | CDS012 | 3UTR329 |
| P452 | 5UTR024 | CDS012 | 3UTR328 |
| P453 | 5UTR024 | CDS012 | 3UTR327 |
| P454 | 5UTR024 | CDS012 | 3UTR326 |
| P455 | 5UTR024 | CDS012 | 3UTR325 |
| P456 | 5UTR024 | CDS012 | 3UTR324 |
| P457 | 5UTR024 | CDS012 | 3UTR323 |
| P458 | 5UTR024 | CDS012 | 3UTR322 |
| P459 | 5UTR024 | CDS012 | 3UTR321 |
| P460 | 5UTR024 | CDS012 | 3UTR320 |
| P461 | 5UTR024 | CDS012 | 3UTR319 |
| P462 | 5UTR024 | CDS012 | 3UTR318 |
| P463 | 5UTR024 | CDS012 | 3UTR317 |
| P464 | 5UTR024 | CDS012 | 3UTR316 |
| P465 | 5UTR024 | CDS012 | 3UTR315 |
| P466 | 5UTR024 | CDS012 | 3UTR314 |
| P467 | 5UTR024 | CDS012 | 3UTR313 |
| P468 | 5UTR024 | CDS012 | 3UTR312 |
| P469 | 5UTR024 | CDS012 | 3UTR311 |
| P470 | 5UTR024 | CDS012 | 3UTR310 |
| P471 | 5UTR024 | CDS012 | 3UTR309 |
| P472 | 5UTR024 | CDS012 | 3UTR308 |
| P473 | 5UTR024 | CDS012 | 3UTR307 |
| P474 | 5UTR024 | CDS012 | 3UTR304 |
| P475 | 5UTR024 | CDS012 | 3UTR303 |
| P476 | 5UTR024 | CDS012 | 3UTR301 |
| P477 | 5UTR024 | CDS012 | 3UTR300 |
| P478 | 5UTR024 | CDS012 | 3UTR299 |
| P479 | 5UTR024 | CDS012 | 3UTR298 |
| P480 | 5UTR024 | CDS012 | 3UTR297 |
| P481 | 5UTR024 | CDS012 | 3UTR296 |
| P482 | 5UTR024 | CDS012 | 3UTR295 |
| P483 | 5UTR024 | CDS012 | 3UTR294 |
| P484 | 5UTR024 | CDS012 | 3UTR293 |
| P485 | 5UTR024 | CDS012 | 3UTR292 |
| P486 | 5UTR024 | CDS012 | 3UTR291 |
| P487 | 5UTR024 | CDS012 | 3UTR290 |
| P488 | 5UTR024 | CDS012 | 3UTR289 |
| P489 | 5UTR024 | CDS012 | 3UTR288 |
| P490 | 5UTR024 | CDS012 | 3UTR287 |
| P491 | 5UTR024 | CDS012 | 3UTR286 |
| P492 | 5UTR024 | CDS012 | 3UTR285 |
| P493 | 5UTR024 | CDS012 | 3UTR284 |
| P494 | 5UTR024 | CDS012 | 3UTR283 |
| P495 | 5UTR024 | CDS012 | 3UTR282 |
| P496 | 5UTR024 | CDS012 | 3UTR281 |
| P497 | 5UTR024 | CDS012 | 3UTR280 |
| P498 | 5UTR024 | CDS012 | 3UTR279 |
| P499 | 5UTR024 | CDS012 | 3UTR278 |
| P500 | 5UTR024 | CDS012 | 3UTR277 |
| P501 | 5UTR024 | CDS012 | 3UTR276 |
| P502 | 5UTR024 | CDS048 | 3UTR022 |
| P503 | 5UTR024 | CDS048 | 3UTR112 |
| P504 | 5UTR024 | CDS048 | 3UTR113 |
| P505 | 5UTR024 | CDS048 | 3UTR122 |
| P506 | 5UTR024 | CDS048 | 3UTR126 |
| P507 | 5UTR024 | CDS048 | 3UTR137 |
| P508 | 5UTR024 | CDS048 | 3UTR141 |
| P509 | 5UTR024 | CDS048 | 3UTR143 |
| P510 | 5UTR024 | CDS048 | 3UTR185 |
| P511 | 5UTR024 | CDS048 | 3UTR188 |
| P512 | 5UTR024 | CDS048 | 3UTR190 |
| P513 | 5UTR024 | CDS048 | 3UTR192 |
| P514 | 5UTR024 | CDS048 | 3UTR195 |
| P515 | 5UTR024 | CDS048 | 3UTR201 |
| P516 | 5UTR030 | CDS048 | 3UTR112 |
| P517 | 5UTR030 | CDS048 | 3UTR113 |
| P518 | 5UTR024 | CDS048 | 3UTR270 |
| P519 | 5UTR117 | CDS048 | 3UTR022 |
| P520 | 5UTR093 | CDS048 | 3UTR022 |
| P521 | 5UTR078 | CDS048 | 3UTR078 |
| P522 | 5UTR078 | CDS048 | 3UTR078 |
| P523 | 5UTR024 | CDS047 | 3UTR112 |
| P524 | 5UTR024 | CDS047 | 3UTR122 |
| P525 | 5UTR024 | CDS047 | 3UTR137 |
| P526 | 5UTR024 | CDS047 | 3UTR141 |
| P527 | 5UTR030 | CDS047 | 3UTR112 |
| P528 | 5UTR030 | CDS047 | 3UTR113 |
| P529 | 5UTR024 | CDS053 | 3UTR022 |
| P530 | 5UTR024 | CDS053 | 3UTR112 |
| P531 | 5UTR024 | CDS053 | 3UTR113 |
| P532 | 5UTR024 | CDS053 | 3UTR122 |
| P533 | 5UTR024 | CDS053 | 3UTR126 |
| P534 | 5UTR024 | CDS053 | 3UTR185 |
| P535 | 5UTR024 | CDS053 | 3UTR188 |
| P536 | 5UTR024 | CDS053 | 3UTR190 |
| P537 | 5UTR024 | CDS053 | 3UTR192 |
| P538 | 5UTR024 | CDS053 | 3UTR195 |
| P539 | 5UTR024 | CDS053 | 3UTR201 |
| P540 | 5UTR024 | CDS053 | 3UTR258 |
| P541 | 5UTR024 | CDS053 | 3UTR259 |
| P542 | 5UTR024 | CDS053 | 3UTR260 |
| P543 | 5UTR024 | CDS053 | 3UTR261 |
| P544 | 5UTR024 | CDS053 | 3UTR262 |
| P545 | 5UTR024 | CDS053 | 3UTR263 |
| P546 | 5UTR024 | CDS053 | 3UTR264 |
| P547 | 5UTR024 | CDS053 | 3UTR265 |
| P548 | 5UTR024 | CDS053 | 3UTR266 |
| P549 | 5UTR024 | CDS053 | 3UTR267 |
| P550 | 5UTR024 | CDS053 | 3UTR268 |
| P551 | 5UTR024 | CDS053 | 3UTR270 |
| P552 | 5UTR024 | CDS053 | 3UTR272 |
| P553 | 5UTR024 | CDS053 | 3UTR275 |
| P554 | 5UTR093 | CDS053 | 3UTR022 |
| P555 | 5UTR093 | CDS053 | 3UTR112 |
| P556 | 5UTR093 | CDS053 | 3UTR113 |
| P557 | 5UTR093 | CDS053 | 3UTR122 |
| P558 | 5UTR093 | CDS053 | 3UTR126 |
| P559 | 5UTR093 | CDS053 | 3UTR185 |
| P560 | 5UTR093 | CDS053 | 3UTR188 |
| P561 | 5UTR093 | CDS053 | 3UTR190 |
| P562 | 5UTR093 | CDS053 | 3UTR192 |
| P563 | 5UTR093 | CDS053 | 3UTR195 |
| P564 | 5UTR093 | CDS053 | 3UTR201 |
| P565 | 5UTR093 | CDS053 | 3UTR258 |
| P566 | 5UTR093 | CDS053 | 3UTR259 |
| P567 | 5UTR093 | CDS053 | 3UTR260 |
| P568 | 5UTR093 | CDS053 | 3UTR261 |
| P569 | 5UTR093 | CDS053 | 3UTR262 |
| P570 | 5UTR093 | CDS053 | 3UTR263 |
| P571 | 5UTR093 | CDS053 | 3UTR264 |
| P572 | 5UTR093 | CDS053 | 3UTR265 |
| P573 | 5UTR093 | CDS053 | 3UTR266 |
| P574 | 5UTR093 | CDS053 | 3UTR267 |
| P575 | 5UTR093 | CDS053 | 3UTR268 |
| P576 | 5UTR093 | CDS053 | 3UTR270 |
| P577 | 5UTR093 | CDS053 | 3UTR272 |
| P578 | 5UTR093 | CDS053 | 3UTR275 |
| P579 | 5UTR103 | CDS053 | 3UTR112 |
| P580 | 5UTR104 | CDS053 | 3UTR112 |
| P581 | 5UTR105 | CDS053 | 3UTR112 |
| P582 | 5UTR111 | CDS053 | 3UTR112 |
| P583 | 5UTR103 | CDS053 | 3UTR113 |
| P584 | 5UTR104 | CDS053 | 3UTR113 |
| P585 | 5UTR105 | CDS053 | 3UTR113 |
| P586 | 5UTR111 | CDS053 | 3UTR113 |
| P587 | 5UTR024 | CDS031 | 3UTR022 |
| P588 | 5UTR030 | CDS031 | 3UTR022 |
| P589 | 5UTR030 | CDS031 | 3UTR112 |
| P590 | 5UTR093 | CDS031 | 3UTR112 |
| P591 | 5UTR030 | CDS031 | 3UTR113 |
| P593 | 5UTR093 | CDS031 | 3UTR113 |
| P594 | 5UTR093 | CDS054 | 3UTR022 |
| P595 | 5UTR093 | CDS054 | 3UTR112 |
| P596 | 5UTR093 | CDS054 | 3UTR122 |
| P597 | 5UTR093 | CDS054 | 3UTR126 |
| P598 | 5UTR093 | CDS054 | 3UTR137 |
| P599 | 5UTR014 | CDS054 | 3UTR137 |
| P600 | 5UTR093 | CDS055 | 3UTR022 |
| P601 | 5UTR093 | CDS055 | 3UTR112 |
| P602 | 5UTR093 | CDS055 | 3UTR113 |
| P603 | 5UTR093 | CDS055 | 3UTR122 |
| P604 | 5UTR093 | CDS055 | 3UTR126 |
| P605 | 5UTR093 | CDS055 | 3UTR185 |
| P606 | 5UTR093 | CDS055 | 3UTR188 |
| P607 | 5UTR093 | CDS055 | 3UTR190 |
| P608 | 5UTR093 | CDS055 | 3UTR270 |
| P609 | 5UTR093 | CDS055 | 3UTR355 |
| P610 | 5UTR093 | CDS055 | 3UTR356 |
| P611 | 5UTR024 | CDS055 | 3UTR112 |
| P612 | 5UTR103 | CDS055 | 3UTR112 |
| P613 | 5UTR104 | CDS055 | 3UTR112 |
| P614 | 5UTR105 | CDS055 | 3UTR112 |
| P615 | 5UTR111 | CDS055 | 3UTR112 |
| P616 | 5UTR123 | CDS055 | 3UTR112 |
| P617 | 5UTR124 | CDS055 | 3UTR112 |
| P618 | 5UTR127 | CDS055 | 3UTR112 |
| P619 | 5UTR128 | CDS055 | 3UTR112 |
| P621 | 5UTR003 | CDS053 | 3UTR001 |
| P622 | 5UTR003 | CDS051 | 3UTR001 |
| P623 | 5UTR003 | CDS051 | 3UTR001 |
| P624 | 5UTR093 | CDS055 | 3UTR022 |
| P625 | 5UTR093 | CDS055 | 3UTR356 |
| P626 | 5UTR103 | CDS055 | 3UTR112 |
| P627 | 5UTR111 | CDS055 | 3UTR112 |
| P628 | 5UTR123 | CDS055 | 3UTR112 |
| P630 | 5UTR093 | CDS054 | 3UTR022 |
| P631 | 5UTR093 | CDS054 | 3UTR112 |
| P632 | 5UTR093 | CDS054 | 3UTR122 |
| P633 | 5UTR093 | CDS054 | 3UTR126 |
| P634 | 5UTR093 | CDS054 | 3UTR137 |
| P635 | 5UTR014 | CDS054 | 3UTR137 |
| P636 | 5UTR093 | CDS055 | 3UTR358 |
| P637 | 5UTR093 | CDS055 | 3UTR359 |
| P638 | 5UTR093 | CDS055 | 3UTR360 |
| P639 | 5UTR093 | CDS055 | 3UTR361 |
| P640 | 5UTR093 | CDS055 | 3UTR362 |
| P641 | 5UTR093 | CDS055 | 3UTR363 |
| P642 | 5UTR093 | CDS055 | 3UTR364 |
| P643 | 5UTR093 | CDS055 | 3UTR365 |
| P644 | 5UTR124 | CDS055 | 3UTR112 |
| P645 | 5UTR127 | CDS055 | 3UTR112 |
| P646 | 5UTR128 | CDS055 | 3UTR112 |
| P647 | 5UTR093 | CDS055 | 3UTR355 |
| P648 | 5UTR104 | CDS055 | 3UTR112 |
| P649 | 5UTR105 | CDS055 | 3UTR112 |
| P650 | 5UTR093 | CDS048 | 3UTR358 |
| P651 | 5UTR093 | CDS048 | 3UTR359 |
| P652 | 5UTR093 | CDS048 | 3UTR360 |
| P653 | 5UTR093 | CDS048 | 3UTR361 |
| P654 | 5UTR093 | CDS048 | 3UTR362 |
| P655 | 5UTR093 | CDS048 | 3UTR363 |
| P656 | 5UTR093 | CDS048 | 3UTR364 |
| P657 | 5UTR093 | CDS048 | 3UTR365 |
| P658 | 5UTR003 | CDS048 | 3UTR001 |
| P659 | 5UTR130 | CDS048 | 3UTR001 |
| P660 | 5UTR093 | CDS058 | 3UTR022 |
| P661 | 5UTR003 | CDS058 | 3UTR001 |
| P662 | 5UTR130 | CDS058 | 3UTR001 |
| P663 | 5UTR093 | CDS058 | 3UTR358 |
| P664 | 5UTR093 | CDS058 | 3UTR359 |
| P665 | 5UTR093 | CDS058 | 3UTR360 |
| P666 | 5UTR093 | CDS058 | 3UTR361 |
| P667 | 5UTR093 | CDS058 | 3UTR362 |
| P668 | 5UTR093 | CDS058 | 3UTR363 |
| P669 | 5UTR093 | CDS058 | 3UTR364 |
| P670 | 5UTR093 | CDS058 | 3UTR365 |
| P671 | 5UTR078 | CDS012 | 3UTR078 |
| P672 | 5UTR003 | CDS012 | 3UTR078 |
| P673 | 5UTR003 | CDS012 | 3UTR078 |
| P674 | 5UTR003 | CDS012 | 3UTR078 |
| P675 | 5UTR003 | CDS048 | 3UTR078 |
| P676 | 5UTR093 | CDS059 | 3UTR356 |
| P677 | 5UTR123 | CDS059 | 3UTR112 |
| P678 | 5UTR111 | CDS059 | 3UTR113 |

TABLE 4
UTR Pairs (UP):

| Registry ID | 5′UTR | 3′UTR |
|---|---|---|
| UP001 | 5UTR022/SEQ ID NO: 20 | 3UTR005/SEQ ID NO: 128 |
| UP002 | 5UTR022/SEQ ID NO: 20 | 3UTR011/SEQ ID NO: 134 |
| UP003 | 5UTR024/SEQ ID NO: 22 | 3UTR022/SEQ ID NO: 145 |
| UP004 | 5UTR024/SEQ ID NO: 22 | 3UTR112/SEQ ID NO: 189 |
| UP005 | 5UTR024/SEQ ID NO: 22 | 3UTR113/SEQ ID NO: 190 |
| UP006 | 5UTR024/SEQ ID NO: 22 | 3UTR122/SEQ ID NO: 199 |
| UP007 | 5UTR024/SEQ ID NO: 22 | 3UTR126/SEQ ID NO: 203 |
| UP008 | 5UTR024/SEQ ID NO: 22 | 3UTR137/SEQ ID NO: 214 |
| UP009 | 5UTR024/SEQ ID NO: 22 | 3UTR141/SEQ ID NO: 218 |
| UP010 | 5UTR024/SEQ ID NO: 22 | 3UTR143/SEQ ID NO: 220 |
| UP011 | 5UTR024/SEQ ID NO: 22 | 3UTR185/SEQ ID NO: 262 |
| UP012 | 5UTR024/SEQ ID NO: 22 | 3UTR187/SEQ ID NO: 264 |
| UP013 | 5UTR024/SEQ ID NO: 22 | 3UTR188/SEQ ID NO: 265 |
| UP014 | 5UTR024/SEQ ID NO: 22 | 3UTR190/SEQ ID NO: 267 |
| UP015 | 5UTR024/SEQ ID NO: 22 | 3UTR192/SEQ ID NO: 269 |
| UP016 | 5UTR024/SEQ ID NO: 22 | 3UTR195/SEQ ID NO: 272 |
| UP017 | 5UTR024/SEQ ID NO: 22 | 3UTR200/SEQ ID NO: 277 |
| UP018 | 5UTR024/SEQ ID NO: 22 | 3UTR201/SEQ ID NO: 278 |
| UP019 | 5UTR024/SEQ ID NO: 22 | 3UTR262/SEQ ID NO: 339 |
| UP020 | 5UTR030/SEQ ID NO: 27 | 3UTR112/SEQ ID NO: 189 |
| UP021 | 5UTR030/SEQ ID NO: 27 | 3UTR113/SEQ ID NO: 190 |
| UP022 | 5UTR073/SEQ ID NO: 69 | 3UTR076/SEQ ID NO: 186 |
| UP023 | 5UTR073/SEQ ID NO: 69 | 3UTR077/SEQ ID NO: 187 |
| UP024 | 5UTR077/SEQ ID NO: 72 | 3UTR005/SEQ ID NO: 128 |
| UP025 | 5UTR093/SEQ ID NO: 88 | 3UTR022/SEQ ID NO: 145 |
| UP026 | 5UTR103/SEQ ID NO: 98 | 3UTR113/SEQ ID NO: 190 |
| UP027 | 5UTR111/SEQ ID NO: 106 | 3UTR113/SEQ ID NO: 190 |
| UP028 | 5UTR103/SEQ ID NO: 98 | 3UTR022/SEQ ID NO: 145 |
| UP029 | 5UTR104/SEQ ID NO: 99 | 3UTR022/SEQ ID NO: 145 |
| UP030 | 5UTR111/SEQ ID NO: 106 | 3UTR022/SEQ ID NO: 145 |
| UP031 | 5UTR117/SEQ ID NO: 112 | 3UTR022/SEQ ID NO: 145 |
| UP032 | 5UTR093/SEQ ID NO: 88 | 3UTR356/SEQ ID NO: 429 |
| UP033 | 5UTR103/SEQ ID NO: 98 | 3UTR112/SEQ ID NO: 189 |
| UP034 | 5UTR111/SEQ ID NO: 106 | 3UTR112/SEQ ID NO: 189 |
| UP035 | 5UTR123/SEQ ID NO: 117 | 3UTR112/SEQ ID NO: 189 |
| UP036 | 5UTR093/SEQ ID NO: 88 | 3UTR113/SEQ ID NO: 190 |
| UP037 | 5UTR104/SEQ ID NO: 99 | 3UTR113/SEQ ID NO: 190 |
| UP038 | 5UTR105/SEQ ID NO: 100 | 3UTR113/SEQ ID NO: 190 |
| UP039 | 5UTR024/SEQ ID NO: 22 | 3UTR261/SEQ ID NO: 338 |
| UP040 | 5UTR093/SEQ ID NO: 88 | 3UTR188/SEQ ID NO: 265 |
| UP041 | 5UTR103/SEQ ID NO: 98 | 3UTR113/SEQ ID NO: 190 |
| UP042 | 5UTR093/SEQ ID NO: 88 | 3UTR357/SEQ ID NO: 430 |
| UP043 | 5UTR129/SEQ ID NO: 123 | 3UTR357/SEQ ID NO: 430 |

1. A synthetic engineered mRNA, comprising an open reading frame (ORF) operably linked to a heterologous 5′ untranslated region (UTR) and a heterologous 3′ UTR, wherein the 5′ UTR and 3′ UTR are set forth as UTR pairs in rows of the following table, and are selected from the group consisting of:

| Registry ID | 5′UTR | 3′UTR |
|---|---|---|
| UP001 | 5UTR022/SEQ ID NO: 20 | 3UTR005/SEQ ID NO: 128 |
| UP002 | 5UTR022/SEQ ID NO: 20 | 3UTR011/SEQ ID NO: 134 |
| UP003 | 5UTR024/SEQ ID NO: 22 | 3UTR022/SEQ ID NO: 145 |
| UP004 | 5UTR024/SEQ ID NO: 22 | 3UTR112/SEQ ID NO: 189 |
| UP005 | 5UTR024/SEQ ID NO: 22 | 3UTR113/SEQ ID NO: 190 |
| UP006 | 5UTR024/SEQ ID NO: 22 | 3UTR122/SEQ ID NO: 199 |
| UP007 | 5UTR024/SEQ ID NO: 22 | 3UTR126/SEQ ID NO: 203 |
| UP008 | 5UTR024/SEQ ID NO: 22 | 3UTR137/SEQ ID NO: 214 |
| UP009 | 5UTR024/SEQ ID NO: 22 | 3UTR141/SEQ ID NO: 218 |
| UP010 | 5UTR024/SEQ ID NO: 22 | 3UTR143/SEQ ID NO: 220 |
| UP011 | 5UTR024/SEQ ID NO: 22 | 3UTR185/SEQ ID NO: 262 |
| UP012 | 5UTR024/SEQ ID NO: 22 | 3UTR187/SEQ ID NO: 264 |
| UP013 | 5UTR024/SEQ ID NO: 22 | 3UTR188/SEQ ID NO: 265 |
| UP014 | 5UTR024/SEQ ID NO: 22 | 3UTR190/SEQ ID NO: 267 |
| UP015 | 5UTR024/SEQ ID NO: 22 | 3UTR192/SEQ ID NO: 269 |
| UP016 | 5UTR024/SEQ ID NO: 22 | 3UTR195/SEQ ID NO: 272 |
| UP017 | 5UTR024/SEQ ID NO: 22 | 3UTR200/SEQ ID NO: 277 |
| UP018 | 5UTR024/SEQ ID NO: 22 | 3UTR201/SEQ ID NO: 278 |
| UP019 | 5UTR024/SEQ ID NO: 22 | 3UTR262/SEQ ID NO: 339 |
| UP020 | 5UTR030/SEQ ID NO: 27 | 3UTR112/SEQ ID NO: 189 |
| UP021 | 5UTR030/SEQ ID NO: 27 | 3UTR113/SEQ ID NO: 190 |
| UP022 | 5UTR073/SEQ ID NO: 69 | 3UTR076/SEQ ID NO: 186 |
| UP023 | 5UTR073/SEQ ID NO: 69 | 3UTR077/SEQ ID NO: 187 |
| UP024 | 5UTR077/SEQ ID NO: 72 | 3UTR005/SEQ ID NO: 128 |
| UP025 | 5UTR093/SEQ ID NO: 88 | 3UTR022/SEQ ID NO: 145 |
| UP026 | 5UTR103/SEQ ID NO: 98 | 3UTR113/SEQ ID NO: 190 |
| UP027 | 5UTR111/SEQ ID NO: 106 | 3UTR113/SEQ ID NO: 190 |
| UP028 | 5UTR103/SEQ ID NO: 98 | 3UTR022/SEQ ID NO: 145 |
| UP029 | 5UTR104/SEQ ID NO: 99 | 3UTR022/SEQ ID NO: 145 |
| UP030 | 5UTR111/SEQ ID NO: 106 | 3UTR022/SEQ ID NO: 145 |
| UP031 | 5UTR117/SEQ ID NO: 112 | 3UTR022/SEQ ID NO: 145 |
| UP032 | 5UTR093/SEQ ID NO: 88 | 3UTR356/SEQ ID NO: 429 |
| UP033 | 5UTR103/SEQ ID NO: 98 | 3UTR112/SEQ ID NO: 189 |
| UP034 | 5UTR111/SEQ ID NO: 106 | 3UTR112/SEQ ID NO: 189 |
| UP035 | 5UTR123/SEQ ID NO: 117 | 3UTR112/SEQ ID NO: 189 |
| UP036 | 5UTR093/SEQ ID NO: 88 | 3UTR113/SEQ ID NO: 190 |
| UP037 | 5UTR104/SEQ ID NO: 99 | 3UTR113/SEQ ID NO: 190 |
| UP038 | 5UTR105/SEQ ID NO: 100 | 3UTR113/SEQ ID NO: 190 |
| UP039 | 5UTR024/SEQ ID NO: 22 | 3UTR261/SEQ ID NO: 338 |
| UP040 | 5UTR093/SEQ ID NO: 88 | 3UTR188/SEQ ID NO: 265 |
| UP041 | 5UTR103/SEQ ID NO: 98 | 3UTR113/SEQ ID NO: 190 |
| UP042 | 5UTR093/SEQ ID NO: 88 | 3UTR357/SEQ ID NO: 430 |
| UP043 | 5UTR129/SEQ ID NO: 123 | 3UTR357/SEQ ID NO: 430. |

2.-9. (canceled)

10. A synthetic engineered mRNA, comprising an open reading frame (ORF) operably linked to a heterologous 5′ untranslated region (UTR) and a heterologous 3′ UTR,
wherein the 5′ UTR is selected from the group consisting of: SEQ ID NOs: 1-123; and/or
wherein the 3′ UTR is selected from the group consisting of: SEQ ID NOs: 124-438.

11. The synthetic engineered mRNA of claim 10, wherein the 5′ UTR is selected from the group consisting of: SEQ ID NOs: 22, 32-34, 35-68, 70-71, 74-75, 90, 92, 97, 103, 111, 115, and 121-122.

12. The synthetic engineered mRNA of claim 10, wherein the 3′ UTR is selected from the group consisting of: SEQ ID NOs: 145, 150-185, 189-197, 199-201, 203-204, 206-209, 211-323, 335-345, 347, 349-350, 352-422, and 428-438.

13. The synthetic engineered mRNA of claim 10, wherein the 5′ UTR and 3′ UTR are set forth as numbered UTR pairs (UP) in rows of Table 4, and are selected from the group consisting of: UP001-UP043.

14.-15. (canceled)

16. The synthetic engineered mRNA of claim 10, wherein the mRNA further comprises a 5′ cap structure.

17. The synthetic engineered mRNA of claim 16, wherein the 5′ cap structure is selected from Cap 1, Cap 2, or m6A Cap 1.

18. The synthetic engineered mRNA of claim 10, wherein the mRNA further comprises a 3′ poly A tail region.

19. The synthetic engineered mRNA of claims 18, the 3′ poly A tail is a length selected from at least 30, 40, 50, 60, 70, 80, 90, 100, 120, 140, 160, 180, or 200 nucleosides.

20. A composition comprising the synthetic engineered mRNA of claim 1, formulated in a lipid nanoparticle (LNP) carrier.

21.-29. (canceled)

30. A method of expressing an engineered synthetic mRNA in a cell, said method comprising introducing the engineered mRNA of claim 1.

31. A method of making a synthetic engineered mRNA, said method comprising constructing a: (a) a 5′ untranslated region (5′UTR); (b) a CDS region encoding a heterologous polypeptide; (c) a 3′ untranslated region (3′UTR); and (d) a 3′ poly A tail region,
wherein the 5′ UTR is selected from the group consisting of: SEQ ID NOs: 1-123, or
wherein the 3′ UTR is selected from the group consisting of: SEQ ID NOs: 124-438; and
wherein said constructing is by one or more of IVT, chemical synthesis, and/or host cell expression.


SEQUENCE LISTING (445 sequences)

SEQ ID NO: 1 - RNA, 39 nt, synthetic construct
  source 1..39: mol_type = other RNA; note = Registry ID 5UTR002
cttccttttgtgactggcggtgaacgagtgcgcagtgcc

SEQ ID NO: 2 - RNA, 50 nt, synthetic construct
  source 1..50: mol_type = other RNA; note = Registry ID 5UTR003
acatttgcttctgacacaactgtgttcactagcaacctcaaacagacacc

SEQ ID NO: 3 - RNA, 84 nt, synthetic construct
  source 1..84: mol_type = other RNA; note = Registry ID 5UTR004
accgccgagaccgcgtccgccccgcgagcacagagcctcgcctttgccgatccgccgcccgtccacacccgccgccagctcacc

SEQ ID NO: 4 - RNA, 125 nt, synthetic construct
  source 1..125: mol_type = other RNA; note = Registry ID 5UTR005
cgcgttattgttctgccgggcggacacgtgacgcgaagcttaccgccgagaccgcgtccgccccgcgagcacagagcctcgcctttgccgatccgccgcccgtccacacccgccgccagctcacc

SEQ ID NO: 5 - RNA, 111 nt, synthetic construct
  source 1..111: mol_type = other RNA; note = Registry ID 5UTR006
tataaaacccggcggcgcaacgcgcagccactgtcgagtcgcgtccacccgcgagcacagcttctttgcagctccttcgttgccggtccacacccgccaccagttcgcccc

SEQ ID NO: 6 - RNA, 152 nt, synthetic construct
  source 1..152: mol_type = other RNA; note = Registry ID 5UTR007
cgcgttattgttctgccgggcggacacgtgacgcgaagctttataaaacccggcggcgcaacgcgcagccactgtcgagtcgcgtccacccgcgagcacagcttctttgcagctccttcgttgccggtccacacccgccaccagttcgcccc

SEQ ID NO: 7 - RNA, 111 nt, synthetic construct
  source 1..111: mol_type = other RNA; note = Registry ID 5UTR008
ggcgaactggtggcgggtgtggaccggcaacgaaggagctgcaaagaagctgtgctcgcgggtggacgcgactcgacagtggctgcgcgttgcgccgccgggttttatacc

SEQ ID NO: 8 - RNA, 152 nt, synthetic construct
  source 1..152: mol_type = other RNA; note = Registry ID 5UTR009
cgcgttattgttctgccgggcggacacgtgacgcgaagcttggcgaactggtggcgggtgtggaccggcaacgaaggagctgcaaagaagctgtgctcgcgggtggacgcgactcgacagtggctgcgcgttgcgccgccgggttttatacc

SEQ ID NO: 9 - RNA, 137 nt, synthetic construct
  source 1..137: mol_type = other RNA; note = Registry ID 5UTR010
agcaccacggcagcaggaggtttcggctaagttggaggtactggccacgactgcatgcccgcgcccgccaggtgatacctccgccggtgacccaggggctctgcgacacaaggagtctgcatgtctaagtgctagac

SEQ ID NO: 10 - RNA, 265 nt, synthetic construct
  source 1..265: mol_type = other RNA; note = Registry ID 5UTR011
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 11 - RNA, 270 nt, synthetic construct
  source 1..270: mol_type = other RNA; note = Registry ID 5UTR012
attaaaggtttataccttcccaggtaacaaaccaaccaactttcgatctcttgtagatctgttctctaaacgaactttaaaatctgtgtggctgtcactcggctgccgcgttattgttctgccgggcggacacgtgacgcgtaactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 12 - RNA, 265 nt, synthetic construct
  source 1..265: mol_type = other RNA; note = Registry ID 5UTR013
attaaaggtttataccttcccaggtaacaaaccaaccaactttcgatctcttgtagatctgttctctaaacgaactttaaaatctgtgtggctgtcactcggctgcttgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 13 - RNA, 199 nt, synthetic construct
  source 1..199: mol_type = other RNA; note = Registry ID 5UTR015
gggaaataagagagaaaagaagagtaagaagaaatataagagccacccgcgttattgttctgccgggcggacacgtgacgcgaagctttataaaacccggcggcgcaacgcgcagccactgtcgagtcgcgtccacccgcgagcacagcttctttgcagctccttcgttgccggtccacacccgccaccagttcgcccc

SEQ ID NO: 14 - RNA, 199 nt, synthetic construct
  source 1..199: mol_type = other RNA; note = Registry ID 5UTR016
cgcgttattgttctgccgggcggacacgtgacgcgaagcttgggaaataagagagaaaagaagagtaagaagaaatataagagccacctataaaacccggcggcgcaacgcgcagccactgtcgagtcgcgtccacccgcgagcacagcttctttgcagctccttcgttgccggtccacacccgccaccagttcgcccc

SEQ ID NO: 15 - RNA, 89 nt, synthetic construct
  source 1..89: mol_type = other RNA; note = Registry ID 5UTR017
cttccttttgtgactggcggtgaacgagtgcgcagtgccacatttgcttctgacacaactgtgttcactagcaacctcaaacagacacc

SEQ ID NO: 16 - RNA, 191 nt, synthetic construct
  source 1..191: mol_type = other RNA; note = Registry ID 5UTR018
cttccttttgtgactggcggtgaacgagtgcgcagtgcccgcgttattgttctgccgggcggacacgtgacgcgaagctttataaaacccggcggcgcaacgcgcagccactgtcgagtcgcgtccacccgcgagcacagcttctttgcagctccttcgttgccggtccacacccgccaccagttcgcccc

SEQ ID NO: 17 - RNA, 61 nt, synthetic construct
  source 1..61: mol_type = other RNA; note = Registry ID 5UTR019
cttccttttgtacatttgcttctgacacaactgtgttcactagcaacctcaaacagacacc

SEQ ID NO: 18 - RNA, 112 nt, synthetic construct
  source 1..112: mol_type = other RNA; note = Registry ID 5UTR020
gggaaataagagagaaaagaagagtaagaagaaatataagagccacccgcctcgccgcctccacatttgcttctgacacaactgtgttcactagcaacctcaaacagacacc

SEQ ID NO: 19 - RNA, 323 nt, synthetic construct
  source 1..323: mol_type = other RNA; note = Registry ID 5UTR021
ttgatcttttaatcttcgttggccacaattaaaacaaaccagatcgtggagctgcgcgatccctttgcataaaaacatatggcttttgctataaaaattatgactgcaaaacaccgggccattaatagcgtgcggagtgatttacgcgttattgttctgccgggcggacacgtgacgcgcgtggccaatgggggcgcgggcgccggcaacttattaggtgactgtacttcacccccccctggtgccaccaagttgttacatgaaatctgcagtttcataatttcggcgggtcgggctgggccggccaggcgcgggctactgca

SEQ ID NO: 20 - RNA, 140 nt, synthetic construct
  source 1..140: mol_type = other RNA; note = Registry ID 5UTR022
gtgaagattgaccatctcacaaaagctgttacgtgcttgtaacacactacgcgcccgttttgtattcgggaagtagttgcgaaaacggtccccttattgcctgacaagctaagggccacccttctttccccaccgccatc

SEQ ID NO: 21 - RNA, 63 nt, synthetic construct
  source 1..63: mol_type = other RNA; note = Registry ID 5UTR023
ggtaatctgcaaatccctggcacccgcctaaaattgccctcatcaaccttctctctattcacg

SEQ ID NO: 22 - RNA, 65 nt, synthetic construct
  source 1..65: mol_type = other RNA; note = Registry ID 5UTR024
ggctctgaaaaaaaaaaaaagacccaagctagctagcgtttaaacttaagctaggtaccgagacc

SEQ ID NO: 23 - RNA, 72 nt, synthetic construct
  source 1..72: mol_type = other RNA; note = Registry ID 5UTR025
ggctctgaaaaaaaaaaaaccgacatttgcttctgacacaactgtgttcactagcaacctcaaacagacacc

SEQ ID NO: 24 - RNA, 28 nt, synthetic construct
  source 1..28: mol_type = other RNA; note = Registry ID 5UTR026
acacgctggaattctagtatactaaacc

SEQ ID NO: 25 - RNA, 62 nt, synthetic construct
  source 1..62: mol_type = other RNA; note = Registry ID 5UTR027
acatttgcttctgacacaactgtgttcactagcaacctcaaacagacaccaaaaaaaaaaaa

SEQ ID NO: 26 - RNA, 238 nt, synthetic construct
  source 1..238: mol_type = other RNA; note = Registry ID 5UTR029
agccctccaggacaggctgcatcagaagaggccatcaagcaggtctgttccaagggcctttgcgtcaggtgggctcaggattccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgcc

SEQ ID NO: 27 - RNA, 101 nt, synthetic construct
  source 1..101: mol_type = other RNA; note = Registry ID 5UTR030
gcttgtctcgctccggggaacgctcggaaactcccggccgccgccacccgcgtctgttctgttacacaagggaagaaaagccgctgccgcactccgagtgt

SEQ ID NO: 28 - RNA, 114 nt, synthetic construct
  source 1..114: mol_type = other RNA; note = Registry ID 5UTR031
atattggagcagcaagaggctgggaagccatcacttaccttgcactgagaaagaagacaaaggcaagttgaaaagcggagaaatagtggcccagtggttgaaaaattgaagcaa

SEQ ID NO: 29 - RNA, 68 nt, synthetic construct
  source 1..68: mol_type = other RNA; note = Registry ID 5UTR032
atattggagcagcaagaggctgggaagccatcacttaccttgcactgagaaagaagacaaaggccagt

SEQ ID NO: 30 - RNA, 14 nt, synthetic construct
  source 1..14: mol_type = other RNA; note = Registry ID 5UTR033
gggagactgccacc

SEQ ID NO: 31 - RNA, 14 nt, synthetic construct
  source 1..14: mol_type = other RNA; note = Registry ID 5UTR034
gggagactgccaag

SEQ ID NO: 32 - RNA, 137 nt, synthetic construct
  source 1..137: mol_type = other RNA; note = Registry ID 5UTR035
agcaccacggcagcaggaggtttcggctaagttggaggtactggccacgactgcttgcccgcgcccgccaggtgatacctccgccggtgacccaggggctctgcgacacaaggagtctgcttgtctaagtgctagac

SEQ ID NO: 33 - RNA, 323 nt, synthetic construct
  source 1..323: mol_type = other RNA; note = Registry ID 5UTR036
ttgatcttttaatcttcgttggccacaattaaaacaaaccagatcgtggagctgcgcgatccctttgcataaaaacatttggcttttgctataaaaattttgactgcaaaacaccgggccattaatagcgtgcggagtgatttacgcgttattgttctgccgggcggacacgtgacgcgcgtggccattgggggcgcgggcgccggcaacttattaggtgactgtacttcacccccccctggtgccaccaagttgttacttgaaatctgcagtttcataatttcggcgggtcgggctgggccggccaggcgcgggctactgca

SEQ ID NO: 34 - RNA, 238 nt, synthetic construct
  source 1..238: mol_type = other RNA; note = Registry ID 5UTR037
agccctccaggacaggctgcatcagaagaggccatcaagcaggtctgttccaagggcctttgcgtcaggtgggctcaggattccagggtggctggaccccaggccccagctctgcagcagggaggacgtggctgggctcgtgaagcttgtgggggtgagcccaggggccccaaggcagggcacctggccttcagcctgcctcagccctgcctgtctcccagatcactgtccttctgcc

SEQ ID NO: 35 - RNA, 50 nt, synthetic construct
  source 1..50: mol_type = other RNA; note = Registry ID 5UTR039
tgagaaagtgtttagtagcaatgatgattcctgtgagtattagtatggtt

SEQ ID NO: 36 - RNA, 50 nt, synthetic construct
  source 1..50: mol_type = other RNA; note = Registry ID 5UTR040
tgagaaagtgtttagtagcaatgatgatctctttaagttttaaaatggtt

SEQ ID NO: 37 - RNA, 50 nt, synthetic construct
  source 1..50: mol_type = other RNA; note = Registry ID 5UTR041
tgagaaagtgtttagtagcaatgatgagccctttaaattttaaaatggtg

SEQ ID NO: 38 - RNA, 50 nt, synthetic construct
  source 1..50: mol_type = other RNA; note = Registry ID 5UTR042
tgagaaagtgtttagtagcaatgatgatctctgtcagttttaaaatggtg

SEQ ID NO: 39 - RNA, 50 nt, synthetic construct
  source 1..50: mol_type = other RNA; note = Registry ID 5UTR043
tgagaaagtgtttagtagcaatgatgatcgcttttggttttaaaatggtg

SEQ ID NO: 40 - RNA, 50 nt, synthetic construct
  source 1..50: mol_type = other RNA; note = Registry ID 5UTR044
tgagaaagtgtttagtagcaatgatgattcctgtgagttttaaaatggtg

SEQ ID NO: 41 - RNA, 50 nt, synthetic construct
  source 1..50: mol_type = other RNA; note = Registry ID 5UTR045
tgagaaagtgtttagtagcaatgatgatctcttgcagttttaaaatggtg

SEQ ID NO: 42 - RNA, 50 nt, synthetic construct
  source 1..50: mol_type = other RNA; note = Registry ID 5UTR046
tgagaaagtgtttagtagcaatgatgattcctgtgagttttaatatggtt

SEQ ID NO: 43 - RNA, 50 nt, synthetic construct
  source 1..50: mol_type = other RNA; note = Registry ID 5UTR047
tgagaaagtgtttagtagcaatgatgatctctgtcagtttagttatggtt

SEQ ID NO: 44 - RNA, 50 nt, synthetic construct
  source 1..50: mol_type = other RNA; note = Registry ID 5UTR048
tgagaaagtgtttagtagcaatgatgaggatactgatttttaaaatggtg

SEQ ID NO: 45 - RNA, 50 nt, synthetic construct
  source 1..50: mol_type = other RNA; note = Registry ID 5UTR049
tgagaaagtgtttagtagcaatgatgattcctgtgagatttaaaatggtg

SEQ ID NO: 46 - RNA, 50 nt, synthetic construct
  source 1..50: mol_type = other RNA; note = Registry ID 5UTR050
tgagaaagtgtttagtagcaatgatgatcgcttattgtttaattatggtt

SEQ ID NO: 47 - RNA, 50 nt, synthetic construct
  source 1..50: mol_type = other RNA; note = Registry ID 5UTR051
tgagaaagtgtttagtagcaatgatgaggacatttgtttttaaaatggtg

SEQ ID NO: 48 - RNA, 50 nt, synthetic construct
  source 1..50: mol_type = other RNA; note = Registry ID 5UTR052
tgagaaagtgtttagtagcaatgatgatctctgtcagtttaattatggtt

SEQ ID NO: 49 - RNA, 50 nt, synthetic construct
  source 1..50: mol_type = other RNA; note = Registry ID 5UTR053
tgagaaagtgtttagtagcaatgatgatctctttaagttttagtatggtt

SEQ ID NO: 50 - RNA, 50 nt, synthetic construct
  source 1..50: mol_type = other RNA; note = Registry ID 5UTR054
tgagaaagtgtttagtagcaatgatgatcgcttacagttttaaaatggtg

SEQ ID NO: 51 - RNA, 50 nt, synthetic construct
  source 1..50: mol_type = other RNA; note = Registry ID 5UTR055
tgagaaagtgtttagtagcaatgatgattcctgtgagatttagtatggtt

SEQ ID NO: 52 - RNA, 50 nt, synthetic construct
  source 1..50: mol_type = other RNA; note = Registry ID 5UTR056
tgagaaagtgtttagtagcaatgatgattcctgtgagtattaaaatggtg

SEQ ID NO: 53 - RNA, 50 nt, synthetic construct
  source 1..50: mol_type = other RNA; note = Registry ID 5UTR057
tgagaaagtgtttagtagcaatgatgaactctgtgagatttaaaatggtg

SEQ ID NO: 54 - RNA, 50 nt, synthetic construct
  source 1..50: mol_type = other RNA; note = Registry ID 5UTR058
tgagaaagtgtttagtagcaatgatgattcctgtgagatttagtatggtg

SEQ ID NO: 55 - RNA, 50 nt, synthetic construct
  source 1..50: mol_type = other RNA; note = Registry ID 5UTR059
tgagaaagtgtttagtagcaatgatgattcctgtgagttttagtatggtt

SEQ ID NO: 56 - RNA, 50 nt, synthetic construct
  source 1..50: mol_type = other RNA; note = Registry ID 5UTR060
tgagaaagtgtttagtagcaatgatgatcgcttttggtttaattatggtt

SEQ ID NO: 57 - RNA, 50 nt, synthetic construct
  source 1..50: mol_type = other RNA; note = Registry ID 5UTR061
tgagagagtgtttagtagcaatgatgatctctttaagttttaaaatggtg

SEQ ID NO: 58 - RNA, 50 nt, synthetic construct
  source 1..50: mol_type = other RNA; note = Registry ID 5UTR062
tgagaaagtgtttagtagcaatgatgaactctgtgagatttagtatggtt

SEQ ID NO: 59 - RNA, 50 nt, synthetic construct
  source 1..50: mol_type = other RNA; note = Registry ID 5UTR063
tgagaaagtgtttagtagcaatgatgattcctgtgagttataaaatggtg

SEQ ID NO: 60 - RNA, 50 nt, synthetic construct
  source 1..50: mol_type = other RNA; note = Registry ID 5UTR064
tgagaaagtgtttagtagcaatgatgatctctttaagttttaaaatggtg

SEQ ID NO: 61 - RNA, 50 nt, synthetic construct
  source 1..50: mol_type = other RNA; note = Registry ID 5UTR065
tgagaaagtgtttagtagcaatgatgaggacatttgttttaattatggtt

SEQ ID NO: 62 - RNA, 50 nt, synthetic construct
  source 1..50: mol_type = other RNA; note = Registry ID 5UTR066
tgagaaagtgtttagtagcaatgatgatcgcttattgtttagttatggtt

SEQ ID NO: 63 - RNA, 50 nt, synthetic construct
  source 1..50: mol_type = other RNA; note = Registry ID 5UTR067
tgagaaagtgtttagtagcaatgatgattcctgtgaattttaaaatggtg

SEQ ID NO: 64 - RNA, 50 nt, synthetic construct
  source 1..50: mol_type = other RNA; note = Registry ID 5UTR068
tgagaaagtgtttagtagcaatgatgatctctttaagttttagaatggtg

SEQ ID NO: 65 - RNA, 50 nt, synthetic construct
  source 1..50: mol_type = other RNA; note = Registry ID 5UTR069
tgagagagtgtttagtagcaatgatgattcctgtgagttttaaaatggtg

SEQ ID NO: 66 - RNA, 50 nt, synthetic construct
  source 1..50: mol_type = other RNA; note = Registry ID 5UTR070
tgagaaagtgtttagtagcaatgatgatccctgtgagatttagtatggtt

SEQ ID NO: 67 - RNA, 50 nt, synthetic construct
  source 1..50: mol_type = other RNA; note = Registry ID 5UTR071
tgagaaagtgtttagtagcaatgatgatctcttgcagtttaattatggtt

SEQ ID NO: 68 - RNA, 50 nt, synthetic construct
  source 1..50: mol_type = other RNA; note = Registry ID 5UTR072
tgagaaagtgtttagtagcaatgatgatctcttacagttttaaaatggtg

SEQ ID NO: 69 - RNA, 90 nt, synthetic construct
  source 1..90: mol_type = other RNA; note = Registry ID 5UTR073
acaaactttcagagacagcagagcacacaagcttctaggacaagagccaggaagaaaccaccggaaggaaccatctcactgtgtgtaaac

SEQ ID NO: 70 - RNA, 50 nt, synthetic construct
  source 1..50: mol_type = other RNA; note = Registry ID 5UTR074
cggagtaatgaagatggtacttacgcatcgcggcaggataggaaggtcgg

SEQ ID NO: 71 - RNA, 50 nt, synthetic construct
  source 1..50: mol_type = other RNA; note = Registry ID 5UTR075
gagttccatctgaggccgttttgtcagatgatatcactgcctcgcaccag

SEQ ID NO: 72 - RNA, 91 nt, synthetic construct
  source 1..91: mol_type = other RNA; note = Registry ID 5UTR077
gtaaaataccaactaattctcgttcgattccggcgaacattctattttaccaacatcggttttttcagtagtaatactgtttttgttcccg

SEQ ID NO: 73 - RNA, 19 nt, synthetic construct
  source 1..19: mol_type = other RNA; note = Registry ID 5UTR078
agacacctctgccctcacc

SEQ ID NO: 74 - RNA, 50 nt, synthetic construct
  source 1..50: mol_type = other RNA; note = Registry ID 5UTR079
tgagaacatatttagtagcaatgatgatatcgtatttagtagttatggtt

SEQ ID NO: 75 - RNA, 50 nt, synthetic construct
  source 1..50: mol_type = other RNA; note = Registry ID 5UTR080
tgagtagagttgtagtagcaatgatgatcgcttatttagtaattatggtt

SEQ ID NO: 76 - RNA, 50 nt, synthetic construct
  source 1..50: mol_type = other RNA; note = Registry ID 5UTR081
tgagtaattatttagtagcaatgatgatatcgtattttgatattatggtt

SEQ ID NO: 77 - RNA, 50 nt, synthetic construct
  source 1..50: mol_type = other RNA; note = Registry ID 5UTR082
tgagaacatatttagttgcaatgatgatcgcgtatcataataatatgatt

SEQ ID NO: 78 - RNA, 50 nt, synthetic construct
  source 1..50: mol_type = other RNA; note = Registry ID 5UTR083
tgagaacatatttagtagcaatgatgaggagatatctaataattatggtt

SEQ ID NO: 79 - RNA, 50 nt, synthetic construct
  source 1..50: mol_type = other RNA; note = Registry ID 5UTR084
tgaggagaaatgtagaagatatgatgaagcctattattgataaaagcatc

SEQ ID NO: 80 - RNA, 50 nt, synthetic construct
  source 1..50: mol_type = other RNA; note = Registry ID 5UTR085
ctcgcatatgcccgtctggctagctattaaatctactacgcgaacataat

SEQ ID NO: 81 - RNA, 50 nt, synthetic construct
  source 1..50: mol_type = other RNA; note = Registry ID 5UTR086
ccgcttttgggggacgtccattggatgttcaagccagtcagacaacaaat

SEQ ID NO: 82 - RNA, 50 nt, synthetic construct
  source 1..50: mol_type = other RNA; note = Registry ID 5UTR087
cccaccatgtcgccgattgtatgtaatggatcgaagtcaatgtgaaatca

SEQ ID NO: 83 - RNA, 50 nt, synthetic construct
  source 1..50: mol_type = other RNA; note = Registry ID 5UTR088
accaccatggcgatgaacgcttacataacatgtggccatgggtcttgcac

SEQ ID NO: 84 - RNA, 62 nt, synthetic construct
  source 1..62: mol_type = other RNA; note = Registry ID 5UTR089
ggctaaaaaaaaaaaaagacccaagctagctagcgtttaaacttaagctaggtaccgagacc

SEQ ID NO: 85 - RNA, 134 nt, synthetic construct
  source 1..134: mol_type = other RNA; note = Registry ID 5UTR090
gtgaagattgaccatctcacaaaagttacgtgcttgtaacacactacgcgcccgttttgtattcgggaagtagttgcgaaaacggtccccttattgcacaagctaagggccacccttctttccccaccgccatc

SEQ ID NO: 86 - RNA, 42 nt, synthetic construct
  source 1..42: mol_type = other RNA; note = Registry ID 5UTR091
acatatccactcctgctctccctcctgcaggtgaccccagcc

SEQ ID NO: 87 - RNA, 41 nt, synthetic construct
  source 1..41: mol_type = other RNA; note = Registry ID 5UTR092
acacatctgctcctgctctctctcctccagcgaccctagcc

SEQ ID NO: 88 - RNA, 68 nt, synthetic construct
  source 1..68: mol_type = other RNA; note = Registry ID 5UTR093
aggatcctctgcaggggagctccgagtgtccacaggaagggaactatcagctcctggcatctgtaagg

SEQ ID NO: 89 - RNA, 80 nt, synthetic construct
  source 1..80: mol_type = other RNA; note = Registry ID 5UTR094
gccaccaagttgttacatgaaatctgcagtttcataatttccgtgggtcgggccgggcgggccaggcgctgggcacggtg

SEQ ID NO: 90 - RNA, 80 nt, synthetic construct
  source 1..80: mol_type = other RNA; note = Registry ID 5UTR095
gccaccaagttgttacgtgaaatctgcagtttcataatttccgtgggtcgggccgggcgggccaggcgctgggcacggtg

SEQ ID NO: 91 - RNA, 243 nt, synthetic construct
  source 1..243: mol_type = other RNA; note = Registry ID 5UTR096
aatcatcttctttaccctggagctgctgctgctgctgctgcttttgcttttggggctgagtttaataagcgagcgagcgagcaagcgagcgcggggggaaaaaggcagagaatgtccgccatctaccctccgctcctgggcgcgctctcattcatagcagcctcttcatgaattacagctgagggggggcggaggaggggggggtaccacacaacaccccagcaaacctccgggcccccaggc

SEQ ID NO: 92 - RNA, 243 nt, synthetic construct
  source 1..243: mol_type = other RNA; note = Registry ID 5UTR097
aatcatcttctttaccctggagctgctgctgctgctgctgcttttgcttttggggctgagtttaataagcgagcgagcgagcaagcgagcgcggggggaaaaaggcagagaatgtccgccatctaccctccgctcctgggcgcgctctcattcatagcagcctcttcttgaattacagctgagggggggcggaggaggggggggtaccacacaacaccccagcaaacctccgggcccccaggc

SEQ ID NO: 93 - RNA, 163 nt, synthetic construct
  source 1..163: mol_type = other RNA; note = Registry ID 5UTR098
gctcacacgcgcgcactcacacacacacacacacacggtggaaggaggcgaataataactcagccatatttcagccgccgccgccgggagctgcgggcacagtccggggacgcggcgagcagcctcggcggccgcacctccgcaaagcgccgcggccgctacg

SEQ ID NO: 94 - RNA, 525 nt, synthetic construct
  source 1..525: mol_type = other RNA; note = Registry ID 5UTR099
actcttgtcagggccgcggcacatgggcggccggatgcgctgagcccggcgctgcggggccgcggagcgctggggagcagcggccgccggcgcggggaggggggtggggtgggacggcgcaccgcctccggtgctggcactaggggctggggtcggcgcggtgtcttctgcccttctgcagccgtcgacatttttttttctttctttttttcaattttgaacattttgcaaaacgaggggttcgaggcaggtgagagcatcctgcacgtcgccggggagcccgcgggcacttggcgcgctctcctgggaccgtctgcactggaaacccgaaagtttttttttaatatatatttttatgcagatgtatttataaagatataagtaatttttttcttcccttttctccaccgccttgagagcgagtacttttggcaaaggacggaggaaaagctcagcaacattttagggggcggttgtttctttcttatttctttttttaaggggaaaaaatttgagtgcatcgcg

SEQ ID NO: 95 - RNA, 234 nt, synthetic construct
  source 1..234: mol_type = other RNA; note = Registry ID 5UTR100
actttcccggtgcactttttctggtgggaggggagagcggagcaggctcacgtgtaaccgcgcaggagcctcctctggcttgagccctttcttgcatttagtaaagataaagacaaggaaaagaagctggatgatgagagtaacagcccgacggtcccccagtcggcattcctggggcctaccttatgggacaaaacccttccctatgacggagatactttccagttggaatac

SEQ ID NO: 96 - RNA, 194 nt, synthetic construct
  source 1..194: mol_type = other RNA; note = Registry ID 5UTR101
acttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcg

SEQ ID NO: 97 - RNA, 194 nt, synthetic construct
  source 1..194: mol_type = other RNA; note = Registry ID 5UTR102
acttctttgggcctcataaacaaccacagaaccacaagttgggtagcctggcagtgtcagaagtctgaacccagcatagtggtcagcaggcaggacgaatcacactgattgcaaaccacagggtttcgcagcgtggtaaaagaaatcattgagtcccccgccttcagaagagggtgcattttcaggaggaagcg

SEQ ID NO: 98 - RNA, 41 nt, synthetic construct
  source 1..41: mol_type = other RNA; note = Registry ID 5UTR103
ctagcttttctcttctgtcaaccccacacgcctttggcaca

SEQ ID NO: 99 - RNA, 37 nt, synthetic construct
  source 1..37: mol_type = other RNA; note = Registry ID 5UTR104
agggacccgcagctcagctacagcacagatcagcacc

SEQ ID NO: 100 - RNA, 36 nt, synthetic construct
  source 1..36: mol_type = other RNA; note = Registry ID 5UTR105
agcactgcctggctccacgtgcctcctggtctcagt

SEQ ID NO: 101 - RNA, 148 nt, synthetic construct
  source 1..148: mol_type = other RNA; note = Registry ID 5UTR106
actggaaaagatagtgaccttaccagggccaaagtttgtagacacaggaattacgaaatggagaagggggagaagtgagctagtggcagcataaaaagaccagcagatgccccacagcactgctcttccagaggcaagaccaaccaag

SEQ ID NO: 102 - RNA, 58 nt, synthetic construct
  source 1..58: mol_type = other RNA; note = Registry ID 5UTR107
aggcacagacaccaaggacagagacgctggctaggccgccctccccactgttaccaac

SEQ ID NO: 103 - RNA, 11 nt, synthetic construct
  source 1..11: mol_type = other RNA; note = Registry ID 5UTR108
ctgctgcaggt

SEQ ID NO: 104 - RNA, 47 nt, synthetic construct
  source 1..47: mol_type = other RNA; note = Registry ID 5UTR109
ctcctcagcttcaggcaccaccactgacctgggacagtgaatcgaca

SEQ ID NO: 105 - RNA, 146 nt, synthetic construct
  source 1..146: mol_type = other RNA; note = Registry ID 5UTR110
aggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgcc

SEQ ID NO: 106 - RNA, 47 nt, synthetic construct
  source 1..47: mol_type = other RNA; note = Registry ID 5UTR111
ctgctcagttcatccctagaggcagctgctccaggaacagaggtgcc

SEQ ID NO: 107 - RNA, 199 nt, synthetic construct
  source 1..199: mol_type = other RNA; note = Registry ID 5UTR112
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaacc

SEQ ID NO: 108 - RNA, 70 nt, synthetic construct
  source 1..70: mol_type = other RNA; note = Registry ID 5UTR113
actataaatagcagccacctctccctggcagacagggacccgcagctcagctacagcacagatcagcacc

SEQ ID NO: 109 - RNA, 146 nt, synthetic construct
  source 1..146: mol_type = other RNA; note = Registry ID 5UTR114
agggaggaaagtgaggattccctgccaaaatgcctgagggcttccctgcctaccacagccctctgtgttcttaaatcctcctgtctgaacagaggccagactctggtttcccccacagcctgtctgtgtctgtcctctgcaaagcc

SEQ ID NO: 110 - RNA, 36 nt, synthetic construct
  source 1..36: mol_type = other RNA; note = Registry ID 5UTR115
aacgaactccatctgggatagcaataacctgtgaaa

SEQ ID NO: 111 - RNA, 148 nt, synthetic construct
  source 1..148: mol_type = other RNA; note = Registry ID 5UTR116
actggaaaagatagtgaccttaccagggccaaagtttgtagacacaggaattacgaattggagaagggggagaagtgagctagtggcagcataaaaagaccagcagaagccccacagcactcctcttccagaggcaagaccaaccaag

SEQ ID NO: 112 - RNA, 55 nt, synthetic construct
  source 1..55: mol_type = other RNA; note = Registry ID 5UTR117
aatcctttctttcagctggagtgctcctcaggagccagccccacccttagaaaag

SEQ ID NO: 113 - RNA, 47 nt, synthetic construct
  source 1..47: mol_type = other RNA; note = Registry ID 5UTR118
gacagtgctgacactacaaggctcggagctccgggcactcagacatc

SEQ ID NO: 114 - RNA, 24 nt, synthetic construct
  source 1..24: mol_type = other RNA; note = Registry ID 5UTR119
gtcctgtggcctctgcagctcagc

SEQ ID NO: 115 - RNA, 146 nt, synthetic construct
  source 1..146: mol_type = other RNA; note = Registry ID 5UTR120
agggaggaaagtgaggattccctgccaaattgcctgagggcttccctgcctaccacagccctctgtgttcttaaatcctcctgtctgaacagaggccagactctggtttcccccacagcctgtctgtgtctgtcctctgcaaagcc

SEQ ID NO: 116 - RNA, 98 nt, synthetic construct
  source 1..98: mol_type = other RNA; note = Registry ID 5UTR122
ctttccggtacctgtgagtcagctaggggagggcagctctcacccaggctgatagttcggtgacctggctttatctactggatgagttccgctgggag

SEQ ID NO: 117 - RNA, 88 nt, synthetic construct
  source 1..88: mol_type = other RNA; note = Registry ID 5UTR123
aactttcccccctcggcgccccaccggctcccgcgcgcctcccctcgcgcccgagcttcgagccaagcagcgtcctggggagcgcgtc

SEQ ID NO: 118 - RNA, 21 nt, synthetic construct
  source 1..21: mol_type = other RNA; note = Registry ID 5UTR124
aggtgtcccgggcgcgccacg

SEQ ID NO: 119 - RNA, 90 nt, synthetic construct
  source 1..90: mol_type = other RNA; note = Registry ID 5UTR125
cttcctccctcatgcctcccttcctcttactctcattcatttcatacacactggctcacacatctactctctctctctatctctctcaga

SEQ ID NO: 120 - RNA, 271 nt, synthetic construct
  source 1..271: mol_type = other RNA; note = Registry ID 5UTR126
gtttcccaagcaagagaggttgttggggaggcttgagtctgacctcctgtcccttgcagcttctgtgcatatccccttacaaacagattagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatggcctaaccctgatcctcttgtcccaca

SEQ ID NO: 121 - RNA, 86 nt, synthetic construct
  source 1..86: mol_type = other RNA; note = Registry ID 5UTR127
ggctctgaaaaaaaaaaaaagacccaagctagctagcgtttaaacttaagctaggtaccgagaccaggtgtcccgggcgcgccacg

SEQ ID NO: 122 - RNA, 86 nt, synthetic construct
  source 1..86: mol_type = other RNA; note = Registry ID 5UTR128
aggtgtcccgggcgcgccacgggctctgaaaaaaaaaaaaagacccaagctagctagcgtttaaacttaagctaggtaccgagacc

SEQ ID NO: 123 - RNA, 98 nt, synthetic construct
  source 1..98: mol_type = other RNA; note = Registry ID 5UTR129
ctttccggtacctgtgagtcagctaggggagggcagctctcacccaggctgatagttcggtgacctggctttatctactggatcagttccgctgggag

SEQ ID NO: 124 - RNA, 134 nt, synthetic construct
  source 1..134: mol_type = other RNA; note = Registry ID 3UTR001
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 125 - RNA, 682 nt, synthetic construct
  source 1..682: mol_type = other RNA; note = Registry ID 3UTR002
gcggactgttactgagctgcgttttacaccctttctttgacaaaacctaacttgcgcagaaaaaaaaaaaataagagacaacattggcatggctttgtttttttaaattttttttaaagtttttttttttttttttttttttttttttaagtttttttgttttgttttggcgcttttgactcaggatttaaaaactggaacggtgaaggcgacagcagttggttggagcaaacatcccccaaagttctacaaatgtggctgaggactttgtacattgttttgtttttttttttttttggttttgtctttttttaatagtcattccaagtatccatgaaataagtggttacaggaagtccctcaccctcccaaaagccacccccactcctaagaggaggatggtcgcgtccatgccctgagtccaccccggggaaggtgacagcattgcttctgtgtaaattatgtactgcaaaaatttttttaaatcttccgccttaatacttcatttttgtttttaatttctgaatggcccaggtctgaggcctcccttttttttgtccccccaacttgatgtatgaaggctttggtctccctgggagggggttgaggtgttgaggcagccagggctggcctgtacactgacttgagaccaataaaagtgcacaccttaccttacacaaac

SEQ ID NO: 126 - RNA, 201 nt, synthetic construct
  source 1..201: mol_type = other RNA; note = Registry ID 3UTR003
gtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 127 - RNA, 839 nt, synthetic construct
  source 1..839: mol_type = other RNA; note = Registry ID 3UTR004
atgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 128 - RNA, 131 nt, synthetic construct
  source 1..131: mol_type = other RNA; note = Registry ID 3UTR005
ggtgcctttgagagtctacttttgctctcttcggaagaacccttaggggttcgtgcatgggcttgcatagcaagtctagatgcgggtaccgtacagtgttgaaaaacactgtaaatctctaaaagagacca

SEQ ID NO: 129 - RNA, 328 nt, synthetic construct
  source 1..328: mol_type = other RNA; note = Registry ID 3UTR006
gaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 130 - RNA, 281 nt, synthetic construct
  source 1..281: mol_type = other RNA; note = Registry ID 3UTR007
ctaactatttgctttgtattttaagattttgtaaatagaaaaatatataaccccactcgtaggtaaggatttattgtatattttatttagttagttattcagtacttacggccctattaccaacgggtattaatcacaaacactttatccccataggattcttttaaatttaaaattttaaataattaacgtcagagtcccatcggggctaacaggtttttcgcacttttcctgctaactgacagaagtgcaatttggtttttgattaatagttgttttct

SEQ ID NO: 131 - RNA, 64 nt, synthetic construct
  source 1..64: mol_type = other RNA; note = Registry ID 3UTR008
cctcgccccggacctgccctcccgccaggtgcacccacctgcaataaatgcagcgaagccggga

SEQ ID NO: 132 - RNA, 115 nt, synthetic construct
  source 1..115: mol_type = other RNA; note = Registry ID 3UTR009
cctcgccccggacctgccctcccgccaggtgcacccacctgcaataaatgcagcgaagccgggacctcgccccggacctgccctcccgccaggtgcacccacctgcaataaatgc

SEQ ID NO: 133 - RNA, 451 nt, synthetic construct
  source 1..451: mol_type = other RNA; note = Registry ID 3UTR010
aaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 134 - RNA, 175 nt, synthetic construct
  source 1..175: mol_type = other RNA; note = Registry ID 3UTR011
ggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctt

SEQ ID NO: 135 - RNA, 174 nt, synthetic construct
  source 1..174: mol_type = other RNA; note = Registry ID 3UTR012
gattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgctt

SEQ ID NO: 136 - RNA, 600 nt, synthetic construct
  source 1..600: mol_type = other RNA; note = Registry ID 3UTR013
gcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatga

SEQ ID NO: 137 - RNA, 111 nt, synthetic construct
  source 1..111: mol_type = other RNA; note = Registry ID 3UTR014
gctggagcctcggtggccatgcttcttgccccttgggcctccccccagcccctcctccccttcctgcacccgtacccccgtggtctttgaataaagtctgagtgggcggca

SEQ ID NO: 138 - RNA, 235 nt, synthetic construct
  source 1..235: mol_type = other RNA; note = Registry ID 3UTR015
gctcgctttcttgctgtccaatttctattaaaggttcctttgttccctaagtccaactactaaactgggggatattatgaagggccttgagcatctggattctgcctaataaaaaacatttattttcattgcaattgccatgtgtatgtgggttcgcccacatactctgatgatccccaatcgtggcgtgtcggcctgcttcggcaggcactggcgccgggatcattcatggcaa

SEQ ID NO: 139 - RNA, 268 nt, synthetic construct
  source 1..268: mol_type = other RNA; note = Registry ID 3UTR016
gctcgctttcttgctgtccaatttctattaaaggttcctttgttccctaagtccaactactaaactgggggatattatgaagggccttgagcatctggattctgcctaataaaaaacatttattttcattgcaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 140 - RNA, 597 nt, synthetic construct
  source 1..597: mol_type = other RNA; note = Registry ID 3UTR017
tcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 141 - RNA, 239 nt, synthetic construct
  source 1..239: mol_type = other RNA; note = Registry ID 3UTR018
ggccaaaggcgtcgagtagacgccaacaacggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcaaaaccaaaccgtcagcgagtagctgacaaaaagaaacaacaacaacaac

SEQ ID NO: 142 - RNA, 255 nt, synthetic construct
  source 1..255: mol_type = other RNA; note = Registry ID 3UTR019
tacggtaatagtgtagtcttctcatcttagtagttagctctctcttatattaagaaaagaaaacaaaaacccccaggtcgctttattttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 143 - RNA, 223 nt, synthetic construct
  source 1..223: mol_type = other RNA; note = Registry ID 3UTR020
tttaacacccttcaggtgtagacccgtcattgtgacgcgtgggttgaggtgccatgaatttgtcattcatggtgcatttatctcaacagttttccctaaccgcgcgttgcgcggcagggtttttactctgagagataaatgcctgctcactaaggtctattagagacattagtacgatccggctaatagtcgctttggatgacctccaaagcggcggattcct

SEQ ID NO: 144 - RNA, 319 nt, synthetic construct
  source 1..319: mol_type = other RNA; note = Registry ID 3UTR021
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 145 - RNA, 60 nt, synthetic construct
  source 1..60: mol_type = other RNA; note = Registry ID 3UTR022
cgtctgcataacttttattatttcttttattaatcaacaaaattttgtttttaacatttc

SEQ ID NO: 146 - RNA, 68 nt, synthetic construct
  source 1..68: mol_type = other RNA; note = Registry ID 3UTR023
taaccctacctcagtcgaattggattgggtcatactgttgtaggggtaaatttttctttaattcggag

SEQ ID NO: 147 - RNA, 457 nt, synthetic construct
  source 1..457: mol_type = other RNA; note = Registry ID 3UTR024
aggccggtcatccttttgacacttcaagtcccgaggataacctcctctcggggttggggggaatcttgggatccagtagtcctccttgaactccatccaacagggtagatttaagagtcatgagactttcattaatcatctcagttgatcagacatggtcgtgtagattctcataacacgggagatcttctagcagtttcagtgaccaacggtgctttccttctccaggaactgataccgaagttgttggacaagccaaggggtgcttcggattactctgtgcttgggcacagaaagaggtcgtagtttgccccttgatagcagattcaacatgaattaactaagaaaggcgatctgcctcccatgaaggacataagcaatagttcacaatcatcttgcatctcagtgaagtgtacataactataaagggctgggtcatctaagcatttcagtcgag

SEQ ID NO: 148 - RNA, 493 nt, synthetic construct
  source 1..493: mol_type = other RNA; note = Registry ID 3UTR027
acattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgtttta

SEQ ID NO: 149 - RNA, 132 nt, synthetic construct
  source 1..132: mol_type = other RNA; note = Registry ID 3UTR028
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 150 - RNA, 128 nt, synthetic construct
  source 1..128: mol_type = other RNA; note = Registry ID 3UTR031
gctcgctttcttgctgtccaatttctggttcctttgttccctaagtccaactactaaactgggggatattatgaagggccttgagcatctggattctgcctaataaaaaacatttattttcattgcaa

SEQ ID NO: 151 - RNA, 664 nt, synthetic construct
  source 1..664: mol_type = other RNA; note = Registry ID 3UTR032
gcggactgttactgagctgcgttttacaccctttctttgacaaaacctaacttgcgcagaaaaaaaaaaaataagagacaacattggcatggctttgtttttttaaattttttttaaagtttttttttttttttttttttttttttttaagtttttttgttttgttttggcgcttttgactcaggatttaaaaactggaacggtgaaggcgacagcagttggttggagcaaacatcccccaaagttctacaaatgtggctgaggactttgtacattgttttgtttttttttttttttggttttgtcttttagtcattccaagtatccatgaaataagtggttacaggaagtccctcaccctcccaaaagccacccccactcctaagaggaggatggtcgcgtccatgccctgagtccaccccggggaaggtgacagcattgcttctgtgtaaattatgtactgcaaaaatttttttaaatcttccgccttaatacttcatttttgttttctgaatggcccaggtctgaggcctcccttttttttgtccccccaacttgatgtatgaaggctttggtctccctgggagggggttgaggtgttgaggcagccagggctggcctgtacactgacttgagaccagtgcacaccttaccttacacaaac

SEQ ID NO: 152 - RNA, 791 nt, synthetic construct
  source 1..791: mol_type = other RNA; note = Registry ID 3UTR033
atgaactcaatctaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaattgaaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaagtatgcagattatttgcccaaatcttcttcagattcagcatttgttctttgccagtctcattttcatcttcttccatggttccacagaagctttgtttcttgggcaagcagaaaattgtacctattttgtatatgtgagatggtgaaaaaaatgagcatgtttggttttccaaaagaacatat

SEQ ID NO: 153 - RNA, 293 nt, synthetic construct
  source 1..293: mol_type = other RNA; note = Registry ID 3UTR034
gaccacacaaggcagatgggctatataaacgttttcgcttttccgtttacgatatatagtctactcttgtgcagaatgaattctcgtaactacatagcacaagtagatgtagttaacctcacatagcaatccagtgtgtaacattagggaggacttgaaagagccaccacattttcaccgaggccacgcggagtacgatcgagtgtacagtgaacaatgctagggagagctgcctatatggaagagccctaatgtggtgctatccccatgtgatagcttcttaggagaatgac

SEQ ID NO: 154 - RNA, 269 nt, synthetic construct
  source 1..269: mol_type = other RNA; note = Registry ID 3UTR035
ctaactatttgctttgtattttaagattttgtaaatagaaaaatatataaccccactcgtaggtaaggagtatattagttagttattcagtacttacggccctattaccaacgggtattaatcacaaacactttatccccataggattcttttaaatttaaaattttaaataattaacgtcagagtcccatcggggctaacaggtttttcgcacttttcctgctaactgacagaagtgcaatttggtttttgattaatagttgttttct

SEQ ID NO: 155 - RNA, 57 nt, synthetic construct
  source 1..57: mol_type = other RNA; note = Registry ID 3UTR036
cctcgccccggacctgccctcccgccaggtgcacccacctgcgcagcgaagccggga

SEQ ID NO: 156 - RNA, 103 nt, synthetic construct
  source 1..103: mol_type = other RNA; note = Registry ID 3UTR037
cctcgccccggacctgccctcccgccaggtgcacccacctgctgcagcgaagccgggacctcgccccggacctgccctcccgccaggtgcacccacctgctgc

SEQ ID NO: 157 - RNA, 564 nt, synthetic construct
  source 1..564: mol_type = other RNA; note = Registry ID 3UTR038
gcggactatgacttagttgcgttacaccctttcttgacaaaacctaacttgcgcagaaaacaagatgagattggcatggctgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgttttagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaattttcttcgccttaatactttttgtttgaatgatgagccttcgtgcccccccttcccccttttttgtcccccaacttgagatgtatgaaggcttttggtctccctgggagtgggtggaggcagccagggcttacctgtacactgacttgagaccagttgagtgcacaccttaaaaatga

SEQ ID NO: 158 - RNA, 105 nt, synthetic construct
  source 1..105: mol_type = other RNA; note = Registry ID 3UTR039
gctggagcctcggtggccatgcttcttgccccttgggcctccccccagcccctcctccccttcctgcacccgtacccccgtggtctttggtctgagtgggcggca

SEQ ID NO: 159 - RNA, 217 nt, synthetic construct
  source 1..217: mol_type = other RNA; note = Registry ID 3UTR040
gctcgctttcttgctgtccaatttctggttcctttgttccctaagtccaactactaaactgggggatattatgaagggccttgagcatctggattctgcctaaacattcattgcaattgccatgtgtatgtgggttcgcccacatactctgatgatccccaatcgtggcgtgtcggcctgcttcggcaggcactggcgccgggatcattcatggcaa

SEQ ID NO: 160 - RNA, 232 nt, synthetic construct
  source 1..232: mol_type = other RNA; note = Registry ID 3UTR041
gctcgctttcttgctgtccaatttctggttcctttgttccctaagtccaactactaaactgggggatattatgaagggccttgagcatctggattctgcctaaacattcattgcaagctcgctttcttgctgtccaatttctggttcctttgttccctaagtccaactactaaactgggggatattatgaagggccttgagcatctggattctgcctaaacattcattgcaa

SEQ ID NO: 161 - RNA, 591 nt, synthetic construct
  source 1..591: mol_type = other RNA; note = Registry ID 3UTR042
tcgacaatcaacctctggattacaaaatttgtgaaagattgactggtattcttaactatgttgctccttttacgctatgtggatacgctgcgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 162 - RNA, 233 nt, synthetic construct
  source 1..233: mol_type = other RNA; note = Registry ID 3UTR043
ggccaaaggcgtcgagtagacgccaacaacggaattgcgggaaaggggtcaacagccgttcagtaccaagtctcaggggaaactttgagatggccttgcaaagggtatgggctgacggacatggtcctaaccacgcagccaagtcctaagtcaacagatcttctgttgatatggatgcagttcaaaaccaaaccgtcagcgagtagctgacaaaaagaaacaacaacaacaac

SEQ ID NO: 163 - RNA, 199 nt, synthetic construct
  source 1..199: mol_type = other RNA; note = Registry ID 3UTR044
ggccaaaggcgtcgagtagacgccaacaacggaattgcgggaaaggggtcaacagccgttcagtaccaagtctcaggggaaactttgagatggccttgcaaagggtatgggctgacggacatggtcctaaccacgcagccaagtcctaagtcaacagatcttctgttgatatggatgcagccgtcagcgagtagctgac

SEQ ID NO: 164 - RNA, 249 nt, synthetic construct
  source 1..249: mol_type = other RNA; note = Registry ID 3UTR045
tacggtaatagtgtagtcttctcatcttagtagttagctctctcttatattaagaaaagaaaacaaaaacccccaggtcgcttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 165 - RNA, 207 nt, synthetic construct
  source 1..207: mol_type = other RNA; note = Registry ID 3UTR046
tacggtaatagtgtagtcttctcatcttagtagttagctctctccccccaggtcgcttgacctgtgttagggacccggtggcagcactgtctagctgcgggcattagactggctagtgctctttgggtaaccacttcccgaaagggtgggctgtggtgaccttccgaactgatagcctccctcctcgcgcggggggggggcctgccc

SEQ ID NO: 166 - RNA, 174 nt, synthetic construct
  source 1..174: mol_type = other RNA; note = Registry ID 3UTR047
tacggtaatagtgtagtcttctcatcttagtagttagctctctccccccaggtcgcttgacctgtgttagggacccggtggcagcactgtctagctgcgggcattagactggctagtgctctttgggtaaccacttcccgaaagggtgggctgtggtgaccttccgaactgata

SEQ ID NO: 167 - RNA, 297 nt, synthetic construct
  source 1..297: mol_type = other RNA; note = Registry ID 3UTR048
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataaccaacaaaattttgtttttaacatttc

SEQ ID NO: 168 - RNA, 266 nt, synthetic construct
  source 1..266: mol_type = other RNA; note = Registry ID 3UTR049
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgc

SEQ ID NO: 169 - RNA, 240 nt, synthetic construct
  source 1..240: mol_type = other RNA; note = Registry ID 3UTR050
ccgctacgccccaatgatccgaccagcctcgatgtacttccgaggaactgatgtgcataatgcatcaggctggtacattagatccccgcttaccgcgggcaatatagcaacacctcgatgtacttccgaggaagcgcagtgcataatgctgcgcagtgttgccacataaccactatattaaccctagcggacgccctcaatgctgaggaagcgtggtgcataatgccacgcagcgtctgc

SEQ ID NO: 170 - RNA, 47 nt, synthetic construct
  source 1..47: mol_type = other RNA; note = Registry ID 3UTR051
cgtctgcataacatttctaatcaacaaaattttgtttttaacatttc

SEQ ID NO: 171 - RNA, 62 nt, synthetic construct
  source 1..62: mol_type = other RNA; note = Registry ID 3UTR052
taaccctacctcagtcgaattggattgggtcatactgttgtaggggtaaatttttctcggag

SEQ ID NO: 172 - RNA, 53 nt, synthetic construct
  source 1..53: mol_type = other RNA; note = Registry ID 3UTR053
taaccctacctcagtcgaattggattgggtcatactgttgtaggggctcggag

SEQ ID NO: 173 - RNA, 410 nt, synthetic construct
  source 1..410: mol_type = other RNA; note = Registry ID 3UTR058
acattacggaaaacacatggtgtgagtccaaagaaggtgttttcctgaagaactgtctattttctcagtcattctctagagtcactgatacacagcctcagtttgcgaatgtagccctttttgtactgccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtggtcaaccttgtttctactgtttta

SEQ ID NO: 174 - RNA, 74 nt, synthetic construct
  source 1..74: mol_type = other RNA; note = Registry ID 3UTR059
gctcgctttcttgctgtcccggttcctttgttccctaagtccaacctaagggccttgagcatctggattctgcc

SEQ ID NO: 175 - RNA, 90 nt, synthetic construct
  source 1..90: mol_type = other RNA; note = Registry ID 3UTR064
gctcgctttcttgctgtccatctggttcctttgttccctaagtccaacctgggggatgaagggccttgagcatctggattctgcctgcaa

SEQ ID NO: 176 - RNA, 492 nt, synthetic construct
  source 1..492: mol_type = other RNA; note = Registry ID 3UTR065
gcggactgttactgagctgcgcaccctttctttgacaacctaacttgcgcagtaagagacaacattggcatggctttgggcgcttgactcaggaactggaacggtgaaggcgacagcagttggttggagcaaacatcccccaaagttctacaaatgtggctgaggactttgtacattgtagtcattccaagtatccatgaaataagtggttacaggaagtccctcaccctcccaaagccacccccactcctaagaggaggatggtcgcgtccatgccctgagtccaccccggggaaggtgacagcattgcttctgtgtaaattatgtactgcatcttccgcctctgaatggcccaggtctgaggcctcccttgtccccccaacttgatgtatgaaggctttggtctccctgggagggggttgaggtgttgaggcagccagggctggcctgtacactgacttgagaccagtgcacaccttaccttacacaaac

SEQ ID NO: 177 - RNA, 456 nt, synthetic construct
  source 1..456: mol_type = other RNA; note = Registry ID 3UTR066
gcggactgttactgagctgcgcaccctttctttgacaacctaacttgcgcagtaagagacaacattggcatggctttgggcgcttgactcaggaactggaacggtgaaggcgacagcagttggttggagcaaacatcccccaaagttctacaaatgtggctgaggactttgtacattgtagtcattccaagtatccatgaaataagtggttacaggaagtccctcaccctcccaaagccacccccactcctaagaggaggatggtcgcgtccatgccctgagtccaccccggggaaggtgacagcattgcttctgtgtaaattatgtactgcatcttccgcctctgaatggcccaggtctgaggcctcccttgtccccccaacttgatgtatgaaggctttggtctccctgggagggggttgaggtgttgaggtgcacaccttaccttacacaaac

SEQ ID NO: 178 - RNA, 442 nt, synthetic construct
  source 1..442: mol_type = other RNA; note = Registry ID 3UTR067
gcggacgcaccctttctttgacaacctaacttgcgcagtaagagacaacattggcatggctttgggcgcttgactcaggaactggaacggtgaaggcgacagcagttggttggagcaaacatcccccaaagttctacaaatgtggctgaggactttgtacattgtagtcattccaagtatccatgaaataagtggttacaggaagtccctcaccctcccaaagccacccccactcctaagaggaggatggtcgcgtccatgccctgagtccaccccggggaaggtgacagcattgcttctgtgtaaattatgtactgcatcttccgcctctgaatggcccaggtctgaggcctcccttgtccccccaacttgatgtatgaaggctttggtctccctgggagggggttgaggtgttgaggtgcacaccttaccttacacaaac

SEQ ID NO: 179 - RNA, 427 nt, synthetic construct
  source 1..427: mol_type = other RNA; note = Registry ID 3UTR068
gcggacgcaccctttctttgacaacctaacttgcgcagtaagagacaacattggcatggctttgggcgcttgactgaacggtgaaggcgattggttggagcaaacatcccccaaagttctacaaatgtggctgaggactttgtacattgtagtcattccaagtatccatgaaataagtggttacaggaagtccctcaccctcccaaagccacccccactcctaagaggaggatggtcgcgtccatgccctgagtccaccccggggaaggtgacagcattgcttctgtgtaaattatgtactgcatcttccgcctctgaatggcccaggtctgaggcctcccttgtccccccaacttgatgtatgaaggctttggtctccctgggagggggttgaggtgttgaggtgcacaccttaccttacacaaac

SEQ ID NO: 180 - RNA, 367 nt, synthetic construct
  source 1..367: mol_type = other RNA; note = Registry ID 3UTR069
gcggacgcaccctttctttgacaacctaacttgcgcagtaagagacaacattggcatggctttgggcgcttgactgaacggtgaaggcgattggttggagcaaacatcccccaaagttctacaaatgtggctgaggactttgtacattgtagtcattccaagtatccatgaaataagtggttacaggaagtccctcaccctcccaaagccacccccactcctaagaggaggatggtcgcgtccatgccctgagtccaccccggggaaggtgaaggcctcccttgtccccccaacttgatgtatgaaggctttggtctccctgggagggggttgaggtgttgaggtgcacaccttaccttacacaaac

SEQ ID NO: 181 - RNA, 242 nt, synthetic construct
  source 1..242: mol_type = other RNA; note = Registry ID 3UTR070
ctaactatttgctttgtattttaagattttgtaaatagaaaaatatataaccccactcgtaggtaaggagtatattagttagttattcagtacttacggccctattaccaacgggtattaatcacaaacactttatccccataggattctcgtcagagtcccatcggggctaacaggtttttcgcacttttcctgctaactgacagaagtgcaatttggtttttgattaatagttgttttct

SEQ ID NO: 182 - RNA, 231 nt, synthetic construct
  source 1..231: mol_type = other RNA; note = Registry ID 3UTR071
ctaactatttgctttgtattttaagattttgtaaatagaaaaatatataaccccactcgtaggtaaggagtatattagttagttattcagtacttacggccctattaccaacgggtattaatcacaaacactttatccccataggattctcgtcagagtcccatcggggctaacaggtttttcgcacttttcctgctaactgacagaagtgcaatttggtagttgttttct

SEQ ID NO: 183 - RNA, 218 nt, synthetic construct
  source 1..218: mol_type = other RNA; note = Registry ID 3UTR072
ctaactatttgctttgtgtaaatagaaaaatatataaccccactcgtaggtaaggagtatattagttagttattcagtacttacggccctattaccaacgggtattaatcacaaacactttatccccataggattctcgtcagagtcccatcggggctaacaggtttttcgcacttttcctgctaactgacagaagtgcaatttggtagttgttttct

SEQ ID NO: 184 - RNA, 182 nt, synthetic construct
  source 1..182: mol_type = other RNA; note = Registry ID 3UTR073
ctaactatttgctttgtgtaaccccactcgtaggtaaggagagttcagtacttacggccctattaccaacgggtattaatcacaaacacccataggattctcgtcagagtcccatcggggctaacaggtttttcgcacttttcctgctaactgacagaagtgcaatttggtagttgttttct

SEQ ID NO: 185 - RNA, 516 nt, synthetic construct
  source 1..516: mol_type = other RNA; note = Registry ID 3UTR074
gcggactatgacttagttgcgttacaccctttcttgacaaaacctaacttgcgcagaaaacaagatgagattggcatggctgttttttttgttttgttttggttttttttttttttttggcttgactcaggaactggaacggtgaaggtgacagcagtcggttggagcgagcatcccccaaagttcacaatgtggccgaggacgcacattgttgtcattccaaatatgagatgcgttgttacaggaagtcccttgccatcctaaaagccaccccacttctctctaaggagaatggcccagtcctctcccaagtccacacaggggaggtgatagcattgctttcgtgtaaattagcaaaattttcttcgccctttttggatgagccttcgtgcccccccttcccccttttttgtcccccaacttgagatgtatgaaggcttttggtctccctgggagtgggtggaggcagccagggcttacctgtacactgacttgagaccagttgagtgcacacga

SEQ ID NO: 186 - RNA, 1252 nt, synthetic construct
  source 1..1252: mol_type = other RNA; note = Registry ID 3UTR076
aaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaa

SEQ ID NO: 187 - RNA, 1680 nt, synthetic construct
  source 1..1680: mol_type = other RNA; note = Registry ID 3UTR077
gttatatattttttaatttaaatttttcatttatcctgagacatataatccaaagtcagcctataaatttctttctgttgctaaaaatcgtcattaggtatctgcctttttggttaaaaaaaaaaggaatagcatcaatagtgagtttgttgtactcatgaccagaaagaccatacatagtttgcccaggaaattctgggtttaagcttgtgtcctatactcttagtaaagttctttgtcactcccagtagtgtcctattttagatgataatttctttgatctccctatttatagttgagaatatagagcatttctaacacatgaatgtcaaagactatattgacttttcaagaaccctactttccttcttattaaacatagctcatctttatatttttaattttattttagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaa

SEQ ID NO: 188 - RNA, 193 nt, synthetic construct
  source 1..193: mol_type = other RNA; note = Registry ID 3UTR078
ggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttta

SEQ ID NO: 189 - RNA, 170 nt, synthetic construct
  source 1..170: mol_type = other RNA; note = Registry ID 3UTR112
atgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagacca

SEQ ID NO: 190 - RNA, 214 nt, synthetic construct
  source 1..214: mol_type = other RNA; note = Registry ID 3UTR113
atgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctt

SEQ ID NO: 191 - RNA, 99 nt, synthetic construct
  source 1..99: mol_type = other RNA; note = Registry ID 3UTR114
atgtctccagttacaactccgcagtggatgtgaagaagccgtctgcataacttttattatttcttttattaatcaacaaaattttgtttttaacatttc

SEQ ID NO: 192 - RNA, 305 nt, synthetic construct
  source 1..305: mol_type = other RNA; note = Registry ID 3UTR115
atgtctccagttacaactccgcagtggatgtgaagaagcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgc

SEQ ID NO: 193 - RNA, 279 nt, synthetic construct
  source 1..279: mol_type = other RNA; note = Registry ID 3UTR116
atgtctccagttacaactccgcagtggatgtgaagaagcccgctacgccccaatgatccgaccagcctcgatgtacttccgaggaactgatgtgcataatgcatcaggctggtacattagatccccgcttaccgcgggcaatatagcaacacctcgatgtacttccgaggaagcgcagtgcataatgctgcgcagtgttgccacataaccactatattaaccctagcggacgccctcaatgctgaggaagcgtggtgcataatgccacgcagcgtctgc

SEQ ID NO: 194 - RNA, 127 nt, synthetic construct
  source 1..127: mol_type = other RNA; note = Registry ID 3UTR117
gcttcctagatagaaaccaaagcagtgcaagattcagttcaaggtcctgaaaaaagaaaaacattttactctgtgtaccttgtgtctttctaaatttctctctccaaaataaagttcaagcattaaa

SEQ ID NO: 195 - RNA, 107 nt, synthetic construct
  source 1..107: mol_type = other RNA; note = Registry ID 3UTR118
gcttcctagatagaaaccaaagcagtgcaagattcagttcaaggtcctgaaaaaagaaaaacattttactctgtgtaccttgtgtctttctaaatttctctctccaa

SEQ ID NO: 196 - RNA, 166 nt, synthetic construct
  source 1..166: mol_type = other RNA; note = Registry ID 3UTR119
atgtctccagttacaactccgcagtggatgtgaagaagcgcttcctagatagaaaccaaagcagtgcaagattcagttcaaggtcctgaaaaaagaaaaacattttactctgtgtaccttgtgtctttctaaatttctctctccaaaataaagttcaagcattaaa

SEQ ID NO: 197 - RNA, 146 nt, synthetic construct
  source 1..146: mol_type = other RNA; note = Registry ID 3UTR120
atgtctccagttacaactccgcagtggatgtgaagaagcgcttcctagatagaaaccaaagcagtgcaagattcagttcaaggtcctgaaaaaagaaaaacattttactctgtgtaccttgtgtctttctaaatttctctctccaa

SEQ ID NO: 198 - RNA, 121 nt, synthetic construct
  source 1..121: mol_type = other RNA; note = Registry ID 3UTR121
gggatgagaacagagagaaatatattcataatttactttatgacctagaaggaaactgtcgtgtgtcctatacattgccatcaactttgtttcctcatctcaaataaagtcctttcagcaa

SEQ ID NO: 199 - RNA, 102 nt, synthetic construct
  source 1..102: mol_type = other RNA; note = Registry ID 3UTR122
gggatgagaacagagagaaatatattcataatttactttatgacctagaaggaaactgtcgtgtgtcctatacattgccatcaactttgtttcctcatctca

SEQ ID NO: 200 - RNA, 160 nt, synthetic construct
  source 1..160: mol_type = other RNA; note = Registry ID 3UTR123
atgtctccagttacaactccgcagtggatgtgaagaagcgggatgagaacagagagaaatatattcataatttactttatgacctagaaggaaactgtcgtgtgtcctatacattgccatcaactttgtttcctcatctcaaataaagtcctttcagcaa

SEQ ID NO: 201 - RNA, 141 nt, synthetic construct
  source 1..141: mol_type = other RNA; note = Registry ID 3UTR124
atgtctccagttacaactccgcagtggatgtgaagaagcgggatgagaacagagagaaatatattcataatttactttatgacctagaaggaaactgtcgtgtgtcctatacattgccatcaactttgtttcctcatctca

SEQ ID NO: 202 - RNA, 115 nt, synthetic construct
  source 1..115: mol_type = other RNA; note = Registry ID 3UTR125
gacagagctctgcggtgtcagggcgagaacccatcttccaaccccggctatttggagacggaaaaactggaattctaacaaggaggagaggagactaaatcacatcaatttgcaa

SEQ ID NO: 203 - RNA, 93 nt, synthetic construct
  source 1..93: mol_type = other RNA; note = Registry ID 3UTR126
gacagagctctgcggtgtcagggcgagaacccatcttccaaccccggctatttggagacggaaaaactggaattctaacaaggaggagaggag

SEQ ID NO: 204 - RNA, 132 nt, synthetic construct
  source 1..132: mol_type = other RNA; note = Registry ID 3UTR127
atgtctccagttacaactccgcagtggatgtgaagaagcgacagagctctgcggtgtcagggcgagaacccatcttccaaccccggctatttggagacggaaaaactggaattctaacaaggaggagaggag

SEQ ID NO: 205 - RNA, 1165 nt, synthetic construct
  source 1..1165: mol_type = other RNA; note = Registry ID 3UTR128
tgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttcta

SEQ ID NO: 206 - RNA, 1140 nt, synthetic construct
  source 1..1140: mol_type = other RNA; note = Registry ID 3UTR129
tgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgcca

SEQ ID NO: 207 - RNA, 976 nt, synthetic construct
  source 1..976: mol_type = other RNA; note = Registry ID 3UTR130
tgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgat

SEQ ID NO: 208 - RNA, 674 nt, synthetic construct
  source 1..674: mol_type = other RNA; note = Registry ID 3UTR131
tgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtga

SEQ ID NO: 209 - RNA, 713 nt, synthetic construct
  source 1..713: mol_type = other RNA; note = Registry ID 3UTR132
atgtctccagttacaactccgcagtggatgtgaagaagctgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtga

SEQ ID NO: 210 - RNA, 4308 nt, synthetic construct
  source 1..4308: mol_type = other RNA; note = Registry ID 3UTR133
gatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaaattaaagtgctgtttagatttattagatcccatatttacttactgctatctactaagtttccttttaattctaccaaccccagataagtaagagtactattaatagaacacagagtgtgtttttgcactgtctgtacctaaagcaataatcctattgtacgctagagcatgctgcctgagtattactagtggacgtaggatattttccctacctaagaatttcactgtcttttaaaaaacaaaaagtaaagtaatgcatttgagcatggccagactattccctaggacaaggaagcagagggaaatgggaggtctaaggatgaggggttaatttatcagtacatgagccaaaaactgcgtcttggattagcctttgacattgatgtgttcggttttgttgttccccttccctcacaccctgcctcgcccccacttttctagttaactttttccatatccctcttgacattcaaaacagttacttaagattcagttttcccactttttggtaatatatatatttttgtgaattatactttgttgtttttaaaaagaaaatcagttgattaagttaataagttgatgttttctaaggccctttttcctagtggtgtcatttttgaatgcctcataaattaatgattctgaagcttatgtttcttattctctgtttgcttttgaacgtatgtgctcttataaagtggacttctgaaaaatgaatgtaaaagacactggtgtatctcagaaggggatggtgttgtcacaaactgtggttaatccaatcaatttaaatgtttactatagaccaaaaggagagattattaaatcgtttaatgtttatacagagtaattataggaagttcttttttgtacagtatttttcagatataaatactgacaatgtattttggaagacatatattatatatagaaaagaggagaggaaaactattccatgttttaaaattatatagcaaagatatatattcaccaatgttgtacagagaagaagtgcttgggggtttttgaagtctttaatattttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaattctgagttcctggaatggcacgttgctgccagtgccccagacagttcttttctaccctgcgggcccgcacgttttatgaggttgatatcggtgctatgtgtttggtttataatttgatagatgtttgactttaaagatgattgttcttttgtttcattaagttgtaaaatgtcaagaaattctgctgttacgacaaagaaacattttacgctagattaaaatatcctttcatcaatgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgcagaaaaaaataaaaagattctaataaaatgtattctcttgtgtgccaggagaggtttcagaaacctacctcgtcttacaaatttaaacactttggagtctgtacaggtgccttatatgtaggtcattgtcacgatacacacacacgaacactccctctggactggctgcctctccatccagggcagttaactagcaaacaaggcagatctgcttcatggagcgggaggccatggcttgactctgagtgatttgggtcaaccggagtcagacgcatgtctgcacgctgcagctattatgagagtccctttgtcatttttcaccttttcatcctaagcatctttcagagattaattatttggccattaacaatgaatccaaatcatatcatactgacatcatctagacatgatttggaaggaacagcttaggacctcctgatgaggtcacattgttgtttcttttaactagacttggcaaagaaaggcaaaaattgaccagcctatctttctgctggtgctgccttaaggaggtagtttgttgaggggagggctgtagatcattacttctttctcttcaggaagtggccactttgaaccattcaaataccacattaggcaagactgtgataggccttttgtcttcaaatacaacaggcctccactgacccatccctcaaagcagaaggaccctttgaggagagtacagatgggattccacagtggggtgggtggaatggaaacctgtactagaccacccagaggttccttctaacccactggtttggtggggaactcacagtaattccaaatgtacaatcagatgtctagggtctgttttcggaagaagcaagaattatcagtggcaccctccccactgcccccagtgtaaaacaatagacattctgtgaaatgcaaagctattctttggtttttctagtagtttatctcattttaccctattcttcctttaaggaaaactcaatctttatcacagtcaattagagcgatcccaaggcatgggaccaggcctgcttgcctatgtgtgatggcaattggagatctggatttagcactggggtctcagcaccctgcaggtgtctgagactaagtgatctgccctccaggtggcgatcaccttctgctcctaggtacccccactggcaaggccaaggtctcctccacgttttttctgcaattaataatgtcatttaaaaaatgagcaaagccttatccgaatcggatatagcaactaaagtcaatacattttgcaggaggctaagtgtaagagtgtgtgtgtgtgtgtgtgcgtgcatgtgtgtgtgtgtgtatgtgtgtgaataagtcgacataaagtctttaattttgagcaccttaccaaacataacaataatccattatccttttggcaacaccacaaagatcgcatctgttaaacaggtacaagttgacatgaggttagtttaattgtacaccatgatattggtggtatttatgctgttaagtccaaacctttatctgtctgttattcttaatgttgaataaactttgaattttttcctttctttcatgtatttttattaacagttggctagcaatggtattctgttcccacctcggtagcaaagagaccatttgtagagattattacctagataataaaatgataatactatataattagtaa

SEQ ID NO: 211 - RNA, 581 nt, synthetic construct
  source 1..581: mol_type = other RNA; note = Registry ID 3UTR134
gatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaa

SEQ ID NO: 212 - RNA, 620 nt, synthetic construct
  source 1..620: mol_type = other RNA; note = Registry ID 3UTR135
atgtctccagttacaactccgcagtggatgtgaagaagcgatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaa

SEQ ID NO: 213 - RNA, 218 nt, synthetic construct
  source 1..218: mol_type = other RNA; note = Registry ID 3UTR136
ggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgagg

SEQ ID NO: 214 - RNA, 257 nt, synthetic construct
  source 1..257: mol_type = other RNA; note = Registry ID 3UTR137
atgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgagg

SEQ ID NO: 215 - RNA, 336 nt, synthetic construct
  source 1..336: mol_type = other RNA; note = Registry ID 3UTR138
ggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctg

SEQ ID NO: 216 - RNA, 167 nt, synthetic construct
  source 1..167: mol_type = other RNA; note = Registry ID 3UTR139
gcttcctagatagaaaccaaagcagtgcaagattcagttcaaggtcctgaaaaaagaaaaacattttactctgtgtaccttgtgtctttctaaatttctctctccaacgtctgcataacttttattatttcttttattaatcaacaaaattttgtttttaacatttc

SEQ ID NO: 217 - RNA, 206 nt, synthetic construct
  source 1..206: mol_type = other RNA; note = Registry ID 3UTR140
atgtctccagttacaactccgcagtggatgtgaagaagcgcttcctagatagaaaccaaagcagtgcaagattcagttcaaggtcctgaaaaaagaaaaacattttactctgtgtaccttgtgtctttctaaatttctctctccaacgtctgcataacttttattatttcttttattaatcaacaaaattttgtttttaacatttc

SEQ ID NO: 218 - RNA, 162 nt, synthetic construct
  source 1..162: mol_type = other RNA; note = Registry ID 3UTR141
gggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttc

SEQ ID NO: 219 - RNA, 201 nt, synthetic construct
  source 1..201: mol_type = other RNA; note = Registry ID 3UTR142
atgtctccagttacaactccgcagtggatgtgaagaagcgggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttc

SEQ ID NO: 220 - RNA, 153 nt, synthetic construct
  source 1..153: mol_type = other RNA; note = Registry ID 3UTR143
gacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttc

SEQ ID NO: 221 - RNA, 192 nt, synthetic construct
  source 1..192: mol_type = other RNA; note = Registry ID 3UTR144
atgtctccagttacaactccgcagtggatgtgaagaagcgacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttc

SEQ ID NO: 222 - RNA, 734 nt, synthetic construct
  source 1..734: mol_type = other RNA; note = Registry ID 3UTR145
tgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgacgtctgcataacttttattatttcttttattaatcaacaaaattttgtttttaacatttc

SEQ ID NO: 223 - RNA, 641 nt, synthetic construct
  source 1..641: mol_type = other RNA; note = Registry ID 3UTR146
gatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaacgtctgcataacttttattatttcttttattaatcaacaaaattttgtttttaacatttc

SEQ ID NO: 224 - RNA, 680 nt, synthetic construct
  source 1..680: mol_type = other RNA; note = Registry ID 3UTR147
atgtctccagttacaactccgcagtggatgtgaagaagcgatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaacgtctgcataacttttattatttcttttattaatcaacaaaattttgtttttaacatttc

SEQ ID NO: 225 - RNA, 278 nt, synthetic construct
  source 1..278: mol_type = other RNA; note = Registry ID 3UTR148
ggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggcgtctgcataacttttattatttcttttattaatcaacaaaattttgtttttaacatttc

SEQ ID NO: 226 - RNA, 317 nt, synthetic construct
  source 1..317: mol_type = other RNA; note = Registry ID 3UTR149
atgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggcgtctgcataacttttattatttcttttattaatcaacaaaattttgtttttaacatttc

SEQ ID NO: 227 - RNA, 99 nt, synthetic construct
  source 1..99: mol_type = other RNA; note = Registry ID 3UTR150
cgtctgcataacttttattatttcttttattaatcaacaaaattttgtttttaacatttcatgtctccagttacaactccgcagtggatgtgaagaagc

SEQ ID NO: 228 - RNA, 105 nt, synthetic construct
  source 1..105: mol_type = other RNA; note = Registry ID 3UTR151
atgtctccagttacaactccgcagtggatgtgaagaagcaaaaaacgtctgcataacttttattatttcttttattaatcaacaaaattttgtttttaacatttc

SEQ ID NO: 229 - RNA, 105 nt, synthetic construct
  source 1..105: mol_type = other RNA; note = Registry ID 3UTR152
cgtctgcataacttttattatttcttttattaatcaacaaaattttgtttttaacatttcaaaaaaatgtctccagttacaactccgcagtggatgtgaagaagc

SEQ ID NO: 230 - RNA, 773 nt, synthetic construct
  source 1..773: mol_type = other RNA; note = Registry ID 3UTR153
atgtctccagttacaactccgcagtggatgtgaagaagctgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgacgtctgcataacttttattatttcttttattaatcaacaaaattttgtttttaacatttc

SEQ ID NO: 231 - RNA, 375 nt, synthetic construct
  source 1..375: mol_type = other RNA; note = Registry ID 3UTR154
atgtctccagttacaactccgcagtggatgtgaagaagcggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctg

SEQ ID NO: 232 - RNA, 396 nt, synthetic construct
  source 1..396: mol_type = other RNA; note = Registry ID 3UTR155
ggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgcgtctgcataacttttattatttcttttattaatcaacaaaattttgtttttaacatttc

SEQ ID NO: 233 - RNA, 435 nt, synthetic construct
  source 1..435: mol_type = other RNA; note = Registry ID 3UTR156
atgtctccagttacaactccgcagtggatgtgaagaagcggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgcgtctgcataacttttattatttcttttattaatcaacaaaattttgtttttaacatttc

SEQ ID NO: 234 - RNA, 657 nt, synthetic construct
  source 1..657: mol_type = other RNA; note = Registry ID 3UTR157
tgccatttgggcgaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtga

SEQ ID NO: 235 - RNA, 696 nt, synthetic construct
  source 1..696: mol_type = other RNA; note = Registry ID 3UTR158
atgtctccagttacaactccgcagtggatgtgaagaagctgccatttgggcgaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtga

SEQ ID NO: 236 - RNA, 717 nt, synthetic construct
  source 1..717: mol_type = other RNA; note = Registry ID 3UTR159
tgccatttgggcgaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgacgtctgcataacttttattatttcttttattaatcaacaaaattttgtttttaacatttc

SEQ ID NO: 237 - RNA, 756 nt, synthetic construct
  source 1..756: mol_type = other RNA; note = Registry ID 3UTR160
atgtctccagttacaactccgcagtggatgtgaagaagctgccatttgggcgaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgacgtctgcataacttttattatttcttttattaatcaacaaaattttgtttttaacatttc

SEQ ID NO: 238 - RNA, 60 nt, synthetic construct
  source 1..60: mol_type = other RNA; note = Registry ID 3UTR161
cgtctggataacttttattatttcttttattaatcaacaaaattttgtttttaacatttc

SEQ ID NO: 239 - RNA, 60 nt, synthetic construct
  source 1..60: mol_type = other RNA; note = Registry ID 3UTR162
cgtctggataacttttattatttctttttttaatcaacaaaattttgtttttaacatttc

SEQ ID NO: 240 - RNA, 60 nt, synthetic construct
  source 1..60: mol_type = other RNA; note = Registry ID 3UTR163
cgtctggataacttttattatttcttttttttatcaacaaaattttgttttaaacatttc

SEQ ID NO: 241 - RNA, 60 nt, synthetic construct
  source 1..60: mol_type = other RNA; note = Registry ID 3UTR164
cgtctggataacttttattatttcttttattattcaacaaatttttgttttaaacatttc

SEQ ID NO: 242 - skipped

SEQ ID NO: 243 - RNA, 47 nt, synthetic construct
  source 1..47: mol_type = other RNA; note = Registry ID 3UTR166
accagatcccggagttggaaaacaatgaaaaggcccccaaggtagtt

SEQ ID NO: 244 - RNA, 24 nt, synthetic construct
  source 1..24: mol_type = other RNA; note = Registry ID 3UTR167
attccaaatgtgaatatatagttt

SEQ ID NO: 245 - RNA, 47 nt, synthetic construct
  source 1..47: mol_type = other RNA; note = Registry ID 3UTR168
tgtgttcagaaaactaggcaggaaagtaggaaaagatctgttaatcg

SEQ ID NO: 246 - skipped

SEQ ID NO: 247 - RNA, 65 nt, synthetic construct
  source 1..65: mol_type = other RNA; note = Registry ID 3UTR170
tatagcgtctgcataacttttattatttcttttattaatcaacaaaattttgtttttaacatttc

SEQ ID NO: 248 - RNA, 65 nt, synthetic construct
  source 1..65: mol_type = other RNA; note = Registry ID 3UTR171
tatagcgtctggataacttttattatttcttttattaatcaacaaaattttgtttttaacatttc

SEQ ID NO: 249 - RNA, 65 nt, synthetic construct
  source 1..65: mol_type = other RNA; note = Registry ID 3UTR172
tatagcgtctggataacttttattatttctttttttaatcaacaaaattttgtttttaacatttc

SEQ ID NO: 250 - RNA, 65 nt, synthetic construct
  source 1..65: mol_type = other RNA; note = Registry ID 3UTR173
tatagcgtctggataacttttattatttcttttttttatcaacaaaattttgttttaaacatttc

SEQ ID NO: 251 - RNA, 65 nt, synthetic construct
  source 1..65: mol_type = other RNA; note = Registry ID 3UTR174
tatagcgtctggataacttttattatttcttttattattcaacaaatttttgttttaaacatttc

SEQ ID NO: 252 - RNA, 107 nt, synthetic construct
  source 1..107: mol_type = other RNA; note = Registry ID 3UTR175
accagatcccggagttggaaaacaatgaaaaggcccccaaggtagttcgtctgcataacttttattatttcttttattaatcaacaaaattttgtttttaacatttc

SEQ ID NO: 253 - RNA, 107 nt, synthetic construct
  source 1..107: mol_type = other RNA; note = Registry ID 3UTR176
accagatcccggagttggaaaacaatgaaaaggcccccaaggtagttcgtctggataacttttattatttcttttattaatcaacaaaattttgtttttaacatttc

SEQ ID NO: 254 - RNA, 107 nt, synthetic construct
  source 1..107: mol_type = other RNA; note = Registry ID 3UTR177
accagatcccggagttggaaaacaatgaaaaggcccccaaggtagttcgtctggataacttttattatttctttttttaatcaacaaaattttgtttttaacatttc

SEQ ID NO: 255 - RNA, 107 nt, synthetic construct
  source 1..107: mol_type = other RNA; note = Registry ID 3UTR178
accagatcccggagttggaaaacaatgaaaaggcccccaaggtagttcgtctggataacttttattatttcttttttttatcaacaaaattttgttttaaacatttc

SEQ ID NO: 256 - RNA, 107 nt, synthetic construct
  source 1..107: mol_type = other RNA; note = Registry ID 3UTR179
accagatcccggagttggaaaacaatgaaaaggcccccaaggtagttcgtctggataacttttattatttcttttattattcaacaaatttttgttttaaacatttc

SEQ ID NO: 257 - RNA, 84 nt, synthetic construct
  source 1..84: mol_type = other RNA; note = Registry ID 3UTR180
attccaaatgtgaatatatagtttcgtctgcataacttttattatttcttttattaatcaacaaaattttgtttttaacatttc

SEQ ID NO: 258 - RNA, 84 nt, synthetic construct
  source 1..84: mol_type = other RNA; note = Registry ID 3UTR181
attccaaatgtgaatatatagtttcgtctggataacttttattatttcttttattaatcaacaaaattttgtttttaacatttc

SEQ ID NO: 259 - RNA, 84 nt, synthetic construct
  source 1..84: mol_type = other RNA; note = Registry ID 3UTR182
attccaaatgtgaatatatagtttcgtctggataacttttattatttctttttttaatcaacaaaattttgtttttaacatttc

SEQ ID NO: 260 - RNA, 84 nt, synthetic construct
  source 1..84: mol_type = other RNA; note = Registry ID 3UTR183
attccaaatgtgaatatatagtttcgtctggataacttttattatttcttttttttatcaacaaaattttgttttaaacatttc

SEQ ID NO: 261 - RNA, 84 nt, synthetic construct
  source 1..84: mol_type = other RNA; note = Registry ID 3UTR184
attccaaatgtgaatatatagtttcgtctggataacttttattatttcttttattattcaacaaatttttgttttaaacatttc

SEQ ID NO: 262 - RNA, 107 nt, synthetic construct
  source 1..107: mol_type = other RNA; note = Registry ID 3UTR185
tgtgttcagaaaactaggcaggaaagtaggaaaagatctgttaatcgcgtctgcataacttttattatttcttttattaatcaacaaaattttgtttttaacatttc

SEQ ID NO: 263 - RNA, 107 nt, synthetic construct
  source 1..107: mol_type = other RNA; note = Registry ID 3UTR186
tgtgttcagaaaactaggcaggaaagtaggaaaagatctgttaatcgcgtctggataacttttattatttcttttattaatcaacaaaattttgtttttaacatttc

SEQ ID NO: 264 - RNA, 107 nt, synthetic construct
  source 1..107: mol_type = other RNA; note = Registry ID 3UTR187
tgtgttcagaaaactaggcaggaaagtaggaaaagatctgttaatcgcgtctggataacttttattatttctttttttaatcaacaaaattttgtttttaacatttc

SEQ ID NO: 265 - RNA, 107 nt, synthetic construct
  source 1..107: mol_type = other RNA; note = Registry ID 3UTR188
tgtgttcagaaaactaggcaggaaagtaggaaaagatctgttaatcgcgtctggataacttttattatttcttttttttatcaacaaaattttgttttaaacatttc

SEQ ID NO: 266 - RNA, 107 nt, synthetic construct
  source 1..107: mol_type = other RNA; note = Registry ID 3UTR189
tgtgttcagaaaactaggcaggaaagtaggaaaagatctgttaatcgcgtctggataacttttattatttcttttattattcaacaaatttttgttttaaacatttc

SEQ ID NO: 267 - RNA, 65 nt, synthetic construct
  source 1..65: mol_type = other RNA; note = Registry ID 3UTR190
aatcacgtctgcataacttttattatttcttttattaatcaacaaaattttgtttttaacatttc

SEQ ID NO: 268 - RNA, 65 nt, synthetic construct
  source 1..65: mol_type = other RNA; note = Registry ID 3UTR191
aatcacgtctggataacttttattatttcttttattaatcaacaaaattttgtttttaacatttc

SEQ ID NO: 269 - RNA, 65 nt, synthetic construct
  source 1..65: mol_type = other RNA; note = Registry ID 3UTR192
aatcacgtctggataacttttattatttctttttttaatcaacaaaattttgtttttaacatttc

SEQ ID NO: 270 - RNA, 65 nt, synthetic construct
  source 1..65: mol_type = other RNA; note = Registry ID 3UTR193
aatcacgtctggataacttttattatttcttttttttatcaacaaaattttgttttaaacatttc

SEQ ID NO: 271 - RNA, 65 nt, synthetic construct
  source 1..65: mol_type = other RNA; note = Registry ID 3UTR194
aatcacgtctggataacttttattatttcttttattattcaacaaatttttgttttaaacatttc

SEQ ID NO: 272 - RNA, 65 nt, synthetic construct
  source 1..65: mol_type = other RNA; note = Registry ID 3UTR195
cgtctgcataacttttattatttcttttattaatcaacaaaattttgtttttaacatttctatag

SEQ ID NO: 273 - RNA, 65 nt, synthetic construct
  source 1..65: mol_type = other RNA; note = Registry ID 3UTR196
cgtctggataacttttattatttcttttattaatcaacaaaattttgtttttaacatttctatag

SEQ ID NO: 274 - RNA, 65 nt, synthetic construct
  source 1..65: mol_type = other RNA; note = Registry ID 3UTR197
cgtctggataacttttattatttctttttttaatcaacaaaattttgtttttaacatttctatag

SEQ ID NO: 275 - RNA, 65 nt, synthetic construct
  source 1..65: mol_type = other RNA; note = Registry ID 3UTR198
cgtctggataacttttattatttcttttttttatcaacaaaattttgttttaaacatttctatag

SEQ ID NO: 276 - RNA, 65 nt, synthetic construct
  source 1..65: mol_type = other RNA; note = Registry ID 3UTR199
cgtctggataacttttattatttcttttattattcaacaaatttttgttttaaacatttctatag

SEQ ID NO: 277 - RNA, 107 nt, synthetic construct
  source 1..107: mol_type = other RNA; note = Registry ID 3UTR200
cgtctgcataacttttattatttcttttattaatcaacaaaattttgtttttaacatttcaccagatcccggagttggaaaacaatgaaaaggcccccaaggtagtt

SEQ ID NO: 278 - RNA, 107 nt, synthetic construct
  source 1..107: mol_type = other RNA; note = Registry ID 3UTR201
cgtctggataacttttattatttcttttattaatcaacaaaattttgtttttaacatttcaccagatcccggagttggaaaacaatgaaaaggcccccaaggtagtt

SEQ ID NO: 279 - RNA, 107 nt, synthetic construct
  source 1..107: mol_type = other RNA; note = Registry ID 3UTR202
cgtctggataacttttattatttctttttttaatcaacaaaattttgtttttaacatttcaccagatcccggagttggaaaacaatgaaaaggcccccaaggtagtt

SEQ ID NO: 280 - RNA, 107 nt, synthetic construct
  source 1..107: mol_type = other RNA; note = Registry ID 3UTR203
cgtctggataacttttattatttcttttttttatcaacaaaattttgttttaaacatttcaccagatcccggagttggaaaacaatgaaaaggcccccaaggtagtt

SEQ ID NO: 281 - RNA, 107 nt, synthetic construct
  source 1..107: mol_type = other RNA; note = Registry ID 3UTR204
cgtctggataacttttattatttcttttattattcaacaaatttttgttttaaacatttcaccagatcccggagttggaaaacaatgaaaaggcccccaaggtagtt

SEQ ID NO: 282 - RNA, 84 nt, synthetic construct
  source 1..84: mol_type = other RNA; note = Registry ID 3UTR205
cgtctgcataacttttattatttcttttattaatcaacaaaattttgtttttaacatttcattccaaatgtgaatatatagttt

SEQ ID NO: 283 - RNA, 84 nt, synthetic construct
  source 1..84: mol_type = other RNA; note = Registry ID 3UTR206
cgtctggataacttttattatttcttttattaatcaacaaaattttgtttttaacatttcattccaaatgtgaatatatagttt

SEQ ID NO: 284 - RNA, 84 nt, synthetic construct
  source 1..84: mol_type = other RNA; note = Registry ID 3UTR207
cgtctggataacttttattatttctttttttaatcaacaaaattttgtttttaacatttcattccaaatgtgaatatatagttt

SEQ ID NO: 285 - RNA, 84 nt, synthetic construct
  source 1..84: mol_type = other RNA; note = Registry ID 3UTR208
cgtctggataacttttattatttcttttttttatcaacaaaattttgttttaaacatttcattccaaatgtgaatatatagttt

SEQ ID NO: 286 - RNA, 84 nt, synthetic construct
  source 1..84: mol_type = other RNA; note = Registry ID 3UTR209
cgtctggataacttttattatttcttttattattcaacaaatttttgttttaaacatttcattccaaatgtgaatatatagttt

SEQ ID NO: 287 - RNA, 107 nt, synthetic construct
  source 1..107: mol_type = other RNA; note = Registry ID 3UTR210
cgtctgcataacttttattatttcttttattaatcaacaaaattttgtttttaacatttctgtgttcagaaaactaggcaggaaagtaggaaaagatctgttaatcg

SEQ ID NO: 288 - RNA, 107 nt, synthetic construct
  source 1..107: mol_type = other RNA; note = Registry ID 3UTR211
cgtctggataacttttattatttcttttattaatcaacaaaattttgtttttaacatttctgtgttcagaaaactaggcaggaaagtaggaaaagatctgttaatcg

SEQ ID NO: 289 - RNA, 107 nt, synthetic construct
  source 1..107: mol_type = other RNA; note = Registry ID 3UTR212
cgtctggataacttttattatttctttttttaatcaacaaaattttgtttttaacatttctgtgttcagaaaactaggcaggaaagtaggaaaagatctgttaatcg

SEQ ID NO: 290 - RNA, 107 nt, synthetic construct
  source 1..107: mol_type = other RNA; note = Registry ID 3UTR213
cgtctggataacttttattatttcttttttttatcaacaaaattttgttttaaacatttctgtgttcagaaaactaggcaggaaagtaggaaaagatctgttaatcg

SEQ ID NO: 291 - RNA, 107 nt, synthetic construct
  source 1..107: mol_type = other RNA; note = Registry ID 3UTR214
cgtctggataacttttattatttcttttattattcaacaaatttttgttttaaacatttctgtgttcagaaaactaggcaggaaagtaggaaaagatctgttaatcg

SEQ ID NO: 292 - RNA, 65 nt, synthetic construct
  source 1..65: mol_type = other RNA; note = Registry ID 3UTR215
cgtctgcataacttttattatttcttttattaatcaacaaaattttgtttttaacatttcaatca

SEQ ID NO: 293 - RNA, 65 nt, synthetic construct
  source 1..65: mol_type = other RNA; note = Registry ID 3UTR216
cgtctggataacttttattatttcttttattaatcaacaaaattttgtttttaacatttcaatca

SEQ ID NO: 294 - RNA, 65 nt, synthetic construct
  source 1..65: mol_type = other RNA; note = Registry ID 3UTR217
cgtctggataacttttattatttctttttttaatcaacaaaattttgtttttaacatttcaatca

SEQ ID NO: 295 - RNA, 65 nt, synthetic construct
  source 1..65: mol_type = other RNA; note = Registry ID 3UTR218
cgtctggataacttttattatttcttttttttatcaacaaaattttgttttaaacatttcaatca

SEQ ID NO: 296 - RNA, 65 nt, synthetic construct
  source 1..65: mol_type = other RNA; note = Registry ID 3UTR219
cgtctggataacttttattatttcttttattattcaacaaatttttgttttaaacatttcaatca

SEQ ID NO: 297 - RNA, 70 nt, synthetic construct
  source 1..70: mol_type = other RNA; note = Registry ID 3UTR220
tatagcgtctgcataacttttattatttcttttattaatcaacaaaattttgtttttaacatttctatag

SEQ ID NO: 298 - RNA, 70 nt, synthetic construct
  source 1..70: mol_type = other RNA; note = Registry ID 3UTR221
tatagcgtctggataacttttattatttcttttattaatcaacaaaattttgtttttaacatttctatag

SEQ ID NO: 299 - RNA, 70 nt, synthetic construct
  source 1..70: mol_type = other RNA; note = Registry ID 3UTR222
tatagcgtctggataacttttattatttctttttttaatcaacaaaattttgtttttaacatttctatag

SEQ ID NO: 300 - RNA, 70 nt, synthetic construct
  source 1..70: mol_type = other RNA; note = Registry ID 3UTR223
tatagcgtctggataacttttattatttcttttttttatcaacaaaattttgttttaaacatttctatag

SEQ ID NO: 301 - RNA, 70 nt, synthetic construct
  source 1..70: mol_type = other RNA; note = Registry ID 3UTR224
tatagcgtctggataacttttattatttcttttattattcaacaaatttttgttttaaacatttctatag

SEQ ID NO: 302 - RNA, 154 nt, synthetic construct
  source 1..154: mol_type = other RNA; note = Registry ID 3UTR225
accagatcccggagttggaaaacaatgaaaaggcccccaaggtagttcgtctgcataacttttattatttcttttattaatcaacaaaattttgtttttaacatttcaccagatcccggagttggaaaacaatgaaaaggcccccaaggtagtt

SEQ ID NO: 303 - RNA, 154 nt, synthetic construct
  source 1..154: mol_type = other RNA; note = Registry ID 3UTR226
accagatcccggagttggaaaacaatgaaaaggcccccaaggtagttcgtctggataacttttattatttcttttattaatcaacaaaattttgtttttaacatttcaccagatcccggagttggaaaacaatgaaaaggcccccaaggtagtt

SEQ ID NO: 304 - RNA, 154 nt, synthetic construct
  source 1..154: mol_type = other RNA; note = Registry ID 3UTR227
accagatcccggagttggaaaacaatgaaaaggcccccaaggtagttcgtctggataacttttattatttctttttttaatcaacaaaattttgtttttaacatttcaccagatcccggagttggaaaacaatgaaaaggcccccaaggtagtt

SEQ ID NO: 305 - RNA, 154 nt, synthetic construct
  source 1..154: mol_type = other RNA; note = Registry ID 3UTR228
accagatcccggagttggaaaacaatgaaaaggcccccaaggtagttcgtctggataacttttattatttcttttttttatcaacaaaattttgttttaaacatttcaccagatcccggagttggaaaacaatgaaaaggcccccaaggtagtt

SEQ ID NO: 306 - RNA, 154 nt, synthetic construct
  source 1..154: mol_type = other RNA; note = Registry ID 3UTR229
accagatcccggagttggaaaacaatgaaaaggcccccaaggtagttcgtctggataacttttattatttcttttattattcaacaaatttttgttttaaacatttcaccagatcccggagttggaaaacaatgaaaaggcccccaaggtagtt

SEQ ID NO: 307 - RNA, 108 nt, synthetic construct
  source 1..108: mol_type = other RNA; note = Registry ID 3UTR230
attccaaatgtgaatatatagtttcgtctgcataacttttattatttcttttattaatcaacaaaattttgtttttaacatttcattccaaatgtgaatatatagttt

SEQ ID NO: 308 - RNA, 108 nt, synthetic construct
  source 1..108: mol_type = other RNA; note = Registry ID 3UTR231
attccaaatgtgaatatatagtttcgtctggataacttttattatttcttttattaatcaacaaaattttgtttttaacatttcattccaaatgtgaatatatagttt

SEQ ID NO: 309 - RNA, 108 nt, synthetic construct
  source 1..108: mol_type = other RNA; note = Registry ID 3UTR232
attccaaatgtgaatatatagtttcgtctggataacttttattatttctttttttaatcaacaaaattttgtttttaacatttcattccaaatgtgaatatatagttt

SEQ ID NO: 310 - RNA, 108 nt, synthetic construct
  source 1..108: mol_type = other RNA; note = Registry ID 3UTR233
attccaaatgtgaatatatagtttcgtctggataacttttattatttcttttttttatcaacaaaattttgttttaaacatttcattccaaatgtgaatatatagttt

SEQ ID NO: 311 - RNA, 108 nt, synthetic construct
  source 1..108: mol_type = other RNA; note = Registry ID 3UTR234
attccaaatgtgaatatatagtttcgtctggataacttttattatttcttttattattcaacaaatttttgttttaaacatttcattccaaatgtgaatatatagttt

SEQ ID NO: 312 - RNA, 154 nt, synthetic construct
  source 1..154: mol_type = other RNA; note = Registry ID 3UTR235
tgtgttcagaaaactaggcaggaaagtaggaaaagatctgttaatcgcgtctgcataacttttattatttcttttattaatcaacaaaattttgtttttaacatttctgtgttcagaaaactaggcaggaaagtaggaaaagatctgttaatcg

SEQ ID NO: 313 - RNA, 154 nt, synthetic construct
  source 1..154: mol_type = other RNA; note = Registry ID 3UTR236
tgtgttcagaaaactaggcaggaaagtaggaaaagatctgttaatcgcgtctggataacttttattatttcttttattaatcaacaaaattttgtttttaacatttctgtgttcagaaaactaggcaggaaagtaggaaaagatctgttaatcg

SEQ ID NO: 314 - RNA, 154 nt, synthetic construct
  source 1..154: mol_type = other RNA; note = Registry ID 3UTR237
tgtgttcagaaaactaggcaggaaagtaggaaaagatctgttaatcgcgtctggataacttttattatttctttttttaatcaacaaaattttgtttttaacatttctgtgttcagaaaactaggcaggaaagtaggaaaagatctgttaatcg

SEQ ID NO: 315 - RNA, 154 nt, synthetic construct
  source 1..154: mol_type = other RNA; note = Registry ID 3UTR238
tgtgttcagaaaactaggcaggaaagtaggaaaagatctgttaatcgcgtctggataacttttattatttcttttttttatcaacaaaattttgttttaaacatttctgtgttcagaaaactaggcaggaaagtaggaaaagatctgttaatcg

SEQ ID NO: 316 - RNA, 154 nt, synthetic construct
  source 1..154: mol_type = other RNA; note = Registry ID 3UTR239
tgtgttcagaaaactaggcaggaaagtaggaaaagatctgttaatcgcgtctggataacttttattatttcttttattattcaacaaatttttgttttaaacatttctgtgttcagaaaactaggcaggaaagtaggaaaagatctgttaatcg

SEQ ID NO: 317 - RNA, 70 nt, synthetic construct
  source 1..70: mol_type = other RNA; note = Registry ID 3UTR240
aatcacgtctgcataacttttattatttcttttattaatcaacaaaattttgtttttaacatttcaatca

SEQ ID NO: 318 - RNA, 70 nt, synthetic construct
  source 1..70: mol_type = other RNA; note = Registry ID 3UTR241
aatcacgtctggataacttttattatttcttttattaatcaacaaaattttgtttttaacatttcaatca

SEQ ID NO: 319 - RNA, 70 nt, synthetic construct
  source 1..70: mol_type = other RNA; note = Registry ID 3UTR242
aatcacgtctggataacttttattatttctttttttaatcaacaaaattttgtttttaacatttcaatca

SEQ ID NO: 320 - RNA, 70 nt, synthetic construct
  source 1..70: mol_type = other RNA; note = Registry ID 3UTR243
aatcacgtctggataacttttattatttcttttttttatcaacaaaattttgttttaaacatttcaatca

SEQ ID NO: 321 - RNA, 70 nt, synthetic construct
  source 1..70: mol_type = other RNA; note = Registry ID 3UTR244
aatcacgtctggataacttttattatttcttttattattcaacaaatttttgttttaaacatttcaatca

SEQ ID NO: 322 - RNA, 188 nt, synthetic construct
  source 1..188: mol_type = other RNA; note = Registry ID 3UTR245
tatagaccagatcccggagttggaaaacaatgaaaaggcccccaaggtagttattccaaatgtgaatatatagttttgtgttcagaaaactaggcaggaaagtaggaaaagatctgttaatcgaatcacgtctgcataacttttattatttcttttattaatcaacaaaattttgtttttaacatttc

SEQ ID NO: 323 - RNA, 414 nt, synthetic construct
  source 1..414: mol_type = other RNA; note = Registry ID 3UTR246
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaacca

SEQ ID NO: 324 - RNA, 112 nt, synthetic construct
  source 1..112: mol_type = other RNA; note = Registry ID 3UTR247
gcttcctcttcactctgctctcaggagatctggctgtgaggccctcagggcagggatacaaagcggggagagggtacacaatgggtatctaataaatacttaagaggtggaa

SEQ ID NO: 325 - RNA, 122 nt, synthetic construct
  source 1..122: mol_type = other RNA; note = Registry ID 3UTR248
caggacacagccttggatcaggacagagacttgggggccatcctgcccctccaacccgacatgtgtacctcagctttttccctcacttgcatcaataaagcttctgtgtttggaacagctaa

SEQ ID NO: 326 - RNA, 165 nt, synthetic construct
  source 1..165: mol_type = other RNA; note = Registry ID 3UTR249
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgaccca

SEQ ID NO: 327 - RNA, 113 nt, synthetic construct
  source 1..113: mol_type = other RNA; note = Registry ID 3UTR250
agtgtccagaccattgtcttccaaccccagctggcctctagaacacccactggccagtcctagagctcctgtccctacccactctttgctacaataaatgctgaatgaatcca

SEQ ID NO: 328 - RNA, 1702 nt, synthetic construct
  source 1..1702: mol_type = other RNA; note = Registry ID 3UTR251
ctgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 329 - RNA, 116 nt, synthetic construct
  source 1..116: mol_type = other RNA; note = Registry ID 3UTR252
ggacctgaagggtgacatcccaggaggggcctctgaaatttcccacaccccagcgcctgtgctgaggactccctccatgtggccccaggtgccaccaataaaaatcctacagaaaa

SEQ ID NO: 330 - RNA, 188 nt, synthetic construct
  source 1..188: mol_type = other RNA; note = Registry ID 3UTR253
gacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatga

SEQ ID NO: 331 - RNA, 144 nt, synthetic construct
  source 1..144: mol_type = other RNA; note = Registry ID 3UTR254
gagccttctgagcccagcgacttctgaagggccccttgcaaagtaatagggcttctgcctaagcctctccctccagccaataggcagctttcttaactatcctaacaagccttggaccaaatggaaataaagctttttgatgca

SEQ ID NO: 332 - RNA, 137 nt, synthetic construct
  source 1..137: mol_type = other RNA; note = Registry ID 3UTR255
gcttcctcttcactctgctctcaggagacctggctatgaggccctcggggcagggatacaaagttagtgaggtctatgtccagagaagctgagatatggcatataataggcatctaataaatgcttaagaggtggaa

SEQ ID NO: 333 - RNA, 598 nt, synthetic construct
  source 1..598: mol_type = other RNA; note = Registry ID 3UTR256
agctaaggttggatgcatggttgcatggatttggggtgtgctatgaggggtggtgtatccttgggagagatataaagtggagggagggagccgtccggtcagtagggcaccaatcccacctccttcattacctcctggccatgattctcctgggagataattctgctctctggagatgttggtaggaaagtttcaagttacgcagctgagaaacagggaccaaatagtgctcctgggtgcattgtcaccgtgggtggccactcaagggtccaagcctctagggccatccttgggctaacaactggggtgggtgtgagcaggtggaaggagcctcagcccatgccattacctcctgcttccttatcaggctgtgtgttaattctgggccagtctacaccctcccacggggtggaaatggcctggaggatgtgagggcacccctcctctgaagatccctgtacacgtggtgttgggactggaaccattatgcggccccataggcctcaggagtcatcccagaagcagtggctgggaggtggtgtcctaagtaaggatctgtgcagaggacaaataaatcagtttttgatttgtcttgaaa

SEQ ID NO: 334 - RNA, 608 nt, synthetic construct
  source 1..608: mol_type = other RNA; note = Registry ID 3UTR257
cagtgcacaatattttcccatctgtattattttttttcagcatgtattacttgacaaagagacactgtgcagagggtgaccacagtctgtaattccccacttcaatacaaagggtgtcgttcttttccaacaaaatagcaatcccttttatttcattgcttttgacttttcaatgggtgtcctaggaaccttttagaaagaaatggactttcatcctggaaatatattaactgttaaaaagaaaacattgaaaatgtgtttagacaacgtcatcccctggcaggctaaagtgctgtatcctttagtaaaattggaggtagcaaacactaaggtgaaaagataatgatctcattgtttattaacctgtattctgtttacatgtctttaaaacagtggttcttaaattgtaagctcaggttcaaagtgttggtaatgcctgattcacaactttgagaaggtagcactggagagaattggaatgggtggcggtaattggtgatacttctttgaatgtagatttccaatcacatctttagtgtctgaatatatccaaatgttttaggatgtatgttacttcttagagagaaataaagcatttttgggaagaa

SEQ ID NO: 335 - RNA, 569 nt, synthetic construct
  source 1..569: mol_type = other RNA; note = Registry ID 3UTR258
agctaaggttggatgcatggttgcatggatttggggtgtgctatgaggggtggtgtatccttgggagagatataaagtggagggagggagccgtccggtcagtagggcaccaatcccacctccttcattacctcctggccatgattctcctgggagataattctgctctctggagatgttggtaggaaagtttcaagttacgcagctgagaaacagggaccaaatagtgctcctgggtgcattgtcaccgtgggtggccactcaagggtccaagcctctagggccatccttgggctaacaactggggtgggtgtgagcaggtggaaggagcctcagcccatgccattacctcctgcttccttatcaggctgtgtgttaattctgggccagtctacaccctcccacggggtggaaatggcctggaggatgtgagggcacccctcctctgaagatccctgtacacgtggtgttgggactggaaccattatgcggccccataggcctcaggagtcatcccagaagcagtggctgggaggtggtgtcctaagtaaggatctgtgcagaggaca

SEQ ID NO: 336 - RNA, 586 nt, synthetic construct
  source 1..586: mol_type = other RNA; note = Registry ID 3UTR259
cagtgcacaatattttcccatctgtattattttttttcagcatgtattacttgacaaagagacactgtgcagagggtgaccacagtctgtaattccccacttcaatacaaagggtgtcgttcttttccaacaaaatagcaatcccttttatttcattgcttttgacttttcaatgggtgtcctaggaaccttttagaaagaaatggactttcatcctggaaatatattaactgttaaaaagaaaacattgaaaatgtgtttagacaacgtcatcccctggcaggctaaagtgctgtatcctttagtaaaattggaggtagcaaacactaaggtgaaaagataatgatctcattgtttattaacctgtattctgtttacatgtctttaaaacagtggttcttaaattgtaagctcaggttcaaagtgttggtaatgcctgattcacaactttgagaaggtagcactggagagaattggaatgggtggcggtaattggtgatacttctttgaatgtagatttccaatcacatctttagtgtctgaatatatccaaatgttttaggatgtatgttacttcttagagaga

SEQ ID NO: 337 - RNA, 90 nt, synthetic construct
  source 1..90: mol_type = other RNA; note = Registry ID 3UTR260
gcttcctcttcactctgctctcaggagatctggctgtgaggccctcagggcagggatacaaagcggggagagggtacacaatgggtatct

SEQ ID NO: 338 - RNA, 160 nt, synthetic construct
  source 1..160: mol_type = other RNA; note = Registry ID 3UTR261
gacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctccc

SEQ ID NO: 339 - RNA, 154 nt, synthetic construct
  source 1..154: mol_type = other RNA; note = Registry ID 3UTR262
aaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaatt

SEQ ID NO: 340 - RNA, 93 nt, synthetic construct
  source 1..93: mol_type = other RNA; note = Registry ID 3UTR263
caggacacagccttggatcaggacagagacttgggggccatcctgcccctccaacccgacatgtgtacctcagctttttccctcacttgcatc

SEQ ID NO: 341 - RNA, 142 nt, synthetic construct
  source 1..142: mol_type = other RNA; note = Registry ID 3UTR264
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatca

SEQ ID NO: 342 - RNA, 92 nt, synthetic construct
  source 1..92: mol_type = other RNA; note = Registry ID 3UTR265
agtgtccagaccattgtcttccaaccccagctggcctctagaacacccactggccagtcctagagctcctgtccctacccactctttgctac

SEQ ID NO: 343 - RNA, 115 nt, synthetic construct
  source 1..115: mol_type = other RNA; note = Registry ID 3UTR266
gcttcctcttcactctgctctcaggagacctggctatgaggccctcggggcagggatacaaagttagtgaggtctatgtccagagaagctgagatatggcatataataggcatct

SEQ ID NO: 344 - RNA, 96 nt, synthetic construct
  source 1..96: mol_type = other RNA; note = Registry ID 3UTR267
ggacctgaagggtgacatcccaggaggggcctctgaaatttcccacaccccagcgcctgtgctgaggactccctccatgtggccccaggtgccacc

SEQ ID NO: 345 - RNA, 125 nt, synthetic construct
  source 1..125: mol_type = other RNA; note = Registry ID 3UTR268
gagccttctgagcccagcgacttctgaagggccccttgcaaagtaatagggcttctgcctaagcctctccctccagccaataggcagctttcttaactatcctaacaagccttggaccaaatgga

SEQ ID NO: 346 - RNA, 219 nt, synthetic construct
  source 1..219: mol_type = other RNA; note = Registry ID 3UTR269
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaa

SEQ ID NO: 347 - RNA, 190 nt, synthetic construct
  source 1..190: mol_type = other RNA; note = Registry ID 3UTR270
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactc

SEQ ID NO: 348 - RNA, 663 nt, synthetic construct
  source 1..663: mol_type = other RNA; note = Registry ID 3UTR271
aaaattaactgctaacttctattgacccacaaagtttcagaaattctctgaaagtttcttccttttttctcttactatatttattgatttcaagtcttctattaaggacatttagccttcaatggaaattaaaactcatttaggactgtatttccaaattactgatatcagagttatttaaaaattgtttatttgaggagataacatttcaactttgttcctaaatatataataataaaatgattgactttatttgcatttttatgaccacttgtcatttattttgtcttcgtaaattattttcattatatcaaatattttagtatgtacttaataaaataggagaacattttagagtttcaaattcccaggtattttccttgtttattacccctaaatcattcctatttaattcttctttttaaatggagaaaattatgtctttttaatatggtttttgttttgttatatattcacaggctggagacgtttaaaagaccgtttcaaaagagatttacttttttaaaggactttatctgaacagagagatataatatttttcctattggacaatggacttgcaaagcttcacttcattttaagagcaaaagaccccatgttgaaaactccataacagttttatgctgatgataatttatctac

SEQ ID NO: 349 - RNA, 127 nt, synthetic construct
  source 1..127: mol_type = other RNA; note = Registry ID 3UTR272
aaaattaactgctaacttctattgacccacaaagtttcagaaattctctgaaagtttcttccttttttctcttactatatttattgatttcaagtcttctattaaggacatttagccttcaatggaa

SEQ ID NO: 350 - RNA, 127 nt, synthetic construct
  source 1..127: mol_type = other RNA; note = Registry ID 3UTR273
aaaattaactgctaacttctattgacccacaaagtttcagaaattctctgaaagtttcttccttttttctcttactatttttattgatttcaagtcttctattaaggacaattagccttcaatggaa

SEQ ID NO: 351 - RNA, 162 nt, synthetic construct
  source 1..162: mol_type = other RNA; note = Registry ID 3UTR274
ggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaa

SEQ ID NO: 352 - RNA, 49 nt, synthetic construct
  source 1..49: mol_type = other RNA; note = Registry ID 3UTR275
ggggccttctgacatgagtctggcctggccccacctcctagttcctcat

SEQ ID NO: 353 - RNA, 164 nt, synthetic construct
  source 1..164: mol_type = other RNA; note = Registry ID 3UTR276
tgtgttcagaaaactaggcaggaaagtaggaaaagatctgttaatcgaatcacgtctgcataacttttattatttcttttattaatcaacaaaattttgtttttaacatttctatagaccagatcccggagttggaaaacaatgaaaaggcccccaaggtagtt

SEQ ID NO: 354 - RNA, 164 nt, synthetic construct
  source 1..164: mol_type = other RNA; note = Registry ID 3UTR277
aatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtttatag

SEQ ID NO: 355 - RNA, 284 nt, synthetic construct
  source 1..284: mol_type = other RNA; note = Registry ID 3UTR278
cgtctgcataacttttattatttcttttattaatcaacaaaattttgtttttaacatttctgtgttcagaaaactaggcaggaaagtaggaaaagatctgttaatcgaatcacgtctgcataacttttattatttcttttattaatcaacaaaattttgtttttaacatttctatagaccagatcccggagttggaaaacaatgaaaaggcccccaaggtagttcgtctgcataacttttattatttcttttattaatcaacaaaattttgtttttaacatttc

SEQ ID NO: 356 - RNA, 284 nt, synthetic construct
  source 1..284: mol_type = other RNA; note = Registry ID 3UTR279
cgtctgcataacttttattatttcttttattaatcaacaaaattttgtttttaacatttcaatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtttatagcgtctgcataacttttattatttcttttattaatcaacaaaattttgtttttaacatttc

SEQ ID NO: 357 - RNA, 174 nt, synthetic construct
  source 1..174: mol_type = other RNA; note = Registry ID 3UTR280
aatcatgtgttcagaaaactaggcaggaaagtaggaaaagatctgttaatcgaatcacgtctgcataacttttattatttcttttattaatcaacaaaattttgtttttaacatttctatagaccagatcccggagttggaaaacaatgaaaaggcccccaaggtagttaatca

SEQ ID NO: 358 - RNA, 258 nt, synthetic construct
  source 1..258: mol_type = other RNA; note = Registry ID 3UTR281
tgtgttcagaaaactaggcaggaaagtaggaaaagatctgttaatcgaatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtttatagtgtgttcagaaaactaggcaggaaagtaggaaaagatctgttaatcg

SEQ ID NO: 359 - RNA, 258 nt, synthetic construct
  source 1..258: mol_type = other RNA; note = Registry ID 3UTR282
accagatcccggagttggaaaacaatgaaaaggcccccaaggtagttaatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtttatagaccagatcccggagttggaaaacaatgaaaaggcccccaaggtagtt

SEQ ID NO: 360 - RNA, 174 nt, synthetic construct
  source 1..174: mol_type = other RNA; note = Registry ID 3UTR283
tatagtgtgttcagaaaactaggcaggaaagtaggaaaagatctgttaatcgaatcacgtctgcataacttttattatttcttttattaatcaacaaaattttgtttttaacatttctatagaccagatcccggagttggaaaacaatgaaaaggcccccaaggtagtttatag

SEQ ID NO: 361 - RNA, 154 nt, synthetic construct
  source 1..154: mol_type = other RNA; note = Registry ID 3UTR284
tgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtt

SEQ ID NO: 362 - RNA, 112 nt, synthetic construct
  source 1..112: mol_type = other RNA; note = Registry ID 3UTR285
tgtgttcagaaaactaggcaggaaagtaggaaaagatctgttaatcgaatcacgtctgcataacttttattatttcttttattaatcaacaaaattttgtttttaacatttc

SEQ ID NO: 363 - RNA, 112 nt, synthetic construct
  source 1..112: mol_type = other RNA; note = Registry ID 3UTR286
aatcatgtgttcagaaaactaggcaggaaagtaggaaaagatctgttaatcgcgtctgcataacttttattatttcttttattaatcaacaaaattttgtttttaacatttc

SEQ ID NO: 364 - RNA, 159 nt, synthetic construct
  source 1..159: mol_type = other RNA; note = Registry ID 3UTR287
tgtgttcagaaaactaggcaggaaagtaggaaaagatctgttaatcgaatcacgtctgcataacttttattatttcttttattaatcaacaaaattttgtttttaacatttcaccagatcccggagttggaaaacaatgaaaaggcccccaaggtagtt

SEQ ID NO: 365 - RNA, 159 nt, synthetic construct
  source 1..159: mol_type = other RNA; note = Registry ID 3UTR288
aatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagtt

SEQ ID NO: 366 - RNA, 154 nt, synthetic construct
  source 1..154: mol_type = other RNA; note = Registry ID 3UTR289
tgtgttcagaaaactaggcaggaaagtaggaaaagatctgttaatcgcgtctggataacttttattatttcttttttttatcaacaaaattttgttttaaacatttcaccagatcccggagttggaaaacaatgaaaaggcccccaaggtagtt

SEQ ID NO: 367 - RNA, 112 nt, synthetic construct
  source 1..112: mol_type = other RNA; note = Registry ID 3UTR290
tgtgttcagaaaactaggcaggaaagtaggaaaagatctgttaatcgaatcacgtctggataacttttattatttcttttttttatcaacaaaattttgttttaaacatttc

SEQ ID NO: 368 - RNA, 112 nt, synthetic construct
  source 1..112: mol_type = other RNA; note = Registry ID 3UTR291
aatcatgtgttcagaaaactaggcaggaaagtaggaaaagatctgttaatcgcgtctggataacttttattatttcttttttttatcaacaaaattttgttttaaacatttc

SEQ ID NO: 369 - RNA, 159 nt, synthetic construct
  source 1..159: mol_type = other RNA; note = Registry ID 3UTR292
tgtgttcagaaaactaggcaggaaagtaggaaaagatctgttaatcgaatcacgtctggataacttttattatttcttttttttatcaacaaaattttgttttaaacatttcaccagatcccggagttggaaaacaatgaaaaggcccccaaggtagtt

SEQ ID NO: 370 - RNA, 159 nt, synthetic construct
  source 1..159: mol_type = other RNA; note = Registry ID 3UTR293
aatcatgtgttcagaaaactaggcaggaaagtaggaaaagatctgttaatcgcgtctggataacttttattatttcttttttttatcaacaaaattttgttttaaacatttcaccagatcccggagttggaaaacaatgaaaaggcccccaaggtagtt

SEQ ID NO: 371 - RNA, 154 nt, synthetic construct
  source 1..154: mol_type = other RNA; note = Registry ID 3UTR294
tgtgttcagaaaactaggcaggaaagtaggaaaagatctgttaatcgcgtctggataacttttattatttctttttttaatcaacaaaattttgtttttaacatttcaccagatcccggagttggaaaacaatgaaaaggcccccaaggtagtt

SEQ ID NO: 372 - RNA, 112 nt, synthetic construct
  source 1..112: mol_type = other RNA; note = Registry ID 3UTR295
tgtgttcagaaaactaggcaggaaagtaggaaaagatctgttaatcgaatcacgtctggataacttttattatttctttttttaatcaacaaaattttgtttttaacatttc

SEQ ID NO: 373 - RNA, 112 nt, synthetic construct
  source 1..112: mol_type = other RNA; note = Registry ID 3UTR296
aatcatgtgttcagaaaactaggcaggaaagtaggaaaagatctgttaatcgcgtctggataacttttattatttctttttttaatcaacaaaattttgtttttaacatttc

SEQ ID NO: 374 - RNA, 159 nt, synthetic construct
  source 1..159: mol_type = other RNA; note = Registry ID 3UTR297
tgtgttcagaaaactaggcaggaaagtaggaaaagatctgttaatcgaatcacgtctggataacttttattatttctttttttaatcaacaaaattttgtttttaacatttcaccagatcccggagttggaaaacaatgaaaaggcccccaaggtagtt

SEQ ID NO: 375 - RNA, 159 nt, synthetic construct
  source 1..159: mol_type = other RNA; note = Registry ID 3UTR298
aatcatgtgttcagaaaactaggcaggaaagtaggaaaagatctgttaatcgcgtctggataacttttattatttctttttttaatcaacaaaattttgtttttaacatttcaccagatcccggagttggaaaacaatgaaaaggcccccaaggtagtt

SEQ ID NO: 376 - RNA, 159 nt, synthetic construct
  source 1..159: mol_type = other RNA; note = Registry ID 3UTR299
tgtgttcagaaaactaggcaggaaagtaggaaaagatctgttaatcgcgtctgcataacttttattatttcttttattaatcaacaaaattttgtttttaacatttctatagaccagatcccggagttggaaaacaatgaaaaggcccccaaggtagtt

SEQ ID NO: 377 - RNA, 117 nt, synthetic construct
  source 1..117: mol_type = other RNA; note = Registry ID 3UTR300
tgtgttcagaaaactaggcaggaaagtaggaaaagatctgttaatcgaatcacgtctgcataacttttattatttcttttattaatcaacaaaattttgtttttaacatttctatag

SEQ ID NO: 378 - RNA, 117 nt, synthetic construct
  source 1..117: mol_type = other RNA; note = Registry ID 3UTR301
aatcatgtgttcagaaaactaggcaggaaagtaggaaaagatctgttaatcgcgtctgcataacttttattatttcttttattaatcaacaaaattttgtttttaacatttctatag

SEQ ID NO: 379 - RNA, 164 nt, synthetic construct
  source 1..164: mol_type = other RNA; note = Registry ID 3UTR303
aatcatgtgttcagaaaactaggcaggaaagtaggaaaagatctgttaatcgcgtctgcataacttttattatttcttttattaatcaacaaaattttgtttttaacatttctatagaccagatcccggagttggaaaacaatgaaaaggcccccaaggtagtt

SEQ ID NO: 380 - RNA, 201 nt, synthetic construct
  source 1..201: mol_type = other RNA; note = Registry ID 3UTR304
tgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 381 - RNA, 206 nt, synthetic construct
  source 1..206: mol_type = other RNA; note = Registry ID 3UTR307
tgtgttcagaaaactaggcaggaaagtaggaaaagatctgttaatcgaatcacgtctgcataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 382 - RNA, 206 nt, synthetic construct
  source 1..206: mol_type = other RNA; note = Registry ID 3UTR308
aatcatgtgttcagaaaactaggcaggaaagtaggaaaagatctgttaatcgcgtctgcataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 383 - RNA, 201 nt, synthetic construct
  source 1..201: mol_type = other RNA; note = Registry ID 3UTR309
tgtgttcagaaaactaggcaggaaagtaggaaaagatctgttaatcgcgtctggataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 384 - RNA, 159 nt, synthetic construct
  source 1..159: mol_type = other RNA; note = Registry ID 3UTR310
tgtgttcagaaaactaggcaggaaagtaggaaaagatctgttaatcgaatcacgtctggataacttttattatttcttttattaatcaacaaaattttgtttttaacatttcaccagatcccggagttggaaaacaatgaaaaggcccccaaggtagtt

SEQ ID NO: 385 - RNA, 159 nt, synthetic construct
  source 1..159: mol_type = other RNA; note = Registry ID 3UTR311
aatcatgtgttcagaaaactaggcaggaaagtaggaaaagatctgttaatcgcgtctggataacttttattatttcttttattaatcaacaaaattttgtttttaacatttcaccagatcccggagttggaaaacaatgaaaaggcccccaaggtagtt

SEQ ID NO: 386 - RNA, 206 nt, synthetic construct
  source 1..206: mol_type = other RNA; note = Registry ID 3UTR312
tgtgttcagaaaactaggcaggaaagtaggaaaagatctgttaatcgaatcacgtctggataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 387 - RNA, 206 nt, synthetic construct
  source 1..206: mol_type = other RNA; note = Registry ID 3UTR313
aatcatgtgttcagaaaactaggcaggaaagtaggaaaagatctgttaatcgcgtctggataacttttattatttcttttattaatcaacaaaattttgtttttaacatttcaccagatcccggagttggaaaacaatgaaaaggcccccaaggtagttaccagatcccggagttggaaaacaatgaaaaggcccccaaggtagtt

SEQ ID NO: 388 - RNA, 264 nt, synthetic construct
  source 1..264: mol_type = other RNA; note = Registry ID 3UTR314
tgtgttcagaaaactaggcaggaaagtaggaaaagatctgttaatcgatgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagaccaaccagatcccggagttggaaaacaatgaaaaggcccccaaggtagtt

SEQ ID NO: 389 - RNA, 222 nt, synthetic construct
  source 1..222: mol_type = other RNA; note = Registry ID 3UTR315
tgtgttcagaaaactaggcaggaaagtaggaaaagatctgttaatcgaatcaatgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagacca

SEQ ID NO: 390 - RNA, 222 nt, synthetic construct
  source 1..222: mol_type = other RNA; note = Registry ID 3UTR316
aatcatgtgttcagaaaactaggcaggaaagtaggaaaagatctgttaatcgatgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagacca

SEQ ID NO: 391 - RNA, 269 nt, synthetic construct
  source 1..269: mol_type = other RNA; note = Registry ID 3UTR317
tgtgttcagaaaactaggcaggaaagtaggaaaagatctgttaatcgaatcaatgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagaccaaccagatcccggagttggaaaacaatgaaaaggcccccaaggtagtt

SEQ ID NO: 392 - RNA, 269 nt, synthetic construct
  source 1..269: mol_type = other RNA; note = Registry ID 3UTR318
aatcatgtgttcagaaaactaggcaggaaagtaggaaaagatctgttaatcgatgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagaccaaccagatcccggagttggaaaacaatgaaaaggcccccaaggtagtt

SEQ ID NO: 393 - RNA, 308 nt, synthetic construct
  source 1..308: mol_type = other RNA; note = Registry ID 3UTR319
tgtgttcagaaaactaggcaggaaagtaggaaaagatctgttaatcgatgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgcttaccagatcccggagttggaaaacaatgaaaaggcccccaaggtagtt

SEQ ID NO: 394 - RNA, 266 nt, synthetic construct
  source 1..266: mol_type = other RNA; note = Registry ID 3UTR320
tgtgttcagaaaactaggcaggaaagtaggaaaagatctgttaatcgaatcaatgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctt

SEQ ID NO: 395 - RNA, 266 nt, synthetic construct
  source 1..266: mol_type = other RNA; note = Registry ID 3UTR321
aatcatgtgttcagaaaactaggcaggaaagtaggaaaagatctgttaatcgatgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctt

SEQ ID NO: 396 - RNA, 313 nt, synthetic construct
  source 1..313: mol_type = other RNA; note = Registry ID 3UTR322
tgtgttcagaaaactaggcaggaaagtaggaaaagatctgttaatcgaatcaatgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgcttaccagatcccggagttggaaaacaatgaaaaggcccccaaggtagtt

SEQ ID NO: 397 - RNA, 313 nt, synthetic construct
  source 1..313: mol_type = other RNA; note = Registry ID 3UTR323
aatcatgtgttcagaaaactaggcaggaaagtaggaaaagatctgttaatcgatgtctccagttacaactccgcagtggatgtgaagaagcggcagcagcggaggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgcttaccagatcccggagttggaaaacaatgaaaaggcccccaaggtagtt

SEQ ID NO: 398 - RNA, 196 nt, synthetic construct
  source 1..196: mol_type = other RNA; note = Registry ID 3UTR324
tgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctcaaccagatcccggagttggaaaacaatgaaaaggcccccaaggtagtt

SEQ ID NO: 399 - RNA, 154 nt, synthetic construct
  source 1..154: mol_type = other RNA; note = Registry ID 3UTR325
tgtgttcagaaaactaggcaggaaagtaggaaaagatctgttaatcgaatcagggatgagaacagagagaaatatattcataatttactttatgacctagaaggaaactgtcgtgtgtcctatacattgccatcaactttgtttcctcatctca

SEQ ID NO: 400 - RNA, 154 nt, synthetic construct
  source 1..154: mol_type = other RNA; note = Registry ID 3UTR326
aatcatgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctca

SEQ ID NO: 401 - RNA, 201 nt, synthetic construct
  source 1..201: mol_type = other RNA; note = Registry ID 3UTR327
tgtgttcagaaaactaggcaggaaagtaggaaaagatctgttaatcgaatcagggatgagaacagagagaaatatattcataatttactttatgacctagaaggaaactgtcgtgtgtcctatacattgccatcaactttgtttcctcatctcaaccagatcccggagttggaaaacaatgaaaaggcccccaaggtagtt

SEQ ID NO: 402 - RNA, 201 nt, synthetic construct
  source 1..201: mol_type = other RNA; note = Registry ID 3UTR328
aatcatgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctcaaccagatcccggagttggaaaacaatgaaaaggcccccaaggtagtt

SEQ ID NO: 403 - RNA, 256 nt, synthetic construct
  source 1..256: mol_type = other RNA; note = Registry ID 3UTR329
tgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttcaccagatcccggagttggaaaacaatgaaaaggcccccaaggtagtt

SEQ ID NO: 404 - RNA, 214 nt, synthetic construct
  source 1..214: mol_type = other RNA; note = Registry ID 3UTR330
tgtgttcagaaaactaggcaggaaagtaggaaaagatctgttaatcgaatcagggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttc

SEQ ID NO: 405 - RNA, 214 nt, synthetic construct
  source 1..214: mol_type = other RNA; note = Registry ID 3UTR331
aatcatgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttc

SEQ ID NO: 406 - RNA, 261 nt, synthetic construct
  source 1..261: mol_type = other RNA; note = Registry ID 3UTR332
tgtgttcagaaaactaggcaggaaagtaggaaaagatctgttaatcgaatcagggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttcaccagatcccggagttggaaaacaatgaaaaggcccccaaggtagtt

SEQ ID NO: 407 - RNA, 261 nt, synthetic construct
  source 1..261: mol_type = other RNA; note = Registry ID 3UTR333
aatcatgtgttcagaaaactaggcaggaaagtaggaaaagatctgttaatcggggatgagaacagagagaaatatattcataatttactttatgacctagaaggaaactgtcgtgtgtcctatacattgccatcaactttgtttcctcatctcacgtctgcataacttttattatttcttttattaatcaacaaaattttgtttttaacatttcaccagatcccggagttggaaaacaatgaaaaggcccccaaggtagtt

SEQ ID NO: 408 - RNA, 187 nt, synthetic construct
  source 1..187: mol_type = other RNA; note = Registry ID 3UTR334
tgtgttcagaaaactaggcaggaaagtaggaaaagatctgttaatcggacagagctctgcggtgtcagggcgagaacccatcttccaaccccggctatttggagacggaaaaactggaattctaacaaggaggagaggagaccagatcccggagttggaaaacaatgaaaaggcccccaaggtagtt

SEQ ID NO: 409 - RNA, 145 nt, synthetic construct
  source 1..145: mol_type = other RNA; note = Registry ID 3UTR335
tgtgttcagaaaactaggcaggaaagtaggaaaagatctgttaatcgaatcagacagagctctgcggtgtcagggcgagaacccatcttccaaccccggctatttggagacggaaaaactggaattctaacaaggaggagaggag

SEQ ID NO: 410 - RNA, 145 nt, synthetic construct
  source 1..145: mol_type = other RNA; note = Registry ID 3UTR336
aatcatgtgttcagaaaactaggcaggaaagtaggaaaagatctgttaatcggacagagctctgcggtgtcagggcgagaacccatcttccaaccccggctatttggagacggaaaaactggaattctaacaaggaggagaggag

SEQ ID NO: 411 - RNA, 192 nt, synthetic construct
  source 1..192: mol_type = other RNA; note = Registry ID 3UTR337
tgtgttcagaaaactaggcaggaaagtaggaaaagatctgttaatcgaatcagacagagctctgcggtgtcagggcgagaacccatcttccaaccccggctatttggagacggaaaaactggaattctaacaaggaggagaggagaccagatcccggagttggaaaacaatgaaaaggcccccaaggtagtt

SEQ ID NO: 412 - RNA, 192 nt, synthetic construct
  source 1..192: mol_type = other RNA; note = Registry ID 3UTR338
aatcatgtgttcagaaaactaggcaggaaagtaggaaaagatctgttaatcggacagagctctgcggtgtcagggcgagaacccatcttccaaccccggctatttggagacggaaaaactggaattctaacaaggaggagaggagaccagatcccggagttggaaaacaatgaaaaggcccccaaggtagtt

SEQ ID NO: 413 - RNA, 247 nt, synthetic construct
  source 1..247: mol_type = other RNA; note = Registry ID 3UTR339
tgtgttcagaaaactaggcaggaaagtaggaaaagatctgttaatcggacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttcaccagatcccggagttggaaaacaatgaaaaggcccccaaggtagtt

SEQ ID NO: 414 - RNA, 205 nt, synthetic construct
  source 1..205: mol_type = other RNA; note = Registry ID 3UTR340
tgtgttcagaaaactaggcaggaaagtaggaaaagatctgttaatcgaatcagacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttc

SEQ ID NO: 415 - RNA, 205 nt, synthetic construct
  source 1..205: mol_type = other RNA; note = Registry ID 3UTR341
aatcatgtgttcagaaaactaggcaggaaagtaggaaaagatctgttaatcggacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttc

SEQ ID NO: 416 - RNA, 252 nt, synthetic construct
  source 1..252: mol_type = other RNA; note = Registry ID 3UTR342
tgtgttcagaaaactaggcaggaaagtaggaaaagatctgttaatcgaatcagacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttcaccagatcccggagttggaaaacaatgaaaaggcccccaaggtagtt

SEQ ID NO: 417 - RNA, 252 nt, synthetic construct
  source 1..252: mol_type = other RNA; note = Registry ID 3UTR343
aatcatgtgttcagaaaactaggcaggaaagtaggaaaagatctgttaatcggacagagctctgcggtgtcagggcgagaacccatcttccaaccccggctatttggagacggaaaaactggaattctaacaaggaggagaggagcgtctgcataacttttattatttcttttattaatcaacaaaattttgtttttaacatttcaccagatcccggagttggaaaacaatgaaaaggcccccaaggtagtt

SEQ ID NO: 418 - RNA, 351 nt, synthetic construct
  source 1..351: mol_type = other RNA; note = Registry ID 3UTR344
tgtgttcagaaaactaggcaggaaagtaggaaaagatctgttaatcgatgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggaccagatcccggagttggaaaacaatgaaaaggcccccaaggtagtt

SEQ ID NO: 419 - RNA, 309 nt, synthetic construct
  source 1..309: mol_type = other RNA; note = Registry ID 3UTR345
tgtgttcagaaaactaggcaggaaagtaggaaaagatctgttaatcgaatcaatgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgagg

SEQ ID NO: 420 - RNA, 309 nt, synthetic construct
  source 1..309: mol_type = other RNA; note = Registry ID 3UTR346
aatcatgtgttcagaaaactaggcaggaaagtaggaaaagatctgttaatcgatgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgagg

SEQ ID NO: 421 - RNA, 356 nt, synthetic construct
  source 1..356: mol_type = other RNA; note = Registry ID 3UTR347
tgtgttcagaaaactaggcaggaaagtaggaaaagatctgttaatcgaatcaatgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggaccagatcccggagttggaaaacaatgaaaaggcccccaaggtagtt

SEQ ID NO: 422 - RNA, 356 nt, synthetic construct
  source 1..356: mol_type = other RNA; note = Registry ID 3UTR348
aatcatgtgttcagaaaactaggcaggaaagtaggaaaagatctgttaatcgatgtctccagttacaactccgcagtggatgtgaagaagcggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggaccagatcccggagttggaaaacaatgaaaaggcccccaaggtagtt

SEQ ID NO: 423 - RNA, 87 nt, synthetic construct
  source 1..87: mol_type = other RNA; note = Registry ID 3UTR350
acctgagactggtggcttctagaagcagccattaccaactgtaccttcccttcttgctcagccaataaatatatcctctttcactca

SEQ ID NO: 424 - RNA, 1310 nt, synthetic construct
  source 1..1310: mol_type = other RNA; note = Registry ID 3UTR351
ccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggtaatgaattaaagaagagaactgcctttggcagagttctataatgtaaacaatatcagactttttttttttataatcaagcctaaaattgtatagacctaaaataaaatgaagtggtgagcttaaccctggaaaatgaatccctctatctctaaagaaaatctctgtgaaacccctatgtggaggcggaattgctctcccagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctctgctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtacattcttcctgattattttctacacatacatgtaaaatatgtctttcttttttaaatagggttgtactatgctgttatgagtggctttaatgaataaacatttgtagcatcctctttaatgggtaaacagca

SEQ ID NO: 425 - RNA, 4140 nt, synthetic construct
  source 1..4140: mol_type = other RNA; note = Registry ID 3UTR352
gcagagaatacggttttggtgtcctgctacaaaaagacatcggtcagtaacgagcacgatgtggaaaaatgagagaagggacacattcaaccctggagagttcaatggctgctgaagctgcctgcttttcactgctgcaaggcctttctgtgtgtgatgtgcatgggagcaacttgttcgtgggtcatcgggaatactagggagaaggtttcattgcccccagggcacttcacagagtgtgctggaggactgagtaagaaatgctgcccatgccaccgcttccggctcctgtgctttccctgaactgggacctttagtggtggccatttagccaccatctttgcaggttgctttgccctggtagggcagtaacattgggtcctgggtctttcatggggtgatgctgggctggctccctcttggtcttcccaggctggggctgaccttcctcgcagagaggccaggtgcaggttgggaatgaggcttgctgagaggggctgtccagttcccagaaggcatatcagtctctgagggcttcctttggggccgggaacttgcgggtttgaggataggagttcacttcatcttctcagctcccatttctactcttaagtttctcagctcccatttctactctcccatggcttaatgcttctttcattttctgtttgttttatacaaatgtcttagttgtacaaataaagtcccaggttaaagataacaaacggctcctgtgacataaacgtgcgaaagcccatctacagcaaagacatcagtgctccgtggaacagaaaccagaaggagaaaatttgtacatcctccttttgcacctgagatcctcatttgcccgacgttgtaggtgtggagagtcctagagaggctaggaagcgcccagggcacccagagccaggctgcaggtgcctcaggcccctctcccagcccactcccaagctgaactagcacgtgttcatttctaccgcgggttgaaccgcagggatccctggcttcaagtcaggcaccaaacagaaggtaggaggcatgaggggttctgtcacatgtctcttcattttcttattgattcttagccttgacagttggagaaggaaaaagcaaggagaaagtgctgagcaggagtggagagtgaagagcaggacctcgtgcctgggtttgaaaaagtgcctgagacccatcttagccgcccctacctgagctttagcagcctaggagctattgcaccataaaacactgcaacctgaggctgcattaacagaagcaccacgtccaggtccagggagaatccagcctcacctgcttttcctggccagctcaaagaaggacattgacaaatcaggttatgttcagttacctgtgacaaccacagaatgtaagaaatgttacaaaaaaaacaggaaaggaaagacgactctggggaaggggcagtgtgaccttctcctggcatctggggtgcggccgcagcagaatgaggtgtggtgggtggagttagaaaggcatagatgcagtgttaccggaaggagggccttgagtgtaagttgtccaggtccttggcattttgaacaaagaactgaacaaaacatacaaagtaataaaggaataaaagcagcaaaaggaagaatttattgaagtgagaaagcactccacatggtaggagtggaccctaagcagatagcccatgggcccaattgcaaagttttctgggttttaagtaccccttttgaggtacctgttggctaccccttatctgggtgaaggatttggtccatggctaatgaaaggctgaggtgaattgacgccctatgcggatgaagggatggcccgtgcttggtctgtggtcaatccagggccgtctccctttccatctgagatgcagtggaagggggagggttgtagggagagtagcctttgatccttggttactcagtgtggggagatggggtttttccttttggtgtatgttaattggccttagatgccctgcccccagacccaggtgttttccgtttgctccagctttgagaagtcagcacaaattggcctcagattccctgcccccagacgtaggtgtttctccttcattcagcacgaattggccttagatgccctgcccccagacccaggtgttttccatttgatccagctttgagaagtcagcacaaattggcctcagattccctgcccccggacatagatgtttctccttgattcagcacgaattggccttagattccctgcccccagaccctagactcctgcctcagcaagaggtgttcaaaagaaacaaaagctctgcccatcagctctgaggacaagggacaggctgccttccaaggctgaggggaggggtgaaagaattggggtcagctggaccagactcctctagggttcgtgtttcaggtgggcccctcacccggcccatcacaaacaaataagagattaaggcctgcagtgcacatggtctgccctccttgaaggctcacacccagtgcacagcggaagtaagaacaggccacaaggcctcttgtcccactgcaggctttgaaatttgaccgtctccctgctgctgtctttgcaattgcatgtacttgttcacaagttctcatggggttgggctgggggcaggggtggtttgcgctcctctgtcagttttctgtgcagcaggcacctgccgaggcgggctcagctccggctgcccagggagctggagcaggctgggctgccaatggtgggggctgcggtgagaaggtctgcacccagcacctgacctttgtttgaagaaggagctgagctgctactacactctatagggccattatgatgaaatatgccccccaaactccatccagctatcttttgactagcaattccacttgttggcatttatctgaaagaaatacatcaaaaagtggcatgcacaaagaaatacgcatcagattgttccttctagtgttgcttactagctaaaaatgggaaataatccaaatgtccatcaaaagggactggtcagataaattcagacacagtctaataagggggtttaggcggctgctaaggaatgagctagatccatatatgctgatatcaaatgatggttatgacaaatgcatatatttttaaaacatctgtatagattattcttaatacaatatatggcacaatatgtagtcactgttaaaattcaagcacaaaagaaaacaagcaaaacaaatatatgttgttagaagttaggatacagttacccttggtccagtggtggtcattaggagggagcatgaggtttgaaggtgagcatttctgcttctcaatctgggaatcacttatatgagtgtgtttcattagtaaaagtcatcaagctgtacactcagatatgttgactttagagctatggatatagatgtaaattatacttcaagaaagagactttttaaaatccaagtacagaacattgtagaaatgtgtaaaattggtatttggaagtttcttcagcaagcacatgggattacagctcacgcctgtaatcccagtactttgggaggcccacacagaatgatcacttgagctcaggagtttgagagcagcctaggcaacatagcaagacctcgactctacaaaaaatttttaaatttttttctgggcatggtggtgcttgcctgtagtcccagctattagggaggatgaggtgggaggatctcctgaccccaggagtttcagactgcagtgagctgtgatcacgccactgcactccagcctgggcaacacagacagaccttgtctcaaaaataaattttttctaaaaagtttcttgtttttttttcttcccatattttttaaaacataaaattgaaatcttgagaatataatacataaaatagagaatatagaaaattacatcttctaatattgttctgcagatttgatcttttttctctaataataaatcaaggcctccgggtatatttgtttgtatatgtacaggaaaaggtctgtaaggatttactttcagctgttaattgcagttactctgcggaatttccacttctgctttgtacagttccttgtgtgaatttttacatcatgtattacttttgtagttggggaaaaagcaattaaaagtttggaaaaaaa

SEQ ID NO: 426 - RNA, 3117 nt, synthetic construct
  source 1..3117: mol_type = other RNA; note = Registry ID 3UTR353
agtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaagaaagaaaggaaaataaaaaatgatagttgccattattaggatttaatatatatccagtgctttgcaagtgctctgcgcaccttgtctcactccatcctgacaataatcctgggaggtgtgtgcaattactacgactactctcttttttatagatcattaaattcagaactaaggagttaagtaacttgtccaagttgttcacacagtgaagggaggggccaagatatgatggctgggagtctaattgcagttccctgagccatgtgcctttctcttcactgaggactgccccattcttgagtgccaaacgtcactagtaacagggtgtgcctagataatttatgatccaaactgagtcagtttggaaagtgaaagggaaacttacatataatccctccgggacaatgagcaaaaactaggactgtccccagacaaatgtgaacatacatatcatcacttaaattaaaatggctatgagaaagaaagagggggagaaacagtcttgcgggtgtgaagtcccatgaccagccatgtcaaaagaaggtaaagaagtcaagaaaaagccatgaagcccatttggtttcatttttctgaaaataggctcaagagggaataaattagaaactcacaatttctcttgtttgttaccaagacagtgattctcttgctgctaccacccaactgcatccgtccatgatctcagaggaaactgtcgctgaccctggacatgggtacgtttgacgagtgagaggaggcatgacccctcccatgtgtatagacactaccccaacctaaattcatccctaaattgtcccaagttctccagcaatagaggctgccacaaacttcagggagaaagagttacaagtacatgcaatgagtgaactgactgtggctacaatcttgaagatatacggaagagacgtattattaatgcttgacatatatcatcttgcctttcttggtctagactgacttctaatgactaactcaaagtcaaggcaactgagtaatgtcagctcagcaaagtgcagcaaacccatctcccacaggcctccaaaccctggctgttcacagaaccacaaagggcagatgctgcacagaaaactagagaaggggtcataggttcatggttttgtttgagatttgttgctactgtttttctgttttgaattttcttctttgttctgtttttactttatttagggggactaggtgtttctgatattttagttttcttgtttgttttgttttgtgttgtctgtgaatggggttttaactgtggatgaatggaccttatctgttggcttaaaggactggtaagatcagaccatcttattcttcaggtgaatgttttactttccaaagtgctctcctctgcaccagcagtaataaatacaatgccataatcccttaggtttgcctagtgcttttgcaattttcaaagcacttccataagcattccttccacctccttgataggcatttatggaaagcctgctacatgtcaatcatactgttaggcacaggggacctaaagacacataaaaggatggcattctgcctcataaattgcaaaacctaatgaaagtgactgcttggtaaacaaattattattatattataaaatgctataaaagagccatattgaaagtgccctgttggagacagggcaaatgccacaaaaatgatgtaaatttacatggaggaaaagtagaatctgcctggtttgtaggcagcagaagacatttttcatcagtgggcaggtgttctttaccttttgtagaaatgggagtcaagtctcaaataggaggctccacaaaatctcatgccaggtctctgataccttattcacagaagttctttgaagtatttattgttattttctttgacttatgggaaaactgggacacaggaagacaggtaaattacccaacctcacacgttaagtcagaactgggagccataattttgtatccctggtataaatagacaatctcttgaagaaatgaagagatgaccatagaaaaacatcgagatatctccagctctaaaatcctttgtttcaatgttgtttggcatatgttatctttggaatttagtgtctgagcctctgtctgttactgtagtatttaaaatgcatgtattataatcatataatcataactgctgttaattcttgattatatacctagggacaatgtgtaatgtaagattactaattggttctgcccaatctcctttcagattttattaggaaaaaaaaataaacctcctgatcggagacaatgtattaatcagaagtgtaaactgccagttctatatagcatgaaatgaaaagacagctaatttggtccaacaaacatgactgggtctagggcacccaggctgattcagctgatttcctaccagcctttgcctcttccttcaatgtggtttccatgggaatttgcttcagaaaagccaagtatgggctgttcagaggtgcacacctgcattttcttagctcttctagaggggctaagagacttggtacgggccaggaagaatatgtggcagagctcctggaaatgatgcagattaggtggcatttttgtcagctctgtggtttattgttgggactattctttaaaatatccattgttcactacagtgaagatctctgatttaaccgtgtactatccacatgcattacaaacatttcgcagagctgcttagtatataagcgtacaatgtatgtaataaccatctcatatttaattaaatggtatagaagaaca

SEQ ID NO: 427 - RNA, 1747 nt, synthetic construct
  source 1..1747: mol_type = other RNA; note = Registry ID 3UTR354
ggtgaggggccttgaagctgggagtggggtttagggacgcgggtctctgcgtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgaacttctcccctatgaggtaggaagaacagttgtttagaaacgaagaaactgaggccacacagctaatgagtgaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcggtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacaggagaccccgcctctacaaaaagtgaaaaaattagccagtgtgtgctgcacacctgtagtcccagctacttaagaggctgagatgggaggatcgcttgagccctggaatgttgaggctacaatgagctgtgattgtgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcccacccccaaggaggtgaaagctgctaccacctctgtgcccccccgcaatgccaccaactggatgggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgagggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaagagcagagactgtgcctctgtttgactgggtttggtaggagtcattttctgcttgctggtgatcactagctgggcagagaaaaaccaaggcatttgtctatgatgctgtccaggaagcctcattcaacaagctgcctaagtcaacctcttcttggaataacctctaaaagcttccgcttagcaggctatgctgagggccaggaaaacccacctaccagcttggacccctcctctcccactctcatgccacgccacgggacc

SEQ ID NO: 428 - RNA, 171 nt, synthetic construct
  source 1..171: mol_type = other RNA; note = Registry ID 3UTR355
atgtctccagttacaactccgcagtggatgtgaagaagcgacagagctctgcggtgtcagggcgagaacccatcttccaaccccggctatttggagacggaaaaactggaattctaacaaggaggagaggagatgtctccagttacaactccgcagtggatgtgaagaagc

SEQ ID NO: 429 - RNA, 209 nt, synthetic construct
  source 1..209: mol_type = other RNA; note = Registry ID 3UTR356
atgtctccagttacaactccgcagtggatgtgaagaagcggtgcctttgagagtctacttttgctctcttcggaagaacccttaggggttcgtgcatgggcttgcatagcaagtctagatgcgggtaccgtacagtgttgaaaaacactgtaaatctctaaaagagaccaatgtctccagttacaactccgcagtggatgtgaagaagc

SEQ ID NO: 430 - RNA, 63 nt, synthetic construct
  source 1..63: mol_type = other RNA; note = Registry ID 3UTR357
acctgagactggtggcttctagaagcagccattaccaactgtaccttcccttcttgctcagcc

SEQ ID NO: 431 - RNA, 169 nt, synthetic construct
  source 1..169: mol_type = other RNA; note = Registry ID 3UTR358
ggtgccagattaattgctgtttattaatggaaattaatgtttcgcggttttgctaggtttttttaacattccagccagtgtaaggacggaattgtgatagggtaaaaatgtagtcctgctaaagagagtcctgctgtccgaatgctgtagctggtgaaagagacctgcg

SEQ ID NO: 432 - RNA, 167 nt, synthetic construct
  source 1..167: mol_type = other RNA; note = Registry ID 3UTR359
ggtgccagcataacaaaaagttttgctattaataaaaatgctttttgtttttcgtgcgtcctgctttttaacattccagaaagatcccagttttaacattaaaaatgcttgtagtcctgctaaagagagtccaaaaaaaaaagtgtagctggtgaaagagacctgcg

SEQ ID NO: 433 - RNA, 173 nt, synthetic construct
  source 1..173: mol_type = other RNA; note = Registry ID 3UTR360
ggtggttttctgcgtctacttttgctgctagaaagatcattaatccagttttgttttgtccaaaaaattaatccagattaatgtcctgctttttgtttttgctaaaaatgcttgtagtcctgctaaagagagtcctgctgtccgaatgctgtagctggtgaaagagacctgcg

SEQ ID NO: 434 - RNA, 178 nt, synthetic construct
  source 1..178: mol_type = other RNA; note = Registry ID 3UTR361
ggtgtgctagaaaaacgcgaggaattgcacaggaaaaaatgctttttgtttttgctttttaacattattaatccagggaagaacaggaattgcacttttaacattaaaaaatgtctccagttacaactccgcagtggatgtgaagaagcgctggtgccagaggacctgcgtgtaagga

SEQ ID NO: 435 - RNA, 169 nt, synthetic construct
  source 1..169: mol_type = other RNA; note = Registry ID 3UTR362
ggtgattaattgcttctacttttgctggatgctggaagaactgctcgcgtttaactgcttgtaccagattaatggaagaactgctgttttgctttttaacattaaaaaggaagtcctgctaaagagagtcctgctgtccgaatgctgtagctggtgaaagagacctgcg

SEQ ID NO: 436 - RNA, 166 nt, synthetic construct
  source 1..166: mol_type = other RNA; note = Registry ID 3UTR363
ggtgccaggtcctctacttttgcaaagatcaagatcaaaaatgctcgcgtgcttctacttttgcgtccaatcatgcttgtaggaagtttttttaacattaaaaattttgtttttgctaaagagagtcctgctgtccgaatgctgtagctggtgaaagagacctgcg

SEQ ID NO: 437 - RNA, 180 nt, synthetic construct
  source 1..180: mol_type = other RNA; note = Registry ID 3UTR364
atgtctccagttacaactccgcagtggatgtgaagaagcaagatcttttaacattgtccagtcctgctggaagaactgcttgtaggaattttgttttggaaaaaaatgctggaagaacttttgtttttgctaaagagagtcctgctgtccgaatgctgtagctggtgaaagagacctgcg

SEQ ID NO: 438 - RNA, 183 nt, synthetic construct
  source 1..183: mol_type = other RNA; note = Registry ID 3UTR365
atgtctccagttacaactccgcagaaagagaattaataaagagattttaacatttctacttttgcgtccccagggaagaactgtagtccaaagatctgctagcataactcgtgcggaaaaaaattttgtttttgctaaaaagtcctgctgtccgaatgctgtagctggtgaaagagacctgcg

SEQ ID NO: 439 - RNA, 12 nt, synthetic construct
  source 1..12: mol_type = other RNA; note = repeating GC-motif
ccgccgccgccg

SEQ ID NO: 440 - RNA, 15 nt, synthetic construct
  source 1..15: mol_type = other RNA; note = repeating GC-motif
ccgccgccgccgccg

SEQ ID NO: 441 - RNA, 18 nt, synthetic construct
  source 1..18: mol_type = other RNA; note = repeating GC-motif
ccgccgccgccgccgccg

SEQ ID NO: 442 - RNA, 21 nt, synthetic construct
  source 1..21: mol_type = other RNA; note = repeating GC-motif
ccgccgccgccgccgccgccg

SEQ ID NO: 443 - RNA, 24 nt, synthetic construct
  source 1..24: mol_type = other RNA; note = repeating GC-motif
ccgccgccgccgccgccgccgccg

SEQ ID NO: 444 - RNA, 27 nt, synthetic construct
  source 1..27: mol_type = other RNA; note = repeating GC-motif
ccgccgccgccgccgccgccgccgccg

SEQ ID NO: 445 - RNA, 30 nt, synthetic construct
  source 1..30: mol_type = other RNA; note = repeating GC-motif
ccgccgccgccgccgccgccgccgccgccg